SEQUENCE LISTING (5 sequences)

SEQ ID NO: 1 - DNA, 56 bp, Artificial Sequence
  Synthetic construct
acgcaattcgactagatcgggtcgatgatcgatcgatgatcgagacagcatagcag

SEQ ID NO: 2 - DNA, 33 bp, Artificial Sequence
  Synthetic construct
caattcgactagatcgatcgacgatcgatcgat

SEQ ID NO: 3 - DNA, 56 bp, Artificial Sequence
  Synthetic construct
nnncaattcgactagatcngatcnnngancgatcnnngatnnnnncngnatnnnnn

SEQ ID NO: 4 - DNA, 34 bp, Artificial Sequence
  Synthetic construct
caattcgactagatcgggtcgatgatcgatcgat

SEQ ID NO: 5 - DNA, 34 bp, Artificial Sequence
  Synthetic construct
caattcgactagatcngatcgacgatcgatcgat

(19) United States
(12) Patent Application Publication
(10) Pub. No.: US 2013/0172206 A1
(43) Pub. Date: Jul. 4, 2013

(54) GENOME-WIDE DETECTION OF GENOMIC REARRANGEMENTS AND USE OF GENOMIC REARRANGEMENTS TO DIAGNOSE GENETIC DISEASE

(71) Applicants: Mohammed Uddin, St. John's (CA); Proton Rahman, St. John's (CA); Kathy Hodgkinson, St. John's (CA); Darren O'Reilly, St. John's (CA); Sandra Luscombe, St. John's (CA)

(72) Inventors: Mohammed Uddin, St. John's (CA); Proton Rahman, St. John's (CA); Kathy Hodgkinson, St. John's (CA); Darren O'Reilly, St. John's (CA); Sandra Luscombe, St. John's (CA)

(21) Appl. No.: 13/715,126

(22) Filed: Dec. 14, 2012

Related U.S. Application Data
(60) Provisional application No. 61/579,214, filed on Dec. 22, 2011.

(51) Int. Cl. C12Q 1/68

(52) U.S. Cl. CPC C12Q 1/6876
USPC 506/9; 506/16; 506/23; 536/24.33

(57) ABSTRACT

The disclosure relates to the genome-wide identification of “rearrangement hotspots”. The disclosure also relates to a microarray chip system for use in detecting genomic rearrangements and a method of manufacturing a microarray chip system useful for detecting genomic rearrangements. The disclosure also relates to methods for detecting genomic rearrangements associated with genetic diseases. The disclosure further relates to methods for using copy number variants in chromosome 2 for detecting Tourette Syndrome.

### CROSS-REFERENCE TO RELATED APPLICATIONS

This application claims benefit under 35 U.S.C. 119(e) to U.S. provisional application No. 61/579,214, filed Dec. 22, 2011, incorporated herein by reference in its entirety.

### INCORPORATION OF SEQUENCE LISTING

A computer readable form of the Sequence Listing “12362-P40825US01_SequenceListing.txt” (8,192 bytes), submitted via EFS-WEB and created on Dec. 14, 2012, is herein incorporated by reference.

### FIELD

“Rearrangement hotspots”, genomic regions with an elevated frequency of genomic rearrangements such as deletions and duplications, are identified genome-wide. The application discloses microarray chips for detecting genomic rearrangements associated with genetic diseases and methods of manufacturing the microarray chips. The application also discloses methods of using copy number variants to diagnose Tourette Syndrome.

### BACKGROUND

Segmental duplications (SDs) or low-copy repeats are blocks of DNA greater than 1 kilobase pair in size which share a high level of sequence homology (>90%) [Bailey J A, (2006); Redon R. et al. (2006); Bailey J A et al. (2002); and Alkan C. (2011)]. The catalogue of SDs comprises approximately 5% of the human genome encompassing 18% of genes [Bailey J A, (2006); Redon R. et al. (2006); Bailey J A et al. (2002); and Alkan C. (2011)]. They are considered antecedents to the formation of copy number variants (CNVs) which comprise approximately 12% of the human genome and are responsible for considerable human genetic variation [Redon R. et al. (2006); Bailey J. A. et al. (2002); Alkan C. et al. (2011); and Sharp A J et al. (2005)]. Emerging evidence suggests that SD regions are frequently associated with known genomic disorders with the vast majority representing novel sites whose genomic architecture is susceptible to disease-causing rearrangements [Sharp A J et al. (2005)]. However, the complexity of their structural architecture in the human genome and, more importantly, their role in disease pathogenesis remains largely elusive.

There is a growing body of evidence suggesting the involvement of multiple events in the origin of genomic rearrangements such as non-allelic homologous recombination (NAHR), non-homologous end joining (NHEJ), fork stalling and template switching (FoSTeS), and microhomology-mediated break-induced replication (MMBIR) [Cu W et al. (2008); Lieber M R et al. (2003); and Zhang F et al. (2009)]. Although the origins of the aforementioned mechanisms are strongly associated with highly homologous regions residing outside of common repeat elements (e.g., transposons) [Conrad F D et al. (2010)], the non-random distribution of highly homologous regions within SDs that are susceptible to such mechanisms remain to be fully elucidated. Moreover, evolutionary conservation of these mechanisms complicates the identification of SD breakpoints due to differing levels of sequence homology.

Genomic disorders arising from microdeletions/duplications can fail to be adequately explained by a single underlying event. The true contribution of NAHR, NEHJ, MMBIR and FoSTeS events to the origin of genomic rearrangement remains elusive, although large-scale studies are beginning to implicate NAHR as one of the primary events contributing to the origin of these genomic copy number changes [Conrad F D et al. (2010) and Mills R E et al. (2011)]. Genomic DNA situated between distal and proximal SDs represents a critical region often reported to be deleted/duplicated due to misalignment of the SDs between homologous chromosomes [Shaikh H T et al. (2007)].

Evidence suggests that the breakpoint architecture of SDs (i.e., distal and proximal) is associated with a higher propensity for NAHR-mediated rearrangement predisposing to an abnormal phenotype [DECIPHER Genomic Aberration Database: http://decipher.sanger.ac.uk/]. In other words, the increased frequency of pathogenic rearrangements is often directly correlated with the structural complexity of the local genomic regions involved. This is consistent with numerous reports indicating that highly homologous regions within SDs influence NAHR-mediated rearrangement events [Conrad F D et al. (2010); Mills R E et al, (2011); and Turner D J et al. (2007)]. These highly homologous regions may be referred to as ‘rearrangement hotspots’. Classic examples of NAHR-mediated genomic rearrangement include genomic disorders such as 3q29 microdeletion/duplication syndrome, globozoospermia, and Williams-Beuren syndrome [Ballif B C et al. (2008); Koscinski I et al. (2011); and Bayes M et al. (2003)].

In a recent report, the detection and validation of 8,599 CNVs using microarrays [Conrad, F D et al. (2010)] and subsequent targeted sequencing on 1067 of these CNV breakpoints [Conrad F D et al. (2010)] revealed extreme homologous regions consistent with NAHR-mediated rearrangements as the primary event in the origin of CNVs.

Array comparative genome hybridization (aCGH) technology, developed in the last decade, affords the capacity to examine the whole human genome on a single chip with a level of resolution dependent only on size and distance between the interrogating probes, a process also known as microarray-based cytogenetics [Diskin S J et al. (2009)]. The resolution afforded by microarray technology is at least 10-fold greater than the best prometaphase chromosome analysis obtained via conventional karyotyping, rendering it a sensitive whole-genome screen for genomic deletions and duplications [Brunetti-Pierri N et al. (2008)].

Genome-wide signatures of ‘rearrangement hotspots’ can facilitate the detection of genomic regions capable of mediating de novo deletions or duplications in humans. In particular, there remains a need for array comparative genome hybridization technology that targets all the vulnerable regions in the genome susceptible to disease-associated rearrangements including rearrangement hotspots, SDs, recently discovered CNVs and telomeric and centromeric chromosomal regions.

Structural variants are a risk factor for neuropsychiatric diseases. For example, Tourette syndrome (TS) is a developmental neuropsychiatric disorder characterized by the presence of both motor and verbal tics. It has a major genetic component but numerous linkage and association studies have not identified any common candidate genes. Copy number variation (CNV) analysis in TS revealed an association with genes previously implicated in autism spectrum disorder although none unique to TS. That no single CNV has been reported to segregate uniquely with TS in affected families provides an opportunity to detect novel CNVs specific to TS through the use of the microarray technology described herein.

### SUMMARY OF THE DISCLOSURE

The application relates to a microarray chip system comprising at least: 500, 750, 1000, 1500, 2000 or 4000 distinct oligonucleotide probes bound to a solid support, wherein each oligonucleotide probe comprises a nucleotide sequence complementary to a rearrangement indicator sequence region of a human genome, the rearrangement indicator sequence regions comprising a rearrangement indicator set that is indicative of risk, or occurrence, of genomic rearrangements.

In another embodiment, the application relates to a microarray chip system comprising at least: 500, 750, 1000, 1500, 2000 or 4000 distinct oligonucleotide probes bound to a solid support, wherein each oligonucleotide probe comprises a nucleotide sequence that hybridizes, optionally under medium or high stringency conditions, to a nucleotide sequence complementary to a rearrangement indicator sequence region of a human genome, the rearrangement indicator sequence regions comprising a rearrangement indicator set that is indicative of risk, or occurrence, of genomic rearrangements.

In one embodiment, the oligonucleotide probes are complementary to genomic sequences not more than 100, 150, 280, 300 or 500 base pairs apart within a single rearrangement indicator sequence region. Optionally, the oligonucleotide probes are complementary to every 100, 150, 280, 300 or 500 bp of a rearrangement indicator sequence region. In another embodiment, the oligonucleotides are 30 to 100 base pairs in length, optionally 45 to 65 base pairs in length. In yet another embodiment, the oligonucleotides are complementary to at least 5, 10, 15, 30, 40 or 60 contiguous nucleotides of the rearrangement indicator sequence regions.

In one embodiment, at least 5, 10, 15, 50, 100, 500 or 1000 oligonucleotide probes comprise a nucleotide sequence complementary to a single rearrangement indicator sequence region.

The application also discloses a system wherein the rearrangement indicator sequence regions comprise at least one rearrangement hotspot. Optionally, the rearrangement hotspot is contained within a segmental duplication. In one embodiment, the rearrangement hotspot comprises at least 10 duplicons.

In one embodiment, the rearrangement hotspots are selected from the genomic regions listed in Table 1. In another embodiment, the rearrangement indicator sequence regions are selected from the genomic regions listed in Tables 2-5.

In one embodiment, the solid support comprises at least one microarray chip and the oligonucleotide probes are arrayed on the at least one microarray chip. Optionally, the solid support comprises at least two microarray chips and the oligonucleotide probes are arrayed on the at least two microarray chips.

The application further discloses the use of a microarray chip system comprising: at least 500, 750, 1000, 1500, 2000 or 4000 distinct oligonucleotide probes bound to a solid support, wherein each oligonucleotide probe comprises a nucleotide sequence complementary to a rearrangement indicator sequence region of a human genome, the rearrangement indicator sequence regions comprising a rearrangement indicator set that is indicative of risk, or occurrence, of genomic rearrangements, wherein the use comprises detecting a genomic rearrangement or the risk of a genomic rearrangement or for identifying a novel genomic rearrangement.

In one embodiment, the genomic rearrangement indicates a genetic disease or the risk of a genetic disease.

In another embodiment, the genomic rearrangement is a copy number variation (CNV). Optionally, the CNV comprises a gene deletion or gene duplication. In further embodiments, the genomic rearrangement comprises a complex rearrangement, optionally multiple duplications and/or deletions and combinations thereof. Optionally, the genomic rearrangement comprises a duplication-deletion-duplication, a deletion-duplication-deletion, a duplication-duplication-deletion or a deletion-deletion-duplication. In other embodiments, the genomic rearrangement comprises a triplication. Optionally, the gene deletion or gene duplication comprises the deletion or duplication of a genomic sequence at least 200 bp, 500 bp, 1000 bp or 2000 bp in length.

In another embodiment the genomic rearrangement indicates a genetic disease in a subject. Optionally, the genomic rearrangement indicates the presence of, or the propensity for, a genetic disease in a subject.

Optionally, the genetic disease is Autism Spectrum Disorder, Psoriasis, Ankylosing Spondylitis or Tourette Syndrome. In a further embodiment, the genetic disease is Autism Spectrum Disorder and the genomic rearrangement is detected in the genes PGAP 1 or LNX1. In another further embodiment, the genetic disease is Ankylosing Spondylitis and the genomic rearrangement is detected in the genes UGT2B17 or UGT2B15.

The application also discloses a method of detecting genomic rearrangements in a subject. In one embodiment, the method comprises:

labeling a DNA test sample from a subject with a first fluorophore;

labeling a DNA reference sample with a second fluorophore;

contacting the labeled samples with the microarray system of the present application and hybridizing the labeled samples to the oligonucleotide probes of the present application; and

identifying a putative genomic rearrangement, wherein a non-equal signal ratio between first fluorophore and the second fluorophore identifies a putative genomic rearrangement.

In one embodiment, the DNA test sample and the DNA reference sample are genomic DNA samples. In another embodiment, the labeled samples are hybridized to the oligonucleotide probes of the disclosure under medium or high stringency hybridization conditions.

In one embodiment, a log 2 ratio of >0.25 or <−0.25 identifies a putative genomic rearrangement.

Optionally, the method further comprises validating the putative genomic rearrangement by quantitative FOR, fluorescence in-situ hybridization (FISH) analysis or karyotyping.

In one aspect of the method, the genomic rearrangement is associated with a genetic disease. In another aspect, the genomic rearrangement is a novel genomic rearrangement.

The application also discloses a method of constructing a microarray chip for detecting copy number variations comprising:

identifying at least 500, 1000, 1500, 2000 or 4000 rearrangement indicator sequence regions;

designing oligonucleotide probes complementary to the rearrangement indicator sequence regions; and

arraying the oligonucleotide probes on at least one microarray chip.

In one embodiment, the method further comprises the use of the chip to detect a genomic rearrangement wherein differential binding of a test genomic DNA sample and a reference genomic DNA sample to at least one oligonucleotide probe indicates a genomic rearrangement. Optionally, higher binding of the test genomic DNA sample than the reference genomic DNA sample to at least one oligonucleotide probe indicates a genomic duplication and higher binding of the reference genomic DNA sample than the test genomic DNA sample to at least one oligonucleotide probe indicates a genomic deletion.

Using the genome-wide rearrangement hotspots and microarray chips described herein, the inventors discovered a novel genomic region on chromosome 2 that is associated with copy number variants that segregate with Tourette Syndrome status.

Accordingly, the application also relates to a method of screening for, diagnosing and/or detecting an increased risk of developing Tourette syndrome in a subject comprising detecting the presence of a Tourette syndrome copy number variant in a Tourette syndrome critical region within a sample of the subject, wherein the presence of a Tourette syndrome copy number variant in a Tourette Syndrome critical region is indicative that the subject has Tourette Syndrome and/or an increased risk of developing Tourette syndrome.

In one embodiment, the Tourette Syndrome copy number variant is detected by one or more of: quantitative PCR, RT FOR, QF-PCR, fluorescent in situ hybribization (FISH), a binding agent, and/or a microarray.

The application also relates to a method of evaluating a genomic DNA from a subject suspected of having or having Tourette Syndrome comprising:

a) obtaining a genomic DNA test sample from the subject,

b) assaying the test sample to determine the presence or number of copies of a Tourette Syndrome copy number variant in the test sample

c) assaying a reference sample to determine the presence or number of copies of the Tourette Syndrome copy number variant in the reference sample,

d) identifying differences between the amount or number of copies of the Tourette Syndrome copy number variant in the Lest sample compared to the reference sample;

wherein differences between the amount or number of copies of the Tourette Syndrome copy number variant in the test sample compared to the reference sample are indicative of whether the subject has Tourette Syndrome or an increased risk of developing Tourette Syndrome.

The application also relates to a method of screening for, diagnosing and/or detecting an increased risk of developing Tourette Syndrome in a human subject comprising:

a) obtaining a sample from the subject;

b) assaying the sample for the presence of and detecting a Tourette Syndrome copy number variant in a Tourette Syndrome critical region thereby identifying the subject as having Tourette Syndrome or an increased risk of developing Tourette Syndrome, the assaying comprising hybridizing a probe and/or primer to the Tourette Syndrome copy number variant.

In one embodiment, the Tourette syndrome critical region is on chromosome 2, optionally located at 2q21.1-21.2.

In another embodiment, the Tourette Syndrome copy number variant is a duplication or a deletion. The duplication is optionally a duplication of genomic sequence corresponding to: chr2:132305299-132343808, chr2:132395155-132526804 or chr2:132305299-132343808 of human genome assembly 19.

In one embodiment, detecting a Tourette Syndrome copy number variant with an increased copy number compared to a reference sequence identifies the subject as having Tourette Syndrome or an increased risk of developing Tourette Syndrome. In one embodiment, a copy number of 4 or more compared to a reference sequence identifies the subject as having Tourette Syndrome or an increased risk of developing Tourette Syndrome. Optionally, the reference sequence is a human genome assembly sequence such as human genome assembly 19 (HG19).

In one embodiment the subject is presymptomatic, has one or more clinical symptoms or clinical features associated with Tourette Syndrome, has been diagnosed with Tourette syndrome and/or has at least one blood relation with Tourette Syndrome.

The application also provides isolated nucleic acids. In some embodiments, the isolated nucleic acids are useful as probes or primers for detecting Tourette Syndrome copy number variants.

Accordingly, in one embodiment, the application provides an isolated nucleic acid, wherein the nucleic acid hybridizes to:

a Tourette Syndrome copy number variant or a portion thereof;

a nucleic acid sequence complementary to a); and/or

a nucleic acid sequence corresponding to a).

Optionally, the Tourette Syndrome copy number variant comprises genomic sequence corresponding to chr2:132395155-132526804, chr2:132305299-132343808 or chr2:132480185-132510827 of human genome assembly 19.

In one embodiment, the isolated nucleic acid is a primer or a probe.

The application also provides a kit for screening for, diagnosing or detecting an increased risk of developing Tourette Syndrome comprising:

(a) a Tourette Syndrome copy number variant detection agent comprising an isolated nucleic acid as described here; and

instructions for use or a container for holding the detection agent of (a).

### BRIEF DESCRIPTION OF THE DRAWINGS

Embodiments of the disclosure will be shown in relation to the drawings in which the following is shown:

FIG. 1 is a schematic illustrating the hierarchical approach used in the present disclosure. mrsFAST was used to obtain read depth distribution of the NA18507 human genome with maximum two mismatch was allowed against the repeat masked reference human genome (build 36). A mean-based approach was utilized to computationally predict the boundaries of regions associated with excessive read depth. Another alignment algorithm MAQ was used to obtain the consensus genome (mapping quality Q>30 and n=2) from the NA18507 genome assembly. The consensus sequence for highly excessive read depth regions was obtained in order to apply a window-based alignment algorithm. The previously identified novel 4.8 Mb sequence from de novo assembly within this genome was also included in the rearrangement analysis.

FIG. 2 depicts segmental duplication (SD) units which represent the most complex rearrangements within the NA18507 human genome. a) A total of 1963 SD complex units (i.e., ≧10 rearrangements) were identified that were significantly different (p<1.0×10⁻⁶) compared with the rest of the NA18507 genome duplicated regions. The plot illustrates the concordance of the predicted autosomal complex regions compared with previous studies [Conrad, 2010; Sudmant, 2010]. b) Genes that completely or partially overlapped with detected SD units in which 73% (41/56) of the most variable genes in three different populations were detected in our analysis of the NA18507 human genome. Among the 1626 genes identified in this study, 10% (i.e., 166/1626) of genes that overlapped with a SD unit revealed extreme inter- and intra-chromosomal rearrangements, 50% of which have been previously validated [Conrad, 2010]. c) Observed gene content transfer between hotspot and non-hotspot agenic SD units. d) Scatter plot illustrating DNV count for hotspot and non-hotspot SD units. e) A histogram illustrating the mean read depth (RD) of the computationally predicted SD unit breakpoints. The dark bars represent the mean read depth for each of the 20,237 SD unit breakpoints and the light bars represent the mean read depth for hotspot regions.

FIG. 3 depicts the physical position of rearrangement hotspots that has been mapped within the proximal/distal breakpoints of a pathogenic deletion (dark horizontal block) or duplication (light horizontal block).

FIG. 4 depicts the landscape of chromosomal rearrangements in the NA18507 human genome. Chromosomal rearrangements located within duplicated regions are plotted against the human genome. Bars represent the signature of intra-chromosomal rearrangements, inter-chromosomal rearrangements and ‘rearrangement hotspots’. Cytobands with duplications for each chromosome and selected genes that completely or partially overlapped with SD units are also indicated.

FIG. 5 depicts a signature of rearrangement hotspots located at a) 16p12.1 and b) 22q11.21. a) A 40 kbp region within 16p12.1 is illustrated with its corresponding derivative copies which were localized by hierarchical analysis. This region consists of the NPIPL3 gene derivatives. The inter- and intra-chromosomal localization of the copies is approximated in the physical map within the chromosome contig (18p11.21). The alignments are coded for chromosomes (i.e., coded rectangles below the read depth plot) and FISH validation is illustrated for both inter- and intra-chromosomal localization. The pathogenic deletions and duplications located within these regions [Nagamani S C, 2011; Heinzen E L, 2010; Weiss L A, 2008; Walters R G, 2010; Ballif B C, 2007; Tokutomi T, 2009] are depicted in dark and light bars, respectively. The bars under the contig represent the approximated inversions previously reported by Antonacci, F. et al., 2010. b) Analysis of a 37 kbp duplicated region within 22q11.21 revealed it is comprised of a core 2.7 kbp tandem duplicon copied from different chromosomes. Black lines represent the read depth (x-axis), shade represents an SD unit, and bars represent the region with common repeat elements. The horizontal blocks (coded according to chromosomes) are the rearrangement (infra/inter) fragments with >90% sequence similarity and >100 bp in length.

FIG. 6 depicts Tourette Syndrome pedigrees. Family A comprises three generations. Pedigrees B and C comprise two (2) trios with affected offspring. Ten (10) unrelated probands with TS are also shown.

FIG. 7 depicts array CGH microarray results from family A. Each data point represents a probe intensity value. The dotted lines illustrate the genomic gains in two distinct genomic blocks (block1 and block2) within the affected samples. The horizontal line for sample ID3002 demonstrates a partial gain.

FIG. 8 depicts a schematic illustrating a 9 MB critical region located at 2q14.3-q21.2 previously detected in a multiplex family with Dystonia and comparison with the micro-duplications reported in this study. The reciprocal micro-duplication and micro-deletion regions detected in TS and ADHD samples are indicated in upper and lower horizontal blocks, respectively. The micro-duplications reported in this study are located at 2q21.1-21.2 and illustrated by the upper horizontal blocks.

### DETAILED DESCRIPTION

The present disclosure relates to the genome-wide identification of “rearrangement hotspots”. These rearrangement hotspots can facilitate the detection of genomic regions capable of mediating genomic rearrangements or aberrations such as de novo deletions or duplications in humans. The disclosure further relates to microarrays that comprehensively target vulnerable or fragile regions in the genome susceptible to disease-associated rearrangements and the use of the microarrays for detecting disease-associated genomic rearrangements. The application also discloses novel copy number variants in chromosome 2 that were identified using the microarrays described herein and co-segregate with Tourette Syndrome status.

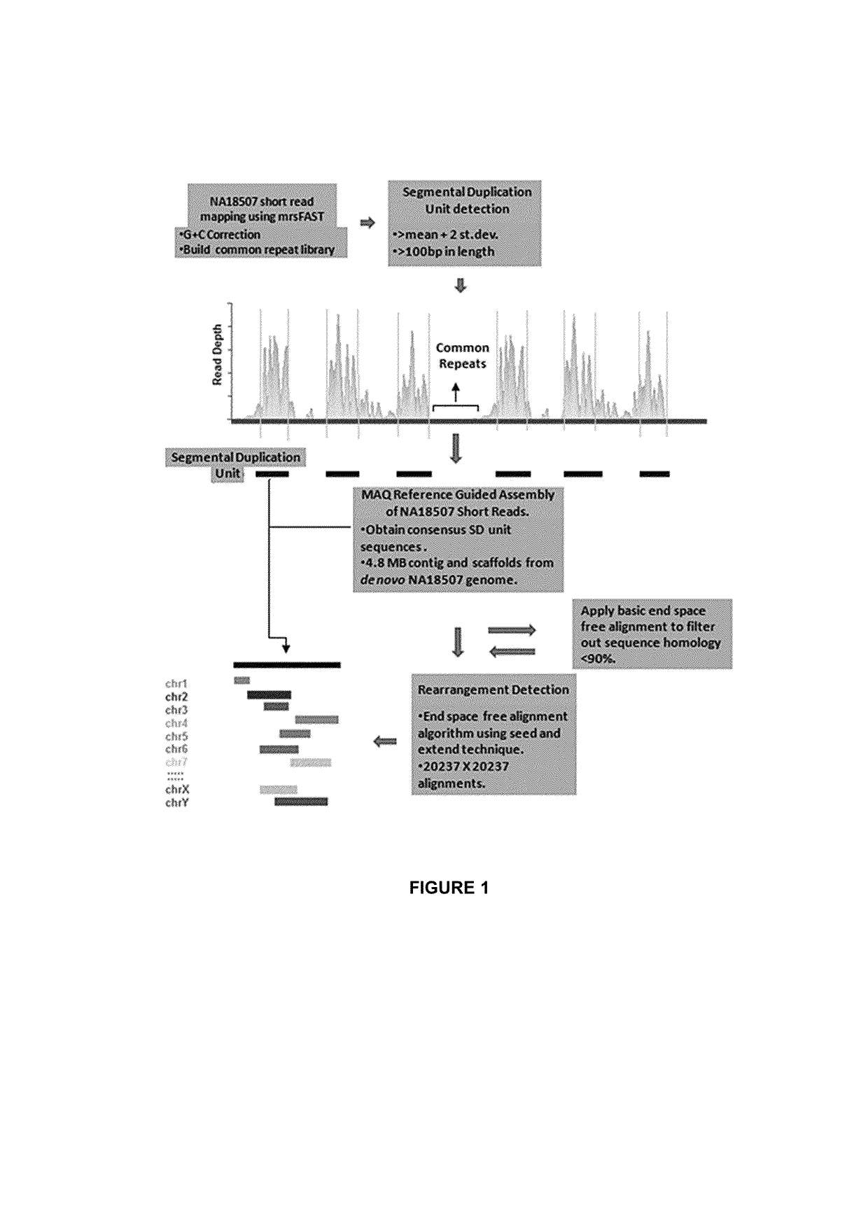

The application describes the identification of genome-wide rearrangement hotspots that often predispose to genomic disorders in humans, mediated predominately by non-allelic homologous recombination. The application discloses a hierarchical approach to detect segmental duplication units using an all-hit mapping algorithm, interrogating every 100 by (GC-corrected read depth window with a 1 by overlap) excluding common repeat elements. Reference-guided assembly was obtained from reads based on the NA18507 human genome and duplicated sequences were extracted from the assembly using detected breakpoints (FIG. 1). To create a genome-wide high resolution map of “rearrangement hotspots”, an end-space free pairwise alignment algorithm was developed integrating a ‘seed and extend’ technique which can accurately investigate the complex structural architecture associated with the technically challenging and problematic nature of segmental duplications. Without being bound by theory, it is believed that highly homologous segmental duplication regions (i.e., rearrangement hotspots) predispose to genomic rearrangements arising from recombination and replication-based events.

In the present disclosure, the terms “genomic rearrangements”, “genomic alterations” and “genomic aberrations” refer to structural modifications, changes and alterations in chromosomal DNA. Common genomic rearrangements include copy number variants (CNVs) including gene duplications and gene deletions. In the present disclosure, the term “copy number variation” is defined as the gain or loss of genomic material compared to a reference sequence.

Additional genomic rearrangements, alterations or aberrations include, but are not limited to, insertions, translocations, recombinations, rearrangements and combinations thereof. The modification or change can vary in size from only a few bases to several kilobases. In some embodiments, the genomic material gained or lost in a genomic rearrangement is greater than 250 bp, 500 bp, 1 KB or 2 KB in size. In a genomic rearrangement, one or more parts of a chromosome are optionally rearranged within a single chromosome (intra-chromosomal) or between chromosomes (inter-chromosomal).

Genomic aberrations, rearrangements and alterations may result from multiple events, including but not limited to, non-allelic homologous recombination (NAHR), non-homologous end-joining (NHEJ), fork stalling and template switching (FoSTes) and microhomology-mediated break induced replication (MMBIR).

Diseases associated with, or indicated by, genomic rearrangements include genetic diseases which arise, at least in part, from genomic aberrations, rearrangements and alterations. Examples of genetic diseases associated with genomic rearrangements include, but are not limited to, 3q29 microdeletion/duplication syndrome, globozoospermia and Williams-Beuren syndrome. Several developmental neurocognitive disorders are caused by recurrent and non-recurrent genomic rearrangement and copy number variants have been identified which are responsible for mental retardation, autism spectrum disorders, developmental delays and multiple congenital anomalies. Copy number variants have also been detected in common autoimmune diseases such as ankylosing spondylitis, psoriasis, psoriatic arthritis, psoriasis vulgaris, rheumatoid arthritis, inflammatory bowel disease and systemic lupus erithmatosis.

Genomic Regions Associated with Genomic Rearrangements

The term “genomic region” refers to a contiguous length of nucleotides in a genome of an organism. A genomic region may be in the range of 10 kb in length to an entire chromosome, for example 100 kb to over 1 MB in length. Genomic regions are also referred to as “breakpoints”.

In the present disclosure, “rearrangement hotspots” are genomic regions susceptible to genomic rearrangements such as gene deletions or gene duplications. Examples of rearrangement hotspots are listed in Table 1. A genomic region susceptible to a genomic rearrangement is optionally a genomic region which is at least 10%, 35%, 50%, 100% more likely to undergo a genomic rearrangement than a genomic region that is not susceptible to genomic rearrangements. Such regions may be termed vulnerable or fragile genomic regions. Rearrangement hotspots can be correlated with increased non-allelic homologous recombination event frequency. In some embodiments of the disclosure, rearrangement hotspots are found within segmental duplications. In one particular embodiment, “rearrangement hotspots” are highly homologous regions within segmental duplication units. “Segmental duplications” (also known as low-copy repeats) are regions of DNA greater than 1 kilobase in size which share a high level of sequence homology, for example, more than 75%, 85% 90% or 95% sequence homology. Segmental duplications include both inter- and intra-chromosomal segmental duplications.

In other embodiments, rearrangement hotspots have a significantly high distribution of duplicons (p<1.0×10⁻⁶) with at least 10 duplicons per hotspot. Rearrangement hotspots optionally range in size from 100 to 15,000 base pairs, optionally 200 to 1000 base pairs.

“Duplicons” are short regions of homologous DNA, optionally greater than 100 bp. Duplicons are optionally located within segmental duplications and are highly homologous sequences located sparsely within the genome. Homologues of the duplicon can be located within the same chromosome or in other chromosomes

A “rearrangement indicator sequence region” is a genomic region identified to have a propensity for genomic rearrangements or known to be involved in genomic rearrangements. Optionally, a “rearrangement indicator sequence region” is a genomic region susceptible to genomic rearrangements such as gene deletions or gene duplications. A “rearrangement indicator sequence region” is optionally a genomic region which is at least 10%, 35%, 50%, 100% more likely to undergo a genomic rearrangement than a genomic region that is not susceptible to genomic rearrangements.

Optionally, a “rearrangement indicator sequence region” is used to identify when a genomic rearrangement has occurred. In one embodiment, oligonucleotides complementary to a “rearrangement indicator sequence region” are used to detect whether a genomic rearrangement has occurred through competitive hybridization of a test genomic DNA sample and a reference DNA sample to the oligonucleotides.

Rearrangement indicator sequence regions include genomic regions comprising or consisting of at least one rearrangement hotspot. A rearrangement indicator sequence region optionally comprises one rearrangement hotspot or multiple rearrangement hotspots. In some embodiments, rearrangement indicator sequence regions are 100 base pairs to 5 MB in length. Rearrangement indicator sequence regions also include genomic regions containing known CNVs and centromeric and telomeric chromosomal regions.

Examples of rearrangement indicator sequence regions are listed in Tables 1-5.

A “rearrangement indicator set” is a set or group of rearrangement indicator sequence regions that together are indicative of risk, or occurrence, of genomic rearrangements. Optionally, a “rearrangement indicator set” is a set or group of rearrangement indicator sequence regions that cumulatively are indicative of risk, or occurrence, of genomic rearrangements. “Risk of genomic rearrangements” refers to the risk that a particular genomic region may undergo a genomic rearrangement. “Occurrence of genomic rearrangements” refers to the presence of a genomic rearrangement in a subject. A “rearrangement indicator set” optionally comprises at least 500, 750, 1000, 1500, 2000 or 4000 rearrangement indicator sequence regions. In one embodiment, a “rearrangement indicator set” comprises at least 500, 750, 1000, 1500, 2000 or 4000 genomic regions that are at least 10%, 35%, 50%, 100% more likely to undergo a genomic rearrangement than a genomic region that is not susceptible to genomic rearrangements.

Oligonucleotides

The present disclosure provides oligonucleotides complementary to nucleotide sequences contained within genomic regions associated with genomic aberrations or rearrangements. The term “oligonucleotide” refers to short single stranded nucleic acid polymers. Oligonucleotides may be made synthetically or enzymatically. Oligonucleotides may range in length from 2 to 200 base pairs.

In one embodiment, the oligonucleotides described herein are used as array probes. The term “oligonucleotide probe” refers to an oligonucleotide designed for use as an array probe. Optionally, the oligonucleotide probes of the present disclosure range from 25-80 base pairs in length, preferably 45-60 base pairs in length.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some nucleotides or portions of the nucleotide sequences of the nucleic acids bind, or it may be complete when total complementarity exists between the single stranded molecules. The degree of complementarity between nucleic acid strands has significant effects on the efficiency and strength of hybridization between nucleic acid strands. In one embodiment of the present disclosure, oligonucleotide probes are complementary to contiguous sequences contained within genomic regions associated with genomic aberrations or rearrangements. In another embodiment, oligonucleotide probes hybridize to contiguous sequences contained within genomic regions associated with genomic aberrations or rearrangements. Optionally, the contiguous sequences are at least 5, 10, 20, 30, 40, 50 or 60 basepairs in length.

The term “hybridization” refers to the specific binding of a nucleic acid to a complementary nucleic acid. In one embodiment of the present disclosure, oligonucleotide probes hybridize under medium stringency hybridization conditions to genomic regions associated with genomic aberrations or rearrangements, for example rearrangement indicator sequence regions. In another embodiment, oligonucleotide probes hybridize under high stringency hybridization conditions to genomic regions associated with genomic aberrations or rearrangements, for example rearrangement indicator sequence regions. The terms “medium stringency hybridization conditions” and “high stringency hybridization conditions” are well known to a person skilled in the art. Examples of hybridization conditions may be found in molecular biology reference texts such as Molecular Cloning: A Laboratory Manual by Sambrook and Russell (3^{rd} Edition, Cold Spring Harbour Press, 2001).

The stringency may be selected based on the conditions used in the wash step. For example, the salt concentration in the wash step can be selected from a high stringency of about 0.2×SSC at 50° C. for 15 minutes. In addition, the temperature in the wash step can be at high stringency conditions, at about 65° C. for 15 minutes.

By “medium stringency hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=31.5° C.−16.6 (Log 10[Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% sequence identity, the final wash temperature will be reduced by about 5° C. Based on these considerations those skilled in the art will be able to readily select appropriate hybridization conditions.

In some embodiments, stringent or high stringency hybridization conditions are selected. By way of example the following conditions may be employed to achieve high stringency hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. for 15 minutes. Moderately stringent hybridization conditions include a washing step in 3×SSC at 42° C. for 15 minutes. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 2000, Third Edition.

According to the methods of the disclosure, oligonucleotide probes may be designed which are complementary to the identified rearrangement indicator sequence regions. Optionally, the oligonucleotide probes are designed to hybridize under medium stringency or high stringency conditions to the rearrangement indicator sequence regions.

In one embodiment, the oligonucleotide probes are complementary to, or hybridize to, the genomic regions set out in Tables 1-5. Optionally, the oligonucleotides may be complementary to, or hybridize to, sequences corresponding to the genomic regions set out in Tables 1-5. It is appreciated that there is variation in human genome sequences and the regions set out in Tables 1-5 specifically reflect human genome NA18507. The present disclosure encompasses regions in other human genomes that correspond to the regions depicted in Tables 1-5.

The term “distinct oligonucleotide probes” refers to oligonucleotide probes that each have different/distinct oligonucleotide sequences. Within the context of the present disclosure, “distinct oligonucleotide probes” each bind to, or are complementary to, different/distinct rearrangement indicator sequence regions.

Within a rearrangement indicator sequence region, the spacing between individual oligonucleotide probes ranges from not more than 100, 150, 280, 300, 500 or 1000 base pairs apart. The mean spacing between oligonucleotides with a single rearrangement indicator sequence region is approximately 280 base pairs, optionally 200 to 350 base pairs.

### Arrays

One aspect of the application provides an array for use in the methods described herein. In one embodiment, the application provides an array comprising a solid support having a plurality of addresses, wherein each address has disposed thereon an oligonucleotide probe that can specifically bind genomic DNA. In some embodiments, an array contains at least one, ten, 100, 1000, 10,000, 100,000, or 1,000,000 features in an area that is less than 20 cm², 10 cm², 5 cm², 1 cm² or less than 1 mm².

The application also provides a “microarray chip system” comprising at least one solid support, optionally a glass slide, upon which oligonucleotides, such as the oligonucleotide probes described herein, have been arrayed. A microarray chip system includes more than one solid support wherein a unique collection of probes are arrayed on each support.

In one embodiment of the disclosure, the microarray system comprises a plurality of oligonucleotide probes bound to at least one solid support, the oligonucleotide probes comprising nucleotide sequences complementary to at least 500, 750, 1000 or 1500 rearrangement indicator sequence regions and wherein the at least 500, 750, 1000 or 1500 rearrangement indicator sequence regions are represented by at least one oligonucleotide probe. Optionally, the oligonucleotide probes hybridize under medium stringency or high stringency hybridization conditions to at least 500, 750, 1000 or 1500 rearrangement indicator sequence regions. In a preferred embodiment, the rearrangement indicator sequence regions are selected from the genomic regions listed in Tables 1-5.

The present disclosure also relates to methods for manufacturing microarray systems. In one embodiment of the disclosure, a method of constructing a microarray chip or microarray chip system for detecting copy number variations comprises identifying at least 500, 1000, 1500 rearrangement indicator sequence regions, designing oligonucleotide probes corresponding to the genomic regions and arraying the oligonucleotide probes on a microarray chip.

In a further embodiment, the oligonucleotides described herein are arrayed on microarray chips. Optionally, the oligonucleotide probes are arrayed on at least 1, at least 2, at least 3 or at least 4 microarray chips. In one embodiment, one million probes are arrayed on two microarray chips for a total of two million probes.

In a preferred embodiment, the oligonucleotide probes are arrayed on the support using inkjet technology, for example, Agilent's Sureprint system.

### Method for Detecting Genomic Rearrangements

The disclosure provides methods for detecting a genomic rearrangement in a subject. Optionally, the disclosure provides for the use of the microarray chips described herein for detecting genomic rearrangements.

The methods of the disclosure further relate to the use of the microarray chips for diagnosing genomic disorders and for diagnosing the propensity to develop a particular disorder. The methods of the disclosure also relate to the use of the microarray chips for identifying a genetic basis for known diseases and for characterizing the specific genomic rearrangements that lead to a particular genetic disorder. In another embodiment of the disclosure, the present microarray chips are used to identify novel genomic rearrangements.

In one embodiment, the method provides competitively hybridizing test DNA samples and reference DNA samples to at least one microarray chip comprising oligonucleotides complementary to at least 500, 750, 1000 or 1500 rearrangement indicator sequence regions. In some embodiments, the methods provide labeling a test DNA sample with a first label and a reference DNA sample with a second label. Optionally, the DNA is genomic DNA.

The term “genomic DNA” refers to deoxyribonucleic acids that are obtained from an organism. The organism may be a human subject, a mouse or any other organism of interest. “Genomic DNA” may be purified, isolated, amplified, fragmented DNA. Optionally, genomic DNA is obtained from biological samples including, but not limited to, cell, tissue, organ, body fluid, excretory samples.

In some embodiments, the test DNA sample is genomic DNA to be tested for genomic rearrangements or aberrations and the reference DNA sample is a standard for detecting differences between the test DNA sample and the reference DNA sample. The test DNA sample may be a genomic DNA sample from a subject believed or suspected to have at least one genomic rearrangement or a disease associated with at least one genomic rearrangement or believed or suspected to have to have at least one genomic rearrangement or a rearrangement for a disease associated with at least one genomic rearrangement. For example, the subject can be a member of a family known to be affected by at least one genomic rearrangement or for a disease associated with at least one genomic rearrangement. In another embodiment, the test DNA sample is a genomic DNA sample from a subject, wherein the subject is to be tested or screened for at least one genomic rearrangement or for a disease associated with at least one genomic rearrangement.

In some embodiments, the reference DNA sample is genomic DNA from a subject who does not have at least one genomic rearrangement or a disease associated with at least one genomic rearrangement.

In a preferred embodiment, the test DNA sample and reference DNA sample are labeled with a substance that allows the quantity of each sample to be detected. Optionally, the labels are fluorescent labels or fluorophores. In one embodiment, the labels are Cy3 and Cy5.

According to the methods of the disclosure, the labeled test DNA and the labeled reference DNA are hybridized competitively to oligonucleotide probes. Optionally, the labeled test DNA and the labeled reference DNA are mixed together prior to hybridization. In one embodiment, labeled test DNA and the labeled reference DNA is hybridized under high stringency or medium stringency conditions to a microarray chip described herein. In a preferred embodiment, the labeled test DNA and the labeled reference DNA is hybridized to at least one microarray comprising oligonucleotide probes complementary to at least 500, 750, 1000 or 1500 rearrangement indicator sequence regions.

The hybridization may be performed under any appropriate hybridization conditions. Preferably, the hybridization is carried out under medium stringency or high stringency hybridization conditions. Optionally, the hybridization is carried out at around 37° C., 48° C. or 60° C. for at least 24, 36, 48, 80 or 86 hours.

Genetic aberrations or rearrangements are optionally detected using the resultant florescent intensities as an indicator.

In one embodiment of the disclosure, the fluorescent intensity on the oligonucleotide probe is measured and genomic rearrangements are detected using fluorescence intensity as an indicator. In one embodiment, in order to detect a genomic rearrangement (for example, a duplication or deletion of the test DNA), the fluorescence intensity ratio of the labeling substance derived from the reference genomic DNA to labeling substance derived from the test genomic DNA is determined from the fluorescence intensity obtained. Fluorescence intensity can be determined, for example, using an image analyzer.

When the ratio of fluorescence intensity of the labeling substance derived from the reference DNA to the labeling substance derived from the test DNA is high, more reference DNA than test DNA has hybridized to the oligonucleotide probe and a potential genomic deletion in the test DNA (a decrease in copy number) is indicated.

When the ratio of fluorescence intensity of the labeling substance derived from the reference DNA to the labeling substance derived from the test DNA is low, more test DNA than reference DNA has hybridized to the oligonucleotide probe and a potential genomic duplication in the test DNA (an increase in copy number) is indicated.

In one embodiment of the disclosure, the analysis parameters for the microarray are as follows:

GC correction is applied on ever 2 kb window of the genome using an ADM-2 algorithm

The derivative of the log spread ration (DLRS) must be <0.3, optionally <0.1; <0.2; <0.3; <0.4 or <0.5 for a sample

The different must be seen in at least 5 probes

The log 2 ratio between the signal corresponding to the test sample and the signal corresponding to the reference sample must be >0.25 or <−0.25, optionally >0.25 or <−0.25, optionally >0.1 or <−0.1; >0.15 or <−0.15; >0.2 or <−0.2; >0.25 or <−0.25; >0.3 or <−0.3; >0.35 or <−0.35; or >0.5 or <−0.5.

In one embodiment of the disclosure, a log 2 ratio of >0.1 or <−0.1; >0.15 or <−0.15; >0.2 or <−0.2; >0.25 or <−0.25; >0.3 or <−0.3; >0.35 or <−0.35; or >0.5 or <−0.5 indicates a putative genomic rearrangement. In another embodiment, a log 2 ratio of >0.25 or <−0.25, indicates a putative genomic rearrangement.

In one embodiment of the disclosure, a method is provided for detecting genomic rearrangements associated or predisposing subjects to developmental neurocognitive disorders (for example, autism spectrum disorder or Tourette syndrome) and complex autoimmune disorders (for example, psoriasis and ankylosing spondylitis).

In one embodiment, the method comprises obtaining genomic DNA from a test subject and genomic DNA from a reference subject. Optionally, the reference subject does not suffer from a developmental neurocognitive disorders (for example, autism spectrum disorder or Tourette syndrome) and/or complex autoimmune disorders (for example, psoriasis and ankylosing spondylitis).

The genomic DNA from the test subject is optionally labeled with a first flourophore, optionally Cy3 or Cy5, and the genomic DNA from the reference subject is optionally labeled with a second flourophore, optionally Cy3 or Cy5. The labeled DNA is competitively hybridized to a microarray chip comprising oligonucleotide probes complementary to genomic regions known to be associated with genomic rearrangements that can indicate a developmental neurocognitive disorder (for example, autism spectrum disorder or Tourette syndrome) and/or a complex autoimmune disorder (for example, psoriasis or ankylosing spondylitis).

In one embodiment, genomic rearrangements in the following genes/regions can be used to indicate developmental neurocognitive disorders (for example, autism spectrum disorder or Tourette syndrome) and/or complex autoimmune disorders (for example, psoriasis and ankylosing spondylitis) or the propensity to develop such a disorder:

| Gene or Genomic region | Associated Disorder |
|---|---|
| 16p11.2 (50 kb in length) | Autism spectrum disorder |
| PGAP-1 gene | Autism spectrum disorder |
| LNX1 gene | Autism spectrum disorder |
| C7orf58 gene | Autism spectrum disorder |
| Within & adjacent to UGT2B17 and | Ankylosing spondylitis |
| UGT2B25 genes | |
| 2q14.3-2q21.3 | Tourette Syndrome |

### Methods of Diagnosing Tourette Syndrome

Using the microarray chips described herein, the inventors discovered a genomic region on chromosome 2 that is associated with novel copy number variants that segregate with Tourette Syndrome status.

Accordingly, the application discloses methods of screening for, diagnosing and/or detecting an increased risk of developing Tourette Syndrome in a subject comprising detecting the presence of a Tourette Syndrome copy number variant in a Tourette syndrome critical region within a sample of the subject, wherein the presence of a Tourette syndrome copy number variant in a Tourette syndrome critical region is indicative that the subject has Tourette syndrome and/or an increased risk of developing Tourette syndrome.

As used herein, Tourette syndrome (TS) refers to a developmental neuropsychiatric disorder characterized by the presence of motor (simple and/or complex) and verbal tics with duration longer than one year [Pauls et al. 1991; Price et al. 1985; State 2011]. TS often manifests with features associated with obsessive compulsive disorder (OCD), attention deficit hyperactivity disorder (ADHD), poor impulse control and other behavioural abnormalities.

As used herein the phrase “screening for, diagnosing or detecting Tourette Syndrome” refers to a method or process of determining if a subject has Tourette Syndrome. Further, the phrase “screening for, diagnosing or detecting a risk of developing a Tourette Syndrome” refers to a method or process of determining if a subject has an increased risk of developing Tourette Syndrome.

As used herein, the term “Tourette Syndrome critical region” refers to a genomic region wherein at least one copy number variant within the genomic region segregates with Tourette Syndrome status. “Segregates with Tourette Syndrome status” indicates that a copy number variant is associated with Tourette Syndrome. For example, a particular copy number variant (for example, a duplication or a deletion) is present in subjects who have Tourette Syndrome but is absent in subjects who do not have Tourette Syndrome.

In one embodiment, the “Tourette Syndrome critical region” is located on chromosome 2. In another embodiment, the “Tourette syndrome critical region” is located at 2q14.3-q21.2. In another embodiment, the “Tourette syndrome critical region” is located at 2q21.1-21.2.

As used herein, a “copy number variant” or CNV is a DNA sequence of one kilobase (kb) or longer (for example, at least 2, 5, 10, 30, 50, 100, 150 or 200 kb in length) that is present at a variable copy number in comparison with a reference genome. Examples of reference genomes include the human genome assemblies such as human genome assembly 19 (HG19) and human genomes NA18507, NA10851, NA15510, NA07048.

Examples of copy number variants include “duplications” where the copy number of the DNA sequence is higher compared to a reference genome and “deletions” where the copy number of the DNA sequence is lower compared to a reference genome.

A “Tourette Syndrome copy number variant” refers to a copy number variant, for example a duplication or a deletion, that is associated with or useful for screening, diagnosing or detecting an increased risk of developing Tourette Syndrome when compared to a reference sequence (for example, human genome assembly 19). A “Tourette Syndrome copy number variant” is present in a higher or lower copy number in a subject with Tourette Syndrome or a subject with an increased risk of Tourette Syndrome compared to a subject not affected by Tourette Syndrome. The Tourette Syndrome copy number variant is in one embodiment inherited e.g. a germline mutation. In another embodiment, the copy number variant is sporadic.

In one embodiment, a “Tourette Syndrome copy number variant” is a duplication, i.e, a stretch of genomic sequence that is present in a higher copy number compared to a reference, or wild-type genomic sequence. In another embodiment, a “Tourette Syndrome copy number variant” is a deletion, i.e., a stretch of genomic sequence that is present in a lower copy number compared to a reference, or wild-type genomic sequence.

A reference genomic sequence is genomic DNA obtained from a subject who does not have Tourette syndrome or an increased risk of Tourette syndrome (also known as an “unaffected sample”). Reference genomic sequences include, but are not limited to, human genome sequences NA18507, NA10851, NA15510 NA07048 and human genome assemblies such as human genome assembly 19 (HG19).

In one embodiment, a “Tourette Syndrome copy number variant” is located on chromosome 2. In another embodiment, the “Tourette Syndrome copy number variant” is located at 2q14.3-q21.2. In another embodiment, the “Tourette Syndrome copy number variant” is located at 2q21.1-21.2. In another embodiment, a “Tourette Syndrome copy number variant” is found within the C2orf27A gene.

In one embodiment, a “Tourette syndrome copy number variant” is a 38 kb duplication located at chromosome 2q21.1 within the genomic region corresponding chr2:13205299-132343808 of human genome assembly 19 (HG19).

In another embodiment, a “Tourette syndrome copy number variant” is a 131 kb duplication located at chromosome 2q21.1 within the genomic region corresponding to chr2:132395155-132526804 of human genome assembly 19 (HG19).

In another embodiment, a “Tourette syndrome copy number variant” is a partial 30 kb duplication located at chromosome 2q21.1 within the genomic region corresponding to chr2:132480185-132510827 of human genome assembly 19 (HG19).

The term “corresponding to” as used herein means situated in a different sequence position but having sequence characteristics in common, including identical, or substantially identical, nucleotide sequence flanking the mutation (eg. substantial identity is optionally at least 75% identity over four or more contiguous nucleotides). For example, “genomic region corresponding to chr2: 132305299-132343808 of human genome assembly 19” refers to a genomic region that is equivalently situated in terms of flanking sequence and relative position to chr2: 132305299-132343808 of human genome assembly 19 but that may be identified by a different genomic position in a different genome assembly or reference sequence. Further “corresponding to” can refer to derived from or related to, for example a nucleic acid corresponding to a gene refers to a nucleic acid derived from the gene such as a transcript and/or an amplified or synthetic copy related to the gene.

The terms “risk” and “increased risk” as used herein refer to a subject having a predisposition to developing a disease e.g. increased risk compared to the average risk of a population. The predisposition is optionally inherited, or optionally acquired (e.g. sporadic mutation). The increased risk is relative to a subject not having a Tourette Syndrome copy number variant.

The term “sample” and “sample of a subject” as used herein refer to any sample of a subject that comprises nucleic acids, for example genomic DNA, and/or includes sequence or sequence data corresponding to genomic sequence. In one embodiment, the sample comprises blood, whole blood or a fraction thereof. In another embodiment, the sample is selected from the group consisting of fresh tissue such as a biopsy, frozen tissue and paraffin embedded tissue.

The term “subject” as used herein includes all members of the animal kingdom including multicellular organisms, including mammals, and preferably means humans.

Tourette Syndrome can be difficult to diagnose. The inventors have determined that the methods described herein identify individuals presymptomatically. Accordingly, in one embodiment, the individual is presymptomatic.

As used herein, “a relative” or “blood relation” is a relative genetically related, or related by birth, and includes without limitation 1^{st}, 2^{nd}, 3^{rd}, 4^{th}, 5^{th}, 6^{th}, 7^{th}, 8^{th}, 9^{th} and 10^{th} degree relations, for example but not limited to parents, children, grandchildren, grandparents, cousins and/or 2^{nd} cousins related by blood.

### Isolated Nucleic Acids and Compositions

Tourette Syndrome copy number variants are readily detected using isolated nucleic acids and/or compositions comprising isolated nucleic acids that are specific for a Tourette Syndrome copy number variant.

Accordingly in one aspect, the application provides isolated nucleic acids useful for detecting Tourette Syndrome copy number variants and compositions and reagents comprising isolated nucleic acids useful for detecting Tourette Syndrome copy number variants. Another aspect provides an isolated nucleic acid molecule comprising a nucleic acid sequence comprising a Tourette Syndrome copy number variant or a portion thereof.

The term “isolated nucleic acid sequence” and/or “oligonucleotide” as used herein refers to a nucleic acid substantially free of cellular material or culture medium when produced by recombinant DNA techniques, or chemical precursors, or other chemicals when chemically synthesized. The term “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded and represent the sense or antisense strand.

One aspect of the application provides an isolated nucleic acid molecule, wherein the isolated nucleic acid molecule hybridizes to:

(a) a Tourette Syndrome copy number variant or a portion thereof;

(b) a nucleic acid sequence complementary to a); and/or

(c) a nucleic acid sequence corresponding to a).

Optionally, the Tourette Syndrome copy number variant comprises genomic sequence chromosome 2, optionally with region 2q21.1-21.2. In one embodiment, the Tourette Syndrome copy number variant corresponds to chr2:132395155-132526804, chr2:132305299-132343808 or chr2:132480185-132510827 of human genome assembly 19.

In one embodiment, the isolated nucleic acid molecule is a probe or a primer used to detect a Tourette Syndrome copy number variant.

The hybridization is optionally under high or medium stringency conditions. Appropriate stringency conditions which promote hybridization are known to those skilled in the art, or can be found in Current Protocols in Molecular Biology, John Wiley & Sons, N.Y. (1989), 6.3.1 6.3.6. High and medium stringency hybridization conditions are also described herein.

In an embodiment, the isolated nucleic acid molecule is useful as a primer. The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less, for example, up to 5, 10, 12 or 15 nucleotides. The factors involved in determining the appropriate length of primer are readily known to one of ordinary skill in the art.

In one embodiment, the primers hybridize under medium or high stringency conditions to the Tourette Syndrome copy number variants described herein and allow amplification of a Tourette Syndrome copy number variant or a portion thereof. As used in relation to a primer, “a portion thereof” of a Tourette Syndrome copy number variant refers to a portion sufficient to prime amplification of the intended template.

In another embodiment, the application describes probes that are useful for detecting a Tourette Syndrome copy number variant.

The term “probe” as used herein refers to a nucleic acid sequence that will hybridize to a nucleic acid target sequence. In one example, the probe hybridizes to a Tourette Syndrome copy number variant or a nucleic acid sequence complementary to Tourette Syndrome copy number variant. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length. As used in relation to a probe, “a portion thereof” of a Tourette Syndrome copy number variant refers to a portion sufficient to specifically hybridize to the intended template.

Another aspect of the application provides an isolated nucleic acid molecule which has at least 75, 80, 85, 90, 95 or 99% sequence identity to a Tourette Syndrome copy number variant or a portion thereof. In another embodiment, an isolated nucleic acid molecule is provided which has at least 75, 80, 85, 90, 95 or 99% sequence identity to the complement of a Tourette Syndrome copy number variant or a portion thereof.

The term “sequence identity” as used herein refers to the percentage of sequence identity between two nucleic acid sequences. To determine the percent identity of two nucleic acid sequences, the sequences are aligned for optimal comparison purposes (e.g., gaps can be introduced in the sequence of a nucleic acid sequence for optimal alignment with a second nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci, U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. To obtain gapped alignments for comparison purposes, Gapped BLAST can be utilized as described in Altschul et al., 1997, Nucleic Acids Res, 25:3389-3402, Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website), The percent identity between two sequences can be determined using techniques similar to those described above, with or without allowing gaps. In calculating percent identity, typically only exact matches are counted.

In certain embodiments the isolated nucleic acid comprises a detectable label, such as a fluorescent or radioactive label.

Another aspect of the disclosure provides a reagent for detecting and/or amplifying a Tourette Syndrome copy number variant, such as the isolated nucleic acid primers described herein.

In one embodiment, a reagent for detecting a Tourette Syndrome copy number variant comprises an isolated nucleic acid molecule comprising:

a) an isolated nucleic acid molecule, wherein the isolated nucleic acid molecule hybridizes to a Tourette Syndrome copy number variant or a portion thereof; or

b) a nucleic acid molecule with at least 80%, 90%, 95%, or 99% sequence identity to a), characterized in that the nucleic add molecule is capable of binding a Tourette Syndrome copy number variant under stringent conditions.

### Detecting Tourette Syndrome Copy Number Variants

A person skilled in the art will appreciate that a number of methods are useful for detecting the presence of a Tourette Syndrome copy number variant. For example a variety of techniques are known in the art for detecting copy number variants within a sample of nucleic acid, including, but not limited to, PCR, RT-PCR, QF-PCR, fluorescent in situ hydridization (FISH) and microarray analysis,

A Tourette Syndrome Copy number variant is optionally detected using the microarrays described herein which are designed to detect copy number variants.

In one embodiment, genomic DNA from a test subject who may have Tourette syndrome or be at a risk of Tourette Syndrome is optionally labeled with a first fluorophore, optionally Cy3 or Cy5, and the genomic DNA from a reference subject or a reference genome is optionally labeled with a second fluorophore, optionally Cy3 or Cy5. The labeled DNA is competitively hybridized to a microarray chip comprising oligonucleotide probes complementary to a Tourette Syndrome critical region. Differential binding of the test genomic DNA sample and a reference genomic DNA sample to at least one oligonucleotide probe complementary to a Tourette Syndrome critical region indicates a Tourette Syndrome copy number variant. For example, higher binding of the test genomic DNA sample than the reference genomic DNA sample to at least one oligonucleotide probe indicates a duplication associated with Tourette Syndrome and higher binding of the reference genomic DNA sample than the test genomic DNA sample to at least one oligonucleotide probe indicates a deletion associated with Tourette Syndrome.

In another embodiment, a Tourette Syndrome copy number variant is optionally detected using Quantitative Fluorescent PCR (QF-PCR). Using this method, primers are used to amplify genomic sequence contained with a Tourette Syndrome copy number variant such as the Tourette Syndrome copy number variants described herein. A person of skill in the art could readily design primers to amplify a copy number variant. The primers are used to amplify genomic sequence from a test subject and a reference standard (for example a reference sequence such as human genome assembly 19). QF-PCR is used to analyze the amount of nucleic acid amplified from the test subject and the reference standard. An increase in amplified sequence from the test subject compared to the reference standard indicates a duplication in the test subject. A decrease in amplified sequence from the test subject compared to the reference standard indicates a deletion in the test subject.

In one embodiment, Tourette Syndrome copy number variants are first detected using a microarray, and then the copy number variant is confirmed using a secondary method such as QF-PCR.

### Kits

Another aspect of the disclosure is a kit for screening for, diagnosing the presence of, or detecting a risk of developing, Tourette Syndrome. In one embodiment, the kit comprises one or more isolated nucleic acid molecules and/or reagents described herein and instructions for use. In another embodiment, the kit comprises one or more isolated nucleic acid molecules and/or reagents described herein and a container for holding or storing the isolated nucleic acid molecules and/or reagents

In an embodiment the kit comprises an isolated nucleic acid molecule or composition that specifically hybridizes to Tourette Syndrome copy number variant, e.g. a probe or a primer. In an embodiment the nucleic acid molecule sequence is complementary to a Tourette Syndrome copy number variant or a portion thereof or the complement thereof. In another embodiment, the nucleic acid molecule comprises a detectable label such as a fluorescent molecule. In a further embodiment, the kit comprises an isolated nucleic acid molecule useful as a primer.

In certain embodiments, the kit is a diagnostic kit for medical use. In other embodiments, the kit is a diagnostic kit for laboratory use.

In another aspect the disclosure provides a commercial package comprising an isolated nucleic acid or reagent described herein and instructions for use.

The following non-limiting examples are illustrative of the present disclosure:

### EXAMPLES

Embodiments of the disclosure will be illustrated in a non-limiting way by reference to the examples below.

### Example 1

### Identification of “Rearrangement Hotspots” within Segmental Duplications in Humans

### Detection of Segmental Duplication (SD) Units

Given that SDs intuitively consist of common repeat elements, SDs were fragmented into multiple smaller SD units which did not overlap with known repeat elements during the read depth-based analysis. In this study, 20,237 non-redundant sets of SD units with at least one inter- or intra-chromosomal rearrangement event were identified, representing 16.65 Mbp of SD units residing outside of common repeat elements in the human genome. At first glance, this total content of SDs may appear small compared with that previously reported [Bailey J A et al, (2002)] and that reported in the database of genomic variants (DGV) which is mainly attributed to methodological differences (i.e., exclusion of common repeats, GC-correction, shorter window length, low read depth threshold). Results from this study and Perry at al [Perry H G at al. (2008)], suggest that previously reported SD breakpoints are overinflated in size, further emphasizing the importance of creating a high-resolution map of ‘rearrangement hotspots’. Read depth distribution for duplicated and non-duplicated regions throughout the genome produced a distinctive distribution pattern with an approximate 7% error rate.

Considering CNVs have a tendency to overlap with nearby SD breakpoints, the results of this study were compared with a recent study which identified common CNV breakpoints in three populations (i.e., 57 Yoruba, 48 European and 54 Asian individuals) [Sudmant P H, 2010]. The detected autosomal SD units greater than 200 by shared 82% concordance (i.e., >50% overlap) with common CNV breakpoints using low coverage short-read data. Moreover, 79% of breakpoints residing within genes with >3 copies as previously reported [Alkan C, 2009], were located within SD breakpoints identified in this study.

Comparison with previous read depth-based reports highlights the advantages of the present hierarchical strategy which include: 1) the use of a 100 by read depth window with a 1 by overlap to detect SD units which enabled the capacity to detect SD units with higher resolution; 2) the use of a lower threshold (i.e., mean+2 standard deviations) than previously reported methods in order to detect homozygous and hemizygous duplications; 3) fragmentation of SDs into smaller SD units in order to separate duplicated regions from common repeated elements while reducing alignment bias for rearrangement analysis and computational time; and 4) integration of end space alignment algorithm with a ‘seed and extend’ clustering technique to the duplicated region of the reference guided assembly sequences to perform an exhaustive search (i.e., 409 million alignments) to identify rearrangement breakpoints.

Compared with copy number gains identified using microarray analysis [Conrad, D F et al. (2010)], sequencing data used in this study revealed that autosomal SD unit breakpoints overlapped 54% with copy number gains [Conrad, D F at al. (2010)], which increased to 67% when compared with 43× coverage [Sudmant P H et al. (2010)]]. Discrepancies are attributed to methodical biases, as detection of structural variants can be specific to different methodical approaches and discrepancies between methods can be as high as 80% [Alkan C et al. (2011)]. The rearrangement analysis within the novel sequence revealed multiple hits within the duplicated sequences (i.e., >90% similarity) that were previously uncharacterized.

### Characterization of Rearrangement Hotspots Within Segmental Duplications

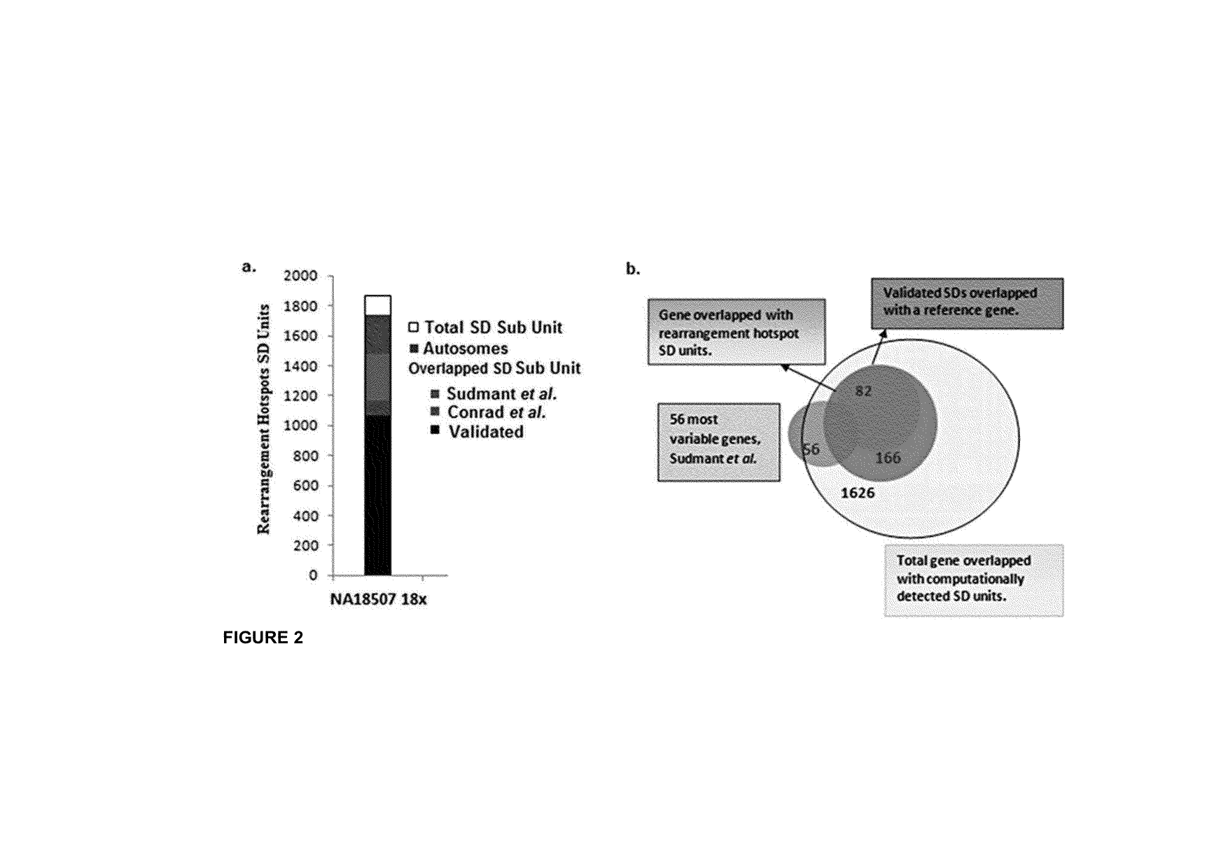

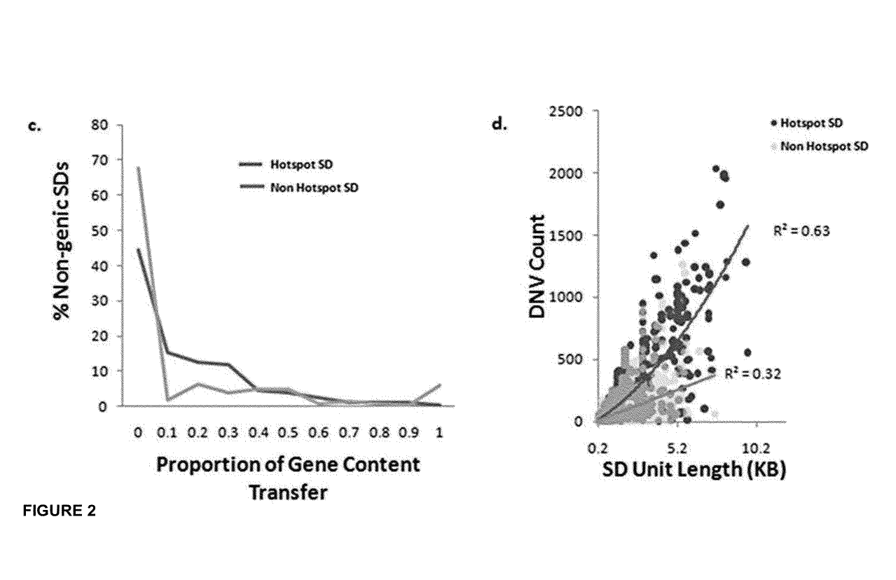

Using 409 million pai/vise alignments, 1963 complex SD units or ‘rearrangement hotspots’ within SDs in the human genome with significantly high distribution of duplicons (p<1.0×10⁻⁶) with at least 10 duplicons per SD unit were identified (FIG. 2a and Table 1). Within these regions, an increase in copy number gain (i.e., increase of 62% in copy number gains within hotspots) with at least 50% overlap with SD units and CNV breakpoints has been observed compared with a previous report [Conrad, D F et al. (2010)]. Importantly, 25% of these ‘rearrangement hotspots’ (i.e., 489/1963) overlapped with 166 unique genes (FIG. 2b) of which 77% (i.e., 375/489) were contained within 82 genes with increased copy number gain that have been previously validated using microarray analysis [Conrad, D F et al. (2010)]. That 25 of these genes are highly variable in copy number within three populations indicates population-specific frequency of the underlying events in the origin of CNVs [Sudmant P H et al. (2010)] which, in turn, implies an increase in frequency of genomic rearrangement events within hotspot regions. However, the extent of gene conversion within the NAHR hotspot is still unknown. Also observed was a relative increase of gene content transfer within agenic hotspot regions (i.e., approximately 50%) compared with the remainder of agenic non-hotspot duplicated regions (i.e., 32%) (FIG. 2c). The finding of elevated levels of gene content transfer is consistent with a previous study which hypothesized such a finding as an apparent feature for hotspots arising from homologous recombination [Gu W et al. (2008)]. Further analysis on duplicated gene variants (DNVs), which is a special type of paralogous sequence variant, was compared between the hotspot and non-hotspot duplicated regions [Ho MR at al. (2010)]. A 3-fold increase in DNVs located within hotspots compared with the remainder of the duplicated regions (p<0.0001) was observed which implies greater diversity within hotspot regions. This finding is attributed, in part, to the accumulation of DNV-derived mutations among derivative homologous sequences within hotspot regions. A strong positive correlation (R²=0.63) between the length and the incidence of DNVs within hotspot regions was also observed (FIG. 2d). Genome-wide read depth comparison revealed that a subset of high read depth regions are positively correlated with rearrangement hotspots (FIG. 2e).

### Distribution of Inter- and Intra-Chromosomal Rearrangements

Segmental duplications (SDs) can be categorized according to the location of the rearrangement considering that recombination events can occur between homologues (i.e, inter-chromosomal) or by looping out within a single homologue (i.e., intra-chromosomal). The analysis revealed that 7% of genes (i.e., 1,626/22,159) overlapped with 5,502 non-redundant SD units which represented 73% (i.e., 41/56) of the most highly variable genes previously identified in the human genome within three populations [Sudmant P H at al. (2010)] (FIG. 2b). 91,971 duplicons (i.e., average of 4.5 duplicons per SD unit) with overlapping breakpoints throughout the SD regions were observed. Extreme inter- and intra-chromosomal rearrangements occurred in 10% of genes (i.e., 166/1626) that overlapped with SD units, of which 50% have been previously validated [Conrad, D F at al. (2010)]. Further analysis revealed that genic regions were enriched with intra-chromosomal recombination, whereas agenic regions evolved through both inter- and intra-chromosomal recombination. Such intra-chromosomal recombination within genic SD units may represent conserved genomic organizations subject to gene conversion and concerted evolution [Bailey J A, 2002; Gu W, 2008; Lieber M R, 2003]. Extreme variation, attributed in part, to SDs has been reported in at least 20% of the copy number variable gene families in three human populations [Sudmant P H et al. (2010)].

Previous cytogenetic studies have demonstrated that pericentromeric and subtelomeric SD regions are strikingly polymorphic and both represent hotbeds for genomic rearrangement [Mefford H et al. (2002) and She X et al. (2004)]. Investigation of recombination within SD units revealed that pericentromeric regions of chromosomes 2, 5, 7, 10, 15, 16, 17, 22 and Y were enriched with inter-chromosomal recombination, whereas only chromosome 11 was associated with intra-chromosomal breakpoints. Subtelomeric regions of chromosomes 1, 2, 4, 7, 9, 10, 11, 16, 19, 20, 22, and X were enriched with inter-chromosomal recombination, whereas chromosomes 3, 6, 12, 13, 14 and Y were associated with extreme intra-chromosomal breakpoints. This idiosyncratic rearrangement pattern suggests that multiple translocations involving distal regions of chromosomes create complex breakpoints within SDs. This is exemplified by the pseudoautosomal region 1 (PAR1) which displayed extensive inter- and intra-chromosomal tandem duplications, consistent with sex chromosome evolution. Another complex region where extensive intra-chromosomal rearrangements were identified is the distal heterochromatic region of the Y chromosome (i.e., Yq12), housing the male specific (MSY) region. In the analysis, both homozygous and hemizygous duplications were detected using read depth information which represents an extension to previous SD analysis [Sudmant P H et al. (2010) and Alkan C et al. (2009)] by the inclusion of sex chromosomes.

Complex rearrangements in multiple gene families where rapid evolution of NBPF, PRAME, RGPD, GAGE, LRRC, TBC1, NPIP and TRIM gene families were identified. Without being bound by theory, this appears to be predominantly attributed to intra-chromosomal gene transfer, whereas other complex gene families (e.g., ANKRD, OR, GUSB, FAM, POTE, ZNF and GOLG) appear to be more diverse with respect to transfer of gene content, occurring both within and between chromosomes. As previously reported [Alkan C et al, (2009)], the DUX family gene was associated with the most copies within the reference genome. The rearrangement analysis of the novel sequence within 10q26.3 region suggests at least 10 additional copies of the DUX4 gene is specific to novel sequences within the NA18507 human genome.

Gene Ontology Analysis within ‘Rearrangement Hotspots’

To investigate the impact of genes residing within ‘rearrangement hotspot’ regions identified in this study and theft relation to complex disease, genes were functionally categorized using PANTHER gene ontology analysis. Genes residing within ‘rearrangement hotspot’ regions appear to be involved in functions associated primarily with nucleic acid metabolism (22%) and cellular processes (16%), although associations also exist for developmental process (9%), cell cycle (9%), and cell communication (8%). This finding is consistent with a previous report in which copy number gains were associated with genes involved in nucleic acid metabolism and developmental processes, whereas copy number losses were enriched for genes involved in cell adhesion [Park H et al. (2010)]. That genes residing in ‘rearrangement hotspot’ regions are consistently associated with functions affecting multiple processes important in normal growth and development, further underscores the critical role that rearrangement hotspots play in the genetic etiology of complex disease.

### Clinical Relevance of ‘Rearrangement Hotspots’

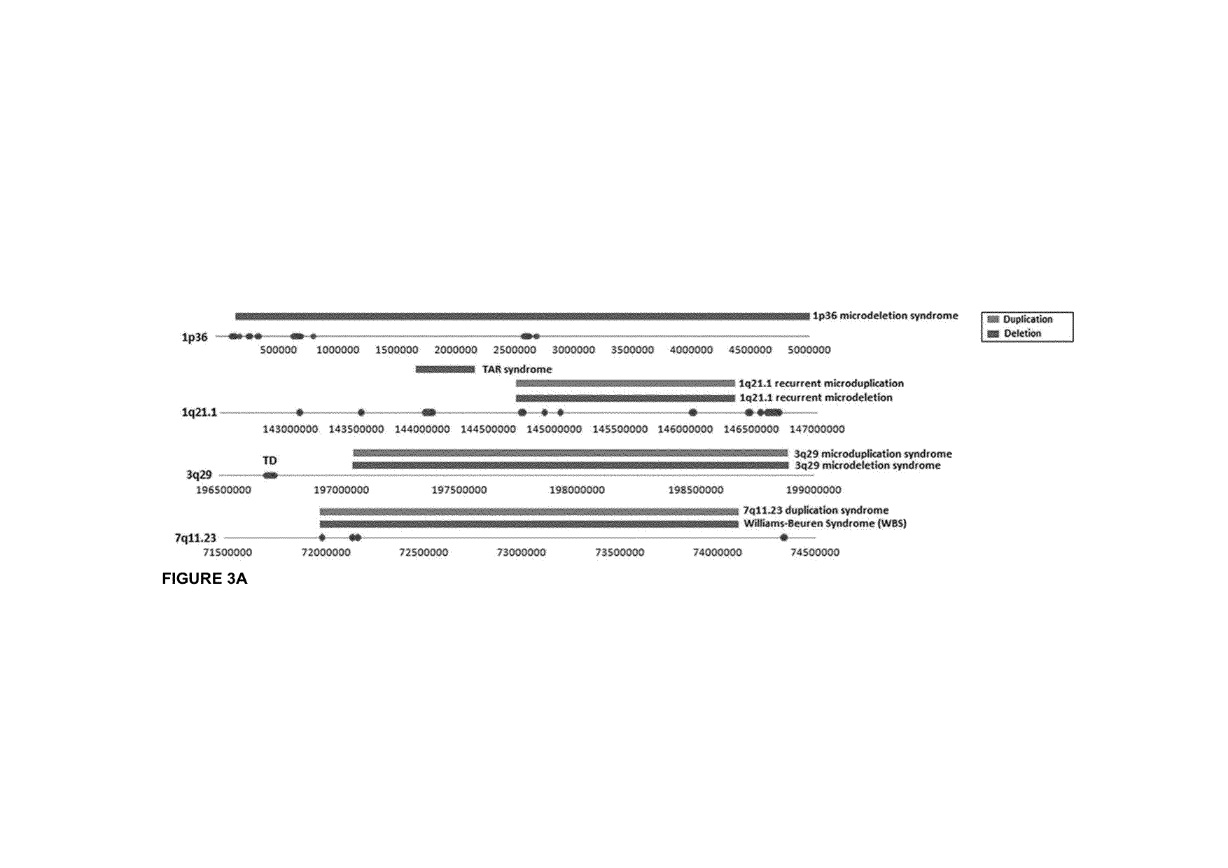

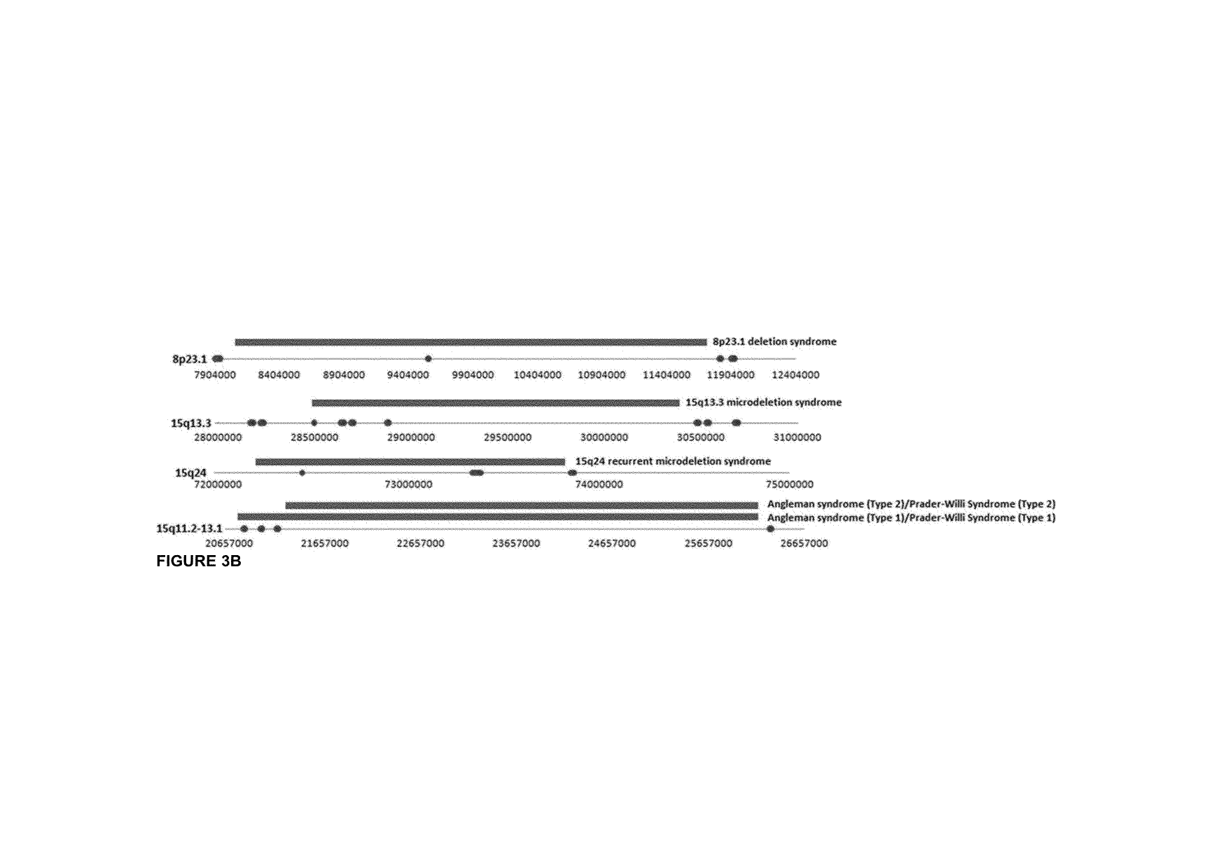

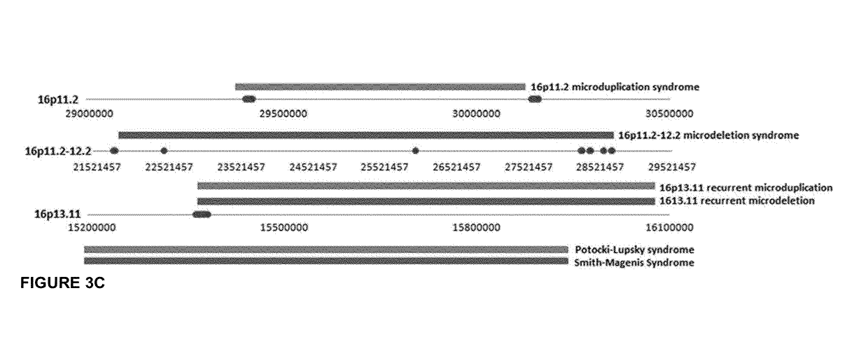

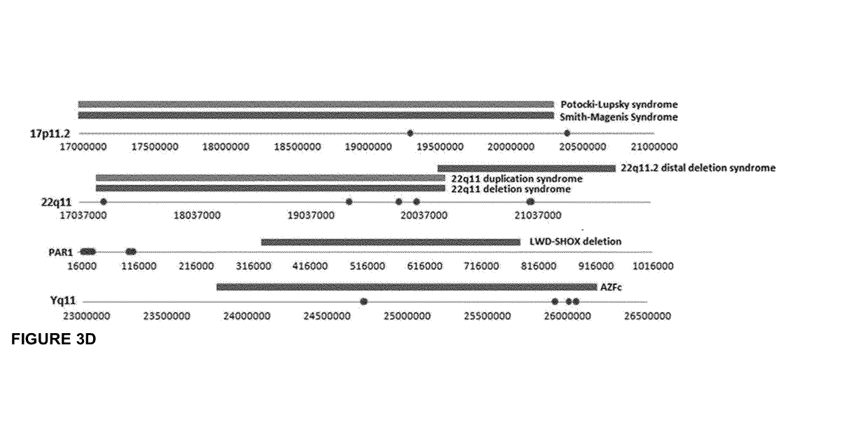

A genome-wide high resolution map of ‘rearrangement hotspots’ has been produced. Without being bound by theory, these ‘rearrangement hotspots’ likely serve as templates for NAHR and consequently may represent an underlying mechanism for development of constitutional and acquired diseases arising from de novo deletions or duplications. A collection of 24 previously identified genomic disorders predominantly mediated by de novo NAHR events are catalogued in the DECIPHER database [DECIPHER Genomic Aberration Database: http://decipher.sanger.ac.uki]. Comparison of the hotspot regions identified in the present study with pathogenic deletions/duplications breakpoints mapped for those genomic disorders constituting only 15 common genomic loci revealed that 20% of the detected hotspots are clustered within proximal and distal SDs that are flanked by these pathogenic deletions/duplications (FIG. 3). This finding indicates a higher rate of NAHR within the genome-wide rearrangement hotspot regions detected in this study.

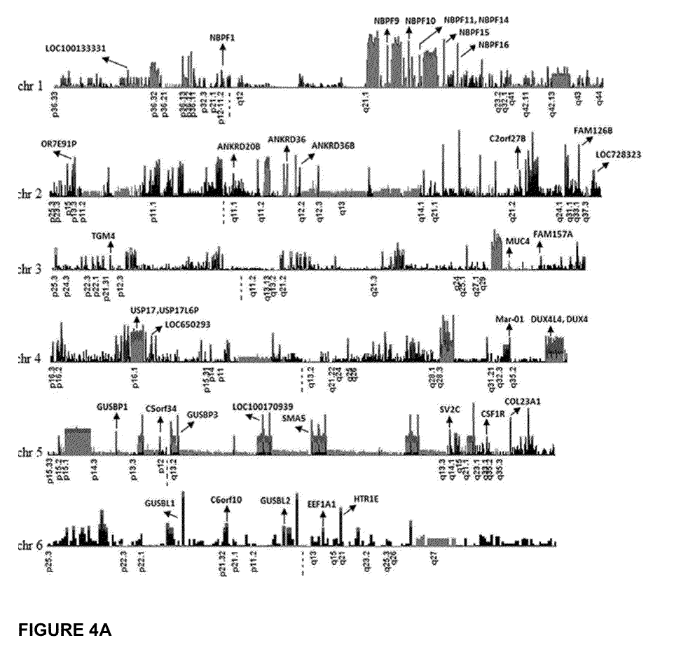

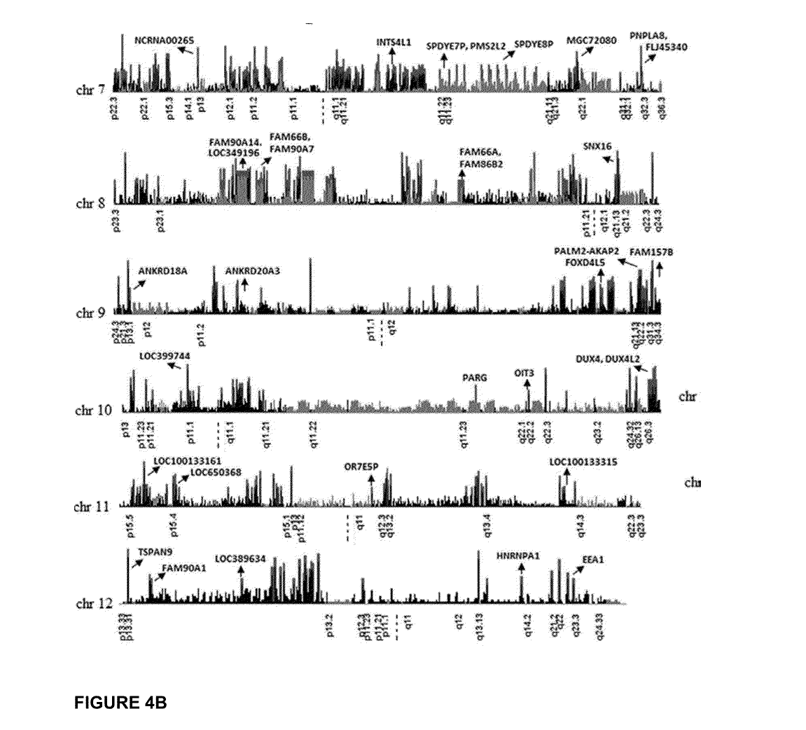

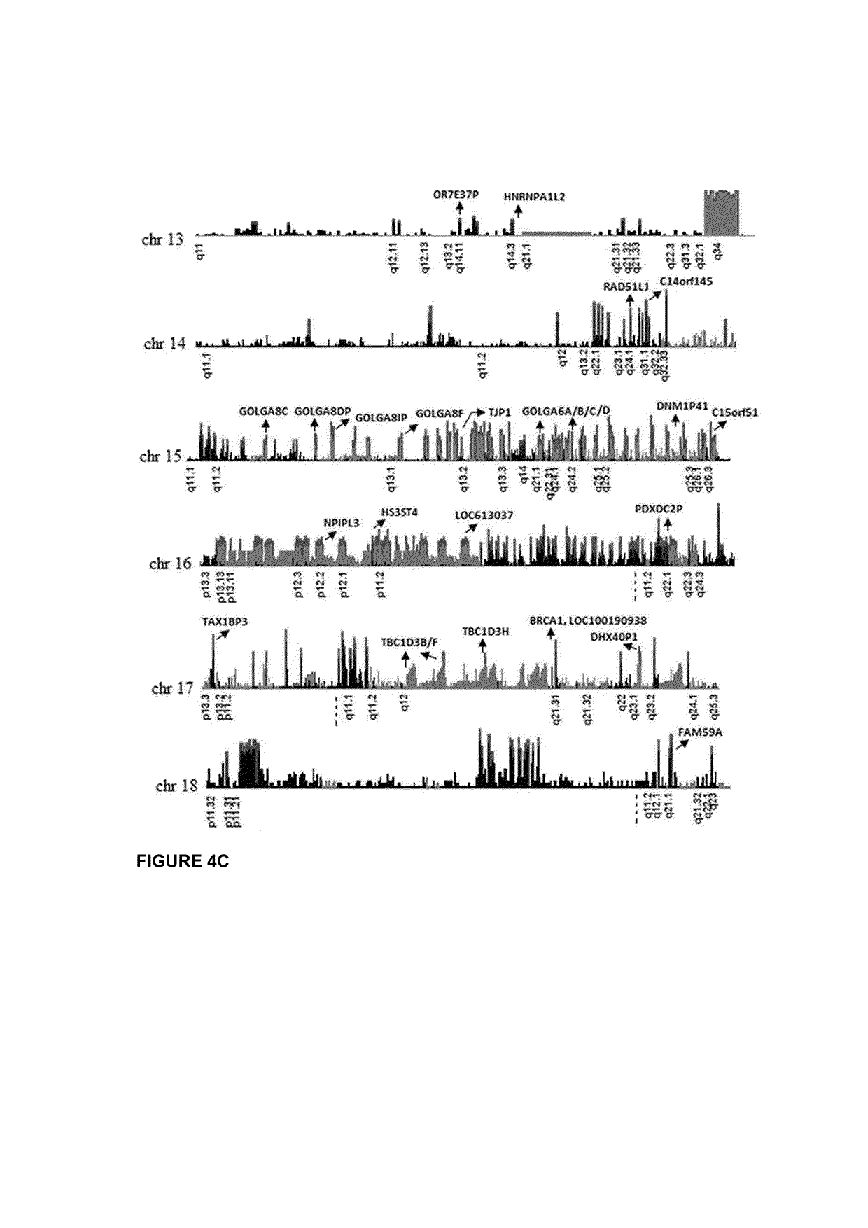

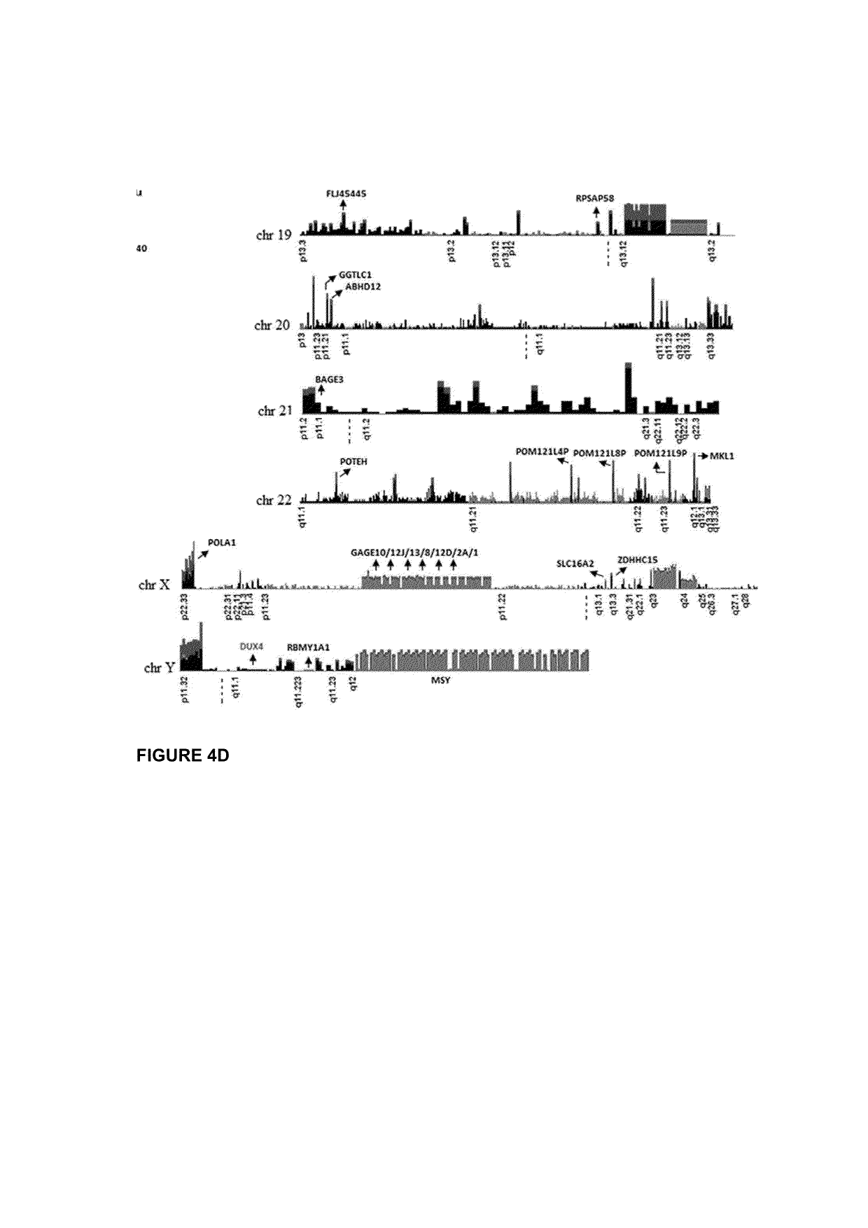

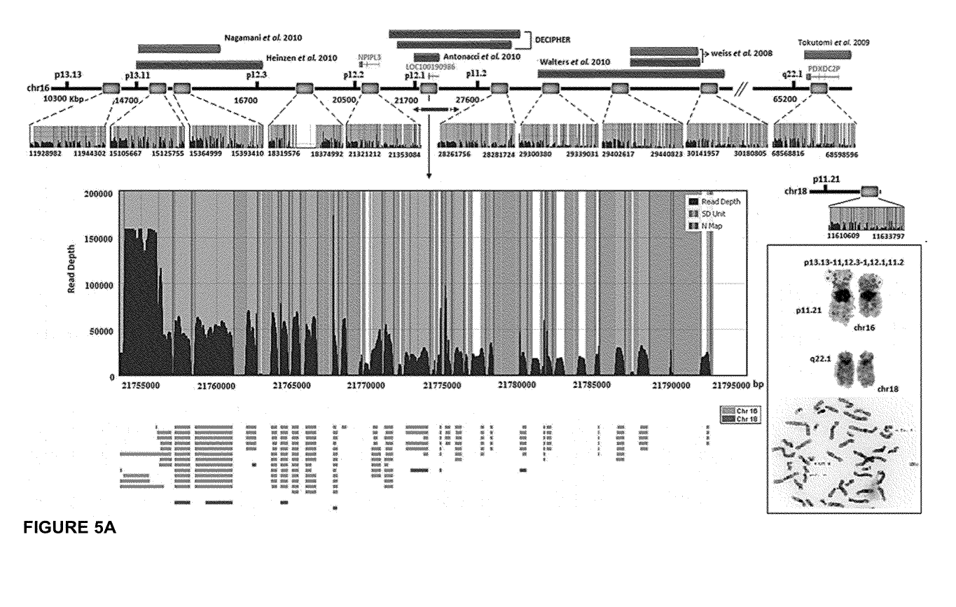

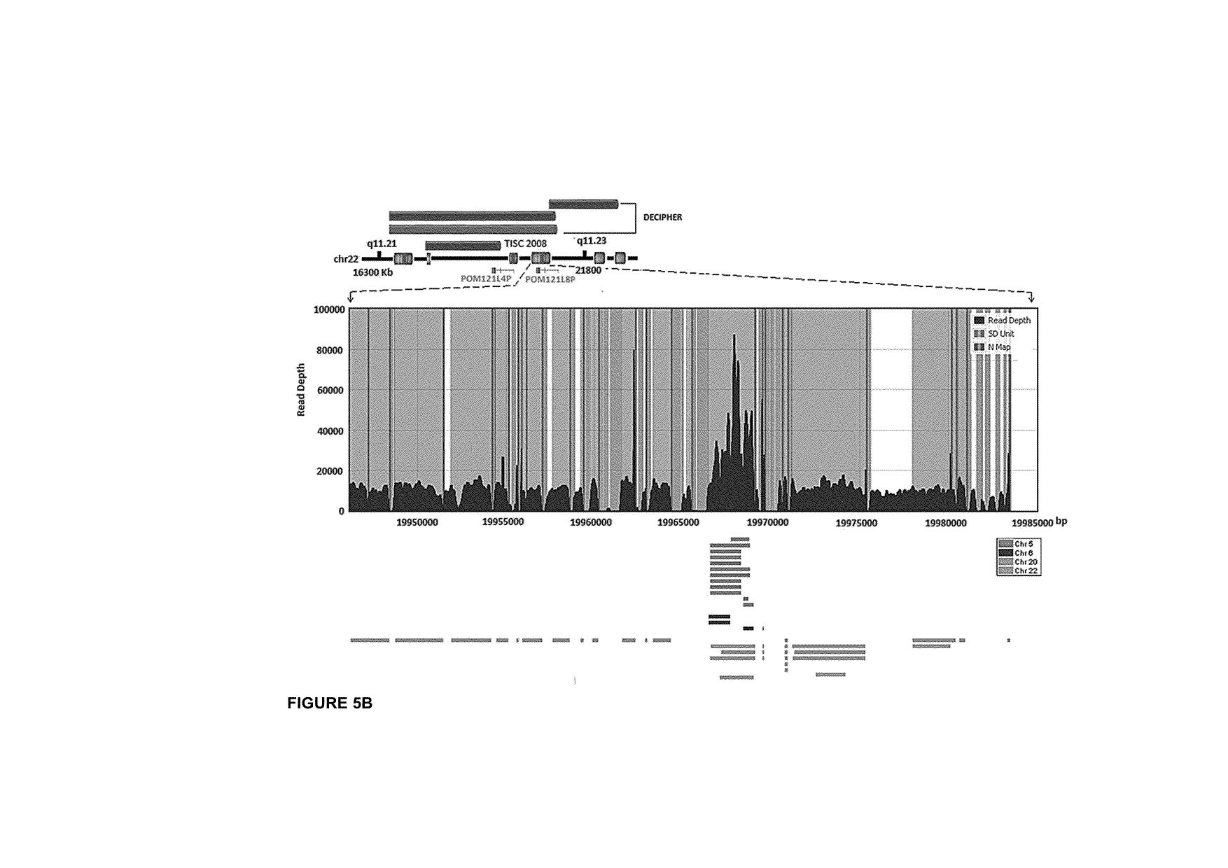

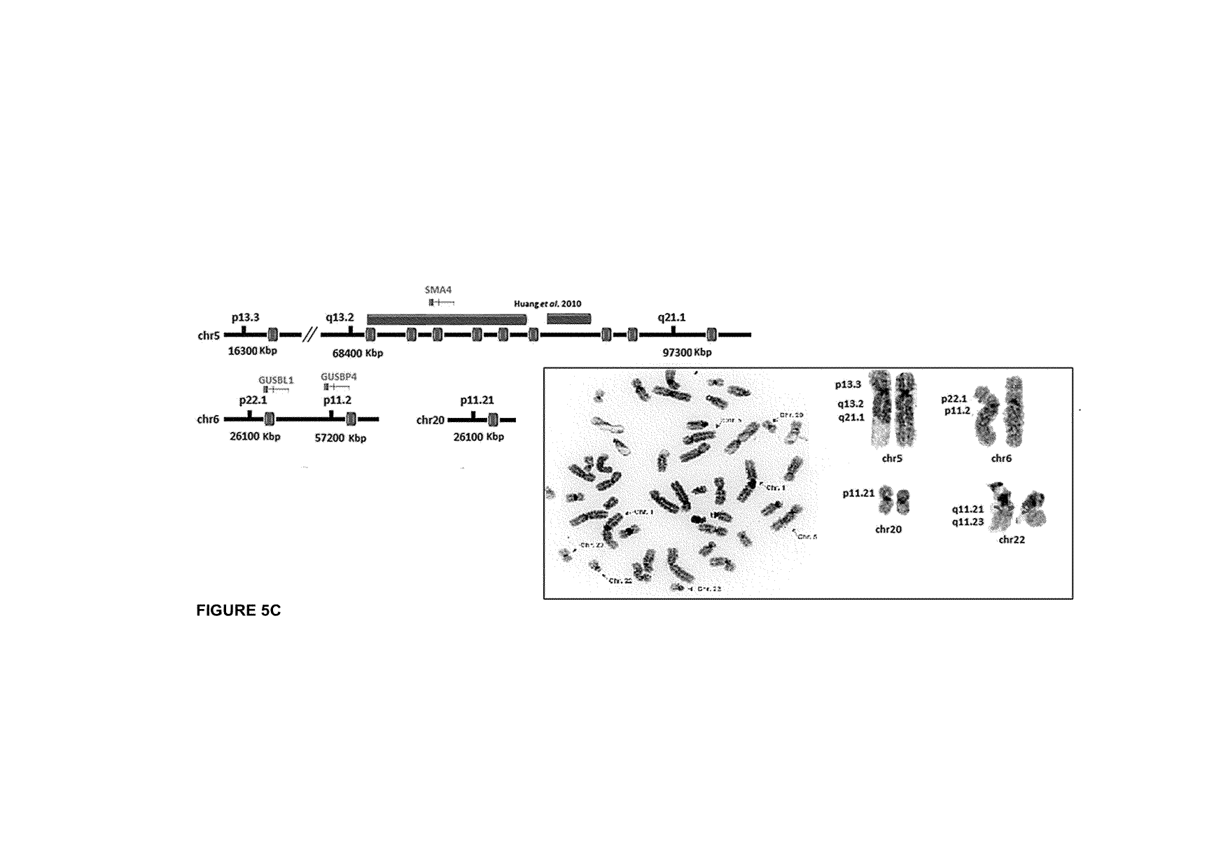

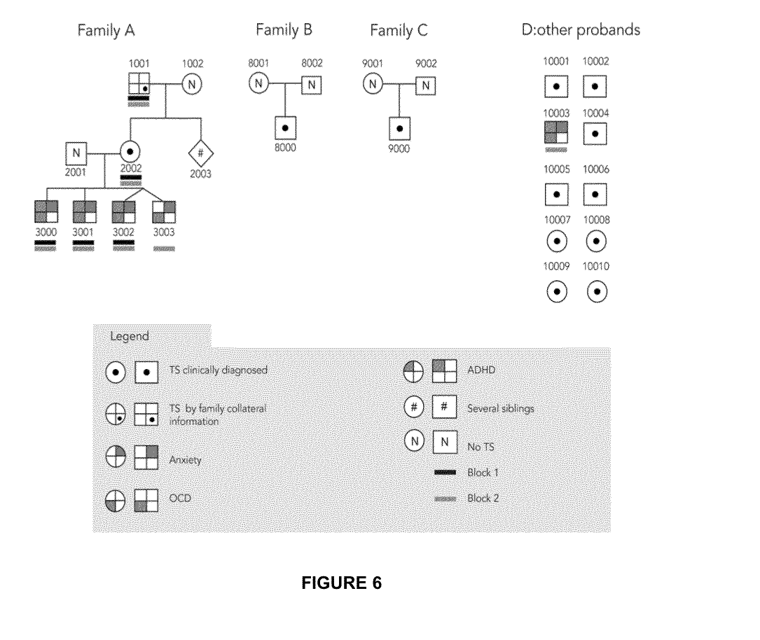

The rearrangement structure of these hotspots based on the present in silico predictions (FIG. 4) reveals the complex architecture associated with SDs. To validate the complexity of these hotspots, FISH analysis was performed on selected regions harbouring hotspot clusters demonstrated 94% (i.e., 17/18) concordance with in silico predictions of co-localization (FIGS. 5a, 5b, and 6). One example of an identified ‘rearrangement hotspot’ is a duplication at the 16p12.1 complex region, which contains an 32 inversion [Park H et al. (2010)], where the alignment localized multiple derivatives of the NPIPL3 gene within chromosomes 16 and 18 (FIG. 5a). The identified breakpoints revealed the presence of derivative copies of the NPIPL3 gene within the short arm of chromosomes 16 and 18, possibly attributed to NAHR-mediated recombination, where pathogenic deletions and duplications have been reported in patients with mental retardation and intellectual disability [Antonacci F et al. (2010); Nagarnani S C et al. (2011); Heinzen E L et al. (2010); Weiss L A et al. (2008); Walters R G et al. (2010); Ballif B C et al. (2007); and Tokutomi T et al. (2009)]. The derivatives are located within the pathogenic deletion breakpoints among the patients with neurodevelopment disorders. Unfortunately, these studies used methodologies unable to localize derivative copies, and consequently the NPIPL3 gene was disregarded as a susceptibility gene. A second complex region, 22q11.21, housed a large duplication consisting of two copies, with the ‘core duplicon’ being copied multiple times in chromosomes 5, 6, 20 and 22 (FIG. 5b). Phenotypes attributed to pathogenic deletions and duplications within chromosomes 5 and 22 [Ensenauer R E et al. (2003) and Huang X S et al. (2010)] revealed breakpoint patterns within a ‘core duplicon’, suggestive of NAHR-mediated duplication.

A third complex region, revealed a previously uncharacterized gene desert within 1q21 indicating a possible harvest region for the NBPF gene family. This 68 Kbp gene desert region revealed extreme intra-chromosomal rearrangement without any signature of inter-chromosomal duplication in our in silico analysis (FIG. 6). The gene fragments from NBPF1,3,9,10,14,15,16,20 and 24 appear to be copied and transferred to 1q21.1 (142867911-142935940) and consequently creating extreme overlapping tandem duplications. The fosmid clone G248P8712C10 covering this region was used on metaphase chromosomes to predict derivative duplicated loci. Multiple signals were obtained within 1p36.12 and 1q21.1 regions, while a weak signal was obtained within the 1p10p13 region which was not detected by our in silica analysis. The donor region located 2 Mb distal from the gene desert transferred gene content to this 68 kbp region which is associated with recurrent pathogenic deletions and duplications implicated in developmental disorders and neuroblastoma [DECIPHER Genomic Aberration Database: http://decipher.sanger.ac.uk/; Diskin S J at al. (2009); and Brunetti-Pierri N at al. (2008)]. Without being bound by theory, gene deserts may represent reservoirs for creation of novel genes and underscores the necessity to further explore this previously ignored region of the human genome. The complexity of tandem duplications (e.g., 1q21.1) can have a direct impact on estimating copy number for a gene (e.g., NBPF). In such cases, the estimation of copy number based solely on read depth may be affected due to the nature of the tandem duplication.

### Materials and Methods

Data Acquisition and Processing

Short read data was obtained for the NA18507 human genome sequenced using reversible terminator chemistry on an Illumina Genome Analyzer [Bently D R, 2008]. The original data consisted of >30× coverage of the genome. More than half of the data was obtained from the Short Read Archive Provisional FTP (NCBK) site (ftp://ftp.ncbi.nih.gov/pub/TraceDB/ShortRead/SRA000271/) with an average read length of approximately 36 bp. The analysis accuracy of this dataset has been previously described [Bently D R et al. (2008)]. The 4.8 Mb novel sequence detected in the NA18507 genome by a previous de novo assembly was also integrated in our rearrangement analysis. The length distribution revealed that the contigs/scaffolds are over fragmented and >80% of the sequence length is <1 kb in length. The NA18507 human genome was selected as it is representative of the ancestral African Euroban population which has been previously shown to contain the most diverse polymorphisms compared with other populations [Sudmant P H et al. (2010) and Alkan C et al. (2009)], rendering it an ideal sample to generate a ‘rearrangement hotspot’ map as the majority of the hotspot regions detected should exist within other populations.

### Short Read Mapping

mrsFAST (micro-read substitution only fast alignment search tool—version 2.3.0.2) was applied which implements an all-to-all algorithm unlike other short read mapping algorithms [Hach F et al. (2010)]. Specifically, it is a fast alignment search tool which uses cache oblivious short read mapping algorithm to align short reads in an individual genome against a repeat masked reference human genome within a user-specified number of mismatches. The short reads were mapped using mrsFAST with a maximum of two mismatches allowed against the repeat masked (UCSC hg18) genome assembly. mrsFAST returns all possible hits in the genome for a short read, allowing the detection of differential read depth distribution within duplicated regions of the human genome. Using the NA18507 human genome (18× coverage), 1.5 billion short reads were processed with 55.78% (i.e., ˜839 million short reads) mapped to the repeat masked human reference genome with the mrsFAST aligner which returned all possible mapping locations of a read; a key requirement to accurately predicting the duplicated regions within the reference genome.

### GC Correction

There exists a known bias with next generation sequencing technology towards GC-rich and GC-poor regions. Moreover, during library preparation using an illumina Genome Analyzer, amplification artefacts are introduced in both GC-poor and GC-rich regions producing an uneven distribution of read coverage [Alkan C et al. (2009)] which has the potential of detecting false positive duplicated regions. To reduce this bias, a simple GC correction method was used. Overlapping windows (i.e., by 1 bp) with length ‘/’ was used for read depth computation. Each read was assigned only once by its starting position and read depth was computed for each chromosomal position. The original mean read depth was calculated for each length (i.e., 100 bp) block using equation (1). G+C percentage for every 100 by window from the reference human genome and the read depth was computed and subsequently interrogated for adjustment. The adjusted read depth was computed using the following equation:

$${RD}_{i} , adjusted = {RD}_{i} \times \frac{m 1}{m 2} \qquad ( 1 )$$

where RDᵢ, adjusted is the read depth after GC correction, RDᵢ is the original read depth computed for i^{th} window, m1 is the overall median of all the windows with 100 by length and m2 is the mean depth for all windows with same GC percentage. All subsequent analysis was carried out on the GC-corrected read depth.

### Read Depth (RD) and Interval Detection

The first step in dissecting SD unit breakpoints using the NA18507 genome from all hit map information was to compute read depth from short read sequence mapping and detect SD intervals that do not overlap with a repeat region of the genome. Read depth was computed for each point after obtaining mapped anchoring positions of the short reads from mrsFAST. A table was built for each chromosome, each containing coordinates where the common repeats are located. The read depth mean was computed for a chromosome from the genome content excluding common repeat regions. For each window with l length (100 bp) an event was determined. Events with excessive read depth and with a deletion were detected using equation (2).

$$event ( l ) = \{ \begin{matrix}2 ( ExcessiveRD ) & if {\sum }_{k = 0}^{l} RD \geq mean + 2 \times st . d \\ 0 ( Deletion ) & if RD < 1 \\ 1 & otherwise ;\end{matrix} \qquad ( 2 )$$

To investigate the interrogating window if it falls within a common repeat elements, a library for the repeat masked regions (masked interspersed repeats, i.e. LINES, SINES, etc.) of the human genome was built. The mean length of the detected SD units was 822 bp. The read depth distribution between the detected duplication subunits and the non-duplicated regions of the genome show significant read depth differences with an approximately 7% error rate.

### NA18507 Short Read Reference Guided Assembly

The current version of mrsFAST does not return the quality of the aligned reads within a consensus genome. Instead, MAQ version 0.7.1 (Mapping and Assembly with Quality) was used which assembles genomes with a specified quality. MAQ searches for the un-gapped match with lowest mismatch score (i.e., maximum of 2) in the first 28 bp. To confidently map alignments, MAQ assigns each alignment a Phred scaled quality score which measures the probability that the true alignment is not the alignment that is detected by MAQ. If a short read maps to multiple positions in the genome, MAQ will randomly pick one position and give the excluded position a mapping quality of zero. The NA18507 genome short reads were mapped and assembled into the reference genome using MAQ allowing at most 2 mismatches.

### Detection of Genomic Re-Arrangements

Using read depth as a measure to detect SD unit breakpoints may produce regions that share <90% sequence identity. To reduce false positive and computational burden after detecting SD unit breakpoints, a basic version of the end space alignment algorithm (without seed and extend approach) was utilized and a pairwise alignment for each of the SD units against the rest of the genome SD units was performed. Only those SD units for rearrangement analysis described in the following section that contained at least one duplicon >100 by with >90% sequence identity were included. 20,237 SD units when every 100 by window was assessed for a possible rearrangement were detected.

### End-Space Free Alignment Algorithm

The ability to detect highly homologous regions between two sequences is essential for duplicon detection. Multiple clusters of non-adjacent duplicons with >90% sequence identity cannot be mapped using basic alignment algorithms. As previously reported, the basic pairwise global alignment algorithm will miss duplicon breakpoints that are non-adjacent within an SD with different thresholds of sequence identity [6]. Semi-global alignment has a tendency to produce pattern-like alignments (see example below), which are not informative for complex regions with multiple duplications. A modified version of the pairwise alignment algorithm was implemented where the alignments are scored ignoring end spaces of the two sequences. Adding the option of end spaces in our alignment does not produce pattern-like alignments and therefore accurately pinpoints the breakpoints of the duplicon with an allowed gap that crosses the threshold of >90% sequence identity. The neutral rate of evolutionary decay suggests that 10% sequence divergence is required to accurately detect duplications that are primate-specific [Gu W et al. (2008)].

### Example

S1:
(SEQ ID NO: 1)
ACGCAATTCGACTAGATCGGGTCGATGATCGATCGATGATCGAGACA
GCATAGCAG
S2:
(SEQ ID NO: 2)
CAATTCGACTAGATCGATCGACGATCGATCGAT
Semi-Global Alignment:
S1:
(SEQ ID NO: 1)
ACGCAATTCGACTAGATCGGGTCGATGATCGATCGATGATCGAGACAGC
ATAGCAG
S2:
(SEQ ID NO: 3)
***CAATTCGACTAGATC*GATC***GA*CGATC***GAT*****C*G*
AT*****
End-Space Free Alignment:
S1:
(SEQ ID NO: 4)
CAATTCGACTAGATCGGGTCGATGATCGATCGAT
S2:
(SEQ ID NO: 5)
CAATTCGACTAGATC*GATCGACGATCGATCGAT

In order to implement the algorithm, a dynamic programming technique was utilized which is a modified version of Smith-Waterman dynamic programming [Smith I F et al. (1981)]. This approach will detect the pairwise alignment relative to a penalty function corresponding to semi-global alignment. The dynamic programming (DP) algorithm was used to compute the above alignments and the backtrack pointer was used to identify the best alignment.

### Dynamic Programming Matrix and Recursive Trace Back

As a core searching algorithm, a penalty function was implemented to complete the dynamic programming matrix M. First, the first column and row was initialized with zeroes which provided forgiving spaces at the beginning of the sequences in order to obtain the highest similarity between the interrogated sequences. The intention was to locate duplicons between a pair of sequences (i.e., s and t) with >90% identity and alignment with minimal gaps to avoid pattern-like structures. “A” was encoded with 1, “G” with 2. “C” with 3 and “T” with 4 to construct the (m+1)×(n+1) DP matrix M, where m and n is the length of two given sequences s and t, respectively. The algorithm uses a dynamic programming technique to fill a matrix M by a look up penalty function from the 5×5 matrix C. A penalty function g(i,j) was introduced for matched alignment with a score of 2. For the mismatches between a pair of bases, a penalty of −2 was introduced for mismatch and −3 for misaligned sequence produced by sequence assembly tools (i.e., MAQ). A −3 penalty was used to reduce the amount of misaligned portions of the sequence into duplicon identification. To allow the algorithm to ignore the end positions of the sequences if it has low similarity, a trace back from the highest value returned by function Sim(s,t) in the matrix M was performed. For any two given sequences (i.e., s and t), a semi-global alignment is an alignment between a substring (in this case duplicon) of s and t.

$$\begin{matrix}a [ i , j ] = max \{ \begin{matrix}a [ i , j - 1 ] - 3 \\ a [ i - 1 , j - 1 ] + g ( i - 1 , j - 1 ) \\ a [ i - 1 , j ] - 3\end{matrix} & ( 3 ) \\ Sim ( s , t ) = max of M & ( 4 )\end{matrix}$$

The memory requirement to fill out DP matrix M is O(mn). The computational time to complete the dynamic programming Matrix M and to determine the maximum value in M for a given pair of sequence s and t with nearly similar length is O(n²) and to trace back starting from the maximum point in the matrix takes O(m+n) time to obtain optimal alignment.

It might be apparent that ignoring end spaces might not detect true breakpoints and for long sequences it might produce really short alignments. Considering that majority of the commonly used alignment search methods (i.e., BLAST, BLAT, and SHRiMP) implement a “seed and extend” method to obtain faster sequence comparison [Altschul S F, 1990; Kent 2002; Yanovsky V, 2008; Mi, 2010], this method was also applied in this study. To perform an exhaustive search within the scope of 100 by windows for any two given segmental unit sequences obtained from NA18507 genome, the dynamic programming algorithm for each 100 by window with 10 by overlaps as “seeds” was applied. The highly similar seeds (>90%) went through the “extend” step and the rest was ignored. As this approach might detect the same breakpoints multiple times if multiple seeding events are obtained from a highly duplicated region, the previously extended duplicon breakpoints from the same SD unit and the overlapping “seeds” was compared and only the maximum extended duplicon was kept. ‘Extend’ is a recursive procedure which extends bi-directionally by 10 bp and the extend step ceases in each direction when further extension does not cross the sequence identity threshold. As a result, the procedure terminates if any further extension of both directions returns <90% sequence identity.

### FISH Validation

Cytogenetic preparations were made from lymphoblastoid culture (obtained from Coriell cell repositories) for the NA18507 sample. The cell suspension was dropped on slides using a thermotone, aged overnight and hybridized with test (i.e., spectrum orange) and control probes. Following post-hybridization washes and 4,6-diamidino-2-phenylindole (DAP1) counterstaining, slides were analyzed using fluorescence microscopy. Pseudocoloring and image editing was performed using Photoshop software. To validate duplicon rearrangement within SD units, three complex regions in the human genome: 1q21.1, 16p12.1 and 22q11.21 were selected. In this study, fosmid genomic clones corresponding to a duplicated locus as a probe against chromosomal metaphase were used. The localization of FISH clones within these regions and the corresponding derivative loci validated >94% (i.e., 17/18) of the in silico co-localization predictions. The FISH technique was unable to provide a precise estimate of rearrangement at the level of 100 bp due to resolution limitations.

### Permutation

The basic analyses were conducted using a permutation procedure to assess statistical significance of 1-sided tests. The rearrangement for each SD unit was permuted randomly between the two groups and test statistics was computed in each permutation. All results reported in this study used 1 million permutations to derive an empirical value.

### Gene Ontology Analysis

Gene ontology data analysis was performed using PANTHER (version 7.0) database [Mi H et al. (2010)]. The biological processes of the hotspots genes were analyzed.

### Example 2

### Microarray Chips for Detecting Genomic Aberrations

A custom aCGH microarray was designed based on the rearrangement hotspots identified in Example 1. In all, approximately 500 MB of the human genomic sequence was covered within a 2×1 million probe (1 M) microarray. The Agilent custom microarray identification numbers are 035313 and 035316.

The genomic regions covered by the microarray were chosen as follows:

a) All the breakpoints (ie. “rearrangement hotspots”) identified in Example 1 were accommodated (Table 1).

b) The location of the hotspots and how far they are from each other was considered. If two hotspots were within 1 MB from each other, the entire region between the two hotspots was included.

c) Known CNV regions previously identified in the literature were included.

d) At least 1 MB of the telomeric and centromeric regions for all chromosomes were also included.

Probes were designed to be 45-60 basepairs in length. Probe spacing ranges between 190-500 by with a mean spacing of 280 by within each genomic region covered by the array.

Tables 2-5 contain the specific coordinates based on the NA18507 human genome corresponding to the 500 MB of genomic sequence covered by the microarray. In all, approximately 10% of the probes correspond to previously detected Copy Number Variants and 90% of the probes correspond to regions susceptible to genomic alteration as based on the computational analysis described above.

TABLE 1
1963 Rearrangement Hotspots. Chromosome coordinates
correspond to the NA18507 human genome.

| chr | start | end |
|---|---|---|
| 1 | 102428 | 103655 |
| 1 | 104523 | 105540 |
| 1 | 108919 | 109527 |
| 1 | 109802 | 110931 |
| 1 | 114730 | 115997 |
| 1 | 121334 | 122466 |
| 1 | 124255 | 128240 |
| 1 | 129970 | 130372 |
| 1 | 166300 | 167381 |
| 1 | 247579 | 248325 |
| 1 | 248747 | 249972 |
| 1 | 251288 | 251855 |
| 1 | 255240 | 255848 |
| 1 | 256123 | 257253 |
| 1 | 315614 | 318942 |
| 1 | 320610 | 322337 |
| 1 | 328559 | 328907 |
| 1 | 329466 | 331722 |
| 1 | 627628 | 628412 |
| 1 | 628793 | 630020 |
| 1 | 530725 | 631903 |
| 1 | 635288 | 635896 |
| 1 | 636171 | 637301 |
| 1 | 638039 | 640059 |
| 1 | 641087 | 642355 |
| 1 | 647698 | 648830 |
| 1 | 650621 | 653735 |
| 1 | 657032 | 657289 |
| 1 | 660440 | 660694 |
| 1 | 663849 | 664106 |
| 1 | 665689 | 666094 |
| 1 | 668071 | 668847 |
| 1 | 680877 | 681646 |
| 1 | 688405 | 689651 |
| 1 | 792445 | 794932 |
| 1 | 2573251 | 2576251 |
| 1 | 2576151 | 2579151 |
| 1 | 2579051 | 2582051 |
| 1 | 2581951 | 2584951 |
| 1 | 2584851 | 2587851 |
| 1 | 2587751 | 2590751 |
| 1 | 2590651 | 2593651 |
| 1 | 2593551 | 2596551 |
| 1 | 2596451 | 2599451 |
| 1 | 2599351 | 2602351 |
| 1 | 2602251 | 2605251 |
| 1 | 2605151 | 2608151 |
| 1 | 2608051 | 2611051 |
| 1 | 2610951 | 2613951 |
| 1 | 2613851 | 2616851 |
| 1 | 2616751 | 2624180 |
| 1 | 2675459 | 2683703 |
| 1 | 8876229 | 8877357 |
| 1 | 16762107 | 16768986 |
| 1 | 16773004 | 16777213 |
| 1 | 16779730 | 16783494 |
| 1 | 16786026 | 16789354 |
| 1 | 16790353 | 16791717 |
| 1 | 21623175 | 21627179 |
| 1 | 21677496 | 21683296 |
| 1 | 24193960 | 24194188 |
| 1 | 26674144 | 26675098 |
| 1 | 40571260 | 40571981 |
| 1 | 51488912 | 51489852 |
| 1 | 54775157 | 54775761 |
| 1 | 102024413 | 102026279 |
| 1 | 113243378 | 113243726 |
| 1 | 116200740 | 115202126 |
| 1 | 120835697 | 120836641 |
| 1 | 120836827 | 120338299 |
| 1 | 120338327 | 120840228 |
| 1 | 120842041 | 120842565 |
| 1 | 120842492 | 120343585 |
| 1 | 141629286 | 141632656 |
| 1 | 141638783 | 141642395 |
| 1 | 142025678 | 142029100 |
| 1 | 142031641 | 142032630 |
| 1 | 142035252 | 142038880 |
| 1 | 142241469 | 142245676 |
| 1 | 142867911 | 142870911 |
| 1 | 142870811 | 142873811 |
| 1 | 142873711 | 142876711 |
| 1 | 142876611 | 142879611 |
| 1 | 142879511 | 142882511 |
| 1 | 142882411 | 142885411 |
| 1 | 142885311 | 142888311 |
| 1 | 142888211 | 142391211 |
| 1 | 142891111 | 142894111 |
| 1 | 142894011 | 142897011 |
| 1 | 142896911 | 142399911 |
| 1 | 142899811 | 142902811 |
| 1 | 142902711 | 142905711 |
| 1 | 142905611 | 142908611 |
| 1 | 142908511 | 142911511 |
| 1 | 142911411 | 142914411 |
| 1 | 142914311 | 142917311 |
| 1 | 142917211 | 142920211 |
| 1 | 142920111 | 142923111 |
| 1 | 142923011 | 142926011 |
| 1 | 142925911 | 142928911 |
| 1 | 142928811 | 142935940 |
| 1 | 143074790 | 143075669 |
| 1 | 143532860 | 143541159 |
| 1 | 144020839 | 144023839 |
| 1 | 144023739 | 144026739 |
| 1 | 144026639 | 144029639 |
| 1 | 144029539 | 144032539 |
| 1 | 144032439 | 144035439 |
| 1 | 144035339 | 144038339 |
| 1 | 144038239 | 144041239 |
| 1 | 144041139 | 144044139 |
| 1 | 144044039 | 144047039 |
| 1 | 144046939 | 144049939 |
| 1 | 144049839 | 144052839 |
| 1 | 144052739 | 144055739 |
| 1 | 144055639 | 144058639 |
| 1 | 144058539 | 144061539 |
| 1 | 144061439 | 144064439 |
| 1 | 144064339 | 144067339 |
| 1 | 144067239 | 144070239 |
| 1 | 144070139 | 144073139 |
| 1 | 144073039 | 144076039 |
| 1 | 144075939 | 144080872 |
| 1 | 144744835 | 144753076 |
| 1 | 144757085 | 144761295 |
| 1 | 144763802 | 144767580 |
| 1 | 144925945 | 144933575 |
| 1 | 145049906 | 145053413 |
| 1 | 146041883 | 146050118 |
| 1 | 146054138 | 146058348 |
| 1 | 146060855 | 146064684 |
| 1 | 146470485 | 146478397 |
| 1 | 146478328 | 146484957 |
| 1 | 146488986 | 146492763 |
| 1 | 146568347 | 146569228 |
| 1 | 146617876 | 146620876 |
| 1 | 146620776 | 146623776 |
| 1 | 146623676 | 146626676 |
| 1 | 146626576 | 145629576 |
| 1 | 146629476 | 146632476 |
| 1 | 146632376 | 146635376 |
| 1 | 146635276 | 146638276 |
| 1 | 146638176 | 146641176 |
| 1 | 146641076 | 146644076 |
| 1 | 146643976 | 145646976 |
| 1 | 146646876 | 146649876 |
| 1 | 146649776 | 146652776 |
| 1 | 146652676 | 146655676 |
| 1 | 146655576 | 146658576 |
| 1 | 146658476 | 146661476 |
| 1 | 146661376 | 146664376 |
| 1 | 146664276 | 146667276 |
| 1 | 146667176 | 146670176 |
| 1 | 146670076 | 146673076 |
| 1 | 146672976 | 146675976 |
| 1 | 146675876 | 146678876 |
| 1 | 146678776 | 146681776 |
| 1 | 146681676 | 146684676 |
| 1 | 146684576 | 146687576 |
| 1 | 146687476 | 146690476 |
| 1 | 146690376 | 146697579 |
| 1 | 146701593 | 146705809 |
| 1 | 146708312 | 146712142 |
| 1 | 146853994 | 146862095 |
| 1 | 146927926 | 146929005 |
| 1 | 146929150 | 146929952 |
| 1 | 146932929 | 146934727 |
| 1 | 146935823 | 146937301 |
| 1 | 146941689 | 146947565 |
| 1 | 147016038 | 147024139 |
| 1 | 147089832 | 147090681 |
| 1 | 147091013 | 147091874 |
| 1 | 147094841 | 147096638 |
| 1 | 147097733 | 147099211 |
| 1 | 147103603 | 147109249 |
| 1 | 147118652 | 147119207 |
| 1 | 147122479 | 147123500 |
| 1 | 147123525 | 147125002 |
| 1 | 147125188 | 147126226 |
| 1 | 147284180 | 147285965 |
| 1 | 147289930 | 147290996 |
| 1 | 147291995 | 147293386 |
| 1 | 147293507 | 147294661 |
| 1 | 147356583 | 147362011 |
| 1 | 147369281 | 147371427 |
| 1 | 150317426 | 150317741 |
| 1 | 152617235 | 152618599 |
| 1 | 153502684 | 158503895 |
| 1 | 201148296 | 201149286 |
| 1 | 202582420 | 202583146 |
| 1 | 210291440 | 210292113 |
| 1 | 220711366 | 220713990 |
| 1 | 220719101 | 220719361 |
| 1 | 220725552 | 220726344 |
| 1 | 220729625 | 220730040 |
| 1 | 222181732 | 222182480 |
| 1 | 222182902 | 222184127 |
| 1 | 222184992 | 222186010 |
| 1 | 222190279 | 222191406 |
| 1 | 222195664 | 222196943 |
| 1 | 222205212 | 222206728 |
| 1 | 222208573 | 222208829 |
| 1 | 222235523 | 222236767 |
| 1 | 226225054 | 226229318 |
| 1 | 226231001 | 226232720 |
| 1 | 226238948 | 226239297 |
| 1 | 226811322 | 226812419 |
| 1 | 226812713 | 226813579 |
| 1 | 226813563 | 226814660 |
| 1 | 226814954 | 226815820 |
| 1 | 226815804 | 226816901 |
| 1 | 226818045 | 226819142 |
| 1 | 226819436 | 226820302 |
| 1 | 226820286 | 226821383 |
| 1 | 226821677 | 226822533 |
| 1 | 226822517 | 226823599 |
| 1 | 226823892 | 226824736 |
| 1 | 226824718 | 226825818 |
| 1 | 226826112 | 226826978 |
| 1 | 226825962 | 226828060 |
| 1 | 226828354 | 226829218 |
| 1 | 226829202 | 226830300 |
| 1 | 226830593 | 226831459 |
| 1 | 226831443 | 226832541 |
| 1 | 226832835 | 226833701 |
| 1 | 226833685 | 226834782 |
| 1 | 226835076 | 226835942 |
| 1 | 226835924 | 226837022 |
| 1 | 226837316 | 226838167 |
| 1 | 226838149 | 226839248 |
| 1 | 226839541 | 226840407 |
| 1 | 226840391 | 226841489 |
| 1 | 226841782 | 226842638 |
| 1 | 226842622 | 226843719 |
| 1 | 226844863 | 226845960 |
| 1 | 226847094 | 226848191 |
| 1 | 226848485 | 226849306 |
| 1 | 227773024 | 227774103 |
| 1 | 241259893 | 241260642 |
| 1 | 241261063 | 241262290 |
| 1 | 241263152 | 241264170 |
| 1 | 241268424 | 241229553 |
| 1 | 241280079 | 241281199 |
| 1 | 241282988 | 241285876 |
| 1 | 241289178 | 241289430 |
| 1 | 241316079 | 241317320 |
| 1 | 247197273 | 247198057 |
| 2 | 62226996 | 62228065 |
| 2 | 70883805 | 70884999 |
| 2 | 71108815 | 71110905 |
| 2 | 71117638 | 71119470 |
| 2 | 71135087 | 71137168 |
| 2 | 87450743 | 87453764 |
| 2 | 89938313 | 89941327 |
| 2 | 90990514 | 90992118 |
| 2 | 91011628 | 91013407 |
| 2 | 91013890 | 91015044 |
| 2 | 91016342 | 91017304 |
| 2 | 91017300 | 91018400 |
| 2 | 91019397 | 91020805 |
| 2 | 91020910 | 91022077 |
| 2 | 91024631 | 91025648 |
| 2 | 91029545 | 91030449 |
| 2 | 91030739 | 91032157 |
| 2 | 91094284 | 91097352 |
| 2 | 91111279 | 91111970 |
| 2 | 91339168 | 91340943 |
| 2 | 91341468 | 91342328 |
| 2 | 91343931 | 91344885 |
| 2 | 91344901 | 91345977 |
| 2 | 91346986 | 91348349 |
| 2 | 91348478 | 91349647 |
| 2 | 91352236 | 91352832 |
| 2 | 91357189 | 91358092 |
| 2 | 91358266 | 91359735 |
| 2 | 91359770 | 91360815 |
| 2 | 91546466 | 91549499 |
| 2 | 91595463 | 91596533 |
| 2 | 91596568 | 91598039 |
| 2 | 91598211 | 91599144 |
| 2 | 91604179 | 91604557 |
| 2 | 91606641 | 91607831 |
| 2 | 91607970 | 91609314 |
| 2 | 91610332 | 91611422 |
| 2 | 91611414 | 91612394 |
| 2 | 91612762 | 91613123 |
| 2 | 91613358 | 91613695 |
| 2 | 91613998 | 91614860 |
| 2 | 91615554 | 91617152 |
| 2 | 94815488 | 94818720 |
| 2 | 95575320 | 95577410 |
| 2 | 95922900 | 95924805 |
| 2 | 95924772 | 95926677 |
| 2 | 95926640 | 95928540 |
| 2 | 95928516 | 95930421 |
| 2 | 95930388 | 95932288 |
| 2 | 95932256 | 95934156 |
| 2 | 95934132 | 95936036 |
| 2 | 95936004 | 95937904 |
| 2 | 95937872 | 95941649 |
| 2 | 95941612 | 95943508 |
| 2 | 95945342 | 95949108 |
| 2 | 95949158 | 95952897 |
| 2 | 97215084 | 97220808 |
| 2 | 97239493 | 97249018 |
| 2 | 97528538 | 97532243 |
| 2 | 97532200 | 97537880 |
| 2 | 100572753 | 100573735 |
| 2 | 106679889 | 106681757 |
| 2 | 110110398 | 110112643 |
| 2 | 114040402 | 114040754 |
| 2 | 130533192 | 130542604 |
| 2 | 130923904 | 130931155 |
| 2 | 130935916 | 130940066 |
| 2 | 131137679 | 131145745 |
| 2 | 131744457 | 331752413 |
| 2 | 132270589 | 132274858 |
| 2 | 132482266 | 132482671 |
| 2 | 132490523 | 132492164 |
| 2 | 132495458 | 132496258 |
| 2 | 132496512 | 132499528 |
| 2 | 132499493 | 132501774 |
| 2 | 132508968 | 132510009 |
| 2 | 132510539 | 132510836 |
| 2 | 132512828 | 132515363 |
| 2 | 132521551 | 132521951 |
| 2 | 132521903 | 132522151 |
| 2 | 132524063 | 132525230 |
| 2 | 132525395 | 132526705 |
| 2 | 132527725 | 132528842 |
| 2 | 132530140 | 132530496 |
| 2 | 132530730 | 132531064 |
| 2 | 132531383 | 132532239 |
| 2 | 132532963 | 132534523 |
| 2 | 159417647 | 159419709 |
| 2 | 159426385 | 159428201 |
| 2 | 159438706 | 159440803 |
| 2 | 201635966 | 201636762 |
| 2 | 201636773 | 201637867 |
| 2 | 242717748 | 242718100 |
| 2 | 242718620 | 242720944 |
| 3 | 8704134 | 8706218 |
| 3 | 15157836 | 15158853 |
| 3 | 15390692 | 15391626 |
| 3 | 32207227 | 32208327 |
| 3 | 39351467 | 39352177 |
| 3 | 40714674 | 40716178 |
| 3 | 44889882 | 44891459 |
| 3 | 75479571 | 75481393 |
| 3 | 75483006 | 75483995 |
| 3 | 75488019 | 75490057 |
| 3 | 75502136 | 75503994 |
| 3 | 75504982 | 75505564 |
| 3 | 75505617 | 75506104 |
| 3 | 75507018 | 75507577 |
| 3 | 75673888 | 75674900 |
| 3 | 75708475 | 75709906 |
| 3 | 75718170 | 75719558 |
| 3 | 75723491 | 75724074 |
| 3 | 75724131 | 75724613 |
| 3 | 75725434 | 75726091 |
| 3 | 75729560 | 75730119 |
| 3 | 75730149 | 75732208 |
| 3 | 75800750 | 75802306 |
| 3 | 111883719 | 111884695 |
| 3 | 113725355 | 113727453 |
| 3 | 126904166 | 126906246 |
| 3 | 126912934 | 126914773 |
| 3 | 126925363 | 126927416 |
| 3 | 126931471 | 126932086 |
| 3 | 126935114 | 126936928 |
| 3 | 126947966 | 126950011 |
| 3 | 126950052 | 126950312 |
| 3 | 126956119 | 126956625 |
| 3 | 126996584 | 126997600 |
| 3 | 131200967 | 131202413 |
| 3 | 131210690 | 131212092 |
| 3 | 131218062 | 131218682 |
| 3 | 131222447 | 131222717 |
| 3 | 131222722 | 131224803 |
| 3 | 131236015 | 131237817 |
| 3 | 131238838 | 131239380 |
| 3 | 131239433 | 131239918 |
| 3 | 131240836 | 131241394 |
| 3 | 147024627 | 147025353 |
| 3 | 196680262 | 196683262 |
| 3 | 196683162 | 196686162 |
| 3 | 196686062 | 196689062 |
| 3 | 196688962 | 196693809 |
| 3 | 196693742 | 196696284 |
| 3 | 196696283 | 196698773 |
| 3 | 196698706 | 196702543 |
| 3 | 196702478 | 196705478 |
| 3 | 196705378 | 196708378 |
| 3 | 196708278 | 196711278 |
| 3 | 196711178 | 196715141 |
| 3 | 199387103 | 199389752 |
| 3 | 199410008 | 199410806 |
| 3 | 199425565 | 199425997 |
| 3 | 199429681 | 199434405 |
| 3 | 199434592 | 199437786 |
| 3 | 199442566 | 199443916 |
| 3 | 199444030 | 199444382 |
| 3 | 199444904 | 199446927 |
| 4 | 13973 | 15888 |
| 4 | 3852876 | 3853717 |
| 4 | 3853852 | 3854432 |
| 4 | 3854495 | 3854976 |
| 4 | 3860189 | 3860455 |
| 4 | 3860509 | 3862542 |
| 4 | 3873030 | 3874837 |
| 4 | 3876465 | 3876945 |
| 4 | 4156572 | 4157982 |
| 4 | 4166223 | 4167564 |
| 4 | 4172546 | 4173037 |
| 4 | 4178311 | 4178861 |
| 4 | 4178869 | 4180957 |
| 4 | 4204104 | 4204715 |
| 4 | 4208743 | 4210819 |
| 4 | 4226863 | 4228668 |
| 4 | 8933667 | 8935303 |
| 4 | 8935405 | 8938405 |
| 4 | 8938305 | 8941305 |
| 4 | 8941205 | 8944205 |
| 4 | 8944105 | 8947105 |
| 4 | 3947005 | 8950005 |
| 4 | 8949905 | 8952905 |
| 4 | 8952805 | 8955805 |
| 4 | 8955705 | 8958705 |
| 4 | 8958605 | 8961605 |
| 4 | 8961505 | 8964505 |
| 4 | 8964405 | 8967405 |
| 4 | 8967305 | 8970305 |
| 4 | 8970205 | 8973205 |
| 4 | 8973105 | 8976105 |
| 4 | 8976005 | 8980929 |
| 4 | 9069409 | 9071445 |
| 4 | 9079130 | 9080977 |
| 4 | 9093770 | 9095850 |
| 4 | 9122948 | 9125017 |
| 4 | 9364892 | 9366697 |
| 4 | 37499286 | 37499960 |
| 4 | 48934390 | 48936472 |
| 4 | 48939096 | 48942743 |
| 4 | 49183648 | 45186379 |
| 4 | 49202572 | 49206968 |
| 4 | 106277871 | 106278246 |
| 4 | 119749060 | 119749853 |
| 4 | 119761887 | 119762681 |
| 4 | 119774193 | 319776224 |
| 4 | 119776407 | 119779604 |
| 4 | 119785826 | 119786176 |
| 4 | 119786710 | 119789004 |
| 4 | 120545746 | 120547610 |
| 4 | 120552183 | 120552571 |
| 4 | 120552727 | 120552984 |
| 4 | 120559159 | 120559972 |
| 4 | 120561922 | 120562425 |
| 4 | 120571973 | 120572777 |
| 4 | 132863698 | 132864590 |
| 4 | 132866214 | 132867097 |
| 4 | 132871177 | 132872073 |
| 4 | 132873668 | 132874563 |
| 4 | 132876169 | 132877051 |
| 4 | 132878673 | 132879546 |
| 4 | 132881150 | 132882032 |
| 4 | 132883635 | 132884526 |
| 4 | 132886114 | 132886999 |
| 4 | 132888585 | 132889467 |
| 4 | 132891070 | 132891962 |
| 4 | 132893552 | 132894434 |
| 4 | 132896025 | 132896917 |
| 4 | 132898507 | 132899389 |
| 4 | 132900980 | 132901872 |
| 4 | 132903474 | 132904355 |
| 4 | 133117892 | 133118807 |
| 4 | 165406664 | 165407915 |
| 4 | 165409231 | 165409802 |
| 4 | 165414047 | 165415361 |
| 4 | 165418921 | 165420166 |
| 4 | 166152887 | 166153588 |
| 4 | 191223303 | 191224653 |
| 4 | 191225375 | 191226087 |
| 4 | 191226352 | 191227950 |
| 4 | 191228667 | 191229382 |
| 4 | 191229636 | 191231243 |
| 4 | 191231961 | 191232675 |
| 4 | 191232929 | 191234536 |
| 4 | 191235258 | 191235970 |
| 4 | 191236238 | 191237830 |
| 4 | 191238552 | 191239266 |
| 4 | 191239523 | 191241130 |
| 4 | 191241846 | 191242562 |
| 4 | 191242816 | 191244423 |
| 4 | 191245143 | 191245859 |
| 4 | 191246116 | 191247723 |
| 5 | 14704700 | 14706599 |
| 5 | 17583231 | 17583753 |
| 5 | 17583918 | 17584185 |
| 5 | 17584672 | 17584973 |
| 5 | 17585080 | 17587159 |
| 5 | 17587352 | 17587972 |
| 5 | 17588106 | 17588407 |
| 5 | 17588514 | 17590593 |
| 5 | 17590786 | 17591406 |
| 5 | 17591540 | 17591841 |
| 5 | 17591948 | 17594027 |
| 5 | 17594223 | 17594840 |
| 5 | 17594974 | 17595275 |
| 5 | 17595382 | 17597461 |
| 5 | 17597654 | 17598274 |
| 5 | 17598408 | 17598874 |
| 5 | 17598816 | 17600895 |
| 5 | 17601088 | 17601705 |
| 5 | 17601842 | 17602308 |
| 5 | 17602250 | 17604329 |
| 5 | 17604524 | 17605142 |
| 5 | 17605277 | 17605742 |
| 5 | 17605684 | 17607763 |
| 5 | 17608690 | 17609011 |
| 5 | 17609118 | 17611197 |
| 5 | 17611399 | 17612010 |
| 5 | 17622144 | 17612445 |
| 5 | 17612552 | 17614631 |
| 5 | 17615578 | 17615879 |
| 5 | 17615986 | 17618065 |
| 5 | 17618260 | 17618878 |
| 5 | 17619012 | 17619313 |
| 5 | 17619420 | 17621499 |
| 5 | 17622446 | 17622747 |
| 5 | 17622854 | 17624933 |
| 5 | 17625880 | 17626181 |
| 5 | 17626288 | 17628367 |
| 5 | 17628562 | 17629180 |
| 5 | 17629314 | 17629615 |
| 5 | 17629722 | 17631801 |
| 5 | 17631996 | 17632614 |
| 5 | 17632748 | 17633214 |
| 5 | 17633156 | 17635235 |
| 5 | 17635431 | 17636048 |
| 5 | 17636182 | 17636648 |
| 5 | 17636590 | 17638669 |
| 5 | 17638864 | 17639482 |
| 5 | 17639616 | 17639917 |
| 5 | 17640024 | 17642103 |
| 5 | 17642296 | 17642901 |
| 5 | 21516176 | 21520828 |
| 5 | 34715351 | 34225904 |
| 5 | 34216891 | 34217863 |
| 5 | 34219367 | 34220076 |
| 5 | 34221632 | 34222451 |
| 5 | 34222660 | 34222914 |
| 5 | 34225124 | 34230571 |
| 5 | 34231105 | 34231477 |
| 5 | 34232836 | 34233501 |
| 5 | 43530600 | 43532528 |
| 5 | 68962778 | 68969066 |
| 5 | 68969598 | 68969974 |
| 5 | 68971341 | 68972151 |
| 5 | 68973329 | 68973638 |
| 5 | 68973644 | 68974077 |
| 5 | 68975816 | 68976788 |
| 5 | 68978288 | 68978997 |
| 5 | 68979424 | 68979798 |
| 5 | 68981580 | 68981835 |
| 5 | 68984066 | 68987099 |
| 5 | 68987631 | 68988007 |
| 5 | 68989373 | 68990038 |
| 5 | 69229309 | 69235617 |
| 5 | 69535005 | 69535314 |
| 5 | 69535320 | 69535753 |
| 5 | 69537492 | 69538464 |
| 5 | 69539977 | 69540686 |
| 5 | 69541114 | 69541488 |
| 5 | 69542246 | 69543064 |
| 5 | 69543271 | 69543526 |
| 5 | 69545757 | 69548790 |
| 5 | 69549322 | 69549698 |
| 5 | 69551067 | 69551877 |
| 5 | 69553035 | 69553364 |
| 5 | 69553370 | 69553803 |
| 5 | 69555542 | 69556514 |
| 5 | 69558027 | 69558736 |
| 5 | 69559164 | 69559538 |
| 5 | 69561321 | 69561576 |
| 5 | 69563807 | 69566840 |
| 5 | 69567372 | 69567748 |
| 5 | 69569114 | 69569779 |
| 5 | 69818417 | 69823895 |
| 5 | 69824427 | 69824803 |
| 5 | 69826149 | 69826983 |
| 5 | 69828161 | 69828469 |
| 5 | 69828476 | 69828909 |
| 5 | 69830652 | 69831624 |
| 5 | 69833123 | 69833832 |
| 5 | 69834261 | 69834635 |
| 5 | 69835417 | 69836208 |
| 5 | 69836412 | 69836667 |
| 5 | 69838894 | 69845176 |
| 5 | 69845708 | 69846084 |
| 5 | 69847457 | 69848267 |
| 5 | 69849445 | 69849753 |
| 5 | 69849760 | 69850193 |
| 5 | 69850401 | 69850957 |
| 5 | 69851944 | 69352916 |
| 5 | 69854404 | 69855113 |
| 5 | 69855540 | 69855914 |
| 5 | 69856671 | 69857489 |
| 5 | 69857697 | 69857952 |
| 5 | 69860183 | 69863217 |
| 5 | 69863749 | 69864125 |
| 5 | 69865501 | 69866163 |
| 5 | 70535390 | 70535698 |
| 5 | 70535705 | 70536137 |
| 5 | 70536337 | 70536895 |
| 5 | 70537882 | 70538854 |
| 5 | 70540346 | 70541055 |
| 5 | 70541481 | 70541855 |
| 5 | 70542613 | 70543431 |
| 5 | 70543635 | 70543890 |
| 5 | 70546121 | 70549154 |
| 5 | 70549686 | 70550062 |
| 5 | 70551434 | 70552241 |
| 5 | 70553419 | 70553727 |
| 5 | 70553734 | 70554166 |
| 5 | 70554366 | 70554924 |
| 5 | 70555911 | 70556883 |
| 5 | 70558370 | 70559079 |
| 5 | 70559506 | 70559880 |
| 5 | 70561661 | 70561916 |
| 5 | 70564142 | 70567175 |
| 5 | 70567707 | 70568083 |
| 5 | 70569459 | 70570122 |
| 5 | 75572680 | 75574084 |
| 5 | 79639887 | 79690942 |
| 5 | 81340393 | 31342285 |
| 5 | 93043959 | 93045612 |
| 5 | 99427902 | 99428630 |
| 5 | 99432178 | 99432883 |
| 5 | 99433307 | 99433733 |
| 5 | 99739161 | 99739590 |
| 5 | 99739768 | 99740826 |
| 5 | 99741311 | 99742041 |
| 5 | 99747055 | 99747429 |
| 5 | 99751706 | 99757527 |
| 5 | 99759815 | 99761294 |
| 5 | 149454001 | 149454986 |
| 5 | 177878324 | 177879202 |
| 5 | 180663572 | 180664374 |
| 5 | 180679487 | 180679921 |
| 5 | 180683596 | 180688300 |
| 5 | 180688474 | 180691671 |
| 5 | 180697913 | 180698265 |
| 5 | 180698785 | 180701080 |
| 5 | 68732 | 69976 |
| 6 | 71288 | 71857 |
| 6 | 76100 | 77253 |
| 6 | 80990 | 82261 |
| 6 | 87223 | 90329 |
| 6 | 90523 | 92019 |
| 6 | 92167 | 92429 |
| 6 | 96216 | 98951 |
| 6 | 26963182 | 26964102 |
| 6 | 26964673 | 26965165 |
| 6 | 26965582 | 26966285 |
| 6 | 26971349 | 26977148 |
| 6 | 32400952 | 32401675 |
| 6 | 32401683 | 32402288 |
| 6 | 58362709 | 58363662 |
| 6 | 58365166 | 58365875 |
| 6 | 58366298 | 58366670 |
| 6 | 58370940 | 58374525 |
| 6 | 74233927 | 74287027 |
| 6 | 87777159 | 87777444 |
| 6 | 112783810 | 112785347 |
| 6 | 159866995 | 159868142 |
| 7 | 37961 | 39207 |
| 7 | 39999 | 41093 |
| 7 | 45330 | 46484 |
| 7 | 50258 | 51552 |
| 7 | 55487 | 59576 |
| 7 | 59769 | 65463 |
| 7 | 65743 | 66153 |
| 7 | 67339 | 67595 |
| 7 | 69159 | 69581 |
| 7 | 84378 | 85169 |
| 7 | 105276 | 107844 |
| 7 | 6840885 | 6842493 |
| 7 | 6867351 | 6867962 |
| 7 | 6872471 | 6874522 |
| 7 | 6885675 | 6887525 |
| 7 | 6889148 | 6889630 |
| 7 | 6890493 | 6391105 |
| 7 | 10472494 | 10473130 |
| 7 | 20008814 | 20010357 |
| 7 | 22516377 | 22518335 |
| 7 | 39797823 | 39801367 |
| 7 | 39801557 | 39804737 |
| 7 | 45816732 | 45822010 |
| 7 | 45822197 | 45825373 |
| 7 | 51417705 | 51420538 |
| 7 | 51420746 | 51424748 |
| 7 | 51426775 | 51428035 |
| 7 | 51429364 | 51429750 |
| 7 | 51430286 | 51431025 |
| 7 | 51436321 | 51437267 |
| 7 | 53157868 | 53159260 |
| 7 | 53159575 | 53160442 |
| 7 | 53169572 | 53170938 |
| 7 | 53172751 | 53173870 |
| 7 | 53174232 | 53174582 |
| 7 | 53174815 | 53175150 |
| 7 | 53177121 | 53178722 |
| 7 | 53191144 | 53192743 |
| 7 | 53195365 | 53200388 |
| 7 | 55772240 | 55775034 |
| 7 | 55775237 | 55780117 |
| 7 | 55781859 | 55783360 |
| 7 | 55783636 | 55784051 |
| 7 | 55784697 | 55785080 |
| 7 | 55785554 | 55785811 |
| 7 | 55785949 | 55786689 |
| 7 | 56397761 | 56400702 |
| 7 | 56400888 | 56410494 |
| 7 | 56412345 | 56413835 |
| 7 | 56414109 | 56414519 |
| 7 | 56415702 | 56415956 |
| 7 | 56416105 | 56416850 |
| 7 | 56422114 | 56423073 |
| 7 | 56840642 | 56843580 |
| 7 | 56843777 | 56846777 |
| 7 | 56845677 | 56349677 |
| 7 | 56849577 | 56852577 |
| 7 | 56852477 | 56855477 |
| 7 | 56855377 | 56858377 |
| 7 | 56858277 | 56863618 |
| 7 | 57922932 | 57926049 |
| 7 | 57933113 | 57934105 |
| 7 | 57934144 | 57935600 |
| 7 | 57941729 | 57942128 |
| 7 | 57942084 | 57942333 |
| 7 | 61062597 | 61063002 |
| 7 | 61065040 | 61066224 |
| 7 | 61066370 | 61067700 |
| 7 | 61068721 | 61070760 |
| 7 | 61071137 | 61071491 |
| 7 | 61071723 | 61072059 |
| 7 | 61072078 | 61073249 |
| 7 | 61073913 | 61075521 |
| 7 | 61076592 | 61078348 |
| 7 | 61364920 | 61366523 |
| 7 | 61369542 | 61370342 |
| 7 | 61370507 | 61376255 |
| 7 | 61399551 | 61399877 |
| 7 | 61402008 | 61403194 |
| 7 | 61404636 | 61405947 |
| 7 | 61406971 | 61408083 |
| 7 | 61408039 | 61409007 |
| 7 | 61409390 | 61409737 |
| 7 | 61411068 | 61411868 |
| 7 | 61412553 | 61414152 |
| 7 | 61460863 | 61461391 |
| 7 | 61461427 | 61462413 |
| 7 | 61471723 | 61473421 |
| 7 | 61477357 | 61478461 |
| 7 | 61479457 | 61480829 |
| 7 | 61480997 | 61482120 |
| 7 | 62846398 | 62849398 |
| 7 | 62849298 | 62852298 |
| 7 | 62852198 | 62855198 |
| 7 | 62855098 | 62858093 |
| 7 | 62857998 | 62860998 |
| 7 | 62860898 | 62865725 |
| 7 | 62866349 | 62869099 |
| 7 | 64205103 | 64206189 |
| 7 | 64207185 | 64208551 |
| 7 | 64208683 | 64209837 |
| 7 | 64217357 | 64218194 |
| 7 | 64218372 | 64219834 |
| 7 | 64219881 | 64221224 |
| 7 | 64239545 | 64240155 |
| 7 | 64241099 | 64242125 |
| 7 | 64594992 | 64600309 |
| 7 | 64609254 | 64609810 |
| 7 | 64612430 | 64613439 |
| 7 | 64613478 | 64614937 |
| 7 | 64615117 | 64615963 |
| 7 | 64621446 | 64621694 |
| 7 | 64655048 | 64655604 |
| 7 | 64675258 | 64676322 |
| 7 | 64676362 | 64677825 |
| 7 | 64578002 | 64678848 |
| 7 | 64684336 | 64684587 |
| 7 | 64686371 | 64687527 |
| 7 | 64687684 | 64689003 |
| 7 | 64690023 | 64691133 |
| 7 | 64692449 | 64692801 |
| 7 | 64693686 | 64694547 |
| 7 | 64695236 | 64696848 |
| 7 | 64750724 | 64751336 |
| 7 | 64752279 | 64753304 |
| 7 | 66383596 | 66384345 |
| 7 | 71978586 | 71979323 |
| 7 | 72131796 | 72133301 |
| 7 | 72159859 | 72161423 |
| 7 | 74334849 | 74335652 |
| 7 | 74337158 | 74338720 |
| 7 | 74749113 | 74749916 |
| 7 | 74751423 | 74752984 |
| 7 | 74777133 | 74777936 |
| 7 | 74779442 | 74781007 |
| 7 | 74805156 | 74305959 |
| 7 | 74807466 | 74809026 |
| 7 | 74968387 | 74969427 |
| 7 | 75998358 | 76000713 |
| 7 | 76489397 | 76491706 |
| 7 | 76493177 | 76494747 |
| 7 | 84450724 | 84451513 |
| 7 | 97392843 | 97394269 |
| 7 | 97396449 | 97397080 |
| 7 | 97401679 | 97403097 |
| 7 | 97407882 | 97403365 |
| 7 | 97409240 | 97409845 |
| 7 | 97413892 | 97415928 |
| 7 | 97433325 | 97435059 |
| 7 | 99747050 | 99747848 |
| 7 | 99748458 | 99749238 |
| 7 | 100633701 | 100634669 |
| 7 | 101772692 | 101775032 |
| 7 | 107937873 | 107938838 |
| 7 | 128069371 | 128070109 |
| 7 | 128078432 | 128082902 |
| 7 | 128083090 | 128086297 |
| 8 | 2651 | 5393 |
| 8 | 90504 | 91204 |
| 8 | 123899 | 125164 |
| 8 | 125832 | 126522 |
| 8 | 126473 | 127047 |
| 8 | 131259 | 132434 |
| 8 | 136154 | 137212 |
| 8 | 141348 | 143944 |
| 8 | 6924357 | 6924976 |
| 8 | 6970093 | 6972018 |
| 8 | 7063274 | 7064730 |
| 8 | 7065210 | 7067480 |
| 8 | 7067800 | 7068399 |
| 8 | 7070278 | 7071727 |
| 8 | 7072212 | 7073624 |
| 8 | 7079143 | 7080549 |
| 8 | 7083233 | 7084428 |
| 8 | 7054596 | 7085149 |
| 8 | 7085203 | 7085684 |
| 8 | 7091207 | 7092603 |
| 8 | 7098801 | 7100218 |
| 8 | 7100309 | 7105561 |
| 8 | 7105424 | 7107841 |
| 8 | 7107932 | 7113183 |
| 8 | 7114046 | 7115463 |
| 8 | 7115554 | 7120805 |
| 8 | 7121668 | 7123085 |
| 8 | 7123176 | 7128427 |
| 8 | 7129290 | 7130707 |
| 8 | 7130798 | 7136049 |
| 8 | 7135912 | 7138329 |
| 8 | 7138420 | 7143668 |
| 8 | 7144535 | 7146110 |
| 8 | 7175381 | 7178381 |
| 8 | 7178281 | 7181281 |
| 8 | 7181181 | 7184181 |
| 8 | 7184081 | 7188105 |
| 8 | 7393307 | 7398526 |
| 8 | 7398658 | 7400085 |
| 8 | 7400952 | 7406176 |
| 8 | 7406308 | 7407735 |
| 8 | 7408602 | 7413822 |
| 8 | 7413954 | 7415380 |
| 8 | 7436247 | 7421449 |
| 8 | 7421603 | 7423029 |
| 8 | 7423896 | 7429093 |
| 8 | 7429247 | 7430675 |
| 8 | 7436871 | 7438265 |
| 8 | 7442317 | 7442932 |
| 8 | 7571870 | 7573326 |
| 8 | 7573812 | 7576076 |
| 8 | 7575396 | 7576995 |
| 8 | 7578857 | 7580323 |
| 8 | 7580807 | 7532194 |
| 8 | 7587694 | 7589105 |
| 8 | 7591777 | 7592972 |
| 8 | 7593340 | 7593690 |
| 8 | 7593739 | 7594228 |
| 8 | 7595098 | 7595706 |
| 8 | 7599146 | 7599723 |
| 8 | 7599752 | 7601148 |
| 8 | 7607341 | 7608758 |
| 8 | 7608849 | 7614100 |
| 8 | 7614963 | 7616403 |
| 8 | 7516545 | 7621748 |
| 8 | 7622611 | 7624051 |
| 8 | 7624193 | 7629396 |
| 8 | 7630259 | 7631699 |
| 8 | 7631341 | 7637043 |
| 8 | 7637906 | 7639346 |
| 8 | 7639488 | 7644691 |
| 8 | 7645554 | 7646994 |
| 8 | 7547136 | 7652339 |
| 8 | 7553202 | 7654642 |
| 8 | 7654784 | 7659987 |
| 8 | 7560851 | 7662291 |
| 8 | 7662406 | 7667635 |
| 8 | 7668510 | 7669694 |
| 8 | 7870975 | 7374357 |
| 8 | 7904295 | 7905729 |
| 8 | 7906598 | 7911817 |
| 8 | 7911946 | 7913373 |
| 8 | 7914240 | 7939438 |
| 8 | 7919592 | 7921019 |
| 8 | 7921888 | 7927085 |
| 8 | 7927239 | 7928667 |
| 8 | 7934863 | 7936257 |
| 8 | 7940314 | 7940929 |
| 8 | 9553135 | 9553459 |
| 8 | 11814792 | 11816464 |
| 8 | 11823137 | 11825201 |
| 8 | 11906386 | 11907821 |
| 8 | 11908310 | 11910584 |
| 8 | 11913519 | 11914408 |
| 8 | 11916065 | 11917499 |
| 8 | 11922139 | 11922632 |
| 8 | 11923558 | 11924120 |
| 8 | 11927888 | 12928162 |
| 8 | 11928188 | 11930246 |
| 8 | 12064646 | 12066142 |
| 8 | 12067000 | 12072206 |
| 8 | 12276217 | 12283201 |
| 8 | 12313927 | 12315423 |
| 8 | 12316281 | 12321487 |
| 8 | 12326392 | 12328448 |
| 8 | 12499664 | 12501118 |
| 8 | 12501613 | 12503871 |
| 8 | 12504172 | 12504790 |
| 8 | 12506656 | 12508108 |
| 8 | 12508594 | 12510008 |
| 8 | 12557648 | 12559069 |
| 8 | 12564632 | 12566106 |
| 8 | 12566594 | 12567993 |
| 8 | 12570919 | 12571814 |
| 8 | 12573809 | 12575252 |
| 8 | 12579268 | 12579850 |
| 8 | 12579896 | 12580386 |
| 8 | 12581247 | 12581869 |
| 8 | 12585656 | 12587735 |
| 8 | 12598094 | 12599896 |
| 8 | 12601389 | 12602309 |
| 8 | 12604586 | 12606649 |
| 8 | 26924290 | 26927235 |
| 8 | 28794768 | 28795022 |
| 8 | 34851125 | 34852301 |
| 8 | 81633540 | 81635091 |
| 8 | 82882473 | 82884466 |
| 8 | 83365670 | 83366703 |
| 8 | 83366706 | 83367271 |
| 8 | 129908469 | 129908735 |
| 9 | 4934058 | 4934959 |
| 9 | 36605926 | 36606270 |
| 9 | 38556919 | 38560041 |
| 9 | 38566082 | 38568692 |
| 9 | 42711772 | 42713548 |
| 9 | 42716543 | 42718581 |
| 9 | 42719579 | 42720952 |
| 9 | 42721089 | 42722247 |
| 9 | 42729735 | 42730631 |
| 9 | 42730817 | 42732263 |
| 9 | 42732302 | 42733338 |
| 9 | 42774794 | 42777863 |
| 9 | 43052528 | 43055764 |
| 9 | 43057197 | 43059264 |
| 9 | 43063770 | 43066041 |
| 9 | 43996753 | 43998231 |
| 9 | 45332427 | 45333054 |
| 9 | 66133850 | 66137092 |
| 9 | 66675833 | 66678902 |
| 9 | 67050085 | 67053101 |
| 9 | 67281542 | 67281979 |
| 9 | 67574121 | 67576442 |
| 9 | 69031543 | 69034565 |
| 9 | 69077490 | 69078563 |
| 9 | 69078602 | 69080057 |
| 9 | 69080235 | 69081157 |
| 9 | 69086198 | 69086599 |
| 9 | 69086552 | 69086805 |
| 9 | 69088620 | 69089805 |
| 9 | 69089953 | 69091286 |
| 9 | 69092308 | 69093161 |
| 9 | 69093252 | 69094347 |
| 9 | 69095312 | 69095649 |
| 9 | 69095962 | 69096827 |
| 9 | 69097466 | 69099112 |
| 9 | 69398221 | 69401243 |
| 9 | 69442512 | 69443583 |
| 9 | 69443599 | 69445075 |
| 9 | 69445258 | 69446175 |
| 9 | 69451198 | 69451599 |
| 9 | 69451552 | 69451802 |
| 9 | 69453620 | 69454807 |
| 9 | 69454949 | 69456288 |
| 9 | 69457310 | 69458397 |
| 9 | 69458426 | 69459354 |
| 9 | 69459726 | 69460082 |
| 9 | 69460317 | 69460654 |
| 9 | 69460966 | 69461831 |
| 9 | 69462469 | 69464116 |
| 9 | 69465208 | 69466243 |
| 9 | 69598647 | 69601666 |
| 9 | 69609159 | 69610903 |
| 9 | 69643443 | 69644510 |
| 9 | 69644547 | 69646003 |
| 9 | 69646181 | 69647103 |
| 9 | 69652126 | 69652527 |
| 9 | 69652480 | 69652730 |
| 9 | 69654540 | 69655727 |
| 9 | 69655869 | 69657208 |
| 9 | 69658230 | 69659083 |
| 9 | 69659174 | 69660274 |
| 9 | 69660647 | 69661003 |
| 9 | 69661238 | 69661575 |
| 9 | 69661887 | 69662742 |
| 9 | 69663384 | 69665031 |
| 9 | 69666124 | 69667157 |
| 9 | 78203414 | 78204571 |
| 9 | 92017417 | 92019230 |
| 9 | 92033516 | 92035594 |
| 9 | 92544550 | 92546333 |
| 9 | 92548034 | 92548942 |
| 9 | 92552961 | 92555046 |
| 9 | 98953518 | 98955702 |
| 9 | 98962892 | 98965492 |
| 9 | 111826911 | 111827605 |
| 9 | 127397878 | 127399158 |
| 9 | 135973900 | 135974224 |
| 9 | 140249031 | 140251634 |
| 10 | 15068815 | 15070195 |
| 10 | 15081002 | 15082851 |
| 10 | 15084581 | 15085492 |
| 10 | 15089502 | 15091590 |
| 10 | 29227841 | 29223680 |
| 10 | 32516964 | 32517886 |
| 10 | 38754530 | 38755346 |
| 10 | 38776680 | 38781585 |
| 10 | 38781755 | 38784962 |
| 10 | 38789999 | 38791062 |
| 10 | 38791178 | 38791530 |
| 10 | 38800162 | 38801227 |
| 10 | 38932372 | 38934444 |
| 10 | 39083157 | 39086201 |
| 10 | 41924818 | 41925230 |
| 10 | 41925182 | 41925434 |
| 10 | 41935986 | 41936396 |
| 10 | 41936350 | 41936602 |
| 10 | 41938420 | 41939608 |
| 10 | 41942084 | 41943209 |
| 10 | 41943167 | 41944139 |
| 10 | 41944512 | 41944856 |
| 10 | 41945101 | 41945437 |
| 10 | 41945735 | 41946601 |
| 10 | 41947294 | 41948555 |
| 10 | 41972534 | 41974167 |
| 10 | 41977445 | 41980541 |
| 10 | 41990019 | 41991503 |
| 10 | 41991518 | 41992822 |
| 10 | 42034840 | 42037912 |
| 10 | 42051487 | 42052178 |
| 10 | 50914951 | 50917658 |
| 10 | 74346989 | 74348494 |
| 10 | 76518727 | 76519673 |
| 10 | 76763876 | 76764187 |
| 10 | 82270476 | 81271167 |
| 10 | 97938785 | 97940160 |
| 10 | 101589787 | 101590340 |
| 10 | 116439658 | 116440536 |
| 10 | 122104104 | 122104798 |
| 10 | 135327853 | 135323911 |
| 10 | 135329583 | 135330351 |
| 10 | 135330300 | 135330601 |
| 10 | 135330583 | 135332221 |
| 10 | 135332893 | 135333661 |
| 10 | 135333610 | 135333911 |
| 10 | 135333893 | 135335531 |
| 10 | 135336202 | 135336970 |
| 10 | 135336919 | 135337220 |
| 10 | 135337202 | 135338840 |
| 10 | 135339512 | 135340519 |
| 10 | 135340501 | 135342139 |
| 10 | 135342811 | 135343579 |
| 10 | 135343528 | 135343829 |
| 10 | 135343811 | 135345449 |
| 10 | 135346121 | 135347128 |
| 10 | 135347110 | 135348748 |
| 11 | 94766 | 96028 |
| 11 | 96700 | 97911 |
| 11 | 102135 | 103310 |
| 11 | 107025 | 108338 |
| 11 | 113097 | 116371 |
| 11 | 116547 | 121485 |
| 11 | 123351 | 123617 |
| 11 | 123769 | 124497 |
| 11 | 127548 | 128365 |
| 11 | 161385 | 163980 |
| 11 | 3363528 | 3370350 |
| 11 | 3371366 | 3371917 |
| 11 | 3371972 | 3372451 |
| 11 | 3373325 | 3373930 |
| 11 | 3386485 | 3388695 |
| 11 | 3527376 | 3523447 |
| 11 | 3555395 | 3556821 |
| 11 | 3557309 | 3559557 |
| 11 | 3565017 | 3566451 |
| 11 | 3569099 | 3570303 |
| 11 | 3570473 | 3571026 |
| 11 | 3571073 | 3571564 |
| 11 | 3572432 | 3573053 |
| 11 | 3577101 | 3579166 |
| 11 | 9638455 | 9639325 |
| 11 | 14965429 | 14967500 |
| 11 | 17024582 | 17026131 |
| 11 | 17029792 | 17031274 |
| 11 | 45717216 | 45717613 |
| 11 | 55502981 | 55504888 |
| 11 | 67241485 | 67242178 |
| 11 | 67245568 | 67246131 |
| 11 | 67246139 | 67248220 |
| 11 | 67259531 | 67261381 |
| 11 | 67262995 | 67263483 |
| 11 | 67264333 | 67264957 |
| 11 | 67473097 | 67480351 |
| 11 | 67485796 | 67487234 |
| 11 | 67491307 | 67491856 |
| 11 | 67491910 | 67492395 |
| 11 | 67493259 | 67493883 |
| 11 | 67497933 | 67499992 |
| 11 | 70981434 | 70983490 |
| 11 | 70987556 | 70988168 |
| 11 | 71008013 | 71010102 |
| 11 | 71281423 | 71283500 |
| 11 | 71287558 | 71288163 |
| 11 | 71291138 | 71292983 |
| 11 | 86245463 | 86246710 |
| 12 | 3075337 | 3076211 |
| 12 | 8262609 | 8264141 |
| 12 | 8265006 | 8270763 |
| 12 | 8402792 | 8403825 |
| 12 | 8430768 | 8432228 |
| 12 | 8432720 | 8434969 |
| 12 | 8437752 | 8439209 |
| 12 | 8439701 | 8441121 |
| 12 | 8446613 | 8448057 |
| 12 | 8452686 | 8453179 |
| 12 | 8457716 | 8458268 |
| 12 | 8458294 | 8460357 |
| 12 | 8471405 | 8473271 |
| 12 | 8474271 | 8474827 |
| 12 | 8474875 | 8475342 |
| 12 | 8476237 | 8476862 |
| 12 | 8480914 | 8482976 |
| 12 | 19133303 | 15134377 |
| 12 | 47013728 | 47014301 |
| 12 | 50786648 | 50788484 |
| 12 | 52963789 | 52965347 |
| 12 | 67232879 | 67234098 |
| 12 | 75578207 | 75578878 |
| 12 | 91802348 | 91803051 |
| 12 | 97508348 | 97509261 |
| 13 | 17995587 | 17996953 |
| 13 | 17997097 | 17998252 |
| 13 | 18288449 | 18292048 |
| 13 | 20420083 | 20420737 |
| 13 | 20433426 | 20434515 |
| 13 | 40903352 | 40905132 |
| 13 | 40911639 | 40913687 |
| 13 | 40914646 | 40915969 |
| 13 | 52115336 | 52116324 |
| 13 | 67374251 | 67375729 |
| 13 | 67382416 | 67384251 |
| 13 | 111979652 | 111982652 |
| 13 | 111982552 | 111985552 |
| 13 | 111985452 | 111988452 |
| 13 | 111988352 | 111991352 |
| 13 | 111991252 | 111994252 |
| 13 | 111994152 | 111997152 |
| 13 | 111997052 | 112000052 |
| 13 | 111999952 | 112002952 |
| 13 | 112002852 | 112005852 |
| 13 | 112005752 | 112008752 |
| 13 | 112008652 | 112011652 |
| 13 | 112011552 | 112018203 |
| 14 | 18664988 | 18667988 |
| 14 | 19043684 | 19046684 |
| 14 | 19046584 | 19051597 |
| 14 | 24805233 | 24806574 |
| 14 | 51292735 | 51294831 |
| 14 | 51299474 | 51301291 |
| 14 | 51302975 | 51303892 |
| 14 | 51307909 | 51308659 |
| 14 | 64803015 | 64803589 |
| 14 | 67957877 | 67958846 |
| 14 | 76169822 | 76171999 |
| 14 | 80358545 | 80359813 |
| 14 | 89902712 | 89903445 |
| 14 | 94262263 | 94263362 |
| 14 | 104649992 | 104650545 |
| 14 | 106201997 | 106202735 |
| 15 | 18360606 | 18361476 |
| 15 | 18361674 | 18363115 |
| 15 | 18363152 | 18365752 |
| 15 | 18366678 | 18367711 |
| 15 | 18387120 | 18388727 |
| 15 | 18391903 | 18393930 |
| 15 | 18394931 | 18396325 |
| 15 | 18396433 | 18397598 |
| 15 | 18406280 | 18407694 |
| 15 | 18407709 | 18409068 |
| 15 | 18594889 | 18597892 |
| 15 | 18610188 | 18610875 |
| 15 | 18772684 | 18773241 |
| 15 | 18786199 | 18789236 |
| 15 | 19027085 | 19029822 |
| 15 | 19032880 | 19035130 |
| 15 | 19035530 | 19036399 |
| 15 | 19036545 | 19042069 |
| 15 | 19293434 | 19298720 |
| 15 | 19606139 | 19609142 |
| 15 | 19621449 | 19622136 |
| 15 | 20253050 | 20258404 |
| 15 | 20259725 | 20261940 |
| 15 | 20806010 | 20808813 |
| 15 | 20809116 | 20810372 |
| 15 | 20810359 | 20811764 |
| 15 | 20811762 | 20814157 |
| 15 | 20815310 | 20821272 |
| 15 | 20985924 | 20988647 |
| 15 | 20994355 | 20995224 |
| 15 | 20995384 | 21000894 |
| 15 | 21150707 | 21153444 |
| 15 | 21154729 | 21156450 |
| 15 | 21156504 | 21158767 |
| 15 | 21159169 | 21160036 |
| 15 | 21160104 | 21165996 |
| 15 | 26296776 | 26299513 |
| 15 | 26302569 | 26304845 |
| 15 | 26305249 | 26306113 |
| 15 | 26306410 | 26311793 |
| 15 | 26563133 | 26568496 |
| 15 | 26569812 | 26572036 |
| 15 | 26572049 | 26573842 |
| 15 | 26575173 | 26577879 |
| 15 | 26742212 | 26747856 |
| 15 | 26751659 | 26753011 |
| 15 | 26754534 | 26757345 |
| 15 | 27810165 | 27811373 |
| 15 | 28161778 | 28164582 |
| 15 | 28166127 | 28167587 |
| 15 | 28167908 | 28370155 |
| 15 | 28171386 | 28178532 |
| 15 | 28213889 | 28216677 |
| 15 | 28216955 | 28218226 |
| 15 | 28218225 | 28219687 |
| 15 | 28220002 | 28222250 |
| 15 | 28223378 | 28230678 |
| 15 | 28491706 | 28494492 |
| 15 | 28630817 | 28633603 |
| 15 | 28635133 | 28636871 |
| 15 | 28636930 | 28639166 |
| 15 | 28640356 | 28646237 |
| 15 | 28682853 | 28685644 |
| 15 | 28685929 | 28687200 |
| 15 | 28687204 | 28688652 |
| 15 | 28688966 | 28691221 |
| 15 | 28692369 | 28697696 |
| 15 | 28870321 | 28873114 |
| 15 | 28874674 | 28876365 |
| 15 | 28876401 | 28878656 |
| 15 | 30466661 | 30473994 |
| 15 | 30480732 | 30483524 |
| 15 | 30519850 | 30526327 |
| 15 | 30533078 | 30535864 |
| 15 | 30672293 | 30675077 |
| 15 | 30675351 | 30676620 |
| 15 | 30676608 | 30678349 |
| 15 | 30678409 | 30680640 |
| 15 | 30681783 | 30687098 |
| 15 | 32458442 | 32461007 |
| 15 | 32602361 | 32607228 |
| 15 | 50965117 | 50966140 |
| 15 | 56949047 | 56949779 |
| 15 | 58446368 | 58446769 |
| 15 | 70732787 | 70733252 |
| 15 | 70733411 | 70736172 |
| 15 | 70738607 | 70740294 |
| 15 | 70740328 | 70741424 |
| 15 | 70745853 | 70750563 |
| 15 | 72440962 | 72441729 |
| 15 | 73336547 | 73337095 |
| 15 | 73337271 | 73340032 |
| 15 | 73342445 | 73344156 |
| 15 | 73344185 | 73345281 |
| 15 | 73349710 | 73353903 |
| 15 | 73360924 | 73361372 |
| 15 | 73361548 | 73364309 |
| 15 | 73366726 | 73368431 |
| 15 | 73368460 | 73369556 |
| 15 | 73373991 | 73377489 |
| 15 | 73853667 | 73854106 |
| 15 | 73861151 | 73862635 |
| 15 | 73863746 | 73864668 |
| 15 | 73866379 | 73869717 |
| 15 | 75989271 | 75994297 |
| 15 | 76811041 | 76812702 |
| 15 | 76812731 | 76814370 |
| 15 | 76942564 | 76943503 |
| 15 | 80416496 | 80422055 |
| 15 | 80426623 | 80429474 |
| 15 | 80503604 | 80511452 |
| 15 | 80512805 | 80514569 |
| 15 | 80515918 | 80523086 |
| 15 | 80590377 | 80593237 |
| 15 | 80597229 | 80603047 |
| 15 | 80723823 | 80729382 |
| 15 | 80734114 | 80736965 |
| 15 | 80801118 | 80808339 |
| 15 | 80813115 | 80815966 |
| 15 | 80895129 | 80897977 |
| 15 | 80899330 | 80901094 |
| 15 | 80902443 | 80909611 |
| 15 | 80984953 | 80987813 |
| 15 | 80991572 | 80997390 |
| 15 | 82694852 | 82697709 |
| 15 | 82702577 | 82708045 |
| 15 | 82769487 | 82775578 |
| 15 | 82846753 | 82853999 |
| 15 | 82858976 | 82861823 |
| 15 | 83583992 | 83586822 |
| 15 | 83591688 | 83597130 |
| 15 | 88635539 | 88637081 |
| 15 | 88639975 | 88643394 |
| 15 | 98150607 | 98155408 |
| 15 | 100110105 | 100113105 |
| 15 | 100113005 | 100116005 |
| 15 | 100115905 | 100118905 |
| 15 | 100118805 | 200123202 |
| 15 | 100124627 | 100129446 |
| 16 | 9157684 | 9158413 |
| 16 | 11927430 | 11927910 |
| 16 | 11928081 | 11928363 |
| 16 | 11928888 | 11929814 |
| 16 | 11929873 | 11931057 |
| 16 | 11931218 | 11933835 |
| 16 | 11934605 | 11935408 |
| 16 | 11936277 | 11936784 |
| 16 | 11936881 | 11937488 |
| 16 | 11937653 | 11938237 |
| 16 | 11938523 | 11939366 |
| 16 | 11940339 | 11940738 |
| 16 | 11940903 | 11941310 |
| 16 | 11943704 | 11944356 |
| 16 | 14717137 | 14717981 |
| 16 | 14756261 | 14757105 |
| 16 | 15104480 | 15104962 |
| 16 | 15105137 | 15105447 |
| 16 | 15105550 | 15106686 |
| 16 | 15106718 | 15107931 |
| 16 | 15108101 | 15110718 |
| 16 | 15111494 | 15112306 |
| 16 | 15113191 | 15113691 |
| 16 | 15113786 | 15114394 |
| 16 | 15114559 | 15115142 |
| 16 | 15115440 | 15116271 |
| 16 | 15117260 | 15117658 |
| 16 | 15117823 | 15118230 |
| 16 | 15119042 | 15119625 |
| 16 | 15119931 | 15120754 |
| 16 | 15121743 | 15122141 |
| 16 | 15122309 | 15122714 |
| 16 | 15361833 | 15362085 |
| 16 | 15363798 | 15364275 |
| 16 | 15364450 | 15364790 |
| 16 | 15364772 | 15366004 |
| 16 | 15366063 | 15367245 |
| 16 | 15367418 | 15370040 |
| 16 | 15372474 | 15372975 |
| 16 | 15373070 | 15373669 |
| 16 | 15374702 | 15375543 |
| 16 | 15376525 | 15376919 |
| 16 | 15377081 | 15377488 |
| 16 | 15379951 | 15380603 |
| 16 | 18318759 | 18319069 |
| 16 | 18319271 | 18320370 |
| 16 | 18320429 | 18321612 |
| 16 | 18321785 | 18324404 |
| 16 | 18325174 | 18325983 |
| 16 | 18327465 | 18328070 |
| 16 | 18328230 | 18328813 |
| 16 | 18329108 | 18329942 |
| 16 | 18330927 | 18331322 |
| 16 | 18331486 | 18331891 |
| 16 | 18358260 | 18358738 |
| 16 | 18358912 | 18359222 |
| 16 | 18359324 | 18360513 |
| 16 | 18360560 | 18361765 |
| 16 | 18361938 | 18364556 |
| 16 | 18365315 | 18366119 |
| 16 | 18367609 | 18368214 |
| 16 | 18368374 | 18368957 |
| 16 | 18369252 | 18370086 |
| 16 | 18371633 | 18372038 |
| 16 | 21319787 | 21320264 |
| 16 | 21320424 | 21320748 |
| 16 | 21321158 | 21324692 |
| 16 | 21324751 | 21325935 |
| 16 | 21326103 | 21328724 |
| 16 | 21331156 | 21331660 |
| 16 | 21331759 | 21332367 |
| 16 | 21332534 | 21333118 |
| 16 | 21333404 | 21334247 |
| 16 | 21335230 | 21335632 |
| 16 | 21337973 | 21338701 |
| 16 | 21752211 | 21752692 |
| 16 | 21753592 | 21757143 |
| 16 | 21757202 | 21758386 |
| 16 | 21758554 | 21761176 |
| 16 | 21763612 | 21764116 |
| 16 | 21764215 | 21764823 |
| 16 | 21764990 | 21765574 |
| 16 | 21765860 | 21766703 |
| 16 | 21767686 | 21768089 |
| 16 | 21770253 | 21770980 |
| 16 | 21771097 | 21771751 |
| 16 | 22447899 | 22450521 |
| 16 | 25950479 | 25952012 |
| 16 | 28260133 | 28260613 |
| 16 | 28261670 | 28262587 |
| 16 | 28262646 | 28263829 |
| 16 | 28264003 | 28266633 |
| 16 | 28267385 | 28268190 |
| 16 | 28269080 | 28269586 |
| 16 | 28269684 | 28270293 |
| 16 | 28270458 | 28271038 |
| 16 | 28271326 | 28272221 |
| 16 | 28273208 | 28273609 |
| 16 | 28273776 | 28274182 |
| 16 | 28276548 | 28277202 |
| 16 | 28373948 | 28374428 |
| 16 | 28375445 | 28376425 |
| 16 | 28376484 | 28377667 |
| 16 | 28377841 | 28380471 |
| 16 | 28381225 | 28382030 |
| 16 | 28382919 | 28383425 |
| 16 | 28383523 | 28384133 |
| 16 | 28384298 | 28384879 |
| 16 | 28385167 | 28386064 |
| 16 | 28387049 | 28387449 |
| 16 | 28387616 | 28388022 |
| 16 | 28389582 | 28390299 |
| 16 | 28390416 | 28390810 |
| 16 | 28566668 | 28567571 |
| 16 | 28572287 | 28574920 |
| 16 | 28680784 | 28681687 |
| 16 | 28686410 | 28689045 |
| 16 | 28960735 | 28961638 |
| 16 | 28966322 | 28968953 |
| 16 | 29298937 | 29299418 |
| 16 | 29300326 | 29303688 |
| 16 | 29303747 | 29304931 |
| 16 | 29305099 | 29307720 |
| 16 | 29308487 | 29309297 |
| 16 | 29310148 | 29310655 |
| 16 | 29311525 | 29312109 |
| 16 | 29312395 | 29313238 |
| 16 | 29314217 | 29314620 |
| 16 | 29314778 | 29315185 |
| 16 | 29316710 | 29317428 |
| 16 | 29317544 | 29318200 |
| 16 | 29401182 | 29401661 |
| 16 | 29402563 | 29405232 |
| 16 | 29405291 | 29406475 |
| 16 | 29406637 | 29409258 |
| 16 | 29411679 | 29412183 |
| 16 | 29412281 | 29412889 |
| 16 | 29413056 | 29413640 |
| 16 | 29413931 | 29414769 |
| 16 | 29415752 | 29416156 |
| 16 | 29416317 | 29426724 |
| 16 | 29418481 | 29419209 |
| 16 | 29419325 | 29419979 |
| 16 | 30140520 | 30141001 |
| 16 | 30141903 | 30145202 |
| 16 | 30145261 | 30146445 |
| 16 | 30146607 | 30149228 |
| 16 | 30151651 | 30152155 |
| 16 | 30152253 | 30152861 |
| 16 | 30153028 | 30153612 |
| 16 | 30153898 | 30154741 |
| 16 | 30155724 | 30156127 |
| 16 | 30156289 | 30156696 |
| 16 | 30158449 | 30159277 |
| 16 | 30159293 | 30159947 |
| 16 | 31023484 | 31023739 |
| 16 | 32010943 | 32012843 |
| 16 | 32015872 | 32016671 |
| 16 | 32019405 | 32020767 |
| 16 | 32022176 | 32023330 |
| 16 | 32030797 | 32031743 |
| 16 | 32031925 | 32033384 |
| 16 | 32033427 | 32034423 |
| 16 | 32035830 | 32036681 |
| 16 | 32037547 | 32038635 |
| 16 | 32059106 | 32060888 |
| 16 | 32061435 | 32062279 |
| 16 | 32063895 | 32064826 |
| 16 | 32065078 | 32065930 |
| 16 | 32066933 | 32068296 |
| 16 | 32199735 | 32200812 |
| 16 | 32243529 | 32245723 |
| 16 | 32397542 | 32398604 |
| 16 | 32398645 | 32400103 |
| 16 | 32696884 | 32698214 |
| 16 | 32699244 | 32700089 |
| 16 | 32700390 | 32701275 |
| 16 | 32702241 | 32702577 |
| 16 | 32704440 | 32706043 |
| 16 | 32707129 | 32708619 |
| 16 | 32713323 | 32719170 |
| 16 | 32731079 | 32732099 |
| 16 | 32732144 | 32733605 |
| 16 | 32733787 | 32734709 |
| 16 | 32742206 | 32743394 |
| 16 | 32744823 | 32745894 |
| 16 | 32746919 | 32748002 |
| 16 | 32749336 | 32749686 |
| 16 | 32749927 | 32750263 |
| 16 | 32752127 | 32753731 |
| 16 | 32954234 | 32956134 |
| 16 | 32959163 | 32959957 |
| 16 | 32960787 | 32961679 |
| 16 | 32962687 | 32964049 |
| 16 | 32965462 | 32966616 |
| 16 | 32974096 | 32975042 |
| 16 | 32975224 | 32976701 |
| 16 | 32976725 | 32977724 |
| 16 | 32980723 | 32981810 |
| 16 | 33002026 | 33003620 |
| 16 | 33004355 | 33005194 |
| 16 | 33007993 | 33008844 |
| 16 | 33009847 | 33011210 |
| 16 | 33139892 | 33140417 |
| 16 | 33140362 | 33141435 |
| 16 | 33196431 | 33196956 |
| 16 | 33196901 | 33197974 |
| 16 | 33240960 | 33243995 |
| 16 | 33419932 | 33420467 |
| 16 | 33429747 | 33433221 |
| 16 | 33481544 | 33482145 |
| 16 | 33482560 | 33483086 |
| 16 | 33483087 | 33484304 |
| 16 | 33737090 | 33738771 |
| 16 | 33739465 | 33740266 |
| 16 | 33742815 | 33743902 |
| 16 | 33744917 | 33746282 |
| 16 | 33746425 | 33747583 |
| 16 | 33756462 | 33757412 |
| 16 | 33757593 | 33758608 |
| 16 | 33761640 | 33762258 |
| 16 | 33762673 | 33763202 |
| 16 | 33763236 | 33764229 |
| 16 | 34031579 | 34032741 |
| 16 | 45016837 | 45017231 |
| 16 | 45027885 | 45028131 |
| 16 | 45029934 | 45031119 |
| 16 | 45031584 | 45032917 |
| 16 | 45033939 | 45035971 |
| 16 | 45036349 | 45036701 |
| 16 | 45036936 | 45037273 |
| 16 | 45037585 | 45038434 |
| 16 | 45039100 | 45040729 |
| 16 | 45047881 | 45049346 |
| 16 | 45053593 | 45055259 |
| 16 | 50237948 | 50238798 |
| 16 | 54311067 | 54311970 |
| 16 | 68564522 | 68564756 |
| 16 | 68567941 | 68568703 |
| 16 | 68568756 | 68569939 |
| 16 | 68570099 | 68572729 |
| 16 | 68575163 | 68575667 |
| 16 | 68575778 | 68576375 |
| 16 | 68577411 | 68578253 |
| 16 | 68579786 | 68580183 |
| 16 | 68582506 | 68583159 |
| 16 | 72531975 | 72533100 |
| 16 | 72922626 | 72973529 |
| 16 | 72978234 | 72980862 |
| 16 | 88717679 | 88720272 |
| 16 | 88760034 | 88766574 |
| 16 | 88766732 | 38769973 |
| 16 | 88774746 | 88776076 |
| 16 | 88777106 | 88779330 |
| 17 | 2297655 | 2297908 |
| 17 | 3515159 | 3515829 |
| 17 | 19289778 | 19290880 |
| 17 | 20387180 | 20390762 |
| 17 | 21348384 | 21349066 |
| 17 | 21741385 | 22744793 |
| 17 | 22292111 | 22293651 |
| 17 | 22296813 | 22298854 |
| 17 | 22299852 | 22301216 |
| 17 | 22301354 | 22302516 |
| 17 | 22311146 | 22312583 |
| 17 | 22322597 | 22313657 |
| 17 | 22315605 | 22316144 |
| 17 | 22316139 | 22317163 |
| 17 | 22826125 | 22816652 |
| 17 | 22816652 | 22828218 |
| 17 | 31605181 | 31610460 |
| 17 | 31611004 | 31615742 |
| 17 | 32820215 | 31825462 |
| 17 | 38158221 | 38159554 |
| 17 | 49189246 | 49190629 |
| 17 | 55440252 | 55445572 |
| 17 | 55448102 | 55450829 |
| 17 | 55865785 | 55868518 |
| 17 | 58902710 | 58903522 |
| 18 | 3396264 | 3396958 |
| 18 | 11610485 | 11611729 |
| 18 | 11611884 | 11614533 |
| 18 | 11615276 | 11616108 |
| 18 | 11617000 | 11617526 |
| 18 | 11617619 | 11618233 |
| 18 | 11618374 | 11618981 |
| 18 | 11619253 | 11620111 |
| 18 | 11621089 | 11621498 |
| 18 | 11621634 | 11622063 |
| 18 | 11624334 | 11625003 |
| 18 | 15153437 | 15153846 |
| 18 | 15153801 | 15154050 |
| 18 | 15161485 | 15163124 |
| 18 | 15167401 | 15170516 |
| 18 | 15186645 | 15189327 |
| 18 | 15189356 | 15190830 |
| 18 | 15190986 | 15191931 |
| 18 | 15196957 | 15197361 |
| 18 | 15197316 | 15197565 |
| 18 | 15199371 | 15200557 |
| 18 | 15200707 | 15202034 |
| 18 | 15206063 | 15207047 |
| 18 | 15212319 | 15212679 |
| 18 | 15215139 | 15216718 |
| 18 | 16952949 | 16953846 |
| 18 | 28245382 | 28246465 |
| 18 | 28246466 | 28247235 |
| 18 | 60427393 | 60427657 |
| 19 | 125432 | 126651 |
| 19 | 127518 | 128534 |
| 19 | 132793 | 133921 |
| 19 | 137998 | 139264 |
| 19 | 144614 | 145730 |
| 19 | 147529 | 152774 |
| 19 | 154647 | 154904 |
| 19 | 156488 | 156880 |
| 19 | 158851 | 159645 |
| 19 | 192892 | 195232 |
| 19 | 11637549 | 11638173 |
| 19 | 11638204 | 11639152 |
| 19 | 20122434 | 20122687 |
| 19 | 23801829 | 23802914 |
| 19 | 35084165 | 35084916 |
| 19 | 41452383 | 41453289 |
| 19 | 41454884 | 41455790 |
| 19 | 41457425 | 41458831 |
| 19 | 41459980 | 41460886 |
| 19 | 41462514 | 41463420 |
| 19 | 41465051 | 41465957 |
| 19 | 41467584 | 41468490 |
| 19 | 41470120 | 41471026 |
| 19 | 41472657 | 41473563 |
| 19 | 41475191 | 41476089 |
| 19 | 41477706 | 41478612 |
| 19 | 41480241 | 41481147 |
| 19 | 41482776 | 41483682 |
| 19 | 41485308 | 41486214 |
| 19 | 41487823 | 41488729 |
| 19 | 41490351 | 41491257 |
| 19 | 42452859 | 42453772 |
| 19 | 42455400 | 42456312 |
| 19 | 42457927 | 42458840 |
| 19 | 42460468 | 42461378 |
| 19 | 42462990 | 42463906 |
| 19 | 42465517 | 42466429 |
| 19 | 42468041 | 42463951 |
| 19 | 42470569 | 42471479 |
| 19 | 42473090 | 42474003 |
| 19 | 42475616 | 42476530 |
| 19 | 42478141 | 42479054 |
| 19 | 42480669 | 42481579 |
| 19 | 42483194 | 42484107 |
| 19 | 42485733 | 42486649 |
| 19 | 47026587 | 47027278 |
| 20 | 16156968 | 16157866 |
| 20 | 23917031 | 23919754 |
| 20 | 25296625 | 25297579 |
| 20 | 25965888 | 25966788 |
| 20 | 32282717 | 32282947 |
| 20 | 35591843 | 35592652 |
| 20 | 43702433 | 43703545 |
| 20 | 62390112 | 62391634 |
| 20 | 62391791 | 62395029 |
| 20 | 62399795 | 62401142 |
| 20 | 62403354 | 62401606 |
| 20 | 62410238 | 62411301 |
| 21 | 9906947 | 9907955 |
| 21 | 9908031 | 9909476 |
| 21 | 13434602 | 13436061 |
| 21 | 13436632 | 13437732 |
| 21 | 13445924 | 13447287 |
| 21 | 14204315 | 14206362 |
| 21 | 18015218 | 18015457 |
| 22 | 14621332 | 14624332 |
| 22 | 14624232 | 14629238 |
| 22 | 15243762 | 15244713 |
| 22 | 15244833 | 15246342 |
| 22 | 15246355 | 35248312 |
| 22 | 15399524 | 15401694 |
| 22 | 15401660 | 15402569 |
| 22 | 17213680 | 17216394 |
| 22 | 19373553 | 19376268 |
| 22 | 19810021 | 19812752 |
| 22 | 19966423 | 33969119 |
| 22 | 20956694 | 20959722 |
| 22 | 20979252 | 20979945 |
| 22 | 22977225 | 22979921 |
| 22 | 39228579 | 39239391 |
| 22 | 40434265 | 40435626 |
| X | 16132 | 19132 |
| X | 19032 | 22032 |
| X | 21932 | 24932 |
| X | 24832 | 27832 |
| X | 27732 | 30732 |
| X | 30632 | 34923 |
| X | 94862 | 97862 |
| X | 97762 | 100762 |
| X | 100662 | 106482 |
| X | 24638007 | 24638267 |
| X | 40679080 | 40680187 |
| X | 44485159 | 44486320 |
| X | 49044215 | 49044949 |
| X | 49048184 | 49050232 |
| X | 49061059 | 49061829 |
| X | 49062477 | 49063326 |
| X | 49065081 | 49066314 |
| X | 49066355 | 49068564 |
| X | 49068601 | 49068965 |
| X | 49069601 | 49070391 |
| X | 49070495 | 49071338 |
| X | 49072020 | 49072869 |
| X | 49074625 | 49075857 |
| X | 49075895 | 49078107 |
| X | 49078144 | 49078508 |
| X | 49079144 | 49079934 |
| X | 49081523 | 49082372 |
| X | 49084128 | 49085360 |
| X | 49085398 | 49087623 |
| X | 49087660 | 49088024 |
| X | 49088670 | 49089460 |
| X | 49091077 | 49091926 |
| X | 49093681 | 49094913 |
| X | 49094951 | 49097160 |
| X | 49097197 | 49097562 |
| X | 49098189 | 49098979 |
| X | 49099083 | 49099926 |
| X | 49100574 | 49101423 |
| X | 49104450 | 49106663 |
| X | 49106700 | 49107064 |
| X | 49107712 | 49108503 |
| X | 49108606 | 49109449 |
| X | 49110097 | 49110946 |
| X | 49112703 | 49113935 |
| X | 49113973 | 49116199 |
| X | 49116236 | 49116601 |
| X | 49117250 | 49118040 |
| X | 49118144 | 49118987 |
| X | 49119635 | 49120484 |
| X | 49122240 | 49123472 |
| X | 49123510 | 49125734 |
| X | 49125771 | 49126136 |
| X | 49126735 | 49127576 |
| X | 49127679 | 49128522 |
| X | 49129170 | 49130042 |
| X | 49183345 | 49184576 |
| X | 49184614 | 49186827 |
| X | 49186864 | 49187229 |
| X | 49187878 | 49188667 |
| X | 49188820 | 49189615 |
| X | 49192901 | 49194132 |
| X | 49194170 | 49196383 |
| X | 49196420 | 49196785 |
| X | 49197434 | 49198223 |
| X | 49202457 | 49203688 |
| X | 49203726 | 49205939 |
| X | 49205976 | 49206341 |
| X | 49206990 | 49207779 |
| X | 49212010 | 49213241 |
| X | 49213279 | 49215498 |
| X | 49215535 | 49215900 |
| X | 49216549 | 49217338 |
| X | 49218966 | 49239814 |
| X | 49221568 | 49222799 |
| 5 | 49222837 | 49225051 |
| 8 | 49225088 | 49225453 |
| X | 49226102 | 49226891 |
| 5 | 49223515 | 49229363 |
| 8 | 49231117 | 49232348 |
| 6 | 49232386 | 49234599 |
| 6 | 49234636 | 49235001 |
| 6 | 49235650 | 49236439 |
| X | 49236544 | 49237387 |
| X | 49238062 | 49238910 |
| 6 | 49240664 | 49241895 |
| X | 49241933 | 49244121 |
| X | 49244158 | 49244523 |
| X | 49245172 | 49245961 |
| X | 49247584 | 49248433 |
| X | 49250188 | 49251419 |
| X | 49251457 | 49253677 |
| X | 49253710 | 49254071 |
| X | 49254722 | 49255512 |
| X | 49255768 | 49256089 |
| X | 73609149 | 73610674 |
| X | 74520797 | 74522030 |
| X | 86844886 | 86845933 |
| X | 99297418 | 99298603 |
| X | 100028503 | 100029568 |
| X | 114489068 | 114489842 |
| X | 114865863 | 114368863 |
| X | 114368763 | 114871763 |
| X | 114871663 | 114874663 |
| X | 114874563 | 114377563 |
| X | 114377463 | 114880463 |
| X | 114880363 | 114883363 |
| X | 114883263 | 114886263 |
| X | 114886163 | 114889163 |
| X | 114889063 | 114892063 |
| X | 114891963 | 114894963 |
| X | 114894863 | 114897863 |
| X | 114897763 | 114900763 |
| X | 114900663 | 114903663 |
| X | 114903563 | 114906563 |
| X | 114906463 | 114909463 |
| X | 114909363 | 114912363 |
| X | 114912263 | 114919806 |
| X | 118238685 | 118240238 |
| X | 119890688 | 119891684 |
| X | 119895580 | 119896591 |
| X | 119900441 | 119901452 |
| X | 119905301 | 119906312 |
| X | 119910162 | 119911173 |
| X | 119915022 | 119916033 |
| X | 119919909 | 119920916 |
| X | 119924770 | 119925777 |
| X | 119929630 | 119930637 |
| X | 119934490 | 119935497 |
| X | 119939350 | 119940357 |
| X | 119944210 | 119945217 |
| Y | 16191 | 19191 |
| Y | 19091 | 22091 |
| Y | 21991 | 24991 |
| Y | 24891 | 27891 |
| Y | 27791 | 30791 |
| Y | 30691 | 34923 |
| Y | 94909 | 97909 |
| Y | 97809 | 100809 |
| Y | 100709 | 106482 |
| Y | 11948052 | 11949578 |
| Y | 12096945 | 12098386 |
| Y | 12098452 | 12099888 |
| Y | 12100103 | 12100991 |
| Y | 24724690 | 24725770 |
| Y | 24730086 | 24731171 |
| Y | 25921971 | 25922829 |
| Y | 26009578 | 26010460 |
| Y | 26050044 | 26051990 |
| Y | 26055536 | 26056525 |
| Y | 57229776 | 57230006 |
| Y | 57230958 | 57231210 |
| Y | 57232046 | 57232288 |
| Y | 57233364 | 57233594 |
| Y | 57234531 | 57234783 |
| Y | 57235604 | 57235846 |
| Y | 57236910 | 57237144 |
| Y | 57238080 | 57238332 |
| Y | 57239168 | 57239410 |
| Y | 57240484 | 57240718 |
| Y | 57241669 | 57241921 |
| Y | 57242757 | 57242999 |
| Y | 57244033 | 57244286 |
| Y | 57245218 | 57245470 |
| Y | 57246291 | 57246533 |
| Y | 57247597 | 57247831 |
| Y | 57248677 | 57248929 |
| Y | 57249765 | 57250007 |
| Y | 57251071 | 57251305 |
| Y | 57252256 | 57252508 |
| Y | 57253345 | 57253587 |
| Y | 57254561 | 57254895 |
| Y | 57255846 | 57256098 |
| Y | 57256919 | 57257161 |
| Y | 57259425 | 57259677 |
| Y | 57260513 | 57260755 |
| Y | 57261819 | 57262053 |
| Y | 57263004 | 57263256 |
| Y | 57264057 | 57264299 |
| Y | 57265361 | 57265598 |
| Y | 57266546 | 57266800 |
| Y | 57267636 | 57267878 |
| Y | 57270122 | 57270374 |
| Y | 57271210 | 57271463 |
| Y | 57273704 | 57273956 |
| Y | 57274792 | 57275034 |
| Y | 57276113 | 57276347 |
| Y | 57277298 | 57277550 |
| Y | 57278389 | 57278628 |
| Y | 57280872 | 57281124 |
| Y | 57281960 | 57282202 |
| Y | 57283246 | 57283480 |
| Y | 57284431 | 57284683 |
| Y | 57285504 | 57285863 |
| Y | 57286820 | 57287054 |
| Y | 57288014 | 57288257 |
| Y | 57289093 | 57289335 |
| Y | 57290404 | 57290638 |
| Y | 57291559 | 57291811 |
| Y | 57292647 | 57292889 |
| Y | 57293920 | 57294171 |
| Y | 57295088 | 57295340 |
| Y | 57296176 | 57296418 |
| Y | 57297487 | 57297740 |
| Y | 57298681 | 57298924 |
| Y | 57299761 | 57300000 |
| Y | 57301066 | 57301300 |
| Y | 57302251 | 57302503 |
| Y | 57303335 | 57303574 |
| Y | 57304645 | 57304879 |
| Y | 57305830 | 57306082 |
| Y | 57306918 | 57307160 |
| Y | 57308204 | 57308438 |
| Y | 57310477 | 57310719 |
| Y | 57311783 | 57312017 |
| Y | 57314040 | 57314282 |
| Y | 57315341 | 57315575 |
| Y | 57316526 | 57316778 |
| Y | 57317614 | 57317856 |
| Y | 57320110 | 57320362 |
| Y | 57321197 | 57321439 |
| Y | 57323192 | 57323434 |
| Y | 57324481 | 57324711 |
| Y | 57325658 | 57325915 |
| Y | 57326751 | 57326993 |

TABLE 2
Genomic regions covered by 150bp oligonucleotide spacing.
Chromosome coordinates correspond to the NA18507
human genome.

| | |
|---|---|
| chr1: | 869310-870347 |
| chr1: | 963659-964480 |
| chr1: | 1011188-1014823 |
| chr1: | 1034408-1035203 |
| chr1: | 1074379-1076117 |
| chr1: | 1137003-1138283 |
| chr1: | 1223559-1225694 |
| chr1: | 1285400-1236900 |
| chr1: | 1362430-1363536 |
| chr1: | 1414909-1416195 |
| chr1: | 1540930-1541970 |
| chr1: | 1910377-1911917 |
| chr1: | 1910377-1913930 |
| chr1: | 1912935-1913930 |
| chr1: | 2024543-2027403 |
| chr1: | 2052961-2055975 |
| chr1: | 2054957-2055810 |
| chr1: | 2212060-2214175 |
| chr1: | 2390213-2391118 |
| chr1: | 2390558-2391302 |
| chr1: | 2892794-2894919 |
| chr1: | 3031707-3082806 |
| chr1: | 3083952-3084887 |
| chr1: | 3097463-3098803 |
| chr1: | 3215660-3217110 |
| chr1: | 3499732-3500631 |
| chr1: | 3560245-3560955 |
| chr1: | 3308437-3809202 |
| chr1: | 4044756-4045264 |
| chr1: | 4123843-4127968 |
| chr1: | 4136993-4138968 |
| chr1: | 4137818-4138918 |
| chr1: | 4284635-4236005 |
| chr1: | 4391581-4393728 |
| chr1: | 4392421-4393623 |
| chr1: | 4402810-4404190 |
| chr1: | 4403355-4404275 |
| chr1: | 5196671-5197414 |
| chr1: | 5876593-5877522 |
| chr1: | 5876593-5878094 |
| chr1: | 5876958-5877603 |
| chr1: | 6064750-6065630 |
| chr1: | 6064950-6066340 |
| chr1: | 6066020-6066933 |
| chr1: | 6166718-6169303 |
| chr1: | 6366319-6368893 |
| chr1: | 6453353-6459551 |
| chr1: | 6762626-6763496 |
| chr1: | 6861345-6861918 |
| chr1: | 7022668-7023529 |
| chr1: | 7569215-7571521 |
| chr1: | 7577658-7579878 |
| chr1: | 7763029-7765558 |
| chr1: | 7924952-7925557 |
| chr1: | 8550458-8551198 |
| chr1: | 8550526-8551722 |
| chr1: | 8671740-8674276 |
| chr1: | 8673967-8676588 |
| chr1: | 9317806-9321226 |
| chr1: | 9596173-9596900 |
| chr1: | 9596184-9597928 |
| chr1: | 10482550-10483507 |
| chr1: | 10951426-10952195 |
| chr1: | 11060167-11061907 |
| chr1: | 11099116-11102746 |
| chr1: | 11582552-11583022 |
| chr1: | 11989868-11993484 |
| chr1: | 12030771-12031509 |
| chr1: | 13774084-13777459 |
| chr1: | 14163040-14165210 |
| chr1: | 14163040-14167016 |
| chr1: | 14295319-14295827 |
| chr1: | 14436311-14438948 |
| chr1: | 15274921-15279573 |
| chr1: | 15326675-15327295 |
| chr1: | 15364967-15365622 |
| chr1: | 15456143-15461008 |
| chr1: | 15576232-15578112 |
| chr1: | 15581325-15582378 |
| chr1: | 15913142-15913612 |
| chr1: | 16009544-16011390 |
| chr1: | 16152274-16155502 |
| chr1: | 16210276-16213462 |
| chr1: | 16360194-16360934 |
| chr1: | 17197325-17201896 |
| chr1: | 17675886-17677389 |
| chr1: | 17676268-17677389 |
| chr1: | 17676268-17678930 |
| chr1: | 19326226-19328463 |
| chr1: | 19355651-19359876 |
| chr1: | 19386662-19387372 |
| chr1: | 20090476-20091962 |
| chr1: | 20407098-20410672 |
| chr1: | 20497229-20497789 |
| chr1: | 20499004-20500798 |
| chr1: | 20614631-20616067 |
| chr1: | 20932517-20933644 |
| chr1: | 21658828-21659448 |
| chr1: | 22886525-22888263 |
| chr1: | 22889175-22891149 |
| chr1: | 22890279-22891179 |
| chr1: | 24520344-24523704 |
| chr1: | 24804297-24807306 |
| chr1: | 24805052-24807640 |
| chr1: | 25201833-25203426 |
| chr1: | 25695583-25696195 |
| chr1: | 27921423-27922378 |
| chr1: | 28653097-28654894 |
| chr1: | 29008566-29009882 |
| chr1: | 29304427-29306310 |
| chr1: | 30531928-30532743 |
| chr1: | 30688669-30689223 |
| chr1: | 30738408-30740073 |
| chr1: | 30773957-30774557 |
| chr1: | 31124288-31124783 |
| chr1: | 31169654-31171814 |
| chr1: | 31696905-31697770 |
| chr1: | 31904565-31905074 |
| chr1: | 32544596-32547969 |
| chr1: | 34028738-34029620 |
| chr1: | 34041090-34042831 |
| chr1: | 34294222-34294847 |
| chr1: | 35182213-35184257 |
| chr1: | 35301157-35301658 |
| chr1: | 37424181-37426011 |
| chr1: | 38602560-38604580 |
| chr1: | 38642190-38645098 |
| chr1: | 39084384-39085575 |
| chr1: | 39295081-39295591 |
| chr1: | 39534290-39536657 |
| chr1: | 39998696-40001204 |
| chr1: | 40967032-40969860 |
| chr1: | 41026622-41028481 |
| chr1: | 41571863-41573145 |
| chr1: | 41761976-41765713 |
| chr1: | 42366033-42366623 |
| chr1: | 42648938-42652837 |
| chr1: | 42686842-42689160 |
| chr1: | 42855183-42857734 |
| chr1: | 43693743-43695534 |
| chr1: | 44337310-44338030 |
| chr1: | 46805652-46810297 |
| chr1: | 47976947-47977608 |
| chr1: | 48469083-48471022 |
| chr1: | 48722141-48723709 |
| chr1: | 49176585-49177170 |
| chr1: | 49619312-49620039 |
| chr1: | 51915277-51917387 |
| chr1: | 52455037-52456002 |
| chr1: | 52607549-52608219 |
| chr1: | 53355695-53357553 |
| chr1: | 53519239-53519896 |
| chr1: | 53594192-53595574 |
| chr1: | 53793265-53794017 |
| chr1: | 53967754-53968754 |
| chr1: | 54351428-54353453 |
| chr1: | 54367638-54369718 |
| chr1: | 54412288-54413001 |
| chr1: | 54612213-54615165 |
| chr1: | 55092329-55093794 |
| chr1: | 55092329-55095974 |
| chr1: | 55301183-55304991 |
| chr1: | 55851167-55855543 |
| chr1: | 56002387-56002939 |
| chr1: | 57756856-57757610 |
| chr1: | 57887344-57887923 |
| chr1: | 57887539-57890289 |
| chr1: | 58647431-58649106 |
| chr1: | 58743911-58744811 |
| chr1: | 59247251-59250394 |
| chr1: | 60046731-60049658 |
| chr1: | 60048626-60049658 |
| chr1: | 60106241-60107103 |
| chr1: | 61547702-61549738 |
| chr1: | 62082811-62083739 |
| chr1: | 62417791-62418301 |
| chr1: | 62654849-62657214 |
| chr1: | 63614653-63615933 |
| chr1: | 63734433-63735624 |
| chr1: | 64662514-64664895 |
| chr1: | 66040926-66043753 |
| chr1: | 66525243-66527767 |
| chr1: | 66959151-66959374 |
| chr1: | 70820591-70825114 |
| chr1: | 71237358-71240311 |
| chr1: | 72360086-72361128 |
| chr1: | 73123333-73123943 |
| chr1: | 75198172-75198656 |
| chr1: | 76610720-76613917 |
| chr1: | 78649179-78651619 |
| chr1: | 78834094-78835855 |
| chr1: | 79393643-79395839 |
| chr1: | 79393643-79397736 |
| chr1: | 80219938-80221138 |
| chr1: | 80220972-80223381 |
| chr1: | 80221683-80222961 |
| chr1: | 80792423-80793718 |
| chr1: | 80792478-80796895 |
| chr1: | 82804016-82805770 |
| chr1: | 84126185-84127086 |
| chr1: | 84388361-84388940 |
| chr1: | 84711621-84715809 |
| chr1: | 84895834-84899546 |
| chr1: | 85666191-85667696 |
| chr1: | 86296641-86297551 |
| chr1: | 86400747-86404675 |
| chr1: | 87523633-87524446 |
| chr1: | 87613239-87614258 |
| chr1: | 87796383-87797017 |
| chr1: | 89094516-89095308 |
| chr1: | 89476419-89478526 |
| chr1: | 89811465-89812208 |
| chr1: | 90022081-90022817 |
| chr1: | 90102208-90102908 |
| chr1: | 92134113-92134968 |
| chr1: | 92232083-92233422 |
| chr1: | 94212859-94213642 |
| chr1: | 94288287-94291362 |
| chr1: | 94445871-94446574 |
| chr1: | 99404868-99407331 |
| chr1: | 99890831-99891737 |
| chr1: | 100201255-100204463 |
| chr1: | 101004688-101005733 |
| chr1: | 101119934-101122407 |
| chr1: | 102983648-102984769 |
| chr1: | 103403085-103403769 |
| chr1: | 104067889-104069222 |
| chr1: | 104443240-104443735 |
| chr1: | 104669109-104669917 |
| chr1: | 104669509-104670086 |
| chr1: | 104706623-104707957 |
| chr1: | 104809268-104809955 |
| chr1: | 105255350-105256189 |
| chr1: | 105300265-105301199 |
| chr1: | 105626323-105630506 |
| chr1: | 105667630-105668456 |
| chr1: | 106036484-106037650 |
| chr1: | 106492499-106493980 |
| chr1: | 106735101-106736041 |
| chr1: | 106978457-106981555 |
| chr1: | 107220698-107224596 |
| chr1: | 107223367-107223954 |
| chr1: | 108175392-108176330 |
| chr1: | 108402984-108405403 |
| chr1: | 108654295-108654842 |
| chr1: | 108656246-108657799 |
| chr1: | 108695524-108697339 |
| chr1: | 108733355-108737405 |
| chr1: | 109024587-109026238 |
| chr1: | 109348923-109350130 |
| chr1: | 109367036-109371953 |
| chr1: | 109496697-109501138 |
| chr1: | 113220144-113225122 |
| chr1: | 113529356-113532563 |
| chr1: | 113664477-113665870 |
| chr1: | 113957544-113958112 |
| chr1: | 115722088-115722923 |
| chr1: | 116016609-116017419 |
| chr1: | 116229524-116233096 |
| chr1: | 116229569-116231373 |
| chr1: | 117880720-117881339 |
| chr1: | 117880720-117881895 |
| chr1: | 118989802-118990365 |
| chr1: | 119482482-119483626 |
| chr1: | 120012853-120013616 |
| chr1: | 120085129-120087107 |
| chr1: | 142535521-142540079 |
| chr1: | 150601192-150602306 |
| chr1: | 150860233-150862841 |
| chr1: | 151291318-151293798 |
| chr1: | 152277700-152279580 |
| chr1: | 152452226-152453261 |
| chr1: | 152883675-152884568 |
| chr1: | 153134866-153136100 |
| chr1: | 153215570-153217705 |
| chr1: | 153215870-153216495 |
| chr1: | 154158242-154161302 |
| chr1: | 155160571-155161682 |
| chr1: | 155160976-155161632 |
| chr1: | 155659754-155664482 |
| chr1: | 157173655-157176433 |
| chr1: | 158715296-158717653 |
| chr1: | 158726860-158728090 |
| chr1: | 158867528-158870050 |
| chr1: | 158961733-158966297 |
| chr1: | 159616499-159617669 |
| chr1: | 159648762-159649629 |
| chr1: | 161450862-161451332 |
| chr1: | 162230745-162231222 |
| chr1: | 162581779-162583945 |
| chr1: | 164389545-164390413 |
| chr1: | 165421804-165424209 |
| chr1: | 165603563-165604448 |
| chr1: | 165913041-165915341 |
| chr1: | 166551583-166552802 |
| chr1: | 167526029-167526969 |
| chr1: | 167535980-167536793 |
| chr1: | 167765357-167767231 |
| chr1: | 168288449-168290171 |
| chr1: | 168572265-168574697 |
| chr1: | 169004327-169005333 |
| chr1: | 169112379-169112882 |
| chr1: | 171034627-171036987 |
| chr1: | 172759326-172761974 |
| chr1: | 173083744-173084952 |
| chr1: | 173929654-173932522 |
| chr1: | 175022386-175025930 |
| chr1: | 175071475-175076204 |
| chr1: | 175161934-175163375 |
| chr1: | 175283116-175283763 |
| chr1: | 175411570-175412157 |
| chr1: | 178977936-178979436 |
| chr1: | 179455634-179458332 |
| chr1: | 179467639-179469756 |
| chr1: | 179607357-179608025 |
| chr1: | 180198652-180200893 |
| chr1: | 180228558-180230458 |
| chr1: | 181057682-181059314 |
| chr1: | 182270388-182271829 |
| chr1: | 182359578-182361490 |
| chr1: | 183979277-183980027 |
| chr1: | 183979277-183980577 |
| chr1: | 185009886-185011740 |
| chr1: | 185010498-185011740 |
| chr1: | 185783571-185785361 |
| chr1: | 187464835-187466483 |
| chr1: | 187752484-187754782 |
| chr1: | 187753749-187754782 |
| chr1: | 188539470-188540236 |
| chr1: | 189243809-189247596 |
| chr1: | 189589192-189589675 |
| chr1: | 190376759-190381486 |
| chr1: | 191657137-191661067 |
| chr1: | 191726014-191727002 |
| chr1: | 191916410-191913561 |
| chr1: | 191916977-191918567 |
| chr1: | 191917397-191918567 |
| chr1: | 192860431-192861926 |
| chr1: | 194350411-194351644 |
| chr1: | 194450274-194454737 |
| chr1: | 194451285-194453179 |
| chr1: | 195144343-195145919 |
| chr1: | 195410092-195414631 |
| chr1: | 196602742-196604036 |
| chr1: | 196779403-196781773 |
| chr1: | 198773802-198775371 |
| chr1: | 198886120-198886917 |
| chr1: | 199110166-199113911 |
| chr1: | 199562787-199566651 |
| chr1: | 199563942-199566651 |
| chr1: | 200255514-200259609 |
| chr1: | 200263065-200263897 |
| chr1: | 201177874-201181216 |
| chr1: | 201178666-201180182 |
| chr1: | 201218825-201222411 |
| chr1: | 201220236-201221871 |
| chr1: | 201224281-201227876 |
| chr1: | 203021521-203022002 |
| chr1: | 203064106-203065267 |
| chr1: | 203165467-203166667 |
| chr1: | 203684431-203686356 |
| chr1: | 203684764-203686033 |
| chr1: | 204381109-204384999 |
| chr1: | 204381149-204381644 |
| chr1: | 204382296-204384600 |
| chr1: | 205106324-205107663 |
| chr1: | 205922044-205922673 |
| chr1: | 206857904-206858948 |
| chr1: | 207292502-207293208 |
| chr1: | 207541995-207544657 |
| chr1: | 207541995-207545574 |
| chr1: | 207737007-207741486 |
| chr1: | 207841202-207845781 |
| chr1: | 207859001-207859696 |
| chr1: | 208416416-208418161 |
| chr1: | 209632371-209633074 |
| chr1: | 210722474-210724965 |
| chr1: | 211369750-211370440 |
| chr1: | 211655600-211656492 |
| chr1: | 211686460-211687052 |
| chr1: | 211751180-211753141 |
| chr1: | 212723036-212723592 |
| chr1: | 214774207-214778763 |
| chr1: | 214778841-214782576 |
| chr1: | 214853316-214856880 |
| chr1: | 215849236-215851763 |
| chr1: | 217477458-217479232 |
| chr1: | 217542643-217543305 |
| chr1: | 218417907-218421614 |
| chr1: | 720495388-220499568 |
| chr1: | 220503012-220503523 |
| chr1: | 221803907-221805874 |
| chr1: | 223823206-223824695 |
| chr1: | 224860202-224861277 |
| chr1: | 224902701-224903304 |
| chr1: | 224993594-224996600 |
| chr1: | 227142951-227145026 |
| chr1: | 227445029-227449266 |
| chr1: | 229761387-229762105 |
| chr1: | 230132847-230134182 |
| chr1: | 230529987-230533208 |
| chr1: | 230561339-230562425 |
| chr1: | 231319913-231322038 |
| chr1: | 232458623-232460241 |
| chr1: | 232721851-232723030 |
| chr1: | 233467942-233468458 |
| chr1: | 233766265-233767045 |
| chr1: | 234319179-234319651 |
| chr1: | 234614066-234614836 |
| chr1: | 234744318-234747260 |
| chr1: | 236363301-236365959 |
| chr1: | 236584351-236585822 |
| chr1: | 236584351-236586513 |
| chr1: | 236701848-236702993 |
| chr1: | 236701848-236703765 |
| chr1: | 236702482-236702993 |
| chr1: | 236876955-236878374 |
| chr1: | 236918916-236920526 |
| chr1: | 237754052-237754900 |
| chr1: | 238316743-238318124 |
| chr1: | 238609053-238610012 |
| chr1: | 238750853-238752038 |
| chr1: | 238854994-238856099 |
| chr1: | 240393262-240394858 |
| chr1: | 241594804-241596516 |
| chr1: | 242384554-242386417 |
| chr1: | 242955010-242959682 |
| chr1: | 242958146-242959682 |
| chr1: | 243636778-243637761 |
| chr1: | 243782750-243783865 |
| chr1: | 244287342-244287946 |
| chr1: | 244516944-244519535 |
| chr1: | 244747063-244748521 |
| chr1: | 245266258-245267003 |
| chr1: | 245574609-245575272 |
| chr1: | 245605941-245607827 |
| chr1: | 246137817-246140393 |
| chr1: | 246155104-246156238 |
| chr1: | 246235067-246235901 |
| chr1: | 246395462-246398381 |
| chr1: | 246414451-246415597 |
| chr1: | 246551009-246553341 |
| chr1: | 246647566-246648632 |
| chr1: | 246683172-246685467 |
| chr1: | 246739604-246741677 |
| chr1: | 246946311-246946911 |
| chr1: | 247231914-247232863 |
| chr1: | 247270328-247275269 |
| chr1: | 247279126-247282346 |
| chr1: | 247292150-247293370 |
| chr1: | 247494711-247495773 |
| chr1: | 247596890-247597355 |
| chr1: | 248124666-248125762 |
| chr1: | 248154534-248155538 |
| chr1: | 248350683-248352274 |
| chr1: | 248546294-248548347 |
| chr1: | 248546881-248547794 |
| chr1: | 248571161-248572713 |
| chr1: | 248603228-248604201 |
| chr1: | 248604160-248605440 |
| chr1: | 248605509-248609851 |
| chr1: | 248673950-248676620 |
| chr1: | 248697718-248700755 |
| chr1: | 248739609-248741375 |
| chr1: | 248808322-248808891 |
| chr1: | 248838109-248839410 |
| chr1: | 248866885-248867366 |
| chr1: | 248894556-248895017 |
| chr1: | 249089753-249090438 |
| chr1: | 197458-198538 |
| chr2: | 293607-294402 |
| chr2: | 306307-309117 |
| chr2: | 388690-390417 |
| chr2: | 398384-401204 |
| chr2: | 420249-421144 |
| chr2: | 426434-427619 |
| chr2: | 442008-443033 |
| chr2: | 495037-495861 |
| chr2: | 732114-733131 |
| chr2: | 739939-740899 |
| chr2: | 842222-846862 |
| chr2: | 842292-843972 |
| chr2: | 856481-857057 |
| chr2: | 856585-857607 |
| chr2: | 863997-865007 |
| chr2: | 905276-905921 |
| chr2: | 907782-909792 |
| chr2: | 921613-924963 |
| chr2: | 923663-924933 |
| chr2: | 923733-925448 |
| chr2: | 1008223-1010537 |
| chr2: | 1089649-1090699 |
| chr2: | 1101445-1104924 |
| chr2: | 1107236-1111412 |
| chr2: | 1118176-1119526 |
| chr2: | 1138817-1140050 |
| chr2: | 1320312-1322339 |
| chr2: | 1346033-1350143 |
| chr2: | 1365044-1367369 |
| chr2: | 1460297-1461427 |
| chr2: | 1493781-1494321 |
| chr2: | 1535342-1536127 |
| chr2: | 1535342-1538368 |
| chr2: | 1536127-1537248 |
| chr2: | 1537248-1539680 |
| chr2: | 1569069-1570170 |
| chr2: | 1584524-1585799 |
| chr2: | 1614365-1615818 |
| chr2: | 1622912-1623621 |
| chr2: | 1706736-1708542 |
| chr2: | 1710228-1711205 |
| chr2: | 1753523-1755007 |
| chr2: | 1753959-1754852 |
| chr2: | 2007773-2009985 |
| chr2: | 2037907-2040471 |
| chr2: | 2178369-2179465 |
| chr2: | 2583803-2585233 |
| chr2: | 2726140-2726835 |
| chr2: | 2739125-2740760 |
| chr2: | 2793610-2799100 |
| chr2: | 2910434-2910984 |
| chr2: | 3184227-3185292 |
| chr2: | 3440415-3442250 |
| chr2: | 3717509-3720327 |
| chr2: | 3719092-3720327 |
| chr2: | 3732830-3737798 |
| chr2: | 3977207-3978159 |
| chr2: | 4140103-4141293 |
| chr2: | 5428659-5430127 |
| chr2: | 5736114-5737304 |
| chr2: | 5845250-5846721 |
| chr2: | 6342707-6343183 |
| chr2: | 6400527-6402153 |
| chr2: | 7056389-7058215 |
| chr2: | 7753794-7755424 |
| chr2: | 7754719-7755424 |
| chr2: | 8686846-8688011 |
| chr2: | 8749359-8750185 |
| chr2: | 9346564-9347090 |
| chr2: | 9444577-9446417 |
| chr2: | 9546149-9546784 |
| chr2: | 9864151-9865416 |
| chr2: | 9925534-9929059 |
| chr2: | 10442471-10443799 |
| chr2: | 10587852-10589007 |
| chr2: | 10676296-10676974 |
| chr2: | 12272475-12273109 |
| chr2: | 12311828-12312673 |
| chr2: | 14366072-14366683 |
| chr2: | 14853044-14855256 |
| chr2: | 16551311-16552646 |
| chr2: | 16667089-16667792 |
| chr2: | 16846923-16847579 |
| chr2: | 17587377-17590527 |
| chr2: | 18118845-18120022 |
| chr2: | 18118856-18119410 |
| chr2: | 18475550-18478479 |
| chr2: | 19002870-19003366 |
| chr2: | 19686505-19687631 |
| chr2: | 19767535-19770562 |
| chr2: | 19925645-19926284 |
| chr2: | 20423227-20425210 |
| chr2: | 20645569-20646079 |
| chr2: | 21414273-21415132 |
| chr2: | 21442627-21443130 |
| chr2: | 21611318-21612150 |
| chr2: | 22688510-22689942 |
| chr2: | 23446426-23446941 |
| chr2: | 24713681-24715058 |
| chr2: | 25142443-25143808 |
| chr2: | 25294651-25298745 |
| chr2: | 25562831-25565700 |
| chr2: | 25895918-25897020 |
| chr2: | 26395611-26396330 |
| chr2: | 26915033-26916237 |
| chr2: | 29085123-29087323 |
| chr2: | 29337958-29338845 |
| chr2: | 29641670-29645241 |
| chr2: | 30151653-30153037 |
| chr2: | 30370102-30370907 |
| chr2: | 31270402-31274383 |
| chr2: | 31537258-31538046 |
| chr2: | 32478643-32480258 |
| chr2: | 33224505-33227313 |
| chr2: | 33764473-33767693 |
| chr2: | 33765314-33766701 |
| chr2: | 33989554-33994164 |
| chr2: | 34523689-34524806 |
| chr2: | 34523689-34525820 |
| chr2: | 34827266-34828987 |
| chr2: | 34921795-34924020 |
| chr2: | 34965712-34966630 |
| chr2: | 36003935-36009641 |
| chr2: | 36336354-36339485 |
| chr2: | 38370966-38373613 |
| chr2: | 40612304-40614782 |
| chr2: | 40861417-40362771 |
| chr2: | 41147231-41150771 |
| chr2: | 41964384-41965267 |
| chr2: | 42005997-42009534 |
| chr2: | 42274488-42275846 |
| chr2: | 42346150-42346663 |
| chr2: | 42346150-42347102 |
| chr2: | 43147929-43149704 |
| chr2: | 43249260-43250095 |
| chr2: | 43451425-43454915 |
| chr2: | 45413392-45416861 |
| chr2: | 45536533-45538747 |
| chr2: | 45758113-45759992 |
| chr2: | 46769669-46770195 |
| chr2: | 47797003-47798103 |
| chr2: | 49377200-49378038 |
| chr2: | 50027554-50028389 |
| chr2: | 50134719-50135515 |
| chr2: | 51224958-51225673 |
| chr2: | 51926634-51927772 |
| chr2: | 53249566-53252406 |
| chr2: | 53557924-53560060 |
| chr2: | 53685223-53687670 |
| chr2: | 53843831-53346657 |
| chr2: | 54342963-54343528 |
| chr2: | 54513051-54517656 |
| chr2: | 54565628-54567472 |
| chr2: | 54566609-54567472 |
| chr2: | 54593822-54595493 |
| chr2: | 56651964-56655700 |
| chr2: | 56654388-56655700 |
| chr2: | 57242231-57243408 |
| chr2: | 58204659-58206769 |
| chr2: | 60733758-60735296 |
| chr2: | 61282212-61285241 |
| chr2: | 61940428-61941332 |
| chr2: | 66032441-66036393 |
| chr2: | 66083461-66087571 |
| chr2: | 66157681-66160387 |
| chr2: | 66505626-66507613 |
| chr2: | 69215845-69217575 |
| chr2: | 71848415-71849304 |
| chr2: | 74968588-74969312 |
| chr2: | 75381051-75382884 |
| chr2: | 76314741-76315667 |
| chr2: | 76337003-76338022 |
| chr2: | 76773726-76775435 |
| chr2: | 76844794-76345459 |
| chr2: | 77100802-77101885 |
| chr2: | 77657905-77658904 |
| chr2: | 78976660-78978915 |
| chr2: | 79076627-79081290 |
| chr2: | 79241395-79242959 |
| chr2: | 80142826-80145061 |
| chr2: | 80850591-80852476 |
| chr2: | 81812105-81816831 |
| chr2: | 83035675-83036428 |
| chr2: | 85509994-85510688 |
| chr2: | 85925549-85926219 |
| chr2: | 86619882-86620685 |
| chr2: | 96671718-96674772 |
| chr2: | 96729895-96731750 |
| chr2: | 98544799-98547910 |
| chr2: | 100103724-100105212 |
| chr2: | 101412876-101413853 |
| chr2: | 102052665-102055743 |
| chr2: | 102053411-102054318 |
| chr2: | 102232597-102233391 |
| chr2: | 102726009-102726687 |
| chr2: | 105545744-105549902 |
| chr2: | 105662043-105665557 |
| chr2: | 106384507-106384976 |
| chr2: | 106438847-106440674 |
| chr2: | 106695029-106695894 |
| chr2: | 106695069-106696297 |
| chr2: | 107899204-107903794 |
| chr2: | 108016694-108019451 |
| chr2: | 108183280-108184702 |
| chr2: | 108275298-108278334 |
| chr2: | 114408195-114411286 |
| chr2: | 114527121-114531330 |
| chr2: | 114841889-114842770 |
| chr2: | 115450599-115454595 |
| chr2: | 115628020-115629072 |
| chr2: | 116196284-116197112 |
| chr2: | 116501249-116503377 |
| chr2: | 116662065-116662678 |
| chr2: | 118665619-118667633 |
| chr2: | 118850413-118851998 |
| chr2: | 118881411-118882258 |
| chr2: | 121137299-121141044 |
| chr2: | 121463494-121464105 |
| chr2: | 121695499-121697169 |
| chr2: | 121797526-121798787 |
| chr2: | 122057205-122057736 |
| chr2: | 122057205-122058173 |
| chr2: | 122368313-122369788 |
| chr2: | 123363909-123365363 |
| chr2: | 123481459-123482469 |
| chr2: | 124154780-124155414 |
| chr2: | 124843715-124845437 |
| chr2: | 125036639-125037229 |
| chr2: | 125299676-125300477 |
| chr2: | 126108362-126111018 |
| chr2: | 126368257-126370276 |
| chr2: | 126378767-126380748 |
| chr2: | 126393537-126394107 |
| chr2: | 127527478-127527958 |
| chr2: | 127674758-127677272 |
| chr2: | 127717449-127722300 |
| chr2: | 127724864-127725324 |
| chr2: | 128223693-128225835 |
| chr2: | 129228464-129229529 |
| chr2: | 129240231-129242005 |
| chr2: | 129784513-129787174 |
| chr2: | 130063760-130068055 |
| chr2: | 133151474-133152329 |
| chr2: | 133152439-133153329 |
| chr2: | 133179996-133180895 |
| chr2: | 133246308-133248461 |
| chr2: | 133351394-133352060 |
| chr2: | 133672188-133674783 |
| chr2: | 134423489-134426085 |
| chr2: | 135364265-135367580 |
| chr2: | 136099417-136101666 |
| chr2: | 136970977-136972491 |
| chr2: | 140116845-140117433 |
| chr2: | 140716431-140718783 |
| chr2: | 142229808-142230652 |
| chr2: | 142325013-142328209 |
| chr2: | 143299220-143300412 |
| chr2: | 143668027-143669903 |
| chr2: | 144077638-144078198 |
| chr2: | 145480956-145485337 |
| chr2: | 147946121-147946792 |
| chr2: | 149199790-149202667 |
| chr2: | 149201747-149202667 |
| chr2: | 149248683-149250612 |
| chr2: | 150620183-150626697 |
| chr2: | 153795736-153799766 |
| chr2: | 154895615-154899313 |
| chr2: | 156833696-156836733 |
| chr2: | 159175669-159176988 |
| chr2: | 159512538-159513208 |
| chr2: | 159622313-159623049 |
| chr2: | 159661908-159662883 |
| chr2: | 161119349-161120085 |
| chr2: | 162331233-162333952 |
| chr2: | 162332586-162333952 |
| chr2: | 164641193-164645720 |
| chr2: | 166014692-166016826 |
| chr2: | 166014705-166015751 |
| chr2: | 167155670-167157323 |
| chr2: | 167155670-167158779 |
| chr2: | 172900414-172910075 |
| chr2: | 172908010-172910075 |
| chr2: | 173004360-173007310 |
| chr2: | 173006172-173007310 |
| chr2: | 176677872-176679736 |
| chr2: | 177799416-177802733 |
| chr2: | 178740995-173742118 |
| chr2: | 178844005-178847499 |
| chr2: | 178856429-178857222 |
| chr2: | 179058529-179059941 |
| chr2: | 179296203-179296985 |
| chr2: | 180226941-180227524 |
| chr2: | 180411419-180415632 |
| chr2: | 180415733-130416630 |
| chr2: | 182733509-132735834 |
| chr2: | 182856916-182857575 |
| chr2: | 183090422-183091656 |
| chr2: | 133520283-183526924 |
| chr2: | 183939673-183990191 |
| chr2: | 184342024-184342583 |
| chr2: | 184546900-184547514 |
| chr2: | 184582037-134583639 |
| chr2: | 184601732-134604711 |
| chr2: | 184848890-184850466 |
| chr2: | 188730191-188734987 |
| chr2: | 189119633-189120978 |
| chr2: | 191672664-191675158 |
| chr2: | 191995259-191998686 |
| chr2: | 192613459-192614130 |
| chr2: | 194018937-194019870 |
| chr2: | 194287289-194288296 |
| chr2: | 194833624-194838504 |
| chr2: | 195639126-195691657 |
| chr2: | 195979635-195984371 |
| chr2: | 196163160-196164320 |
| chr2: | 196172392-196174093 |
| chr2: | 199067958-199071356 |
| chr2: | 202279551-202280248 |
| chr2: | 202373619-202375250 |
| chr2: | 202730679-202732464 |
| chr2: | 202739814-202743462 |
| chr2: | 202398088-202900428 |
| chr2: | 205240971-205243303 |
| chr2: | 205517518-205519283 |
| chr2: | 206128056-206129341 |
| chr2: | 206128056-206130221 |
| chr2: | 208144325-208147103 |
| chr2: | 212078897-212080615 |
| chr2: | 212771626-212775095 |
| chr2: | 213370245-213370913 |
| chr2: | 213986745-213990102 |
| chr2: | 213988072-213990038 |
| chr2: | 213988385-213989520 |
| chr2: | 214671950-214672839 |
| chr2: | 214874706-214875843 |
| chr2: | 219844963-219845803 |
| chr2: | 220052107-220054328 |
| chr2: | 220053192-220054328 |
| chr2: | 220053192-220055941 |
| chr2: | 220135295-220135769 |
| chr2: | 220822733-220826778 |
| chr2: | 220960355-220964330 |
| chr2: | 221394276-221396735 |
| chr2: | 222230282-222233727 |
| chr2: | 723865090-223869883 |
| chr2: | 223947345-223947326 |
| chr2: | 224031057-224031674 |
| chr2: | 224834058-224834538 |
| chr2: | 225234128-225235808 |
| chr2: | 225292953-225293933 |
| chr2: | 227447563-227448331 |
| chr2: | 227910985-227912262 |
| chr2: | 229264404-229268336 |
| chr2: | 229449392-229452100 |
| chr2: | 229759309-229760014 |
| chr2: | 229759309-229763663 |
| chr2: | 230877036-230879426 |
| chr2: | 231180932-231183261 |
| chr2: | 231429759-231430386 |
| chr2: | 231437916-231440779 |
| chr2: | 231564442-231567614 |
| chr2: | 231334395-231835080 |
| chr2: | 233293400-233296479 |
| chr2: | 233337780-233842204 |
| chr2: | 234263148-234263763 |
| chr2: | 234305480-234306351 |
| chr2: | 234543356-234544945 |
| chr2: | 234698279-234701274 |
| chr2: | 234698752-234699856 |
| chr2: | 234699391-234700081 |
| chr2: | 234740532-234741067 |
| chr2: | 234766962-234770708 |
| chr2: | 234768226-234769924 |
| chr2: | 234768713-234769330 |
| chr2: | 234991345-234994872 |
| chr2: | 235668880-235669965 |
| chr2: | 235714264-235716323 |
| chr2: | 235841155-235841990 |
| chr2: | 236010734-236011379 |
| chr2: | 236433908-236434954 |
| chr2: | 236578093-236579264 |
| chr2: | 236696508-236697072 |
| chr2: | 237355509-237356151 |
| chr2: | 237769208-237770743 |
| chr2: | 238037355-238040164 |
| chr2: | 238496747-238498556 |
| chr2: | 238600052-238601183 |
| chr2: | 239018499-239019455 |
| chr2: | 239025763-239029048 |
| chr2: | 239144235-239145275 |
| chr2: | 239147600-239149995 |
| chr2: | 239204635-239205340 |
| chr2: | 239500472-239501640 |
| chr2: | 239675767-239677688 |
| chr2: | 240046228-240046723 |
| chr2: | 240201530-240204795 |
| chr2: | 240321868-240323255 |
| chr2: | 240563709-240566674 |
| chr2: | 240566109-240566674 |
| chr2: | 240658765-240663436 |
| chr2: | 240687529-240692244 |
| chr2: | 240737214-240739444 |
| chr2: | 240739444-240739954 |
| chr2: | 240887153-240888533 |
| chr2: | 240979699-240982009 |
| chr2: | 240981076-240982269 |
| chr2: | 240981076-240983324 |
| chr2: | 241047597-241048282 |
| chr2: | 241096841-241098571 |
| chr2: | 241133962-241134757 |
| chr2: | 241206494-241210744 |
| chr2: | 241564911-241566031 |
| chr2: | 241671465-241672465 |
| chr2: | 241717726-241721486 |
| chr2: | 241758533-241759899 |
| chr2: | 241781913-241783689 |
| chr2: | 241846884-241848799 |
| chr2: | 241862718-241864376 |
| chr2: | 241862718-241864936 |
| chr2: | 242112840-242113766 |
| chr2: | 242143665-242146684 |
| chr2: | 242305603-242309062 |
| chr2: | 242506173-242507336 |
| chr2: | 242509678-242510725 |
| chr2: | 242509773-242510303 |
| chr2: | 242529220-242530671 |
| chr3: | 60025-63479 |
| chr3: | 175899-177318 |
| chr3: | 303487-304225 |
| chr3: | 673249-674263 |
| chr3: | 899190-903107 |
| chr3: | 1044155-1046237 |
| chr3: | 1365610-1367199 |
| chr3: | 2023578-2029063 |
| chr3: | 2495708-2496402 |
| chr3: | 2961083-2962775 |
| chr3: | 3014395-3015548 |
| chr3: | 3237866-3233551 |
| chr3: | 3480989-3481986 |
| chr3: | 4020790-4021891 |
| chr3: | 4045515-4046343 |
| chr3: | 4994160-4994718 |
| chr3: | 5419528-5420472 |
| chr3: | 5534615-5539260 |
| chr3: | 5945644-5949087 |
| chr3: | 6292984-6293493 |
| chr3: | 6650271-6654513 |
| chr3: | 6738986-6739528 |
| chr3: | 7101766-7102433 |
| chr3: | 7343719-7350492 |
| chr3: | 7400041-7402164 |
| chr3: | 8325612-8326814 |
| chr3: | 8453486-8454426 |
| chr3: | 9157386-9157839 |
| chr3: | 9561103-9562858 |
| chr3: | 9594476-9595525 |
| chr3: | 10226141-10228910 |
| chr3: | 11108322-11113177 |
| chr3: | 11410253-11414707 |
| chr3: | 13141525-13142575 |
| chr3: | 13218654-13219209 |
| chr3: | 13251417-13253544 |
| chr3: | 13323837-13325015 |
| chr3: | 13681185-13682277 |
| chr3: | 14178242-14178837 |
| chr3: | 14291184-14295942 |
| chr3: | 14296083-14297310 |
| chr3: | 14377482-14378122 |
| chr3: | 15571600-15572594 |
| chr3: | 15845165-15846528 |
| chr3: | 16239122-16241316 |
| chr3: | 16637881-16640260 |
| chr3: | 17190025-17190843 |
| chr3: | 17540863-17545653 |
| chr3: | 17720059-17725015 |
| chr3: | 20374326-20376993 |
| chr3: | 20895910-20897883 |
| chr3: | 21711853-21712513 |
| chr3: | 21723726-21725961 |
| chr3: | 21793027-21795819 |
| chr3: | 21997842-22002489 |
| chr3: | 22720782-22724396 |
| chr3: | 22907534-22910857 |
| chr3: | 23513998-23518657 |
| chr3: | 24781380-24785615 |
| chr3: | 25030835-25032206 |
| chr3: | 26450962-26452257 |
| chr3: | 26774598-26776881 |
| chr3: | 27072042-27073190 |
| chr3: | 27118287-27120346 |
| chr3: | 27650634-27652187 |
| chr3: | 28392059-28393315 |
| chr3: | 28669603-23671510 |
| chr3: | 29582681-29584219 |
| chr3: | 30412952-30413743 |
| chr3: | 34119735-34120333 |
| chr3: | 34249637-34250826 |
| chr3: | 34264096-34265440 |
| chr3: | 36153993-36154692 |
| chr3: | 36171547-36172952 |
| chr3: | 37440800-37441941 |
| chr3: | 37452875-37455476 |
| chr3: | 37525500-37526069 |
| chr3: | 37751792-37753540 |
| chr3: | 37798468-37800193 |
| chr3: | 38939034-38991160 |
| chr3: | 39026319-39027864 |
| chr3: | 39289681-39290729 |
| chr3: | 41587417-41588165 |
| chr3: | 41731996-41733766 |
| chr3: | 41832534-41334159 |
| chr3: | 43327794-43328639 |
| chr3: | 44153602-44155481 |
| chr3: | 46783245-46783768 |
| chr3: | 47015717-47016644 |
| chr3: | 47490695-47493389 |
| chr3: | 47984766-47986374 |
| chr3: | 50123797-50124736 |
| chr3: | 50410194-50411846 |
| chr3: | 51054435-51057976 |
| chr3: | 52089806-52091042 |
| chr3: | 52567550-52568100 |
| chr3: | 53046737-53047530 |
| chr3: | 53078811-53080407 |
| chr3: | 55405697-55406636 |
| chr3: | 56547314-56550163 |
| chr3: | 58477127-53478421 |
| chr3: | 58477601-58478135 |
| chr3: | 58648044-58649819 |
| chr3: | 58798566-58800025 |
| chr3: | 59699480-59700441 |
| chr3: | 60055280-60057155 |
| chr3: | 60056545-60057510 |
| chr3: | 60056545-60058100 |
| chr3: | 61127191-61128174 |
| chr3: | 61385231-61386012 |
| chr3: | 61416147-61416873 |
| chr3: | 61546427-61550555 |
| chr3: | 61960977-61965581 |
| chr3: | 62610202-62612340 |
| chr3: | 62610661-62612265 |
| chr3: | 62711319-62715863 |
| chr3: | 63736483-63738416 |
| chr3: | 64429785-64430260 |
| chr3: | 64533422-64535867 |
| chr3: | 65111737-65119791 |
| chr3: | 65802399-65803002 |
| chr3: | 66550125-66551991 |
| chr3: | 67562171-67563622 |
| chr3: | 68336698-63337512 |
| chr3: | 69179922-69181540 |
| chr3: | 69873735-69874204 |
| chr3: | 69881179-69881876 |
| chr3: | 69881179-69884045 |
| chr3: | 71630675-71633038 |
| chr3: | 71774158-71774787 |
| chr3: | 71802175-71804230 |
| chr3: | 71802645-71803690 |
| chr3: | 71803265-71804456 |
| chr3: | 72496252-72496712 |
| chr3: | 73366365-73367705 |
| chr3: | 73366993-73367570 |
| chr3: | 73672992-73674813 |
| chr3: | 77811897-77812764 |
| chr3: | 78589771-78591653 |
| chr3: | 80061822-80064536 |
| chr3: | 80062894-80064158 |
| chr3: | 81223222-81223961 |
| chr3: | 81596296-81599606 |
| chr3: | 82203276-82204006 |
| chr3: | 82540540-82541475 |
| chr3: | 82868716-82873170 |
| chr3: | 82909359-82910772 |
| chr3: | 83017240-83020084 |
| chr3: | 83859915-83863621 |
| chr3: | 84105007-84107362 |
| chr3: | 84699605-84703015 |
| chr3: | 88664755-88665910 |
| chr3: | 94512353-94513631 |
| chr3: | 94512417-94512997 |
| chr3: | 96532044-96533939 |
| chr3: | 98410249-98414880 |
| chr3: | 98899096-98902405 |
| chr3: | 99006725-99007297 |
| chr3: | 99243238-99244869 |
| chr3: | 99628795-99629618 |
| chr3: | 99922687-99924933 |
| chr3: | 99924156-99924635 |
| chr3: | 100521677-100522512 |
| chr3: | 100669310-100670862 |
| chr3: | 101251512-101253932 |
| chr3: | 101357579-101359974 |
| chr3: | 103265539-103266944 |
| chr3: | 103758304-103759475 |
| chr3: | 104033961-104035387 |
| chr3: | 104278144-104279042 |
| chr3: | 104416307-104419298 |
| chr3: | 104664966-104667893 |
| chr3: | 105696596-105697873 |
| chr3: | 105697422-105697873 |
| chr3: | 107037948-107040302 |
| chr3: | 107150084-107150935 |
| chr3: | 107416750-107419221 |
| chr3: | 107741071-107742093 |
| chr3: | 108400963-108402264 |
| chr3: | 108599443-103599903 |
| chr3: | 109415099-109416599 |
| chr3: | 109646932-109647971 |
| chr3: | 111243676-111247239 |
| chr3: | 111713641-111714358 |
| chr3: | 113666273-113667425 |
| chr3: | 114288642-114290871 |
| chr3: | 114866053-114866608 |
| chr3: | 114960066-114960764 |
| chr3: | 115248226-115249779 |
| chr3: | 116523398-116525984 |
| chr3: | 123166736-123169061 |
| chr3: | 123197451-123197931 |
| chr3: | 123315417-123316796 |
| chr3: | 123718889-123722252 |
| chr3: | 124507505-124509676 |
| chr3: | 124605427-124606454 |
| chr3: | 124774333-124774963 |
| chr3: | 124936344-124936985 |
| chr3: | 125399009-125399944 |
| chr3: | 125672461-125675639 |
| chr3: | 125676779-125678941 |
| chr3: | 125846122-125850897 |
| chr3: | 125855009-125855480 |
| chr3: | 126075813-126076570 |
| chr3: | 126194061-126196104 |
| chr3: | 126194281-126195236 |
| chr3: | 126234394-126235747 |
| chr3: | 126580268-126534636 |
| chr3: | 126701538-126705395 |
| chr3: | 126701548-126703305 |
| chr3: | 126873033-126874731 |
| chr3: | 127347693-127348493 |
| chr3: | 127404339-127406550 |
| chr3: | 127529618-127530127 |
| chr3: | 128379304-128380241 |
| chr3: | 123430438-128432730 |
| chr3: | 129062403-129064913 |
| chr3: | 129063678-129064913 |
| chr3: | 129075827-129080132 |
| chr3: | 129234674-129236224 |
| chr3: | 129324035-129326706 |
| chr3: | 129657252-129657873 |
| chr3: | 130131959-130132390 |
| chr3: | 131602136-131602919 |
| chr3: | 131674530-131676201 |
| chr3: | 132521290-132521762 |
| chr3: | 132597850-132598735 |
| chr3: | 133292719-133293670 |
| chr3: | 133969286-133969906 |
| chr3: | 134043427-134044513 |
| chr3: | 134647088-134648608 |
| chr3: | 134842590-134844395 |
| chr3: | 135371923-135373460 |
| chr3: | 136024648-136026104 |
| chr3: | 137385663-137386738 |
| chr3: | 139653743-139654307 |
| chr3: | 139699689-139701126 |
| chr3: | 139700180-139701329 |
| chr3: | 139923606-139924666 |
| chr3: | 140267503-140270906 |
| chr3: | 140544427-140547049 |
| chr3: | 140777010-140778650 |
| chr3: | 140909357-140911954 |
| chr3: | 141683977-141685710 |
| chr3: | 141849954-141852078 |
| chr3: | 142610479-142612181 |
| chr3: | 144750495-144750997 |
| chr3: | 144888503-144889003 |
| chr3: | 145644997-145647435 |
| chr3: | 146415598-146417349 |
| chr3: | 146427474-146428257 |
| chr3: | 146483675-146486789 |
| chr3: | 146484705-146489124 |
| chr3: | 149268335-149270102 |
| chr3: | 150124574-150128485 |
| chr3: | 150430568-150431293 |
| chr3: | 152042202-152043122 |
| chr3: | 152611324-152612018 |
| chr3: | 156092052-156093564 |
| chr3: | 158051035-158052187 |
| chr3: | 158287531-158288286 |
| chr3: | 158808243-158813097 |
| chr3: | 158898209-158900072 |
| chr3: | 159257020-159257642 |
| chr3: | 159461407-159463400 |
| chr3: | 159588856-159589573 |
| chr3: | 159876701-159877179 |
| chr3: | 160234097-160235752 |
| chr3: | 160972703-160973492 |
| chr3: | 161570656-161571443 |
| chr3: | 161812944-161817727 |
| chr3: | 161813324-161816020 |
| chr3: | 161815094-161816630 |
| chr3: | 161918004-161920162 |
| chr3: | 162717329-162722217 |
| chr3: | 162764944-162769062 |
| chr3: | 163737909-163740820 |
| chr3: | 164125407-164126040 |
| chr3: | 165654526-165655559 |
| chr3: | 166053963-166055973 |
| chr3: | 166054970-166055973 |
| chr3: | 166184441-166185936 |
| chr3: | 166844371-166848423 |
| chr3: | 167248409-167249502 |
| chr3: | 167831308-167832552 |
| chr3: | 168428983-168432734 |
| chr3: | 169600109-169601379 |
| chr3: | 170062901-170063712 |
| chr3: | 170074085-170075315 |
| chr3: | 171892570-171893063 |
| chr3: | 171910737-171911646 |
| chr3: | 174077096-174080641 |
| chr3: | 174201951-174202701 |
| chr3: | 175080455-175083615 |
| chr3: | 175180529-175181082 |
| chr3: | 176076245-176076699 |
| chr3: | 176076245-176077384 |
| chr3: | 176513603-176515846 |
| chr3: | 176906700-176908902 |
| chr3: | 177294490-177297536 |
| chr3: | 177362981-177363669 |
| chr3: | 177383124-177383607 |
| chr3: | 177753630-177756939 |
| chr3: | 178550206-178550330 |
| chr3: | 178567620-178568168 |
| chr3: | 180851942-180852627 |
| chr3: | 182733130-182733634 |
| chr3: | 183091309-133091912 |
| chr3: | 183380570-133381096 |
| chr3: | 183542711-183543702 |
| chr3: | 183800397-183800937 |
| chr3: | 184097709-184098649 |
| chr3: | 184470036-184473260 |
| chr3: | 184470878-184472738 |
| chr3: | 184766850-184768075 |
| chr3: | 185565387-135567881 |
| chr3: | 186094720-136096878 |
| chr3: | 186182129-186183122 |
| chr3: | 186584101-186585071 |
| chr3: | 187732845-187734911 |
| chr3: | 188364606-188865515 |
| chr3: | 189737261-189740574 |
| chr3: | 189839998-189840977 |
| chr3: | 190512145-190517112 |
| chr3: | 190689251-190690181 |
| chr3: | 190786995-190787938 |
| chr3: | 191011230-191013641 |
| chr3: | 191504640-191507642 |
| chr3: | 191760587-191762218 |
| chr3: | 191890175-191891243 |
| chr3: | 192122774-192123485 |
| chr3: | 192389299-192392171 |
| chr3: | 193851791-193852969 |
| chr3: | 194068079-194069113 |
| chr3: | 194100947-194103096 |
| chr3: | 194350431-194351426 |
| chr3: | 194383922-194384551 |
| chr3: | 194398753-194400303 |
| chr3: | 194457040-194459279 |
| chr4: | 568952-570319 |
| chr4: | 628597-629577 |
| chr4: | 637593-642278 |
| chr4: | 662139-663599 |
| chr4: | 708453-711488 |
| chr4: | 725431-725961 |
| chr4: | 738454-741519 |
| chr4: | 752085-753790 |
| chr4: | 920166-921216 |
| chr4: | 961685-962130 |
| chr4: | 1003313-1005958 |
| chr4: | 1040137-1040662 |
| chr4: | 1047354-1049229 |
| chr4: | 1080327-1081362 |
| chr4: | 1088851-1090526 |
| chr4: | 1230368-1231788 |
| chr4: | 1241724-1244283 |
| chr4: | 1289224-1292044 |
| chr4: | 1289779-1290279 |
| chr4: | 1291389-1292044 |
| chr4: | 1327540-1328325 |
| chr4: | 1387978-1392638 |
| chr4: | 1388033-1389448 |
| chr4: | 1391243-1392518 |
| chr4: | 1419822-1420997 |
| chr4: | 1419822-1423095 |
| chr4: | 1547247-1549083 |
| chr4: | 1602780-1603940 |
| chr4: | 1672645-1674460 |
| chr4: | 1872295-1874273 |
| chr4: | 2060387-2061782 |
| chr4: | 2263111-2264500 |
| chr4: | 2397764-2398509 |
| chr4: | 2420088-2420693 |
| chr4: | 2512933-2513513 |
| chr4: | 2537341-2539317 |
| chr4: | 3310705-3312370 |
| chr4: | 3342927-3343659 |
| chr4: | 3427373-3428043 |
| chr4: | 3492020-3493441 |
| chr4: | 4414439-4415839 |
| chr4: | 4568068-4568575 |
| chr4: | 4819256-4819706 |
| chr4: | 5387243-5388232 |
| chr4: | 5762029-5763562 |
| chr4: | 5895101-5898874 |
| chr4: | 6038365-6040295 |
| chr4: | 6139940-6142846 |
| chr4: | 6441951-6443056 |
| chr4: | 6473891-6474466 |
| chr4: | 6564807-6565999 |
| chr4: | 6624523-6625639 |
| chr4: | 6651264-6652717 |
| chr4: | 6651908-6652913 |
| chr4: | 6682328-6685317 |
| chr4: | 6784040-6785591 |
| chr4: | 6881592-6882047 |
| chr4: | 6897509-6899599 |
| chr4: | 7043979-7045994 |
| chr4: | 7146020-7149702 |
| chr4: | 7166758-7167330 |
| chr4: | 7320094-7321723 |
| chr4: | 7443255-7444420 |
| chr4: | 7453227-7457688 |
| chr4: | 7483641-7484611 |
| chr4: | 7498262-7498976 |
| chr4: | 7529269-7529859 |
| chr4: | 7635259-7635784 |
| chr4: | 7707737-7708677 |
| chr4: | 7836458-7837826 |
| chr4: | 7919890-7920820 |
| chr4: | 7950857-7951577 |
| chr4: | 7971253-7974932 |
| chr4: | 8271224-8272065 |
| chr4: | 8731849-8732689 |
| chr4: | 9863690-9865968 |
| chr4: | 9876370-9877136 |
| chr4: | 10136109-10137099 |
| chr4: | 10147105-10147731 |
| chr4: | 10725729-10726215 |
| chr4: | 10726064-10726780 |
| chr4: | 11041558-11042673 |
| chr4: | 11633421-11634137 |
| chr4: | 11666590-11670675 |
| chr4: | 12326817-12328077 |
| chr4: | 13380896-13382166 |
| chr4: | 15003336-15007025 |
| chr4: | 15223121-15223663 |
| chr4: | 15606201-15607407 |
| chr4: | 15626950-15627587 |
| chr4: | 15705044-15706579 |
| chr4: | 16153415-16154347 |
| chr4: | 16227560-16228537 |
| chr4: | 17036105-17040105 |
| chr4: | 19057400-19057988 |
| chr4: | 20158205-20162417 |
| chr4: | 20407568-20408857 |
| chr4: | 21118712-21119251 |
| chr4: | 21137385-21138371 |
| chr4: | 21273362-21275206 |
| chr4: | 21457508-21458519 |
| chr4: | 21782010-21782635 |
| chr4: | 22663167-22666182 |
| chr4: | 22922122-22923739 |
| chr4: | 23590382-23595380 |
| chr4: | 24294220-24295310 |
| chr4: | 24966463-24967101 |
| chr4: | 26254902-26256281 |
| chr4: | 26290477-26295344 |
| chr4: | 26438714-26441364 |
| chr4: | 26955889-26957635 |
| chr4: | 27552999-27554059 |
| chr4: | 28421456-28422091 |
| chr4: | 28511836-28512931 |
| chr4: | 29226602-29231444 |
| chr4: | 30151543-30152198 |
| chr4: | 31319953-31320731 |
| chr4: | 32137762-32138938 |
| chr4: | 32321456-32324114 |
| chr4: | 32574576-32575127 |
| chr4: | 33031826-33032327 |
| chr4: | 33085147-33085621 |
| chr4: | 34956263-34958315 |
| chr4: | 35240199-35242883 |
| chr4: | 35240199-35244409 |
| chr4: | 35495327-35496895 |
| chr4: | 35873512-35875450 |
| chr4: | 36020547-36021121 |
| chr4: | 38264499-38265328 |
| chr4: | 38264499-38265681 |
| chr4: | 38664781-38666883 |
| chr4: | 38735403-38736569 |
| chr4: | 39046303-39047018 |
| chr4: | 39814336-39816566 |
| chr4: | 39978483-39979638 |
| chr4: | 40025327-40026129 |
| chr4: | 40076687-40079703 |
| chr4: | 41406467-41407809 |
| chr4: | 43146361-43150236 |
| chr4: | 43751616-43752082 |
| chr4: | 44305927-44310334 |
| chr4: | 46202818-46205478 |
| chr4: | 47356563-47359763 |
| chr4: | 55219544-55220358 |
| chr4: | 56266121-56267944 |
| chr4: | 56609458-56610582 |
| chr4: | 57772270-57775584 |
| chr4: | 58720380-58723843 |
| chr4: | 58722649-58723902 |
| chr4: | 58961771-58962290 |
| chr4: | 58974884-58975724 |
| chr4: | 60291553-60294222 |
| chr4: | 60816211-60817763 |
| chr4: | 60816438-60817151 |
| chr4: | 61330130-61332041 |
| chr4: | 61330130-61334710 |
| chr4: | 61500207-61502149 |
| chr4: | 61939167-61942248 |
| chr4: | 61999347-62000415 |
| chr4: | 62284123-62285279 |
| chr4: | 62498775-62499695 |
| chr4: | 65097006-65097906 |
| chr4: | 65719076-65719562 |
| chr4: | 67659411-67661750 |
| chr4: | 68264246-68266790 |
| chr4: | 68352971-68353997 |
| chr4: | 68380886-68383240 |
| chr4: | 70415661-70416616 |
| chr4: | 70466797-70470796 |
| chr4: | 70467621-70469086 |
| chr4: | 73000703-73003881 |
| chr4: | 74555362-74556762 |
| chr4: | 74942304-74945441 |
| chr4: | 75081451-75083637 |
| chr4: | 76318232-76319852 |
| chr4: | 76743255-76744325 |
| chr4: | 76948569-76949047 |
| chr4: | 76954997-76955641 |
| chr4: | 77731099-77732614 |
| chr4: | 77782783-77787668 |
| chr4: | 79218331-79218938 |
| chr4: | 79299637-79300649 |
| chr4: | 79456127-79456647 |
| chr4: | 79697089-79697707 |
| chr4: | 80360421-80362812 |
| chr4: | 80361568-80362718 |
| chr4: | 81123904-81124539 |
| chr4: | 81508721-81509954 |
| chr4: | 81531281-81535875 |
| chr4: | 82054064-82055060 |
| chr4: | 83482579-83483249 |
| chr4: | 83636469-83641241 |
| chr4: | 84041643-84042902 |
| chr4: | 85053987-85057210 |
| chr4: | 86571290-86572932 |
| chr4: | 86976391-86980012 |
| chr4: | 87148074-87152319 |
| chr4: | 88923625-88929511 |
| chr4: | 90427830-90428865 |
| chr4: | 90562958-90563578 |
| chr4: | 91287888-91292489 |
| chr4: | 91931552-91935848 |
| chr4: | 92160322-92162441 |
| chr4: | 93567393-93569980 |
| chr4: | 93569065-93570266 |
| chr4: | 93857363-93859600 |
| chr4: | 95637798-95638672 |
| chr4: | 95678933-95679943 |
| chr4: | 96246527-96247542 |
| chr4: | 96304014-96304577 |
| chr4: | 97721702-97722302 |
| chr4: | 99680853-99682900 |
| chr4: | 99813534-99818241 |
| chr4: | 100870803-100871423 |
| chr4: | 101599647-101601485 |
| chr4: | 101960122-101961205 |
| chr4: | 101960217-101960859 |
| chr4: | 102081201-102084993 |
| chr4: | 103422213-103422943 |
| chr4: | 104894092-104894697 |
| chr4: | 106940112-106943263 |
| chr4: | 107234806-107235341 |
| chr4: | 107604246-107609234 |
| chr4: | 108510936-108514906 |
| chr4: | 108788900-108790803 |
| chr4: | 108790260-108790984 |
| chr4: | 109347282-109348853 |
| chr4: | 109716845-109717704 |
| chr4: | 109720672-109722010 |
| chr4: | 109944146-109945151 |
| chr4: | 109944691-109945399 |
| chr4: | 110354603-110355647 |
| chr4: | 110690151-110690798 |
| chr4: | 111004040-111004990 |
| chr4: | 113230885-113232191 |
| chr4: | 115179688-115182405 |
| chr4: | 115928721-115929257 |
| chr4: | 115928721-115931894 |
| chr4: | 116314517-116315560 |
| chr4: | 117367648-117368494 |
| chr4: | 119125696-119130309 |
| chr4: | 120552839-120556129 |
| chr4: | 120963461-120965106 |
| chr4: | 122872273-122873108 |
| chr4: | 123411439-123412392 |
| chr4: | 125727333-125730018 |
| chr4: | 128982058-128983393 |
| chr4: | 130280835-130282934 |
| chr4: | 130585201-130587579 |
| chr4: | 130954417-130954973 |
| chr4: | 132122607-132123600 |
| chr4: | 132490504-132493598 |
| chr4: | 132971855-132973023 |
| chr4: | 133178892-133182382 |
| chr4: | 133181379-133181873 |
| chr4: | 133181379-133182910 |
| chr4: | 133528888-133529393 |
| chr4: | 134173565-134176766 |
| chr4: | 135432950-135435721 |
| chr4: | 135433031-135434946 |
| chr4: | 136508295-136511660 |
| chr4: | 136679962-136681323 |
| chr4: | 137073707-137075912 |
| chr4: | 138966428-138967210 |
| chr4: | 140476662-140477287 |
| chr4: | 140528423-140530958 |
| chr4: | 141537561-141538017 |
| chr4: | 141700938-141701813 |
| chr4: | 142230577-142232905 |
| chr4: | 143857915-143858576 |
| chr4: | 145319066-145319927 |
| chr4: | 146101009-146102047 |
| chr4: | 146438834-146440998 |
| chr4: | 146901846-146904372 |
| chr4: | 146924599-146927658 |
| chr4: | 147206826-147208457 |
| chr4: | 147329726-147332828 |
| chr4: | 148020787-148022707 |
| chr4: | 148515649-148519825 |
| chr4: | 149099862-149101623 |
| chr4: | 149365409-149367008 |
| chr4: | 149426625-149430059 |
| chr4: | 152252450-152253943 |
| chr4: | 152603171-152603631 |
| chr4: | 152603171-152607993 |
| chr4: | 152790136-152794781 |
| chr4: | 152990384-152993935 |
| chr4: | 154232562-154234690 |
| chr4: | 155267671-155270413 |
| chr4: | 156594205-156598282 |
| chr4: | 157305301-157306948 |
| chr4: | 157680494-157681444 |
| chr4: | 158430569-158431376 |
| chr4: | 158728286-158731906 |
| chr4: | 159689985-159690865 |
| chr4: | 159800004-159800721 |
| chr4: | 160022637-160025605 |
| chr4: | 162449507-162451582 |
| chr4: | 163277598-163281991 |
| chr4: | 163370263-163372635 |
| chr4: | 163394008-163396453 |
| chr4: | 163933654-163934957 |
| chr4: | 163955348-163957861 |
| chr4: | 164613720-164614893 |
| chr4: | 165037467-165040286 |
| chr4: | 166003442-166004889 |
| chr4: | 166128437-166129207 |
| chr4: | 166157948-166159055 |
| chr4: | 167004318-167004862 |
| chr4: | 167108947-167111990 |
| chr4: | 167501126-167501835 |
| chr4: | 168276636-168280052 |
| chr4: | 168795363-163797352 |
| chr4: | 169116481-169118430 |
| chr4: | 169630248-169631274 |
| chr4: | 170287043-170288245 |
| chr4: | 171051974-171054884 |
| chr4: | 171070583-171073502 |
| chr4: | 171072084-171073258 |
| chr4: | 171268818-171273749 |
| chr4: | 172374567-172377504 |
| chr4: | 172489413-172492301 |
| chr4: | 172988680-172992916 |
| chr4: | 174090014-174091177 |
| chr4: | 175625198-175626963 |
| chr4: | 176262865-176264641 |
| chr4: | 176539353-176540191 |
| chr4: | 177049571-177050852 |
| chr4: | 177049571-177053233 |
| chr4: | 177311685-177312605 |
| chr4: | 177851143-177852369 |
| chr4: | 173052183-178054174 |
| chr4: | 179497413-179498906 |
| chr4: | 179520642-179522448 |
| chr4: | 179532897-179534922 |
| chr4: | 179905715-179906435 |
| chr4: | 181135089-181139235 |
| chr4: | 181270503-181274316 |
| chr4: | 181471179-181473753 |
| chr4: | 181735804-181740723 |
| chr4: | 181737157-181739601 |
| chr4: | 182056558-182057193 |
| chr4: | 182160284-182162955 |
| chr4: | 183570071-183571999 |
| chr4: | 183753621-183754146 |
| chr4: | 183838144-183838686 |
| chr4: | 183985931-183988803 |
| chr4: | 184019345-184021259 |
| chr4: | 184147524-184151481 |
| chr4: | 184257932-184258522 |
| chr4: | 184311887-184315201 |
| chr4: | 184674469-184675941 |
| chr4: | 184674469-184676528 |
| chr4: | 184985565-184989506 |
| chr4: | 185394275-185394974 |
| chr4: | 185639514-185640068 |
| chr4: | 185746453-185747593 |
| chr4: | 185779357-185780182 |
| chr4: | 185886223-185889108 |
| chr4: | 186100260-186102198 |
| chr4: | 186130661-186131326 |
| chr4: | 186441628-186444084 |
| chr4: | 186954358-186956731 |
| chr4: | 186955123-186955903 |
| chr4: | 186977832-186979152 |
| chr4: | 187025686-187026688 |
| chr4: | 187093513-187098202 |
| chr4: | 187159979-187161265 |
| chr4: | 187163641-187164421 |
| chr4: | 187292343-187293748 |
| chr4: | 187353524-187357809 |
| chr4: | 187397105-187400035 |
| chr4: | 187843727-187844200 |
| chr4: | 187987822-187988467 |
| chr4: | 188041841-188043451 |
| chr4: | 189072012-189075938 |
| chr4: | 189189983-189190918 |
| chr5: | 2251836-2252406 |
| chr5: | 2279465-2280940 |
| chr5: | 2490114-2491757 |
| chr5: | 2490433-2491693 |
| chr5: | 2518710-2519182 |
| chr5: | 2539267-2542312 |
| chr5: | 2712700-2715340 |
| chr5: | 2772346-2776105 |
| chr5: | 2819106-2821221 |
| chr5: | 2862645-2863380 |
| chr5: | 2873710-2875151 |
| chr5: | 2874347-2875000 |
| chr5: | 3594547-3596541 |
| chr5: | 3635532-3639513 |
| chr5: | 3637075-3639413 |
| chr5: | 3659736-3660509 |
| chr5: | 4325180-4325851 |
| chr5: | 4465031-4466632 |
| chr5: | 4842976-4844522 |
| chr5: | 5107888-5109888 |
| chr5: | 5139974-5140869 |
| chr5: | 5678837-5680889 |
| chr5: | 5685176-5686358 |
| chr5: | 6370038-6371749 |
| chr5: | 6712503-6715218 |
| chr5: | 7396023-7396945 |
| chr5: | 7561828-7562288 |
| chr5: | 7836944-7840867 |
| chr5: | 7917069-7920516 |
| chr5: | 8017606-8020646 |
| chr5: | 8018714-8020646 |
| chr5: | 8214057-8215449 |
| chr5: | 8255770-8260730 |
| chr5: | 8272604-8276725 |
| chr5: | 8775955-8780362 |
| chr5: | 9545673-9546476 |
| chr5: | 10273006-10274951 |
| chr5: | 10273930-10274951 |
| chr5: | 10527384-10532113 |
| chr5: | 10562851-10565401 |
| chr5: | 10563701-10564831 |
| chr5: | 10638792-10640693 |
| chr5: | 10639032-10640182 |
| chr5: | 10649351-10650331 |
| chr5: | 10681175-10682755 |
| chr5: | 10690562-10691133 |
| chr5: | 10760952-10761943 |
| chr5: | 11147104-11148545 |
| chr5: | 11309884-11312983 |
| chr5: | 11384681-11385476 |
| chr5: | 11903847-11904592 |
| chr5: | 12056625-12058012 |
| chr5: | 12427290-12428783 |
| chr5: | 12427934-12428975 |
| chr5: | 12851702-12853682 |
| chr5: | 14871530-14872696 |
| chr5: | 15228040-15230988 |
| chr5: | 15719329-15720964 |
| chr5: | 16124833-16125678 |
| chr5: | 16150030-16150746 |
| chr5: | 16179167-16180231 |
| chr5: | 16870107-16870711 |
| chr5: | 18343775-18346331 |
| chr5: | 18873274-18875040 |
| chr5: | 18921537-18922000 |
| chr5: | 19374821-19376411 |
| chr5: | 19375246-19376411 |
| chr5: | 19375775-19376411 |
| chr5: | 20127204-20131123 |
| chr5: | 20832594-20834793 |
| chr5: | 23902936-23905357 |
| chr5: | 25821844-25823788 |
| chr5: | 25889777-25891892 |
| chr5: | 26091134-26091637 |
| chr5: | 26235137-26236946 |
| chr5: | 26385157-26383663 |
| chr5: | 27524059-27526393 |
| chr5: | 27804494-27805727 |
| chr5: | 28491007-28495614 |
| chr5: | 28538468-28539596 |
| chr5: | 28932254-28932999 |
| chr5: | 29686887-29689930 |
| chr5: | 29955652-29956102 |
| chr5: | 30827917-30830110 |
| chr5: | 30918057-30918602 |
| chr5: | 31347286-31348235 |
| chr5: | 31352521-31354208 |
| chr5: | 31890989-31894858 |
| chr5: | 32312478-32313163 |
| chr5: | 33111548-33112302 |
| chr5: | 33111623-33112685 |
| chr5: | 33596890-33597545 |
| chr5: | 33688706-33689638 |
| chr5: | 34656279-34656839 |
| chr5: | 34656279-34657421 |
| chr5: | 34690581-34691607 |
| chr5: | 34929085-34930121 |
| chr5: | 35713141-35714352 |
| chr5: | 36710490-36711062 |
| chr5: | 37839636-37840756 |
| chr5: | 37972566-37974544 |
| chr5: | 38556267-38557056 |
| chr5: | 39074004-39075326 |
| chr5: | 39968955-39971775 |
| chr5: | 41901871-41903659 |
| chr5: | 42092017-42095899 |
| chr5: | 42628248-42631270 |
| chr5: | 42629813-42630393 |
| chr5: | 45191689-45193819 |
| chr5: | 45527011-45527970 |
| chr5: | 46144779-46145551 |
| chr5: | 46273453-46275837 |
| chr5: | 46328868-46332425 |
| chr5: | 50234326-50239097 |
| chr5: | 51577447-51579537 |
| chr5: | 52458851-52459701 |
| chr5: | 52497292-52500173 |
| chr5: | 53313527-53819228 |
| chr5: | 54103757-54104736 |
| chr5: | 54247310-54248034 |
| chr5: | 56110736-56112454 |
| chr5: | 56247092-56248201 |
| chr5: | 57392021-57392591 |
| chr5: | 57776769-57778784 |
| chr5: | 57777544-57778200 |
| chr5: | 57955598-57959465 |
| chr5: | 58079408-58080680 |
| chr5: | 58079408-58081505 |
| chr5: | 60131840-60132920 |
| chr5: | 60400950-60402280 |
| chr5: | 60625377-60629756 |
| chr5: | 61237615-61239254 |
| chr5: | 61574731-61576375 |
| chr5: | 61575679-61576375 |
| chr5: | 61748125-61749393 |
| chr5: | 63698350-63701355 |
| chr5: | 64604277-64604851 |
| chr5: | 65028128-65030322 |
| chr5: | 65221310-65222431 |
| chr5: | 65268455-65270712 |
| chr5: | 65891606-65892900 |
| chr5: | 67103393-67105197 |
| chr5: | 67509507-67513143 |
| chr5: | 67950589-67951668 |
| chr5: | 68319571-68320758 |
| chr5: | 72742598-72744615 |
| chr5: | 73569872-73570356 |
| chr5: | 76373231-76374070 |
| chr5: | 76505870-76507356 |
| chr5: | 76554592-76555182 |
| chr5: | 77943975-77945033 |
| chr5: | 77945123-77945900 |
| chr5: | 78109802-78111790 |
| chr5: | 78280505-78281289 |
| chr5: | 80255351-80256937 |
| chr5: | 81241439-81242075 |
| chr5: | 82902998-82904143 |
| chr5: | 86115169-86117832 |
| chr5: | 86215517-86217204 |
| chr5: | 86245998-86247122 |
| chr5: | 87378956-87382124 |
| chr5: | 90499770-90502087 |
| chr5: | 90624301-90625878 |
| chr5: | 92919794-92920972 |
| chr5: | 93903386-93907012 |
| chr5: | 94956239-94956904 |
| chr5: | 95966616-95967159 |
| chr5: | 97232199-97232647 |
| chr5: | 97401542-97402755 |
| chr5: | 101096357-101097276 |
| chr5: | 102235698-102236280 |
| chr5: | 105003859-105007711 |
| chr5: | 105894419-105895597 |
| chr5: | 106089288-106090342 |
| chr5: | 106324680-106326429 |
| chr5: | 107198470-107199258 |
| chr5: | 107794869-107795891 |
| chr5: | 107931438-107932396 |
| chr5: | 108689436-108690510 |
| chr5: | 108868298-108869831 |
| chr5: | 110283534-110285488 |
| chr5: | 111806260-111807625 |
| chr5: | 112217824-112218717 |
| chr5: | 112477787-112478379 |
| chr5: | 113567250-113568581 |
| chr5: | 114012646-114017244 |
| chr5: | 114208438-114211584 |
| chr5: | 114258317-114260539 |
| chr5: | 116222098-116224202 |
| chr5: | 116974607-116975201 |
| chr5: | 117669937-117672867 |
| chr5: | 117877496-117878269 |
| chr5: | 117878269-117880183 |
| chr5: | 117880229-117881004 |
| chr5: | 119377904-119382657 |
| chr5: | 119380179-119382860 |
| chr5: | 120649604-120652173 |
| chr5: | 121132551-121135934 |
| chr5: | 123545097-123545857 |
| chr5: | 124546073-124546585 |
| chr5: | 124950285-124952730 |
| chr5: | 126126121-126127463 |
| chr5: | 127172409-127173299 |
| chr5: | 127335788-127336854 |
| chr5: | 127407815-127410881 |
| chr5: | 127409618-127410881 |
| chr5: | 127901938-127903315 |
| chr5: | 128882146-128883221 |
| chr5: | 129613947-129618215 |
| chr5: | 130332362-130332858 |
| chr5: | 132095302-132099413 |
| chr5: | 132685179-132685926 |
| chr5: | 133134470-133136491 |
| chr5: | 133238217-133239073 |
| chr5: | 134258873-134261321 |
| chr5: | 134262351-134263766 |
| chr5: | 135116971-135119875 |
| chr5: | 135344653-135346248 |
| chr5: | 136854920-136856297 |
| chr5: | 139367934-139369303 |
| chr5: | 140214368-140215013 |
| chr5: | 140214544-140215658 |
| chr5: | 140532219-140536789 |
| chr5: | 140593138-140594950 |
| chr5: | 141438287-141440068 |
| chr5: | 141730914-141731901 |
| chr5: | 142270493-142272229 |
| chr5: | 143406475-143409434 |
| chr5: | 144871931-144872750 |
| chr5: | 146286268-146287496 |
| chr5: | 146286336-146288707 |
| chr5: | 146286879-146287496 |
| chr5: | 146841560-146843928 |
| chr5: | 147553094-147554199 |
| chr5: | 147553094-147555199 |
| chr5: | 149011108-149013018 |
| chr5: | 150177643-150181585 |
| chr5: | 150789803-150792683 |
| chr5: | 150880463-150883183 |
| chr5: | 150954478-150955343 |
| chr5: | 151249350-151252921 |
| chr5: | 151514688-151518838 |
| chr5: | 151843033-151846488 |
| chr5: | 151956953-151961217 |
| chr5: | 153584015-153587325 |
| chr5: | 153802582-153806769 |
| chr5: | 154393341-154398169 |
| chr5: | 155311775-155312583 |
| chr5: | 157291193-157292957 |
| chr5: | 158895413-158898555 |
| chr5: | 159349801-159351114 |
| chr5: | 159922267-159923580 |
| chr5: | 160196257-160198957 |
| chr5: | 160196262-160200098 |
| chr5: | 160589023-160590070 |
| chr5: | 161543172-161543860 |
| chr5: | 162861740-162863314 |
| chr5: | 163265301-163266216 |
| chr5: | 163665328-163666420 |
| chr5: | 164141471-164143124 |
| chr5: | 164226760-164227335 |
| chr5: | 164722302-164725972 |
| chr5: | 166402717-166403337 |
| chr5: | 166859011-166859683 |
| chr5: | 168380196-168380777 |
| chr5: | 168909711-168912964 |
| chr5: | 169440270-169441128 |
| chr5: | 170129779-170131495 |
| chr5: | 172252393-172253118 |
| chr5: | 172973002-172974722 |
| chr5: | 173090243-173091240 |
| chr5: | 173168913-173170626 |
| chr5: | 174436001-174440560 |
| chr5: | 174552602-174553201 |
| chr5: | 174819779-174820408 |
| chr5: | 175345811-175350298 |
| chr5: | 178109175-178113319 |
| chr5: | 178112138-178113209 |
| chr5: | 178118507-178119042 |
| chr5: | 178218220-178220656 |
| chr5: | 178258606-173260382 |
| chr5: | 178348371-178353189 |
| chr5: | 178515570-178516543 |
| chr5: | 173704380-178705192 |
| chr5: | 173338751-178840537 |
| chr5: | 173338942-178839317 |
| chr5: | 178856573-178857063 |
| chr5: | 180041765-180045486 |
| chr5: | 180042160-180043160 |
| chr5: | 180044221-180045651 |
| chr5: | 180343918-180344517 |
| chr5: | 180432660-180433980 |
| chr5: | 180515151-180518739 |
| chr5: | 180560515-180563121 |
| chr5: | 180562293-180562792 |
| chr5: | 180565882-180567331 |
| chr5: | 180569063-180570051 |
| chr6: | 371057-372402 |
| chr6: | 515610-519014 |
| chr6: | 513319-520809 |
| chr6: | 595051-596361 |
| chr6: | 601031-602700 |
| chr6: | 666425-667789 |
| chr6: | 675417-677340 |
| chr6: | 774091-774831 |
| chr6: | 775571-776271 |
| chr6: | 861925-863775 |
| chr6: | 909726-911131 |
| chr6: | 1532294-1537291 |
| chr6: | 1532883-1535337 |
| chr6: | 2200729-2202604 |
| chr6: | 2207443-2203325 |
| chr6: | 2567325-2572089 |
| chr6: | 3086109-3087023 |
| chr6: | 3090971-3094511 |
| chr6: | 3144812-3147616 |
| chr6: | 3145126-3146356 |
| chr6: | 3145966-3147203 |
| chr6: | 3181776-3182650 |
| chr6: | 3254453-3256193 |
| chr6: | 3255707-3256193 |
| chr6: | 3294341-3295626 |
| chr6: | 3516517-3517592 |
| chr6: | 3619461-3621865 |
| chr6: | 3712745-3713741 |
| chr6: | 3716205-3719680 |
| chr6: | 3718484-3719613 |
| chr6: | 3885175-3887768 |
| chr6: | 4254023-4257185 |
| chr6: | 4476189-4478552 |
| chr6: | 4569272-4572127 |
| chr6: | 4913712-4919227 |
| chr6: | 5112412-5112918 |
| chr6: | 5135173-5136002 |
| chr6: | 5822036-5822683 |
| chr6: | 5898383-5893838 |
| chr6: | 6742181-6745012 |
| chr6: | 6837741-6838388 |
| chr6: | 6953602-6954514 |
| chr6: | 7271950-7273959 |
| chr6: | 7315493-7317718 |
| chr6: | 7316213-7316809 |
| chr6: | 7442236-7443508 |
| chr6: | 7442596-7443244 |
| chr6: | 7781529-7782585 |
| chr6: | 7792953-7793650 |
| chr6: | 8166301-8170839 |
| chr6: | 8337688-8341533 |
| chr6: | 9177164-9177921 |
| chr6: | 9273882-9274893 |
| chr6: | 9762148-9765456 |
| chr6: | 10658038-10659505 |
| chr6: | 10741109-10745173 |
| chr6: | 12009115-12009764 |
| chr6: | 13190995-13192104 |
| chr6: | 13191100-13192936 |
| chr6: | 13298917-13300100 |
| chr6: | 14145565-14146785 |
| chr6: | 14145565-14147546 |
| chr6: | 14739480-14740812 |
| chr6: | 14740872-14745737 |
| chr6: | 14745232-14745727 |
| chr6: | 14839487-14841627 |
| chr6: | 15248316-15249040 |
| chr6: | 16579342-16579878 |
| chr6: | 17727745-17728382 |
| chr6: | 17987505-17938416 |
| chr6: | 18021157-18022202 |
| chr6: | 18402120-18402881 |
| chr6: | 19041798-19044881 |
| chr6: | 19685567-19686553 |
| chr6: | 19837789-19839772 |
| chr6: | 21980579-21982955 |
| chr6: | 22051145-22054684 |
| chr6: | 22052774-22053728 |
| chr6: | 23180770-23163756 |
| chr6: | 23434445-23435008 |
| chr6: | 23743278-23746060 |
| chr6: | 23881964-23866955 |
| chr6: | 25312436-25315638 |
| chr6: | 25437846-25438342 |
| chr6: | 25450845-25452339 |
| chr6: | 30715634-30716191 |
| chr6: | 30755746-30756386 |
| chr6: | 31023136-31023601 |
| chr6: | 31193076-31194314 |
| chr6: | 31233995-31235828 |
| chr6: | 31237024-31238044 |
| chr6: | 31237024-31240317 |
| chr6: | 31263068-31266105 |
| chr6: | 31266725-31269500 |
| chr6: | 31270366-31271970 |
| chr6: | 31275981-31279433 |
| chr6: | 31285646-31287494 |
| chr6: | 31287831-31289437 |
| chr6: | 31289501-31290103 |
| chr6: | 31289501-31290778 |
| chr6: | 31302807-31304380 |
| chr6: | 31313270-31313755 |
| chr6: | 31313785-31318416 |
| chr6: | 31313467-31319195 |
| chr6: | 31323767-31326087 |
| chr6: | 31324841-31325710 |
| chr6: | 31336878-31337463 |
| chr6: | 31337788-31341413 |
| chr6: | 31347970-31343587 |
| chr6: | 31348364-31350868 |
| chr6: | 31361700-31363838 |
| chr6: | 31389915-31390550 |
| chr6: | 31947907-31952402 |
| chr6: | 32145042-32148107 |
| chr6: | 32429916-32432577 |
| chr6: | 32431698-32432577 |
| chr6: | 32455080-32459039 |
| chr6: | 32455453-32460365 |
| chr6: | 32459039-32460285 |
| chr6: | 32486911-32488645 |
| chr6: | 32483730-32489739 |
| chr6: | 32497355-32498474 |
| chr6: | 32501231-32502270 |
| chr6: | 32501231-32505192 |
| chr6: | 32503892-32505192 |
| chr6: | 32505269-32505894 |
| chr6: | 32505269-32507766 |
| chr6: | 32517657-32518616 |
| chr6: | 32522596-32523210 |
| chr6: | 32549028-32550505 |
| chr6: | 32549028-32552693 |
| chr6: | 32550496-32551944 |
| chr6: | 32559879-32560794 |
| chr6: | 32580572-32531638 |
| chr6: | 32580572-32583014 |
| chr6: | 32537772-32590142 |
| chr6: | 32592141-32595110 |
| chr6: | 32604279-32605566 |
| chr6: | 32605566-32606717 |
| chr6: | 32626056-32627176 |
| chr6: | 32627176-32629976 |
| chr6: | 32631070-32632695 |
| chr6: | 32635699-32636195 |
| chr6: | 32651960-32652913 |
| chr6: | 32686290-32687181 |
| chr6: | 32750657-32751983 |
| chr6: | 32776711-32779836 |
| chr6: | 32778252-32779877 |
| chr6: | 32778912-32779836 |
| chr6: | 33022798-33023588 |
| chr6: | 33051704-33055345 |
| chr6: | 33098599-33101930 |
| chr6: | 33100747-33101930 |
| chr6: | 33249085-33250420 |
| chr6: | 33583939-33585879 |
| chr6: | 33796274-33797809 |
| chr6: | 33797309-33797809 |
| chr6: | 33941657-33943036 |
| chr6: | 34111896-34113220 |
| chr6: | 34202138-34206094 |
| chr6: | 34317011-34319617 |
| chr6: | 35464170-35465801 |
| chr6: | 35626437-35629745 |
| chr6: | 35628665-35629745 |
| chr6: | 35655914-35656759 |
| chr6: | 36640176-36642182 |
| chr6: | 36951876-36955062 |
| chr6: | 37616371-37618598 |
| chr6: | 37851244-37852590 |
| chr6: | 39068929-39072849 |
| chr6: | 40434224-40436393 |
| chr6: | 40661008-40662134 |
| chr6: | 41395325-41396369 |
| chr6: | 41395487-41397485 |
| chr6: | 41514169-41514893 |
| chr6: | 41701674-41703587 |
| chr6: | 43978749-43979544 |
| chr6: | 44008476-44011596 |
| chr6: | 44511814-44515528 |
| chr6: | 44692192-44693108 |
| chr6: | 45767599-45769428 |
| chr6: | 45768255-45768945 |
| chr6: | 46304244-46307620 |
| chr6: | 47564740-47565704 |
| chr6: | 47564740-47566899 |
| chr6: | 47622413-47623462 |
| chr6: | 47906745-47908100 |
| chr6: | 47982579-47984615 |
| chr6: | 48096907-48100853 |
| chr6: | 48440355-48442495 |
| chr6: | 48991246-48994994 |
| chr6: | 49972755-49973255 |
| chr6: | 51199473-51200145 |
| chr6: | 51666983-51669348 |
| chr6: | 51668468-51669564 |
| chr6: | 51668375-51669514 |
| chr6: | 51668375-51670174 |
| chr6: | 51736168-51736776 |
| chr6: | 52793243-52796624 |
| chr6: | 54553918-54557383 |
| chr6: | 54662548-54665910 |
| chr6: | 55413583-55417605 |
| chr6: | 55876773-55878437 |
| chr6: | 55904070-55909060 |
| chr6: | 55964651-55965665 |
| chr6: | 56265335-56265821 |
| chr6: | 56898470-56399029 |
| chr6: | 56942932-56946395 |
| chr6: | 57623308-57628243 |
| chr6: | 58606544-58608219 |
| chr6: | 58776769-58780132 |
| chr6: | 63048614-63049898 |
| chr6: | 63597659-63598898 |
| chr6: | 63597659-63599606 |
| chr6: | 64667010-64668280 |
| chr6: | 65344691-65349418 |
| chr6: | 65347537-65349418 |
| chr6: | 65348371-65349107 |
| chr6: | 65711019-65715055 |
| chr6: | 65823251-65823958 |
| chr6: | 65909901-65910680 |
| chr6: | 66075001-66075482 |
| chr6: | 66402716-66404693 |
| chr6: | 67040903-67043254 |
| chr6: | 67042153-67043215 |
| chr6: | 67430539-67433180 |
| chr6: | 67943345-67946918 |
| chr6: | 69138565-69139360 |
| chr6: | 69471173-69471669 |
| chr6: | 69687686-69691720 |
| chr6: | 70014088-70018507 |
| chr6: | 71333319-71334191 |
| chr6: | 71690697-71691390 |
| chr6: | 71860706-71862638 |
| chr6: | 71998410-71999070 |
| chr6: | 72560288-72560780 |
| chr6: | 72863860-72868084 |
| chr6: | 73918942-73919882 |
| chr6: | 74340320-74342249 |
| chr6: | 74504214-74506345 |
| chr6: | 74832500-74833451 |
| chr6: | 75718864-75722640 |
| chr6: | 75721030-75722640 |
| chr6: | 77097492-77100534 |
| chr6: | 77450122-77452808 |
| chr6: | 78009962-78011477 |
| chr6: | 79521092-79524874 |
| chr6: | 30159451-80161340 |
| chr6: | 82117220-82118542 |
| chr6: | 82176187-82177524 |
| chr6: | 84735617-84739227 |
| chr6: | 88731181-88732674 |
| chr6: | 88801193-88802457 |
| chr6: | 89276056-89276506 |
| chr6: | 89491224-89492206 |
| chr6: | 89841950-89846943 |
| chr6: | 89921631-89922223 |
| chr6: | 90062279-90062904 |
| chr6: | 91005093-91006843 |
| chr6: | 91358818-91360324 |
| chr6: | 93574781-93578936 |
| chr6: | 93596266-93599321 |
| chr6: | 94734926-94735762 |
| chr6: | 95193393-95194392 |
| chr6: | 95289038-95291914 |
| chr6: | 96387639-96389399 |
| chr6: | 97468155-97470816 |
| chr6: | 99389160-99390831 |
| chr6: | 100034554-100035273 |
| chr6: | 100706818-100707478 |
| chr6: | 101193766-101196508 |
| chr6: | 101381140-101384880 |
| chr6: | 101482513-101487418 |
| chr6: | 101934557-101939024 |
| chr6: | 102314598-102319512 |
| chr6: | 102418494-102419108 |
| chr6: | 102815049-102815935 |
| chr6: | 102880342-102883148 |
| chr6: | 102911687-102912317 |
| chr6: | 105260784-105262877 |
| chr6: | 105388369-105389996 |
| chr6: | 105756744-105757280 |
| chr6: | 106154644-106157451 |
| chr6: | 106403851-106406231 |
| chr6: | 106697399-106701478 |
| chr6: | 106769705-106770767 |
| chr6: | 106981224-106982723 |
| chr6: | 107435508-107436934 |
| chr6: | 107544786-107546306 |
| chr6: | 108031352-108032517 |
| chr6: | 108183702-108186314 |
| chr6: | 108184097-108185247 |
| chr6: | 108184461-108185507 |
| chr6: | 108878970-108883212 |
| chr6: | 110569724-110571891 |
| chr6: | 111943994-111944614 |
| chr6: | 112422759-112424378 |
| chr6: | 112527156-112527907 |
| chr6: | 112527565-112529432 |
| chr6: | 112645085-112645753 |
| chr6: | 112875196-112879455 |
| chr6: | 113701811-113703135 |
| chr6: | 113777044-113778155 |
| chr6: | 114224029-114225246 |
| chr6: | 116170476-116172922 |
| chr6: | 119013104-119014016 |
| chr6: | 119039478-119040493 |
| chr6: | 120928642-120929589 |
| chr6: | 122080412-122081653 |
| chr6: | 122675849-122676381 |
| chr6: | 123592309-123593359 |
| chr6: | 126183269-126187152 |
| chr6: | 128432770-128437573 |
| chr6: | 128827511-128829555 |
| chr6: | 131814511-131816116 |
| chr6: | 132106620-132108756 |
| chr6: | 132114969-132118530 |
| chr6: | 132375570-132378995 |
| chr6: | 132707651-132712477 |
| chr6: | 133745934-133747363 |
| chr6: | 133949701-133950467 |
| chr6: | 135062081-135062747 |
| chr6: | 135160528-135164777 |
| chr6: | 136826484-136827744 |
| chr6: | 136851673-136852933 |
| chr6: | 141055120-141055737 |
| chr6: | 141377844-141380266 |
| chr6: | 141548633-141549965 |
| chr6: | 141549338-141549965 |
| chr6: | 142110591-142114931 |
| chr6: | 143615919-143619253 |
| chr6: | 143869378-143872377 |
| chr6: | 144195319-144199855 |
| chr6: | 144198734-144199855 |
| chr6: | 145281001-145282036 |
| chr6: | 145547525-145551165 |
| chr6: | 145705398-145706500 |
| chr6: | 147933952-147936609 |
| chr6: | 148396488-148397171 |
| chr6: | 148725574-148727395 |
| chr6: | 149197483-149200186 |
| chr6: | 149241022-149242084 |
| chr6: | 149629115-149633318 |
| chr6: | 150375285-150376585 |
| chr6: | 150518893-150519503 |
| chr6: | 150643454-150647802 |
| chr6: | 151819794-151820817 |
| chr6: | 151990558-151991178 |
| chr6: | 152389988-152392279 |
| chr6: | 152391226-152392210 |
| chr6: | 152941269-152942852 |
| chr6: | 153226679-153227738 |
| chr6: | 153226933-153227388 |
| chr6: | 153299316-153299827 |
| chr6: | 153958535-153961476 |
| chr6: | 154619798-154621694 |
| chr6: | 155118224-155123031 |
| chr6: | 155918182-155919705 |
| chr6: | 156052027-156053277 |
| chr6: | 156160525-156162079 |
| chr6: | 156730059-156730564 |
| chr6: | 157468452-157469014 |
| chr6: | 157731452-157735387 |
| chr6: | 157912574-157913486 |
| chr6: | 159125849-159126674 |
| chr6: | 159522729-159523334 |
| chr6: | 159673628-159674093 |
| chr6: | 161566070-161567418 |
| chr6: | 161726247-161728479 |
| chr6: | 161726953-161727789 |
| chr6: | 161727225-161727789 |
| chr6: | 161727258-161728794 |
| chr6: | 161811876-161814807 |
| chr6: | 162066925-162067590 |
| chr6: | 162137079-162137621 |
| chr6: | 162150845-162152544 |
| chr6: | 162384846-162386527 |
| chr6: | 162578556-162580303 |
| chr6: | 162583740-162587157 |
| chr6: | 163750285-163750850 |
| chr6: | 163755135-163755860 |
| chr6: | 163776750-163777745 |
| chr6: | 163834591-163336760 |
| chr6: | 164353113-164354288 |
| chr6: | 164543239-164547423 |
| chr6: | 165269422-165273754 |
| chr6: | 166999036-167000159 |
| chr6: | 167094453-167095253 |
| chr6: | 167149339-167150785 |
| chr6: | 167161735-167163265 |
| chr6: | 167162275-167162975 |
| chr6: | 167197318-167198244 |
| chr6: | 167488131-167489148 |
| chr6: | 167542576-167545986 |
| chr6: | 167543051-167544426 |
| chr6: | 167698628-167699330 |
| chr6: | 167729592-167730816 |
| chr6: | 168717001-168718791 |
| chr6: | 168732713-168733242 |
| chr6: | 168732713-168733853 |
| chr6: | 168757171-168760685 |
| chr6: | 168778630-168781584 |
| chr6: | 168839281-168890921 |
| chr6: | 168961145-168963177 |
| chr6: | 168972485-168974090 |
| chr6: | 168992913-168995956 |
| chr6: | 168996756-168997221 |
| chr6: | 169012882-169017826 |
| chr6: | 169014193-169016252 |
| chr6: | 169054220-169055964 |
| chr6: | 169126143-169126733 |
| chr6: | 169239853-169241373 |
| chr6: | 169246944-169248094 |
| chr6: | 169309878-169310613 |
| chr6: | 169329244-169332877 |
| chr6: | 169331263-169333042 |
| chr6: | 169390720-169391990 |
| chr6: | 169424741-169425421 |
| chr6: | 169525561-169530071 |
| chr6: | 169591832-169592872 |
| chr6: | 169633222-169635277 |
| chr6: | 169633686-169634341 |
| chr7: | 140248-140733 |
| chr7: | 177624-178164 |
| chr7: | 192015-193970 |
| chr7: | 201720-202851 |
| chr7: | 206526-207381 |
| chr7: | 215278-216453 |
| chr7: | 220963-222038 |
| chr7: | 224068-226043 |
| chr7: | 590970-592505 |
| chr7: | 675289-676119 |
| chr7: | 682436-683052 |
| chr7: | 916684-917434 |
| chr7: | 929180-930475 |
| chr7: | 973155-974065 |
| chr7: | 976694-977404 |
| chr7: | 993160-994633 |
| chr7: | 1040871-1043666 |
| chr7: | 1113244-1113980 |
| chr7: | 1154668-1157433 |
| chr7: | 1235629-1236600 |
| chr7: | 1275951-1276837 |
| chr7: | 1311430-1313780 |
| chr7: | 1311680-1312770 |
| chr7: | 1312800-1313820 |
| chr7: | 1458008-1458510 |
| chr7: | 1560690-1562832 |
| chr7: | 1812316-1817155 |
| chr7: | 1942334-1945740 |
| chr7: | 1977227-1978103 |
| chr7: | 2029476-2032431 |
| chr7: | 2030276-2031861 |
| chr7: | 2074851-2075991 |
| chr7: | 2417089-2417789 |
| chr7: | 2417194-2418399 |
| chr7: | 2757395-2759983 |
| chr7: | 2965347-2965887 |
| chr7: | 3135771-3138088 |
| chr7: | 3339551-3340091 |
| chr7: | 3339551-3341631 |
| chr7: | 3654478-3655481 |
| chr7: | 3959832-3960502 |
| chr7: | 4002521-4003496 |
| chr7: | 4015269-4017573 |
| chr7: | 4080568-4082559 |
| chr7: | 4081254-4082299 |
| chr7: | 4105563-4106213 |
| chr7: | 4158181-4160230 |
| chr7: | 4159127-4160165 |
| chr7: | 4303105-4304385 |
| chr7: | 4416591-4420281 |
| chr7: | 4842033-4843310 |
| chr7: | 7924165-7926172 |
| chr7: | 8463594-8465549 |
| chr7: | 8992886-8994434 |
| chr7: | 9515385-9515998 |
| chr7: | 9521401-9523934 |
| chr7: | 9676190-9680419 |
| chr7: | 11281903-11282480 |
| chr7: | 11602763-11604286 |
| chr7: | 11920594-11922688 |
| chr7: | 11946500-11946946 |
| chr7: | 12016234-12018681 |
| chr7: | 12049725-12052297 |
| chr7: | 12122220-12122980 |
| chr7: | 12524654-12525532 |
| chr7: | 12566154-12568348 |
| chr7: | 13278045-13280863 |
| chr7: | 13866579-13871138 |
| chr7: | 14250688-14252165 |
| chr7: | 14829840-14832085 |
| chr7: | 14837460-14841270 |
| chr7: | 15089246-15092130 |
| chr7: | 15413774-15418443 |
| chr7: | 15635015-15638178 |
| chr7: | 15658587-15659078 |
| chr7: | 16171796-16173975 |
| chr7: | 16700143-16702252 |
| chr7: | 16876288-16878794 |
| chr7: | 17813669-17814724 |
| chr7: | 17844487-17845419 |
| chr7: | 18702426-18703257 |
| chr7: | 19627181-19628138 |
| chr7: | 20748835-20753546 |
| chr7: | 21830214-21830716 |
| chr7: | 22434322-22436723 |
| chr7: | 23139915-23144237 |
| chr7: | 23186675-23188052 |
| chr7: | 23186675-23191648 |
| chr7: | 23454320-23455185 |
| chr7: | 24038230-24040059 |
| chr7: | 24151961-24154305 |
| chr7: | 24359616-24363485 |
| chr7: | 24911075-24911630 |
| chr7: | 25360557-25362570 |
| chr7: | 25456940-25457588 |
| chr7: | 25641352-25642036 |
| chr7: | 26219370-26222359 |
| chr7: | 26251715-26252962 |
| chr7: | 26305273-26308249 |
| chr7: | 26654807-26655980 |
| chr7: | 27412567-27413053 |
| chr7: | 27943319-27944393 |
| chr7: | 28574039-28575217 |
| chr7: | 28574315-28574878 |
| chr7: | 28706478-28707108 |
| chr7: | 28949580-28950202 |
| chr7: | 29688613-29690151 |
| chr7: | 29715007-29717393 |
| chr7: | 30855922-30856777 |
| chr7: | 31053161-31053831 |
| chr7: | 31315488-31318931 |
| chr7: | 31590239-31591957 |
| chr7: | 31712956-31715980 |
| chr7: | 32549714-32551159 |
| chr7: | 33177800-33179356 |
| chr7: | 38384509-38388845 |
| chr7: | 38625960-38626516 |
| chr7: | 46113771-46115484 |
| chr7: | 46829765-46832724 |
| chr7: | 47089580-47092655 |
| chr7: | 47799598-47801500 |
| chr7: | 48587610-48588210 |
| chr7: | 48743911-48744667 |
| chr7: | 49586875-49588989 |
| chr7: | 50104797-50106153 |
| chr7: | 50343207-50344407 |
| chr7: | 50860377-50861113 |
| chr7: | 51594399-51598231 |
| chr7: | 51851085-51853814 |
| chr7: | 52008355-52008834 |
| chr7: | 52425593-52429130 |
| chr7: | 52963105-52965169 |
| chr7: | 52963788-52965049 |
| chr7: | 52964360-52964921 |
| chr7: | 54596489-54597530 |
| chr7: | 66858458-66859603 |
| chr7: | 66966364-66968758 |
| chr7: | 67131178-67132149 |
| chr7: | 68262140-68262774 |
| chr7: | 70420989-70425839 |
| chr7: | 70619910-70624629 |
| chr7: | 71578779-71579668 |
| chr7: | 72044598-72046394 |
| chr7: | 72269468-72273243 |
| chr7: | 73829106-73831236 |
| chr7: | 73829958-73831236 |
| chr7: | 78339246-78339861 |
| chr7: | 78671664-78674753 |
| chr7: | 79948934-79950361 |
| chr7: | 81441480-81442585 |
| chr7: | 81441678-81442448 |
| chr7: | 81685304-81689008 |
| chr7: | 81814174-81815178 |
| chr7: | 83050418-83051171 |
| chr7: | 85609847-85612229 |
| chr7: | 85611178-85612130 |
| chr7: | 87308361-87310824 |
| chr7: | 87668826-87672870 |
| chr7: | 88903880-88905477 |
| chr7: | 89250721-89252521 |
| chr7: | 89810459-89812588 |
| chr7: | 90426083-90427640 |
| chr7: | 90478554-90479569 |
| chr7: | 91358557-91360407 |
| chr7: | 91562340-91564755 |
| chr7: | 93226362-93226935 |
| chr7: | 93541799-93542525 |
| chr7: | 96116326-96117391 |
| chr7: | 96543223-96546445 |
| chr7: | 98228411-98230024 |
| chr7: | 98228554-98231051 |
| chr7: | 98740131-98741285 |
| chr7: | 99322829-99327194 |
| chr7: | 99461451-99463547 |
| chr7: | 101118504-101118985 |
| chr7: | 101457194-101460796 |
| chr7: | 101724388-101727498 |
| chr7: | 103772400-103774548 |
| chr7: | 104928324-104931171 |
| chr7: | 107499907-107500432 |
| chr7: | 107908807-107909334 |
| chr7: | 110428390-110432691 |
| chr7: | 110430966-110432322 |
| chr7: | 110484747-110485512 |
| chr7: | 111065337-111067231 |
| chr7: | 111232211-111232995 |
| chr7: | 112243450-112244619 |
| chr7: | 113726889-113728602 |
| chr7: | 116033231-116034314 |
| chr7: | 118831213-118834705 |
| chr7: | 119144787-119145778 |
| chr7: | 119232822-119236164 |
| chr7: | 119657064-119659044 |
| chr7: | 120647816-120648879 |
| chr7: | 121083326-121084249 |
| chr7: | 121033584-121085279 |
| chr7: | 121217627-121219617 |
| chr7: | 121220580-121225270 |
| chr7: | 121340905-121343117 |
| chr7: | 122870917-122871998 |
| chr7: | 122871306-122873163 |
| chr7: | 124113222-124117647 |
| chr7: | 124115496-124117956 |
| chr7: | 124656282-124656998 |
| chr7: | 125558328-125562966 |
| chr7: | 127214985-127217698 |
| chr7: | 128729006-128732161 |
| chr7: | 128770266-128771937 |
| chr7: | 131078728-131080804 |
| chr7: | 131560447-131562547 |
| chr7: | 134231533-134234119 |
| chr7: | 134310556-134312115 |
| chr7: | 134542220-134543286 |
| chr7: | 137573105-137573771 |
| chr7: | 137931981-137932705 |
| chr7: | 137995548-137996346 |
| chr7: | 139206050-139210360 |
| chr7: | 139875470-139377013 |
| chr7: | 144770276-144775153 |
| chr7: | 144916993-144921583 |
| chr7: | 145268675-145269174 |
| chr7: | 147011522-147013175 |
| chr7: | 147102850-147104109 |
| chr7: | 147194510-147195154 |
| chr7: | 147282459-147285145 |
| chr7: | 148073126-148076330 |
| chr7: | 148395807-148396877 |
| chr7: | 149847642-149848528 |
| chr7: | 150346277-150347170 |
| chr7: | 150811616-150812941 |
| chr7: | 150812236-150812966 |
| chr7: | 151229573-151231573 |
| chr7: | 152579024-152579889 |
| chr7: | 152986363-152987563 |
| chr7: | 153106266-153111170 |
| chr7: | 153263315-153263949 |
| chr7: | 153328112-153329223 |
| chr7: | 153879401-153879911 |
| chr7: | 153919794-153922552 |
| chr7: | 153919794-153923440 |
| chr7: | 154266246-154267496 |
| chr7: | 154447469-154449434 |
| chr7: | 154451816-154456154 |
| chr7: | 154709811-154713998 |
| chr7: | 154885265-154889486 |
| chr7: | 154889187-154891019 |
| chr7: | 154931600-154933065 |
| chr7: | 154932005-154932505 |
| chr7: | 154962754-154965370 |
| chr7: | 155138256-155141554 |
| chr7: | 155199575-155201290 |
| chr7: | 155200355-155201290 |
| chr7: | 155407598-155411723 |
| chr7: | 155946587-155948678 |
| chr7: | 156774604-156776907 |
| chr7: | 157234044-157235464 |
| chr7: | 157439473-157443223 |
| chr7: | 157517836-157519276 |
| chr7: | 157523886-157527011 |
| chr7: | 157539436-157542571 |
| chr7: | 157649972-157652807 |
| chr7: | 157719644-157720554 |
| chr7: | 157733108-157734369 |
| chr7: | 157733434-157734029 |
| chr7: | 157787302-157787997 |
| chr7: | 157850892-157854419 |
| chr7: | 157876755-157879120 |
| chr7: | 157879025-157883151 |
| chr7: | 157891582-157892454 |
| chr7: | 157896898-157901133 |
| chr7: | 157939459-157942723 |
| chr7: | 157939583-157944461 |
| chr7: | 157951135-157952085 |
| chr7: | 157980919-157981449 |
| chr7: | 158037863-158039667 |
| chr7: | 158046631-158047401 |
| chr7: | 158065846-158067216 |
| chr7: | 158152547-158154186 |
| chr7: | 158188025-158188545 |
| chr7: | 158199766-158200406 |
| chr7: | 158217813-158220687 |
| chr7: | 158288898-158289728 |
| chr7: | 158313998-158317393 |
| chr7: | 158314318-158315258 |
| chr7: | 158316098-158317253 |
| chr7: | 158394847-158395392 |
| chr7: | 158460031-158461077 |
| chr7: | 158500546-153504942 |
| chr7: | 158521478-153525074 |
| chr7: | 158521706-158522576 |
| chr7: | 158521921-158523480 |
| chr7: | 158631329-158632171 |
| chr7: | 158707734-158710455 |
| chr7: | 158743175-158743644 |
| chr7: | 158879800-158883680 |
| chr7: | 158881560-158882192 |
| chr7: | 158921835-158922745 |
| chr7: | 158944911-158945506 |
| chr7: | 158995372-158996472 |
| chr7: | 159001262-159001847 |
| chr8: | 1573805-1575062 |
| chr8: | 1575157-1577002 |
| chr8: | 1590011-1592330 |
| chr8: | 1658804-1659750 |
| chr8: | 1746416-1747051 |
| chr8: | 1746416-1748254 |
| chr8: | 1787196-1790527 |
| chr8: | 1797568-1798743 |
| chr8: | 1835579-1838789 |
| chr8: | 1837004-1838364 |
| chr8: | 1867121-1868311 |
| chr8: | 1938559-1939424 |
| chr8: | 1939694-1942449 |
| chr8: | 1949350-1951397 |
| chr8: | 2033596-2034346 |
| chr8: | 2044360-2044890 |
| chr8: | 2085486-2086176 |
| chr8: | 2085861-2086926 |
| chr8: | 2119676-2122791 |
| chr8: | 2121086-2122791 |
| chr8: | 2195166-2195636 |
| chr8: | 2215297-2216207 |
| chr8: | 2329212-2329751 |
| chr8: | 2339509-2340386 |
| chr8: | 2588163-2593045 |
| chr8: | 2652254-2652949 |
| chr8: | 2887168-2890520 |
| chr8: | 3096059-3101036 |
| chr8: | 3139355-3140920 |
| chr8: | 3140205-3140920 |
| chr8: | 3181371-3182221 |
| chr8: | 3405775-3406530 |
| chr8: | 3411237-3412987 |
| chr8: | 3529630-3530170 |
| chr8: | 3566776-3568330 |
| chr8: | 3580046-3581683 |
| chr8: | 3581023-3581683 |
| chr8: | 3785864-3790280 |
| chr8: | 3789113-3790280 |
| chr8: | 3814308-3816551 |
| chr8: | 4027449-4029234 |
| chr8: | 4049447-4051991 |
| chr8: | 4122900-4124927 |
| chr8: | 4122968-4123586 |
| chr8: | 4446085-4449641 |
| chr8: | 4449132-4449771 |
| chr8: | 4449199-4450231 |
| chr8: | 4798362-4800295 |
| chr8: | 4980137-4981111 |
| chr8: | 5475437-5476527 |
| chr8: | 5642685-5645829 |
| chr8: | 5750384-5750993 |
| chr8: | 5764849-5765516 |
| chr8: | 5879973-5882126 |
| chr8: | 5881320-5882771 |
| chr8: | 6104316-6105059 |
| chr8: | 6159807-6160359 |
| chr8: | 6229691-6232076 |
| chr8: | 6638547-6639292 |
| chr8: | 6708301-6713175 |
| chr8: | 8286359-8290837 |
| chr8: | 8749942-8751417 |
| chr8: | 9614977-9615592 |
| chr8: | 10883638-10884662 |
| chr8: | 11245574-11247131 |
| chr8: | 11312680-11314577 |
| chr8: | 11491858-11495529 |
| chr8: | 11736855-11738771 |
| chr8: | 13892568-13893545 |
| chr8: | 14038331-14033800 |
| chr8: | 14341163-14342343 |
| chr8: | 14345479-14347082 |
| chr8: | 14501601-14503253 |
| chr8: | 15142528-15143922 |
| chr8: | 15815873-15818513 |
| chr8: | 16322187-16326583 |
| chr8: | 16821836-16822298 |
| chr8: | 17580657-17581823 |
| chr8: | 17737400-17738241 |
| chr8: | 17750240-17752329 |
| chr8: | 17751257-17752242 |
| chr8: | 17762272-17763647 |
| chr8: | 18124121-18128422 |
| chr8: | 18813696-18814311 |
| chr8: | 19091441-19092038 |
| chr8: | 19318926-19319576 |
| chr8: | 19373431-19376616 |
| chr8: | 19587584-19590311 |
| chr8: | 19854644-19855520 |
| chr8: | 19871323-19872028 |
| chr8: | 20783606-20784742 |
| chr8: | 21167753-21171778 |
| chr8: | 21276631-21278147 |
| chr8: | 21646187-21648409 |
| chr8: | 22189156-22192080 |
| chr8: | 23406986-23407907 |
| chr8: | 24089297-24091839 |
| chr8: | 24442840-24446857 |
| chr8: | 24593533-24595534 |
| chr8: | 24725446-24726578 |
| chr8: | 25066730-25070660 |
| chr8: | 25120499-25124656 |
| chr8: | 25550822-25551618 |
| chr8: | 27381785-27383010 |
| chr8: | 27683090-27689082 |
| chr8: | 27740214-27740888 |
| chr8: | 30604354-30605592 |
| chr8: | 30878199-30879453 |
| chr8: | 35055998-35059337 |
| chr8: | 35432477-35433678 |
| chr8: | 36370007-36371867 |
| chr8: | 36370378-36370928 |
| chr8: | 38447079-38448375 |
| chr8: | 38447851-38448375 |
| chr8: | 39242813-39244412 |
| chr8: | 39885677-39890411 |
| chr8: | 40318176-40313801 |
| chr8: | 40469744-40470288 |
| chr8: | 40774718-40779534 |
| chr8: | 43631346-43636069 |
| chr8: | 47199741-47202809 |
| chr8: | 47330565-47335505 |
| chr8: | 47405751-47408006 |
| chr8: | 47406795-47407647 |
| chr8: | 50008794-50009344 |
| chr8: | 51225015-51223269 |
| chr8: | 51555987-51558277 |
| chr8: | 53700979-53701627 |
| chr8: | 53896635-53897201 |
| chr8: | 54122580-54123630 |
| chr8: | 54190924-54195796 |
| chr8: | 54194628-54195307 |
| chr8: | 55209098-55211264 |
| chr8: | 55737013-55737990 |
| chr8: | 59682320-59684458 |
| chr8: | 59684003-59684458 |
| chr8: | 60135561-60136492 |
| chr8: | 60451739-60454634 |
| chr8: | 63304511-63305607 |
| chr8: | 63423337-63424699 |
| chr8: | 63806089-63809086 |
| chr8: | 66091851-66094608 |
| chr8: | 66759486-66760364 |
| chr8: | 67120890-67121680 |
| chr8: | 68546758-68551563 |
| chr8: | 68893729-68894374 |
| chr8: | 68904053-68905047 |
| chr8: | 69185324-69188803 |
| chr8: | 69689769-69690246 |
| chr8: | 70320240-70325203 |
| chr8: | 71451758-71452491 |
| chr8: | 71654956-71657674 |
| chr8: | 72214710-72217673 |
| chr8: | 72646710-72647839 |
| chr8: | 73325692-73330633 |
| chr8: | 78125163-78126207 |
| chr8: | 79545392-79548500 |
| chr8: | 79856923-79859124 |
| chr8: | 81251961-81252431 |
| chr8: | 83857713-83858447 |
| chr8: | 85254447-85255119 |
| chr8: | 85524784-85527776 |
| chr8: | 85736528-85740278 |
| chr8: | 87099571-87100857 |
| chr8: | 87648215-87649641 |
| chr8: | 87671249-87672604 |
| chr8: | 88512308-88512766 |
| chr8: | 89098166-89100420 |
| chr8: | 89926789-89929890 |
| chr8: | 91191012-91191851 |
| chr8: | 91201431-91202706 |
| chr8: | 94072329-94073603 |
| chr8: | 95647007-95647921 |
| chr8: | 95725289-95727158 |
| chr8: | 96079304-96081691 |
| chr8: | 96452101-96452781 |
| chr8: | 96875068-96879582 |
| chr8: | 97262970-97263424 |
| chr8: | 97520067-97521908 |
| chr8: | 97520755-97521959 |
| chr8: | 98862081-93363316 |
| chr8: | 99314189-99315584 |
| chr8: | 99357214-99359367 |
| chr8: | 99935526-99936475 |
| chr8: | 100698324-100701511 |
| chr8: | 103053958-103056707 |
| chr8: | 103288097-103288853 |
| chr8: | 103570938-103572525 |
| chr8: | 103994756-103996586 |
| chr8: | 104758530-104762943 |
| chr8: | 106579704-106580596 |
| chr8: | 106796546-106797356 |
| chr8: | 107234068-107235610 |
| chr8: | 107232865-107283690 |
| chr8: | 107512907-107513857 |
| chr8: | 107857322-107862232 |
| chr8: | 108504879-103506388 |
| chr8: | 108781337-103782133 |
| chr8: | 109235843-109238135 |
| chr8: | 110922211-110922856 |
| chr8: | 111939993-111944199 |
| chr8: | 112294084-112296293 |
| chr8: | 112351139-112351957 |
| chr8: | 112552331-112554291 |
| chr8: | 114056559-114058727 |
| chr8: | 115079446-115080682 |
| chr8: | 115865417-115867698 |
| chr8: | 116763148-116763654 |
| chr8: | 118005483-118008163 |
| chr8: | 118874053-118875618 |
| chr8: | 119214373-119214987 |
| chr8: | 119445015-119446781 |
| chr8: | 120109065-120111913 |
| chr8: | 120325182-120328062 |
| chr8: | 120326621-120328062 |
| chr8: | 120735415-120785993 |
| chr8: | 121762946-121764231 |
| chr8: | 121872606-121874392 |
| chr8: | 122239675-122240424 |
| chr8: | 122239675-122240718 |
| chr8: | 123058996-123060940 |
| chr8: | 125628575-125629271 |
| chr8: | 127192227-127194539 |
| chr8: | 127401211-127404894 |
| chr8: | 128243750-128249755 |
| chr8: | 129616548-129617056 |
| chr8: | 129762926-129766230 |
| chr8: | 130140866-130145246 |
| chr8: | 131061175-131062625 |
| chr8: | 131370202-131371248 |
| chr8: | 131850764-131852855 |
| chr8: | 132091573-132092929 |
| chr8: | 132882216-132882726 |
| chr8: | 133060525-133061660 |
| chr8: | 133313367-133314213 |
| chr8: | 134328398-134330552 |
| chr8: | 134363649-134364139 |
| chr8: | 135022565-135023116 |
| chr8: | 135509628-135511923 |
| chr8: | 135833551-135837365 |
| chr8: | 136218418-136220592 |
| chr8: | 136469355-136470368 |
| chr8: | 137160192-137163874 |
| chr8: | 138125896-138127381 |
| chr8: | 138742958-133743856 |
| chr8: | 138800907-138802397 |
| chr8: | 138817123-138819373 |
| chr8: | 138822712-138825768 |
| chr8: | 138911640-138912197 |
| chr8: | 139173685-139175125 |
| chr8: | 139925030-139925710 |
| chr8: | 140160652-140161401 |
| chr8: | 140257618-140259153 |
| chr8: | 140264248-140266903 |
| chr8: | 140603791-140607020 |
| chr8: | 140759587-140761010 |
| chr8: | 140767060-140769620 |
| chr8: | 141166885-141167490 |
| chr8: | 141204925-141207002 |
| chr8: | 141645130-141646365 |
| chr8: | 142002613-142005570 |
| chr8: | 142255021-142255546 |
| chr8: | 142345193-142345983 |
| chr8: | 142439695-142440540 |
| chr8: | 142502375-142503320 |
| chr8: | 142560394-142561674 |
| chr8: | 142560839-142561424 |
| chr8: | 142640591-142645056 |
| chr8: | 142893582-142395248 |
| chr8: | 142953171-142955911 |
| chr8: | 143063594-143066241 |
| chr8: | 143132496-143183201 |
| chr8: | 143243439-143245919 |
| chr8: | 143323262-143323710 |
| chr8: | 143397471-143398771 |
| chr8: | 143471859-143472794 |
| chr8: | 143824316-143827246 |
| chr8: | 143841182-143842759 |
| chr8: | 143979268-143980201 |
| chr8: | 144056412-144057092 |
| chr8: | 144117438-144121875 |
| chr8: | 144153615-144155420 |
| chr8: | 144248046-144250143 |
| chr8: | 144292694-144293768 |
| chr8: | 144386337-144387967 |
| chr8: | 144389333-144390783 |
| chr8: | 144454331-144456157 |
| chr8: | 144455066-144455634 |
| chr8: | 144634308-144636248 |
| chr8: | 144679592-144681484 |
| chr8: | 144743555-144744738 |
| chr8: | 145504924-145505401 |
| chr8: | 145537207-145537712 |
| chr8: | 145560219-145562289 |
| chr8: | 145574486-145575006 |
| chr8: | 145603693-145607843 |
| chr8: | 145717692-145720787 |
| chr8: | 145756440-145757575 |
| chr8: | 145781917-145783178 |
| chr8: | 145786480-145788245 |
| chr8: | 145825565-145828779 |
| chr8: | 145912242-145913429 |
| chr8: | 145924300-145926735 |
| chr8: | 146171782-146172877 |
| chr9: | 10485-10965 |
| chr9: | 1163464-1163990 |
| chr9: | 1527196-1527745 |
| chr9: | 1639550-1640161 |
| chr9: | 1716975-1720283 |
| chr9: | 1718048-1719043 |
| chr9: | 2148145-2151403 |
| chr9: | 2286439-2288022 |
| chr9: | 2348841-2349573 |
| chr9: | 4206045-4209485 |
| chr9: | 4208648-4209502 |
| chr9: | 4258320-4259510 |
| chr9: | 4376094-4377181 |
| chr9: | 4376094-4377777 |
| chr9: | 4397534-4398316 |
| chr9: | 4454463-4458263 |
| chr9: | 4501098-4503959 |
| chr9: | 4599944-4600533 |
| chr9: | 4654974-4656940 |
| chr9: | 4725396-4723151 |
| chr9: | 5641648-5642301 |
| chr9: | 5750200-5751316 |
| chr9: | 6011447-6014308 |
| chr9: | 6013304-6014308 |
| chr9: | 8344499-3344982 |
| chr9: | 8640898-8641854 |
| chr9: | 8640898-8642335 |
| chr9: | 8883454-8884212 |
| chr9: | 9340141-9340948 |
| chr9: | 9517036-9517778 |
| chr9: | 9817010-9818405 |
| chr9: | 10020070-10022636 |
| chr9: | 10059703-10060468 |
| chr9: | 10404571-10405103 |
| chr9: | 10831583-10834501 |
| chr9: | 11076314-11077076 |
| chr9: | 11106326-11109319 |
| chr9: | 11224400-11228073 |
| chr9: | 11506417-11509213 |
| chr9: | 11692221-11694401 |
| chr9: | 12363573-12369413 |
| chr9: | 13065810-13069741 |
| chr9: | 13571339-13575947 |
| chr9: | 14518139-14519477 |
| chr9: | 14686873-14638481 |
| chr9: | 15020755-15022389 |
| chr9: | 15157502-15159344 |
| chr9: | 15815327-15817193 |
| chr9: | 16289392-16290247 |
| chr9: | 16882532-16884965 |
| chr9: | 17260551-17262869 |
| chr9: | 17764515-17765573 |
| chr9: | 17910038-17911633 |
| chr9: | 19273398-19274277 |
| chr9: | 19358477-19359692 |
| chr9: | 19547521-19548233 |
| chr9: | 19671928-19673142 |
| chr9: | 19888822-19889291 |
| chr9: | 20212052-20213471 |
| chr9: | 21734362-21734837 |
| chr9: | 22666435-22666902 |
| chr9: | 24145509-24150358 |
| chr9: | 25030864-25031737 |
| chr9: | 25722880-25724737 |
| chr9: | 26530630-26531474 |
| chr9: | 27200615-27203679 |
| chr9: | 28661946-28665145 |
| chr9: | 28846246-28847699 |
| chr9: | 29093580-29097377 |
| chr9: | 29259551-29260715 |
| chr9: | 29591826-29592351 |
| chr9: | 30903182-30909486 |
| chr9: | 31291284-31292768 |
| chr9: | 32066344-32066839 |
| chr9: | 32882687-32883305 |
| chr9: | 33817099-33817701 |
| chr9: | 34957784-34953637 |
| chr9: | 34987518-34988462 |
| chr9: | 35115200-35116195 |
| chr9: | 35170847-35175142 |
| chr9: | 35372369-35375249 |
| chr9: | 35605071-35605941 |
| chr9: | 35728110-35732416 |
| chr9: | 35913117-35914447 |
| chr9: | 35980780-35982060 |
| chr9: | 71895230-71896481 |
| chr9: | 72762238-72763468 |
| chr9: | 73442098-73444722 |
| chr9: | 73443111-73444722 |
| chr9: | 73593904-73597196 |
| chr9: | 74667716-74668597 |
| chr9: | 74883343-74834154 |
| chr9: | 75805885-75808434 |
| chr9: | 76060814-76062850 |
| chr9: | 76744822-76746896 |
| chr9: | 76789673-76790804 |
| chr9: | 78089309-78092133 |
| chr9: | 78740242-78741687 |
| chr9: | 78919551-78922079 |
| chr9: | 79555768-79558107 |
| chr9: | 79602100-79602772 |
| chr9: | 79602100-79603442 |
| chr9: | 80059063-80063119 |
| chr9: | 80231633-80233458 |
| chr9: | 80645502-80647711 |
| chr9: | 80646280-80647390 |
| chr9: | 81120028-81121261 |
| chr9: | 81595355-81596026 |
| chr9: | 82907614-82909107 |
| chr9: | 32982168-82985074 |
| chr9: | 83387014-83387655 |
| chr9: | 83427080-83431735 |
| chr9: | 83742177-83742849 |
| chr9: | 83967945-83970647 |
| chr9: | 84690765-84691991 |
| chr9: | 87178250-87182344 |
| chr9: | 87352531-87353316 |
| chr9: | 87618304-87618855 |
| chr9: | 87775156-87776206 |
| chr9: | 88018385-88019045 |
| chr9: | 88473454-88476250 |
| chr9: | 88722266-88724783 |
| chr9: | 89154952-89155776 |
| chr9: | 89560746-89562196 |
| chr9: | 89587570-89588978 |
| chr9: | 89758176-89759443 |
| chr9: | 89922640-89923420 |
| chr9: | 90065726-90066315 |
| chr9: | 90132121-90132961 |
| chr9: | 90177231-90178746 |
| chr9: | 90265846-90268320 |
| chr9: | 91148990-91150155 |
| chr9: | 91286628-91233762 |
| chr9: | 91538505-91539074 |
| chr9: | 91562872-91565048 |
| chr9: | 91673346-91675056 |
| chr9: | 91863195-91869753 |
| chr9: | 91984764-91986292 |
| chr9: | 92176053-92177915 |
| chr9: | 92768618-92769372 |
| chr9: | 93603341-93605009 |
| chr9: | 94017894-94019712 |
| chr9: | 94558813-94560842 |
| chr9: | 94563381-94566086 |
| chr9: | 96433251-96435331 |
| chr9: | 96470344-96471499 |
| chr9: | 96499413-96503324 |
| chr9: | 96500204-96501594 |
| chr9: | 96663073-96663603 |
| chr9: | 96679706-96680751 |
| chr9: | 97316907-97320096 |
| chr9: | 97334132-97335064 |
| chr9: | 97334587-97335064 |
| chr9: | 97462576-97464526 |
| chr9: | 98336522-98337367 |
| chr9: | 99248804-99249640 |
| chr9: | 99846075-99849809 |
| chr9: | 101000519-101000987 |
| chr9: | 101000519-101001605 |
| chr9: | 101309035-101311127 |
| chr9: | 102136735-102190546 |
| chr9: | 103760513-103762208 |
| chr9: | 104222718-104223864 |
| chr9: | 104496753-104498220 |
| chr9: | 105326948-105328173 |
| chr9: | 105775718-105778476 |
| chr9: | 106568334-106569735 |
| chr9: | 106856702-106857182 |
| chr9: | 107596215-107597836 |
| chr9: | 107793850-107803275 |
| chr9: | 108372342-108373320 |
| chr9: | 108536106-108538162 |
| chr9: | 109276422-109279898 |
| chr9: | 109739582-109742073 |
| chr9: | 110326518-110327223 |
| chr9: | 110427686-110431924 |
| chr9: | 112032692-112083437 |
| chr9: | 112519563-112520230 |
| chr9: | 112830199-112831132 |
| chr9: | 113175922-113179364 |
| chr9: | 113477175-113480142 |
| chr9: | 114165858-114170224 |
| chr9: | 116397774-116398539 |
| chr9: | 116506433-116509946 |
| chr9: | 117700663-117702283 |
| chr9: | 117830951-117833948 |
| chr9: | 117831511-117832302 |
| chr9: | 118195950-118197377 |
| chr9: | 119513146-119515902 |
| chr9: | 121033696-121034881 |
| chr9: | 121525629-121526643 |
| chr9: | 121534269-121537340 |
| chr9: | 122526465-122528741 |
| chr9: | 124226945-124227758 |
| chr9: | 126028415-126031141 |
| chr9: | 126028680-126030380 |
| chr9: | 126108476-126110276 |
| chr9: | 126166842-126168741 |
| chr9: | 126503014-126503509 |
| chr9: | 126617107-126617793 |
| chr9: | 126733957-126736352 |
| chr9: | 128508565-128510625 |
| chr9: | 129138961-129140677 |
| chr9: | 129209923-129210699 |
| chr9: | 129246789-129248224 |
| chr9: | 129246849-129249166 |
| chr9: | 129488171-129490861 |
| chr9: | 129519224-129523039 |
| chr9: | 129520606-129521995 |
| chr9: | 129520606-129523039 |
| chr9: | 130033154-130034740 |
| chr9: | 130181615-130186062 |
| chr9: | 130633204-130633984 |
| chr9: | 130813149-130816091 |
| chr9: | 130829609-130830219 |
| chr9: | 131701029-131701920 |
| chr9: | 132026730-132023020 |
| chr9: | 132101397-132102942 |
| chr9: | 132157306-132160726 |
| chr9: | 132215318-132216179 |
| chr9: | 132215388-132217103 |
| chr9: | 132215563-132220239 |
| chr9: | 132526505-132529784 |
| chr9: | 132614062-132614648 |
| chr9: | 133030707-133035158 |
| chr9: | 133333542-133387504 |
| chr9: | 133423382-133425644 |
| chr9: | 133752424-133752987 |
| chr9: | 133847185-133348450 |
| chr9: | 134269071-134270742 |
| chr9: | 134391024-134391858 |
| chr9: | 134618065-134622151 |
| chr9: | 136065678-136067375 |
| chr9: | 136171270-136175678 |
| chr9: | 136293884-136294879 |
| chr9: | 136533481-136534433 |
| chr9: | 136617313-136618084 |
| chr9: | 136625238-136626128 |
| chr9: | 136682384-136683165 |
| chr9: | 136692344-136693634 |
| chr9: | 136756194-136757458 |
| chr9: | 136856554-136858557 |
| chr9: | 136862625-136863975 |
| chr9: | 136876972-136879859 |
| chr9: | 136932368-136933856 |
| chr9: | 137217729-137219331 |
| chr9: | 137353025-137359300 |
| chr9: | 137415555-137416727 |
| chr9: | 137464650-137466825 |
| chr9: | 137495552-137496651 |
| chr9: | 137545390-137547020 |
| chr9: | 137545470-137549948 |
| chr9: | 137739895-137742885 |
| chr9: | 137742885-137744615 |
| chr9: | 138009914-138011314 |
| chr9: | 138176739-138178434 |
| chr9: | 138194166-138195707 |
| chr9: | 138214099-138217618 |
| chr9: | 138338293-138339244 |
| chr9: | 138440853-138441388 |
| chr9: | 138469428-138472049 |
| chr9: | 138479150-138480200 |
| chr9: | 138728106-138730926 |
| chr9: | 138798437-138800137 |
| chr9: | 139009980-139011258 |
| chr9: | 139054198-139058843 |
| chr9: | 139325482-139326427 |
| chr9: | 139429951-139430436 |
| chr9: | 139492730-139493260 |
| chr9: | 139662736-139665108 |
| chr9: | 139664193-139665136 |
| chr9: | 139995796-139998627 |
| chr9: | 140221018-140224869 |
| chr9: | 140222106-140222928 |
| chr9: | 140223009-140224284 |
| chr9: | 140223009-140225119 |
| chr9: | 140253772-140255871 |
| chr9: | 140273026-140274007 |
| chr9: | 140334417-140335797 |
| chr9: | 140344836-140346737 |
| chr9: | 140459557-140463142 |
| chr9: | 140610540-140611050 |
| chr9: | 140712977-140714207 |
| chr9: | 140735535-140737220 |
| chr9: | 140735845-140736610 |
| chr9: | 140739557-140740947 |
| chr9: | 140783394-140785354 |
| chr9: | 140912358-140914593 |
| chr10: | 1342801-1343614 |
| chr10: | 1363859-1369904 |
| chr10: | 1410813-1411921 |
| chr10: | 1480300-1482425 |
| chr10: | 1501289-1503104 |
| chr10: | 1506312-1507047 |
| chr10: | 1582580-1585280 |
| chr10: | 1600267-1601352 |
| chr10: | 1633931-1636079 |
| chr10: | 1634481-1635471 |
| chr10: | 1657661-1660888 |
| chr10: | 1658596-1660016 |
| chr10: | 1668101-1668606 |
| chr10: | 1741370-1744179 |
| chr10: | 2066869-2067445 |
| chr10: | 2543312-2544056 |
| chr10: | 2562864-2566440 |
| chr10: | 2622300-2623852 |
| chr10: | 2874446-2878346 |
| chr10: | 3053434-3055743 |
| chr10: | 3054340-3056094 |
| chr10: | 3114717-3116364 |
| chr10: | 3127046-3128011 |
| chr10: | 3163069-3165871 |
| chr10: | 3356310-3357470 |
| chr10: | 3573693-3575449 |
| chr10: | 3574351-3575364 |
| chr10: | 4290100-4291628 |
| chr10: | 4437636-4441411 |
| chr10: | 4708559-4710493 |
| chr10: | 4857721-4858391 |
| chr10: | 4910041-4910707 |
| chr10: | 5163668-5164362 |
| chr10: | 5249890-5250922 |
| chr10: | 5376187-5378525 |
| chr10: | 5396324-5397499 |
| chr10: | 5602128-5604507 |
| chr10: | 5889940-5892692 |
| chr10: | 6639150-6641560 |
| chr10: | 6876772-6879786 |
| chr10: | 7013209-7013829 |
| chr10: | 7077043-7078318 |
| chr10: | 7077509-7078318 |
| chr10: | 7708906-7709656 |
| chr10: | 7995740-7997161 |
| chr10: | 8008694-8009217 |
| chr10: | 8719219-8719872 |
| chr10: | 9631544-9632353 |
| chr10: | 11407180-11409355 |
| chr10: | 11955137-11958436 |
| chr10: | 12816901-12818144 |
| chr10: | 12893184-12894079 |
| chr10: | 13056556-13060607 |
| chr10: | 13570056-13571415 |
| chr10: | 13818913-13819374 |
| chr10: | 14533519-14536029 |
| chr10: | 15793093-15793813 |
| chr10: | 17016283-17018715 |
| chr10: | 17310698-17315438 |
| chr10: | 18224755-18225316 |
| chr10: | 18503647-18504164 |
| chr10: | 18670711-18671270 |
| chr10: | 20650085-20653247 |
| chr10: | 23015569-23016682 |
| chr10: | 23635447-23636434 |
| chr10: | 23721447-23721937 |
| chr10: | 24847820-24848480 |
| chr10: | 25638194-25638834 |
| chr10: | 25905114-25905885 |
| chr10: | 26139994-26143360 |
| chr10: | 26180284-26183269 |
| chr10: | 26350474-26352066 |
| chr10: | 26596407-26597670 |
| chr10: | 26608229-26611060 |
| chr10: | 26685115-26689761 |
| chr10: | 33492469-33493303 |
| chr10: | 34096533-34097062 |
| chr10: | 34754598-34756371 |
| chr10: | 52718467-52719986 |
| chr10: | 53699496-53701148 |
| chr10: | 54015661-54018328 |
| chr10: | 54526625-54529250 |
| chr10: | 54883289-54884635 |
| chr10: | 55269926-55271567 |
| chr10: | 55294558-55296490 |
| chr10: | 56682780-56684286 |
| chr10: | 57282681-57284560 |
| chr10: | 57353031-57357864 |
| chr10: | 58952196-58953534 |
| chr10: | 59209759-59211887 |
| chr10: | 59743706-59744545 |
| chr10: | 60272266-60272924 |
| chr10: | 60279739-60281400 |
| chr10: | 61361947-61365057 |
| chr10: | 61641666-61642417 |
| chr10: | 62427308-62428431 |
| chr10: | 62620379-62623956 |
| chr10: | 65280902-65281816 |
| chr10: | 65509239-65511124 |
| chr10: | 66225988-66226732 |
| chr10: | 66398595-66400760 |
| chr10: | 66398818-66399839 |
| chr10: | 67030166-67032331 |
| chr10: | 67229436-67233694 |
| chr10: | 67342470-67344175 |
| chr10: | 67343105-67344175 |
| chr10: | 67583133-67583696 |
| chr10: | 68416945-68417668 |
| chr10: | 69039315-69040236 |
| chr10: | 70560007-70562515 |
| chr10: | 70586985-70588121 |
| chr10: | 71797616-71801119 |
| chr10: | 72432020-72432603 |
| chr10: | 72448818-72450243 |
| chr10: | 72448818-72451301 |
| chr10: | 72856602-72858361 |
| chr10: | 73156217-73158587 |
| chr10: | 73327600-73328301 |
| chr10: | 73710631-73711569 |
| chr10: | 73710631-73714995 |
| chr10: | 73862072-73866048 |
| chr10: | 77745578-77746088 |
| chr10: | 79396508-79398661 |
| chr10: | 79397487-79398556 |
| chr10: | 79685742-79687048 |
| chr10: | 80143402-80144378 |
| chr10: | 80240397-80241413 |
| chr10: | 81002132-81003538 |
| chr10: | 81136784-81138699 |
| chr10: | 81154264-81155586 |
| chr10: | 81175047-81177037 |
| chr10: | 81204631-81206516 |
| chr10: | 82213675-82214570 |
| chr10: | 82524387-82528748 |
| chr10: | 83634119-83635215 |
| chr10: | 84127870-84130301 |
| chr10: | 85350573-85351997 |
| chr10: | 85522557-85524038 |
| chr10: | 85522722-85523501 |
| chr10: | 86681295-86683924 |
| chr10: | 86812965-86814651 |
| chr10: | 87242686-87243965 |
| chr10: | 87243121-87243723 |
| chr10: | 88125263-88127528 |
| chr10: | 89456312-89457005 |
| chr10: | 89652835-89653712 |
| chr10: | 91643009-91644203 |
| chr10: | 91998471-92002033 |
| chr10: | 92087717-92088311 |
| chr10: | 92461885-92465365 |
| chr10: | 92617266-92618168 |
| chr10: | 92896847-92897596 |
| chr10: | 92922326-92923774 |
| chr10: | 93633411-93634535 |
| chr10: | 93668098-93668730 |
| chr10: | 95388745-95390055 |
| chr10: | 95461475-95462364 |
| chr10: | 95545512-95546307 |
| chr10: | 95594298-95596661 |
| chr10: | 95605796-95609228 |
| chr10: | 95608663-95609228 |
| chr10: | 96162411-96163346 |
| chr10: | 96870857-96874954 |
| chr10: | 96871191-96872545 |
| chr10: | 99835164-99837474 |
| chr10: | 100338511-100339880 |
| chr10: | 100375776-100378966 |
| chr10: | 100688852-100689884 |
| chr10: | 102631248-102633068 |
| chr10: | 102727035-102727615 |
| chr10: | 103356021-103356579 |
| chr10: | 104004822-104005332 |
| chr10: | 105308399-105311973 |
| chr10: | 106273034-106277299 |
| chr10: | 106275105-106277444 |
| chr10: | 106833173-106834565 |
| chr10: | 107950687-107951600 |
| chr10: | 107995861-107997704 |
| chr10: | 108030313-108032528 |
| chr10: | 109163882-109164575 |
| chr10: | 109638670-109639668 |
| chr10: | 110916979-110919295 |
| chr10: | 111145790-111146838 |
| chr10: | 114112105-114116670 |
| chr10: | 115427132-115431770 |
| chr10: | 115541979-115543315 |
| chr10: | 115545216-115546176 |
| chr10: | 116270120-116271533 |
| chr10: | 117803673-117804640 |
| chr10: | 117809201-117810551 |
| chr10: | 118766138-118768926 |
| chr10: | 120529823-120530743 |
| chr10: | 120575912-120576531 |
| chr10: | 120895883-120897496 |
| chr10: | 121836401-121837815 |
| chr10: | 121991986-121992450 |
| chr10: | 122226923-122228712 |
| chr10: | 122548272-122550440 |
| chr10: | 122548272-122553255 |
| chr10: | 123197463-123198811 |
| chr10: | 124216755-124217355 |
| chr10: | 125051817-125053115 |
| chr10: | 125222047-125222877 |
| chr10: | 125399972-125400476 |
| chr10: | 125640494-125641084 |
| chr10: | 126188285-126192415 |
| chr10: | 126195215-126196530 |
| chr10: | 126298892-126299652 |
| chr10: | 127667815-127668600 |
| chr10: | 128274934-128275750 |
| chr10: | 128555567-128556642 |
| chr10: | 128565092-128565627 |
| chr10: | 128588918-128592240 |
| chr10: | 128988601-128989234 |
| chr10: | 129145693-129146869 |
| chr10: | 129183917-129184392 |
| chr10: | 129195221-129195941 |
| chr10: | 129830202-129831283 |
| chr10: | 129882287-129883090 |
| chr10: | 130836752-130837284 |
| chr10: | 131303674-131308469 |
| chr10: | 131933569-131934204 |
| chr10: | 132177236-132178692 |
| chr10: | 132257683-132258500 |
| chr10: | 132271741-132272326 |
| chr10: | 132317264-132320978 |
| chr10: | 132370579-132372330 |
| chr10: | 132532862-132534966 |
| chr10: | 132557316-132559141 |
| chr10: | 132583699-132584249 |
| chr10: | 132636032-132638736 |
| chr10: | 132710567-132711192 |
| chr10: | 132840099-132841315 |
| chr10: | 132908725-132913146 |
| chr10: | 132988416-132991199 |
| chr10: | 133026399-133027469 |
| chr10: | 133087806-133088471 |
| chr10: | 133142518-133143048 |
| chr10: | 133150223-133150802 |
| chr10: | 133183673-133185248 |
| chr10: | 133455273-133457280 |
| chr10: | 133526896-133528326 |
| chr10: | 133528251-133530036 |
| chr10: | 133528873-133530036 |
| chr10: | 133607580-133609179 |
| chr10: | 133655196-133657081 |
| chr10: | 133730023-133734042 |
| chr10: | 133765487-133767411 |
| chr10: | 133972134-133975599 |
| chr10: | 134004306-134005756 |
| chr10: | 134171768-134172825 |
| chr10: | 134175659-134176764 |
| chr10: | 134177604-134178429 |
| chr10: | 134178029-134178541 |
| chr10: | 134200119-134202696 |
| chr10: | 134249488-134250033 |
| chr10: | 134318810-134320350 |
| chr10: | 134335351-134336056 |
| chr11: | 321355-322535 |
| chr11: | 321355-325773 |
| chr11: | 325828-326678 |
| chr11: | 363050-363945 |
| chr11: | 384918-385658 |
| chr11: | 400680-403286 |
| chr11: | 410458-412783 |
| chr11: | 430660-431765 |
| chr11: | 519226-520281 |
| chr11: | 530383-531133 |
| chr11: | 585072-585663 |
| chr11: | 734804-736599 |
| chr11: | 741240-744515 |
| chr11: | 858239-859589 |
| chr11: | 964758-965226 |
| chr11: | 975177-975832 |
| chr11: | 1015774-1019739 |
| chr11: | 1016439-1019154 |
| chr11: | 1058219-1060093 |
| chr11: | 1059183-1060093 |
| chr11: | 1078800-1079645 |
| chr11: | 1315233-1316278 |
| chr11: | 1315803-1316278 |
| chr11: | 1372794-1374900 |
| chr11: | 1433291-1433906 |
| chr11: | 1438764-1439879 |
| chr11: | 1450570-1454480 |
| chr11: | 1531327-1531954 |
| chr11: | 1654439-1656641 |
| chr11: | 1662707-1663607 |
| chr11: | 1681495-1682690 |
| chr11: | 1691557-1692452 |
| chr11: | 1935490-1936950 |
| chr11: | 2145754-2148494 |
| chr11: | 2320812-2321557 |
| chr11: | 2439638-2441010 |
| chr11: | 2449575-2451230 |
| chr11: | 2558030-2558805 |
| chr11: | 2958909-2960173 |
| chr11: | 6115326-6117545 |
| chr11: | 6115560-6119632 |
| chr11: | 6116819-6117759 |
| chr11: | 6121495-6121968 |
| chr11: | 6189710-6193096 |
| chr11: | 6520140-6520706 |
| chr11: | 6676169-6678265 |
| chr11: | 6881538-6881988 |
| chr11: | 7134606-7136738 |
| chr11: | 7135871-7136551 |
| chr11: | 7276056-7277166 |
| chr11: | 7685919-7686909 |
| chr11: | 7846698-7847592 |
| chr11: | 8088194-8089132 |
| chr11: | 8199940-8201700 |
| chr11: | 8200989-8201455 |
| chr11: | 8215395-8215955 |
| chr11: | 9323861-9324546 |
| chr11: | 11301415-11305548 |
| chr11: | 11366403-11368258 |
| chr11: | 11379555-11380986 |
| chr11: | 11822726-11824492 |
| chr11: | 11875723-11876683 |
| chr11: | 12401732-12402397 |
| chr11: | 13298989-13299689 |
| chr11: | 14273814-14274284 |
| chr11: | 14380039-14381016 |
| chr11: | 15523863-15525293 |
| chr11: | 15524070-15524712 |
| chr11: | 16760318-16761128 |
| chr11: | 17510136-17510847 |
| chr11: | 17593003-17594228 |
| chr11: | 18071446-18072290 |
| chr11: | 18097605-18100066 |
| chr11: | 18735744-18736576 |
| chr11: | 18735744-18738459 |
| chr11: | 19424300-19426122 |
| chr11: | 19492212-19495601 |
| chr11: | 19578851-19580967 |
| chr11: | 20137687-20138718 |
| chr11: | 20753671-20754252 |
| chr11: | 20847788-20849955 |
| chr11: | 21423046-21425035 |
| chr11: | 21669216-21670000 |
| chr11: | 21847301-21849377 |
| chr11: | 22470895-22472785 |
| chr11: | 23374665-23378555 |
| chr11: | 23581954-23582461 |
| chr11: | 23591195-23592147 |
| chr11: | 23749542-23753466 |
| chr11: | 24284653-24288207 |
| chr11: | 24285707-24237508 |
| chr11: | 24286175-24286994 |
| chr11: | 24502860-24504929 |
| chr11: | 24302976-24804016 |
| chr11: | 25073714-25083528 |
| chr11: | 25083584-25085733 |
| chr11: | 25750530-25753590 |
| chr11: | 25773021-25773886 |
| chr11: | 26607958-26610913 |
| chr11: | 26658526-26659684 |
| chr11: | 29139377-29140406 |
| chr11: | 29139538-29140067 |
| chr11: | 29530041-29530788 |
| chr11: | 29967574-29968449 |
| chr11: | 30172028-30176852 |
| chr11: | 31393778-31397476 |
| chr11: | 31967121-31968888 |
| chr11: | 34015216-34018311 |
| chr11: | 34172092-34174187 |
| chr11: | 34203744-34204819 |
| chr11: | 34251608-34253343 |
| chr11: | 34372810-34373616 |
| chr11: | 35539359-35542439 |
| chr11: | 36434693-36436965 |
| chr11: | 36661279-36661736 |
| chr11: | 36777802-36778429 |
| chr11: | 36806216-36806894 |
| chr11: | 38026884-38029392 |
| chr11: | 38300670-38301123 |
| chr11: | 39032506-39033837 |
| chr11: | 39226351-39227536 |
| chr11: | 40334409-40338838 |
| chr11: | 40682342-40685431 |
| chr11: | 40683334-40684496 |
| chr11: | 41818239-41819027 |
| chr11: | 41946498-41947956 |
| chr11: | 42814115-42818470 |
| chr11: | 42968030-42970953 |
| chr11: | 44014155-44015971 |
| chr11: | 44113563-44115322 |
| chr11: | 44818895-44820050 |
| chr11: | 45429883-45431588 |
| chr11: | 45430342-45431588 |
| chr11: | 45430488-45431088 |
| chr11: | 48249024-48250324 |
| chr11: | 48327672-48328178 |
| chr11: | 48600856-48604301 |
| chr11: | 48735367-48736791 |
| chr11: | 51571810-51574251 |
| chr11: | 56439780-56440270 |
| chr11: | 56946787-56948827 |
| chr11: | 57143327-57146038 |
| chr11: | 57246703-57247328 |
| chr11: | 57760689-57761546 |
| chr11: | 58167064-58168268 |
| chr11: | 58457501-58461931 |
| chr11: | 58594529-58596021 |
| chr11: | 58595421-58596021 |
| chr11: | 58631790-58636511 |
| chr11: | 59383294-59383871 |
| chr11: | 60228243-60229314 |
| chr11: | 60952128-60954546 |
| chr11: | 63535644-63536914 |
| chr11: | 64085418-64087410 |
| chr11: | 64407674-64409875 |
| chr11: | 65267136-65270528 |
| chr11: | 65642100-65643241 |
| chr11: | 65738152-65739062 |
| chr11: | 66712149-66713260 |
| chr11: | 66843375-66844907 |
| chr11: | 67153088-67155398 |
| chr11: | 67330357-67332003 |
| chr11: | 67435244-67438519 |
| chr11: | 68067841-68069050 |
| chr11: | 68227511-68228757 |
| chr11: | 68434046-68435051 |
| chr11: | 68835696-68837291 |
| chr11: | 68843734-68844904 |
| chr11: | 69941754-69942319 |
| chr11: | 69982591-69984115 |
| chr11: | 69982946-69983585 |
| chr11: | 71952267-71953590 |
| chr11: | 72243512-72247951 |
| chr11: | 72350800-72352250 |
| chr11: | 75557315-75558660 |
| chr11: | 75967239-75969847 |
| chr11: | 76107247-76111858 |
| chr11: | 76672837-76674867 |
| chr11: | 76906030-76906635 |
| chr11: | 78890548-78891589 |
| chr11: | 79226186-79226921 |
| chr11: | 79872898-79873794 |
| chr11: | 80598543-80601866 |
| chr11: | 80711432-80712276 |
| chr11: | 81187983-81189467 |
| chr11: | 81675900-81676916 |
| chr11: | 83464992-83467903 |
| chr11: | 86285969-86286746 |
| chr11: | 86303268-86306521 |
| chr11: | 86305189-86306521 |
| chr11: | 86624273-86624960 |
| chr11: | 86753735-86756555 |
| chr11: | 87922261-87923024 |
| chr11: | 88236083-88236773 |
| chr11: | 88964495-88967904 |
| chr11: | 90193730-90196151 |
| chr11: | 90568117-90571066 |
| chr11: | 91370036-91371578 |
| chr11: | 91370801-91371578 |
| chr11: | 92704480-92707592 |
| chr11: | 93021137-93022285 |
| chr11: | 93636188-93640796 |
| chr11: | 93638553-93641193 |
| chr11: | 93640235-93640796 |
| chr11: | 96002119-96003404 |
| chr11: | 96750140-96753363 |
| chr11: | 97517707-97521255 |
| chr11: | 97915320-97917346 |
| chr11: | 98473450-98476544 |
| chr11: | 99185381-99186963 |
| chr11: | 99491185-99491960 |
| chr11: | 101072598-101074046 |
| chr11: | 102303536-102307481 |
| chr11: | 102751245-102752765 |
| chr11: | 102751538-102752155 |
| chr11: | 103916372-103917321 |
| chr11: | 103916372-103918482 |
| chr11: | 104005959-104006418 |
| chr11: | 104316078-104320734 |
| chr11: | 105209367-105212273 |
| chr11: | 105413356-105415892 |
| chr11: | 106786102-106789524 |
| chr11: | 107126718-107127564 |
| chr11: | 107238660-107243470 |
| chr11: | 107784298-107788439 |
| chr11: | 108802515-108804125 |
| chr11: | 109396844-109400893 |
| chr11: | 110488938-110490511 |
| chr11: | 110488938-110492858 |
| chr11: | 110890145-110890850 |
| chr11: | 111290449-111291024 |
| chr11: | 112423999-112424924 |
| chr11: | 112614179-112614645 |
| chr11: | 112698802-112699502 |
| chr11: | 112987389-112990026 |
| chr11: | 114436934-114438106 |
| chr11: | 115017787-115019687 |
| chr11: | 115659753-115660652 |
| chr11: | 116216735-116219440 |
| chr11: | 116669316-116673672 |
| chr11: | 116735840-116737098 |
| chr11: | 118282037-118282576 |
| chr11: | 118478787-118482752 |
| chr11: | 120073338-120074664 |
| chr11: | 120387801-120388381 |
| chr11: | 120547915-120548385 |
| chr11: | 121055909-121057105 |
| chr11: | 121351217-121352687 |
| chr11: | 121457093-121457602 |
| chr11: | 121584567-121586152 |
| chr11: | 124078778-124079439 |
| chr11: | 124980066-124981880 |
| chr11: | 125074977-125079432 |
| chr11: | 125078514-125079063 |
| chr11: | 125286253-125289616 |
| chr11: | 126751066-126755936 |
| chr11: | 127275027-127279956 |
| chr11: | 127749574-127752012 |
| chr11: | 127749990-127750936 |
| chr11: | 128085735-128086685 |
| chr11: | 128311665-128315437 |
| chr11: | 128343749-128345943 |
| chr11: | 128682723-128683383 |
| chr11: | 129148770-129149850 |
| chr11: | 129939516-129941209 |
| chr11: | 130225717-130230483 |
| chr11: | 130599994-130601511 |
| chr11: | 130920059-130922057 |
| chr11: | 131201892-131205232 |
| chr11: | 131550823-131551753 |
| chr11: | 131596235-131597167 |
| chr11: | 131938892-131941336 |
| chr11: | 132034508-132036971 |
| chr11: | 132216888-132217382 |
| chr11: | 133961558-133964741 |
| chr11: | 134007359-134008460 |
| chr11: | 134278124-134278804 |
| chr11: | 134282032-134282537 |
| chr11: | 134359195-134359690 |
| chr11: | 134393058-134394802 |
| chr11: | 134393473-134394215 |
| chr11: | 134533823-134534398 |
| chr11: | 134605113-134605868 |
| chr11: | 134605913-134607643 |
| chr11: | 134709366-134710272 |
| chr11: | 134721627-134722217 |
| chr11: | 134732906-134734086 |
| chr11: | 134732951-134733626 |
| chr11: | 134803540-134806050 |
| chr11: | 134909300-134912835 |
| chr12: | 292234-296699 |
| chr12: | 528651-531517 |
| chr12: | 620082-622490 |
| chr12: | 751959-752547 |
| chr12: | 1304444-1305402 |
| chr12: | 1365641-1366425 |
| chr12: | 1639122-1640662 |
| chr12: | 1702491-1704405 |
| chr12: | 1770132-1770672 |
| chr12: | 3377309-3379044 |
| chr12: | 3570827-3572178 |
| chr12: | 3786431-3788548 |
| chr12: | 3862911-3863450 |
| chr12: | 4043920-4044686 |
| chr12: | 4107971-4109977 |
| chr12: | 5082144-5083210 |
| chr12: | 5221874-5223429 |
| chr12: | 6037426-6041991 |
| chr12: | 6037703-6039616 |
| chr12: | 6040671-6041857 |
| chr12: | 6146518-6148317 |
| chr12: | 6292454-6293174 |
| chr12: | 6292454-6295944 |
| chr12: | 6399054-6400911 |
| chr12: | 6722544-6723348 |
| chr12: | 6875240-6877171 |
| chr12: | 7348744-7350002 |
| chr12: | 7322584-7823833 |
| chr12: | 14214085-14216716 |
| chr12: | 16018705-16021112 |
| chr12: | 16420125-16420982 |
| chr12: | 16420125-16421322 |
| chr12: | 17444759-17445930 |
| chr12: | 17607369-17608052 |
| chr12: | 17607719-17608445 |
| chr12: | 18723077-18725110 |
| chr12: | 20243337-20244142 |
| chr12: | 21815499-21816988 |
| chr12: | 22129601-22131196 |
| chr12: | 22248615-22251479 |
| chr12: | 22322309-22326692 |
| chr12: | 22344132-22345047 |
| chr12: | 23939531-23940048 |
| chr12: | 24940809-24941724 |
| chr12: | 25594844-25595322 |
| chr12: | 25634023-25637090 |
| chr12: | 25955888-25959149 |
| chr12: | 26111540-26112100 |
| chr12: | 27788701-27789400 |
| chr12: | 27788701-27792463 |
| chr12: | 27932805-27933945 |
| chr12: | 28095570-28098265 |
| chr12: | 29161450-29164823 |
| chr12: | 29844411-29846441 |
| chr12: | 29956430-29960234 |
| chr12: | 30152094-30155590 |
| chr12: | 30307157-30307634 |
| chr12: | 30478293-30480653 |
| chr12: | 30498226-30501743 |
| chr12: | 30504450-30506351 |
| chr12: | 32330884-32335632 |
| chr12: | 34751309-34755067 |
| chr12: | 38921304-38923456 |
| chr12: | 40499468-40500027 |
| chr12: | 40817762-40321672 |
| chr12: | 40874832-40875986 |
| chr12: | 41028057-41028974 |
| chr12: | 41685767-41687157 |
| chr12: | 44394412-44399214 |
| chr12: | 44691907-44693244 |
| chr12: | 45903142-45904437 |
| chr12: | 45906192-45909598 |
| chr12: | 46472995-46475636 |
| chr12: | 46631214-46632060 |
| chr12: | 47028397-47033327 |
| chr12: | 48334654-48335149 |
| chr12: | 50097299-50098198 |
| chr12: | 50355200-50355978 |
| chr12: | 50973674-50975546 |
| chr12: | 51216724-51218337 |
| chr12: | 51949222-51950663 |
| chr12: | 55780615-55784310 |
| chr12: | 56002278-56004219 |
| chr12: | 56031874-56034678 |
| chr12: | 56211376-56212111 |
| chr12: | 56270232-56271795 |
| chr12: | 56524824-56527928 |
| chr12: | 57110564-57115506 |
| chr12: | 57374477-57376512 |
| chr12: | 58028104-58028966 |
| chr12: | 58509503-58513705 |
| chr12: | 58637852-58639969 |
| chr12: | 59968566-59970851 |
| chr12: | 59969980-59970710 |
| chr12: | 59970136-59970851 |
| chr12: | 60102386-60104047 |
| chr12: | 60521846-60525048 |
| chr12: | 60753340-60757908 |
| chr12: | 60933877-60936005 |
| chr12: | 61091794-61093075 |
| chr12: | 61092240-61093241 |
| chr12: | 62393980-62394479 |
| chr12: | 63576104-63576698 |
| chr12: | 64237320-64238205 |
| chr12: | 66609223-66609834 |
| chr12: | 66756262-66756819 |
| chr12: | 66791981-66794122 |
| chr12: | 66908659-66909227 |
| chr12: | 66994805-66995301 |
| chr12: | 67927318-67928133 |
| chr12: | 69255768-69256516 |
| chr12: | 69862143-69862791 |
| chr12: | 70289731-70290746 |
| chr12: | 70679884-70631895 |
| chr12: | 71532675-71533665 |
| chr12: | 71602377-71604481 |
| chr12: | 71820546-71821922 |
| chr12: | 71886438-71889651 |
| chr12: | 72189067-72190616 |
| chr12: | 72222160-72222750 |
| chr12: | 72356896-72357977 |
| chr12: | 73220084-73221080 |
| chr12: | 74103832-74105581 |
| chr12: | 74104882-74105482 |
| chr12: | 74210782-74211383 |
| chr12: | 74644626-74647793 |
| chr12: | 75112236-75115472 |
| chr12: | 76712926-76775464 |
| chr12: | 77674050-77674714 |
| chr12: | 78098828-78100745 |
| chr12: | 79197474-79198900 |
| chr12: | 80523442-80531906 |
| chr12: | 80823902-80828210 |
| chr12: | 82139696-82144689 |
| chr12: | 83266497-83270119 |
| chr12: | 84593122-84595697 |
| chr12: | 84593981-84595697 |
| chr12: | 84612580-84614669 |
| chr12: | 85036189-85040796 |
| chr12: | 85363284-85364486 |
| chr12: | 86604717-86609023 |
| chr12: | 87530594-87533470 |
| chr12: | 90101940-90103405 |
| chr12: | 90487573-90492061 |
| chr12: | 90972054-90974603 |
| chr12: | 91241715-91244778 |
| chr12: | 91658381-91659870 |
| chr12: | 91722089-91723878 |
| chr12: | 93210459-93211061 |
| chr12: | 94443438-94447358 |
| chr12: | 94541853-94544147 |
| chr12: | 95285621-95286981 |
| chr12: | 95803068-95305054 |
| chr12: | 96010028-96012527 |
| chr12: | 96036930-96038220 |
| chr12: | 96037276-96037882 |
| chr12: | 96587781-96588872 |
| chr12: | 97220875-97221409 |
| chr12: | 97992057-97993577 |
| chr12: | 98669920-98672979 |
| chr12: | 98955468-98959360 |
| chr12: | 99288344-99289315 |
| chr12: | 100288397-100291934 |
| chr12: | 100972593-100975980 |
| chr12: | 101235463-101236159 |
| chr12: | 101887335-101888136 |
| chr12: | 102102734-102107123 |
| chr12: | 103324956-103326622 |
| chr12: | 104283731-104287745 |
| chr12: | 104286626-104287745 |
| chr12: | 105680990-105681599 |
| chr12: | 105949857-105951376 |
| chr12: | 105950809-105951776 |
| chr12: | 106330998-106381563 |
| chr12: | 106532468-106533798 |
| chr12: | 106640836-106642276 |
| chr12: | 107289526-107292688 |
| chr12: | 108459954-103461278 |
| chr12: | 108459954-108462413 |
| chr12: | 108460725-108463832 |
| chr12: | 108362197-108862962 |
| chr12: | 108363916-108866406 |
| chr12: | 109251040-109251675 |
| chr12: | 110099238-110100303 |
| chr12: | 110137501-110140446 |
| chr12: | 110151456-10152801 |
| chr12: | 110718803-10719498 |
| chr12: | 111045062-11049139 |
| chr12: | 111471755-111472879 |
| chr12: | 111890488-111691092 |
| chr12: | 111856140-111856590 |
| chr12: | 111946784-111947252 |
| chr12: | 111976545-111979880 |
| chr12: | 112035871-112037545 |
| chr12: | 113011798-113012757 |
| chr12: | 113019508-113020029 |
| chr12: | 113474512-113478812 |
| chr12: | 114104651-114106201 |
| chr12: | 114129677-114130142 |
| chr12: | 114268069-114268707 |
| chr12: | 114501487-114502418 |
| chr12: | 114725918-114726590 |
| chr12: | 115086017-115087294 |
| chr12: | 115291607-115292123 |
| chr12: | 115643472-115643948 |
| chr12: | 115690081-115692005 |
| chr12: | 115890881-115692005 |
| chr12: | 116526479-116528480 |
| chr12: | 116714932-116715904 |
| chr12: | 117087243-117088170 |
| chr12: | 117841095-117643946 |
| chr12: | 118499231-118500113 |
| chr12: | 118936859-118937762 |
| chr12: | 119328798-119329610 |
| chr12: | 113328798-119332851 |
| chr12: | 120426639-120427988 |
| chr12: | 121802747-121604132 |
| chr12: | 122138748-122142224 |
| chr12: | 122185141-122186306 |
| chr12: | 122185231-122185766 |
| chr12: | 123139888-123142070 |
| chr12: | 123140691-123141675 |
| chr12: | 123140691-123142779 |
| chr12: | 123185095-123188038 |
| chr12: | 123719423-123719876 |
| chr12: | 124017022-124017617 |
| chr12: | 124395903-124397052 |
| chr12: | 124496106-124499990 |
| chr12: | 124725468-124726750 |
| chr12: | 125024616-125026611 |
| chr12: | 125166039-125168723 |
| chr12: | 125381825-125386596 |
| chr12: | 125633706-125690251 |
| chr12: | 125897142-125897731 |
| chr12: | 126186603-126187438 |
| chr12: | 126571933-126573178 |
| chr12: | 127067704-127069032 |
| chr12: | 127608120-127609883 |
| chr12: | 127810511-127811140 |
| chr12: | 128789254-128790714 |
| chr12: | 128811304-128811773 |
| chr12: | 129130092-129133653 |
| chr12: | 129192724-129193464 |
| chr12: | 129229872-129233010 |
| chr12: | 129328949-129331653 |
| chr12: | 129386507-129389135 |
| chr12: | 129572323-129574163 |
| chr12: | 129572813-129573803 |
| chr12: | 129755758-129758863 |
| chr12: | 130058374-130062264 |
| chr12: | 130076903-130077539 |
| chr12: | 130295296-130298442 |
| chr12: | 130339120-130343774 |
| chr12: | 130822389-130825865 |
| chr12: | 130863910-130865125 |
| chr12: | 130876616-130878081 |
| chr12: | 130967290-130967785 |
| chr12: | 131024076-131024586 |
| chr12: | 131130639-131131399 |
| chr12: | 131131444-131133952 |
| chr12: | 131147950-131150260 |
| chr12: | 131232972-131233747 |
| chr12: | 131236017-131236657 |
| chr12: | 131266545-131267175 |
| chr12: | 131366738-131367453 |
| chr12: | 131454765-131455231 |
| chr12: | 131503111-131506191 |
| chr12: | 131530439-131531659 |
| chr12: | 131575372-131575938 |
| chr12: | 132185592-132186377 |
| chr12: | 132619129-132621029 |
| chr12: | 132633387-132635342 |
| chr12: | 132696696-132697751 |
| chr12: | 132858343-132858833 |
| chr12: | 132876605-132877764 |
| chr12: | 132879995-132882390 |
| chr12: | 132957340-132961749 |
| chr12: | 132959664-132961604 |
| chr12: | 132963865-132965665 |
| chr12: | 132964015-132965025 |
| chr12: | 132973631-132974716 |
| chr12: | 132988574-132990459 |
| chr12: | 133049423-133051218 |
| chr12: | 133085197-133089146 |
| chr12: | 133168838-133171008 |
| chr12: | 133174600-133175155 |
| chr12: | 133305972-133306974 |
| chr12: | 133352717-133353287 |
| chr12: | 133393209-133395644 |
| chr12: | 133445406-133446022 |
| chr13: | 20650177-20651772 |
| chr13: | 20825292-20829144 |
| chr13: | 20998352-20998937 |
| chr13: | 21680279-21682087 |
| chr13: | 21700455-21701287 |
| chr13: | 21727231-21729991 |
| chr13: | 21727881-21729226 |
| chr13: | 21896409-21899439 |
| chr13: | 21951294-21951839 |
| chr13: | 22418421-22421732 |
| chr13: | 22863619-22864438 |
| chr13: | 23325233-23326926 |
| chr13: | 23635061-23637471 |
| chr13: | 23675455-23676035 |
| chr13: | 23764388-23769266 |
| chr13: | 24287850-24288892 |
| chr13: | 24404939-24405959 |
| chr13: | 26770842-26772017 |
| chr13: | 27050139-27052101 |
| chr13: | 29750941-29751706 |
| chr13: | 31212102-31215166 |
| chr13: | 33139412-33140253 |
| chr13: | 34132861-34135198 |
| chr13: | 34192603-34193366 |
| chr13: | 35562555-35564757 |
| chr13: | 36283703-36286076 |
| chr13: | 36622354-36625704 |
| chr13: | 36791777-36795319 |
| chr13: | 37679949-37681397 |
| chr13: | 38045699-38049225 |
| chr13: | 38048444-38049331 |
| chr13: | 39057318-39060191 |
| chr13: | 39528095-39529620 |
| chr13: | 39747517-39751641 |
| chr13: | 39933627-39935387 |
| chr13: | 39934494-39935387 |
| chr13: | 39982147-39983164 |
| chr13: | 40783968-40788231 |
| chr13: | 42237228-42241744 |
| chr13: | 43053216-43053830 |
| chr13: | 43300241-43302595 |
| chr13: | 43923410-43924117 |
| chr13: | 45042562-45043120 |
| chr13: | 48782466-48785802 |
| chr13: | 49001179-49002743 |
| chr13: | 49533529-49536855 |
| chr13: | 50195734-50197062 |
| chr13: | 51444474-51446281 |
| chr13: | 52347717-52350532 |
| chr13: | 53419990-53422747 |
| chr13: | 53774609-53776399 |
| chr13: | 54258413-54261345 |
| chr13: | 55513571-55515456 |
| chr13: | 57787164-57788271 |
| chr13: | 58370100-58373807 |
| chr13: | 59722888-59724536 |
| chr13: | 61553073-61554006 |
| chr13: | 61958421-61959182 |
| chr13: | 62020087-62020847 |
| chr13: | 62654714-62657816 |
| chr13: | 62654958-62658528 |
| chr13: | 62784922-62787550 |
| chr13: | 62785210-62736126 |
| chr13: | 65342512-65344495 |
| chr13: | 65352104-65353482 |
| chr13: | 65525723-65529946 |
| chr13: | 67174817-67176098 |
| chr13: | 67174817-67177510 |
| chr13: | 69199279-69202937 |
| chr13: | 69390607-69391623 |
| chr13: | 69936886-69941819 |
| chr13: | 70125498-70126920 |
| chr13: | 70402384-70403675 |
| chr13: | 72477226-72480754 |
| chr13: | 72522655-72525124 |
| chr13: | 72807610-72812583 |
| chr13: | 72845782-72846891 |
| chr13: | 73632682-73634039 |
| chr13: | 74708584-74709924 |
| chr13: | 76209699-76211118 |
| chr13: | 76609091-76612717 |
| chr13: | 77459103-77460910 |
| chr13: | 78271646-78272691 |
| chr13: | 78455918-78459669 |
| chr13: | 78764995-78767834 |
| chr13: | 79175030-79176563 |
| chr13: | 79175746-79177653 |
| chr13: | 32693766-82694749 |
| chr13: | 83788250-83792614 |
| chr13: | 84050585-84051606 |
| chr13: | 84481393-84481953 |
| chr13: | 85712855-85713875 |
| chr13: | 85800866-85803357 |
| chr13: | 85936715-85937337 |
| chr13: | 86579429-86580263 |
| chr13: | 36673819-86676141 |
| chr13: | 89421959-89423177 |
| chr13: | 90246501-90250197 |
| chr13: | 90910980-90914017 |
| chr13: | 91024169-91027489 |
| chr13: | 91538766-91540040 |
| chr13: | 91999635-92001437 |
| chr13: | 93244573-93245965 |
| chr13: | 95196987-95201267 |
| chr13: | 95253055-95253914 |
| chr13: | 95363104-95365838 |
| chr13: | 96020368-96024016 |
| chr13: | 96742204-96743379 |
| chr13: | 97991827-97992339 |
| chr13: | 98085373-98087217 |
| chr13: | 98086068-98086863 |
| chr13: | 98103933-98108402 |
| chr13: | 98529967-98532990 |
| chr13: | 98628602-98630563 |
| chr13: | 98628707-98629603 |
| chr13: | 98794717-98796369 |
| chr13: | 99228371-99229954 |
| chr13: | 99254423-99257248 |
| chr13: | 99738375-99739915 |
| chr13: | 100637889-100638358 |
| chr13: | 100738215-100740367 |
| chr13: | 101894148-101896522 |
| chr13: | 103599045-103603424 |
| chr13: | 103985308-103985904 |
| chr13: | 103985308-103987671 |
| chr13: | 103991442-103992182 |
| chr13: | 104606315-104607654 |
| chr13: | 104807581-104809911 |
| chr13: | 105085891-105086406 |
| chr13: | 105737066-105738167 |
| chr13: | 105737066-105739408 |
| chr13: | 105782589-105784676 |
| chr13: | 105783903-105784676 |
| chr13: | 106250145-106250595 |
| chr13: | 106373893-106375230 |
| chr13: | 106374147-106377760 |
| chr13: | 106449360-106449930 |
| chr13: | 106636986-106637466 |
| chr13: | 107086692-107088642 |
| chr13: | 107187433-107188848 |
| chr13: | 108560375-108561368 |
| chr13: | 108560750-108561368 |
| chr13: | 108947067-108951812 |
| chr13: | 109361772-109362397 |
| chr13: | 109412933-109413667 |
| chr13: | 109688312-109688868 |
| chr13: | 110221333-110222422 |
| chr13: | 110323939-110324621 |
| chr13: | 110417828-110419333 |
| chr13: | 110417938-110418828 |
| chr13: | 110663770-110667708 |
| chr13: | 110664264-110665673 |
| chr13: | 110959300-110961245 |
| chr13: | 111007676-111008171 |
| chr13: | 111037655-111038402 |
| chr13: | 111133233-111134383 |
| chr13: | 111145807-111146677 |
| chr13: | 111329244-111330419 |
| chr13: | 111565996-111568215 |
| chr13: | 111704324-111704992 |
| chr13: | 111962346-111963876 |
| chr13: | 111992225-111994204 |
| chr13: | 112173560-112174452 |
| chr13: | 112179165-112179981 |
| chr13: | 112669257-112670619 |
| chr13: | 112691053-112691802 |
| chr13: | 112800343-112804424 |
| chr13: | 112817722-112819429 |
| chr13: | 112849023-112852226 |
| chr13: | 112850705-112851882 |
| chr13: | 112868939-112869719 |
| chr13: | 113194047-113195062 |
| chr13: | 113264138-113267599 |
| chr13: | 113325212-113326045 |
| chr13: | 113421186-113421982 |
| chr13: | 113440036-113441096 |
| chr13: | 113498341-113500165 |
| chr13: | 113499575-113500165 |
| chr13: | 113517051-113520215 |
| chr13: | 113527117-113527904 |
| chr13: | 113665573-113667742 |
| chr13: | 113679108-113680578 |
| chr13: | 113760520-113765278 |
| chr13: | 113761610-113763416 |
| chr13: | 113816059-113817354 |
| chr13: | 113861685-113864645 |
| chr13: | 113990693-113991993 |
| chr13: | 113997203-113997926 |
| chr13: | 114000312-114002462 |
| chr13: | 114032162-114036100 |
| chr13: | 114033291-114034971 |
| chr13: | 114050440-114054248 |
| chr13: | 114067026-114067501 |
| chr13: | 114088660-114091685 |
| chr13: | 114195644-114196734 |
| chr13: | 114217032-114220057 |
| chr14: | 22392716-22393508 |
| chr14: | 22419825-22421059 |
| chr14: | 22582683-22583753 |
| chr14: | 22774843-22775374 |
| chr14: | 22794441-22798214 |
| chr14: | 22881700-22882230 |
| chr14: | 23938380-23938839 |
| chr14: | 24420325-24423668 |
| chr14: | 24446478-24447204 |
| chr14: | 25262297-25263072 |
| chr14: | 25364369-25365119 |
| chr14: | 25497781-25499192 |
| chr14: | 25536352-25537322 |
| chr14: | 25758226-25759165 |
| chr14: | 26283447-26284779 |
| chr14: | 26284099-26284779 |
| chr14: | 26590764-26593002 |
| chr14: | 27477257-27478305 |
| chr14: | 28869884-28873157 |
| chr14: | 29127037-29128032 |
| chr14: | 30817294-30819425 |
| chr14: | 32363648-32364163 |
| chr14: | 33129777-33130735 |
| chr14: | 33310318-33311585 |
| chr14: | 33570778-33571378 |
| chr14: | 33806918-33808020 |
| chr14: | 33941759-33942545 |
| chr14: | 34514052-34514846 |
| chr14: | 34715517-34717474 |
| chr14: | 36986331-36990958 |
| chr14: | 37429275-37429870 |
| chr14: | 37769653-37771198 |
| chr14: | 37770441-37771042 |
| chr14: | 38133554-38137781 |
| chr14: | 38744204-38744647 |
| chr14: | 38801335-38803614 |
| chr14: | 40890580-40891142 |
| chr14: | 42547016-42547530 |
| chr14: | 43182920-43185287 |
| chr14: | 45611118-45612852 |
| chr14: | 46320743-46321274 |
| chr14: | 46589016-46591084 |
| chr14: | 49382296-49384571 |
| chr14: | 49671873-49672378 |
| chr14: | 49941331-49943135 |
| chr14: | 50825123-50827047 |
| chr14: | 50826164-50827047 |
| chr14: | 51761566-51762332 |
| chr14: | 53874885-53875427 |
| chr14: | 54316303-54317567 |
| chr14: | 54710391-54713684 |
| chr14: | 56556987-56557460 |
| chr14: | 57823946-57824687 |
| chr14: | 60781936-60782807 |
| chr14: | 61058086-61059437 |
| chr14: | 63061408-63065739 |
| chr14: | 63513629-63514333 |
| chr14: | 63724095-63724654 |
| chr14: | 65606570-65607919 |
| chr14: | 65709258-65712968 |
| chr14: | 67984105-67985120 |
| chr14: | 71630023-71630732 |
| chr14: | 73063644-73064443 |
| chr14: | 73087442-73089482 |
| chr14: | 73268914-73270522 |
| chr14: | 73528106-73531599 |
| chr14: | 73546218-73546897 |
| chr14: | 74001783-74003666 |
| chr14: | 74925373-74927056 |
| chr14: | 75520959-75525448 |
| chr14: | 75784656-75785956 |
| chr14: | 75894414-75895289 |
| chr14: | 78295292-78297017 |
| chr14: | 78714506-78715237 |
| chr14: | 78969803-78970603 |
| chr14: | 79554308-79554912 |
| chr14: | 79981215-79982050 |
| chr14: | 80225544-80230355 |
| chr14: | 80653033-80654322 |
| chr14: | 80823216-80826109 |
| chr14: | 81878227-81879687 |
| chr14: | 81878227-81880580 |
| chr14: | 82499126-82503353 |
| chr14: | 82995806-82997433 |
| chr14: | 83154545-83155396 |
| chr14: | 83434741-83437523 |
| chr14: | 83520386-83523344 |
| chr14: | 83792853-83793493 |
| chr14: | 85139162-85139627 |
| chr14: | 85276926-85278588 |
| chr14: | 85404211-85407087 |
| chr14: | 86149716-86153603 |
| chr14: | 86313040-86313495 |
| chr14: | 86563625-86571765 |
| chr14: | 87052879-87054477 |
| chr14: | 89883196-89883872 |
| chr14: | 90244742-90245292 |
| chr14: | 91759239-91760208 |
| chr14: | 93106150-93107200 |
| chr14: | 93524467-93527075 |
| chr14: | 93581225-93582877 |
| chr14: | 93712512-93713151 |
| chr14: | 94454182-94455476 |
| chr14: | 95416094-95420075 |
| chr14: | 96081162-96082694 |
| chr14: | 96084227-96088370 |
| chr14: | 96831346-96832651 |
| chr14: | 97175244-97175809 |
| chr14: | 97803690-97804482 |
| chr14: | 98153253-98156034 |
| chr14: | 98220933-98223131 |
| chr14: | 98744578-98745685 |
| chr14: | 98811772-98815932 |
| chr14: | 98813822-98816856 |
| chr14: | 98815453-98815973 |
| chr14: | 99185088-99186268 |
| chr14: | 99503860-99505301 |
| chr14: | 99640536-99642292 |
| chr14: | 100069393-100070925 |
| chr14: | 100259392-100260052 |
| chr14: | 100277697-100278908 |
| chr14: | 100427794-100429238 |
| chr14: | 100704501-100707023 |
| chr14: | 101151972-101153252 |
| chr14: | 101486543-101487628 |
| chr14: | 101711224-101713218 |
| chr14: | 101923778-101925933 |
| chr14: | 102026707-102027542 |
| chr14: | 102178144-102180104 |
| chr14: | 102573178-102574378 |
| chr14: | 102950459-102950984 |
| chr14: | 103988029-103989824 |
| chr14: | 104312355-104315015 |
| chr14: | 104683385-104684025 |
| chr14: | 104716917-104718536 |
| chr14: | 104896095-104897845 |
| chr14: | 105010877-105013347 |
| chr14: | 105019051-105020159 |
| chr14: | 105186290-105187785 |
| chr14: | 105186665-105187720 |
| chr14: | 105218866-105219411 |
| chr14: | 105273954-105274874 |
| chr15: | 25333047-25334436 |
| chr15: | 25464499-25466669 |
| chr15: | 25931396-25932394 |
| chr15: | 26187966-26188649 |
| chr15: | 26218220-26219110 |
| chr15: | 26281256-26281756 |
| chr15: | 26322622-26324060 |
| chr15: | 26401039-26401865 |
| chr15: | 26749198-26752398 |
| chr15: | 27112235-27113345 |
| chr15: | 27389625-27394450 |
| chr15: | 27470043-27471667 |
| chr15: | 31617831-31621038 |
| chr15: | 31724141-31725901 |
| chr15: | 31775453-31776533 |
| chr15: | 31966827-31967481 |
| chr15: | 32161626-32163268 |
| chr15: | 33863708-33866938 |
| chr15: | 33865333-33866938 |
| chr15: | 34200448-34201748 |
| chr15: | 34213597-34214458 |
| chr15: | 35950162-35950952 |
| chr15: | 36019819-36020495 |
| chr15: | 36160126-36162205 |
| chr15: | 39372596-39373276 |
| chr15: | 39744398-39744844 |
| chr15: | 40009652-40012222 |
| chr15: | 40011435-40012222 |
| chr15: | 42721271-42723558 |
| chr15: | 43972090-43972578 |
| chr15: | 43980367-43982050 |
| chr15: | 43981201-43982436 |
| chr15: | 43993398-43995607 |
| chr15: | 44136482-44138796 |
| chr15: | 44137459-44138411 |
| chr15: | 44137459-44138796 |
| chr15: | 45115951-45118625 |
| chr15: | 45318919-45323774 |
| chr15: | 45366165-45369038 |
| chr15: | 45519773-45521540 |
| chr15: | 45982592-45983057 |
| chr15: | 46235283-46235839 |
| chr15: | 46255014-46257489 |
| chr15: | 48340571-48343259 |
| chr15: | 49544776-49547871 |
| chr15: | 50086726-50091324 |
| chr15: | 51518901-51519954 |
| chr15: | 53514691-53515746 |
| chr15: | 53678644-53679174 |
| chr15: | 53678778-53679782 |
| chr15: | 53880998-53881772 |
| chr15: | 53974540-53976097 |
| chr15: | 54199944-54203158 |
| chr15: | 54532412-54533030 |
| chr15: | 54890805-54893062 |
| chr15: | 55139281-55140013 |
| chr15: | 55655607-55656732 |
| chr15: | 56447994-56448904 |
| chr15: | 58155364-58156039 |
| chr15: | 58776459-58777765 |
| chr15: | 61057066-61057558 |
| chr15: | 61350765-61351267 |
| chr15: | 61613320-61614839 |
| chr15: | 61613320-61616465 |
| chr15: | 62706082-62707770 |
| chr15: | 63482021-63483103 |
| chr15: | 63710337-63711399 |
| chr15: | 64994365-64995015 |
| chr15: | 65590022-65591547 |
| chr15: | 65714619-65715664 |
| chr15: | 65815222-65819056 |
| chr15: | 65816884-65819122 |
| chr15: | 67868592-67869228 |
| chr15: | 68731293-68732618 |
| chr15: | 70255707-70257984 |
| chr15: | 70387774-70391690 |
| chr15: | 70515375-70515822 |
| chr15: | 70595182-70595651 |
| chr15: | 71881604-71882880 |
| chr15: | 71882007-71882880 |
| chr15: | 72016482-72017489 |
| chr15: | 72386304-72388125 |
| chr15: | 72571691-72575838 |
| chr15: | 73659979-73661419 |
| chr15: | 73668771-73670846 |
| chr15: | 73924279-73925101 |
| chr15: | 74044169-74048692 |
| chr15: | 74044459-74046147 |
| chr15: | 80136733-80137203 |
| chr15: | 81292494-81295779 |
| chr15: | 82396887-82401477 |
| chr15: | 82483011-82486833 |
| chr15: | 84071561-84072231 |
| chr15: | 84086193-84089121 |
| chr15: | 84087000-84088368 |
| chr15: | 84541192-84543810 |
| chr15: | 84542835-84543810 |
| chr15: | 84605109-84606784 |
| chr15: | 85949729-85954292 |
| chr15: | 86056915-86059174 |
| chr15: | 86300360-86301135 |
| chr15: | 86555019-86557156 |
| chr15: | 87905785-87910752 |
| chr15: | 89393696-89400546 |
| chr15: | 89464468-89467429 |
| chr15: | 89549193-89549807 |
| chr15: | 91701479-91702567 |
| chr15: | 91839671-91840376 |
| chr15: | 91981784-91983405 |
| chr15: | 92292727-92294446 |
| chr15: | 92577248-92579208 |
| chr15: | 92674890-92676996 |
| chr15: | 92675335-92676331 |
| chr15: | 93662127-93662820 |
| chr15: | 93826071-93828428 |
| chr15: | 93826474-93827334 |
| chr15: | 93876542-93877247 |
| chr15: | 94106256-94106741 |
| chr15: | 94886323-94888595 |
| chr15: | 95116060-95116964 |
| chr15: | 96043434-96046011 |
| chr15: | 96581687-96582994 |
| chr15: | 96655409-96656098 |
| chr15: | 96873503-96874703 |
| chr15: | 97804333-97303455 |
| chr15: | 98532661-98536459 |
| chr15: | 98596546-98599761 |
| chr15: | 98760503-98762195 |
| chr15: | 98781345-98784315 |
| chr15: | 98842321-98843853 |
| chr15: | 99190120-99192053 |
| chr15: | 99190762-99193816 |
| chr15: | 99551813-99556731 |
| chr15: | 99552905-99554050 |
| chr15: | 99574727-99575462 |
| chr15: | 99617580-99618290 |
| chr15: | 99644218-99646022 |
| chr15: | 99656576-99657411 |
| chr15: | 100466542-100468947 |
| chr15: | 100514033-100517018 |
| chr15: | 100765728-100766363 |
| chr15: | 100796238-100797008 |
| chr15: | 101094448-101098808 |
| chr15: | 101259109-101260569 |
| chr15: | 101341523-101342332 |
| chr15: | 101419325-101420645 |
| chr15: | 101626147-101626712 |
| chr15: | 101635978-101636673 |
| chr15: | 101670755-101671294 |
| chr15: | 101754633-101755112 |
| chr15: | 101790443-101793744 |
| chr15: | 101791299-101792468 |
| chr15: | 101930024-101930549 |
| chr15: | 101999742-102000532 |
| chr15: | 102028912-102030796 |
| chr15: | 102195205-102196035 |
| chr16: | 131670-132210 |
| chr16: | 209785-211113 |
| chr16: | 213274-213830 |
| chr16: | 222858-226404 |
| chr16: | 224519-225801 |
| chr16: | 235963-237268 |
| chr16: | 381320-385726 |
| chr16: | 3682890-3684166 |
| chr16: | 3929935-3932568 |
| chr16: | 3930106-3931145 |
| chr16: | 4165746-4166877 |
| chr16: | 4365208-4367246 |
| chr16: | 4394287-4394872 |
| chr16: | 4698295-4698860 |
| chr16: | 4896827-4898203 |
| chr16: | 5426665-5429606 |
| chr16: | 6780415-6781896 |
| chr16: | 6780434-6784276 |
| chr16: | 6836369-6837668 |
| chr16: | 6836945-6837571 |
| chr16: | 7261443-7262321 |
| chr16: | 7363013-7363664 |
| chr16: | 7812325-7812847 |
| chr16: | 7822468-7824380 |
| chr16: | 8121955-8124996 |
| chr16: | 8470340-8473945 |
| chr16: | 8483141-8484145 |
| chr16: | 8583777-8585833 |
| chr16: | 8688238-8689422 |
| chr16: | 8755543-8756734 |
| chr16: | 8756136-8756734 |
| chr16: | 9029629-9030851 |
| chr16: | 12641319-12641874 |
| chr16: | 12641319-12642939 |
| chr16: | 12656532-12659433 |
| chr16: | 12671039-12674109 |
| chr16: | 12672309-12674109 |
| chr16: | 12673216-12674386 |
| chr16: | 12678973-12680315 |
| chr16: | 12710942-12711492 |
| chr16: | 12710942-12712501 |
| chr16: | 13294345-13296518 |
| chr16: | 17975255-17975943 |
| chr16: | 19353070-19355571 |
| chr16: | 19409243-19414207 |
| chr16: | 19461187-19463057 |
| chr16: | 19988474-19992561 |
| chr16: | 20496042-20500268 |
| chr16: | 23047869-23049560 |
| chr16: | 23159867-23160877 |
| chr16: | 23761054-23762164 |
| chr16: | 24143869-24148051 |
| chr16: | 25340110-25343094 |
| chr16: | 26822964-26823771 |
| chr16: | 26822964-26825674 |
| chr16: | 26879504-26882674 |
| chr16: | 26916943-26919512 |
| chr16: | 27075295-27075920 |
| chr16: | 27880394-27882444 |
| chr16: | 28073830-28075416 |
| chr16: | 35184285-35187824 |
| chr16: | 47177258-47178248 |
| chr16: | 48418477-48419724 |
| chr16: | 48509288-48510957 |
| chr16: | 48510311-48510957 |
| chr16: | 48549888-48554334 |
| chr16: | 48905314-48905795 |
| chr16: | 49288332-49289064 |
| chr16: | 49888583-49893071 |
| chr16: | 49889088-49889838 |
| chr16: | 51031430-51031901 |
| chr16: | 51047907-51049147 |
| chr16: | 52412502-52413995 |
| chr16: | 53164491-53165486 |
| chr16: | 55091883-55093996 |
| chr16: | 55159144-55159668 |
| chr16: | 55884209-55884784 |
| chr16: | 56458907-56459557 |
| chr16: | 56615506-56617016 |
| chr16: | 57126071-57127182 |
| chr16: | 57326683-57327207 |
| chr16: | 57366114-57367105 |
| chr16: | 57570016-57571105 |
| chr16: | 57725385-57728105 |
| chr16: | 58034054-58034733 |
| chr16: | 58059528-58061343 |
| chr16: | 58231457-58232073 |
| chr16: | 58462760-58463374 |
| chr16: | 58497642-58498427 |
| chr16: | 58647258-58649679 |
| chr16: | 58673606-58676357 |
| chr16: | 58945790-58948218 |
| chr16: | 59468949-59471345 |
| chr16: | 60466710-60467372 |
| chr16: | 62239244-62239917 |
| chr16: | 64589295-64590845 |
| chr16: | 65286449-65287474 |
| chr16: | 66736384-66738808 |
| chr16: | 67565133-67567676 |
| chr16: | 67595471-67598299 |
| chr16: | 67934212-67939052 |
| chr16: | 68863801-68866809 |
| chr16: | 70338596-70341204 |
| chr16: | 70977863-70980318 |
| chr16: | 71756826-71758273 |
| chr16: | 72090194-72094156 |
| chr16: | 72318729-72320874 |
| chr16: | 72318979-72319969 |
| chr16: | 73091650-73093207 |
| chr16: | 73331083-73331713 |
| chr16: | 73614456-73616963 |
| chr16: | 73699426-73700760 |
| chr16: | 73915193-73915723 |
| chr16: | 75532377-75533032 |
| chr16: | 76250287-76251108 |
| chr16: | 76449921-76451278 |
| chr16: | 76539133-76544029 |
| chr16: | 76598954-76601513 |
| chr16: | 76716393-76719158 |
| chr16: | 77091007-77092939 |
| chr16: | 77224589-77227011 |
| chr16: | 77977799-77980008 |
| chr16: | 78171663-78172310 |
| chr16: | 78373657-78375265 |
| chr16: | 78383004-78383519 |
| chr16: | 78527053-78528842 |
| chr16: | 78876959-78877951 |
| chr16: | 79059606-79063025 |
| chr16: | 79835005-79836394 |
| chr16: | 79835005-79837826 |
| chr16: | 80191183-80191913 |
| chr16: | 80191198-80193029 |
| chr16: | 80702889-80705178 |
| chr16: | 80837943-80838497 |
| chr16: | 81069171-81070144 |
| chr16: | 81238675-81240848 |
| chr16: | 81244593-81248668 |
| chr16: | 81478084-81479610 |
| chr16: | 83004724-83005499 |
| chr16: | 83149311-83150129 |
| chr16: | 83689965-83693101 |
| chr16: | 83890584-83895007 |
| chr16: | 83935532-83936540 |
| chr16: | 84032383-84035461 |
| chr16: | 84033807-84035341 |
| chr16: | 84069421-84070072 |
| chr16: | 84070062-84070511 |
| chr16: | 84084136-84086001 |
| chr16: | 84278562-84279412 |
| chr16: | 84430199-84431668 |
| chr16: | 84501349-84504913 |
| chr16: | 84670994-84671574 |
| chr16: | 84778077-84779230 |
| chr16: | 84778476-84778965 |
| chr16: | 84812451-84813727 |
| chr16: | 85040399-85043171 |
| chr16: | 85241546-85243054 |
| chr16: | 85302316-85304661 |
| chr16: | 85303436-85304396 |
| chr16: | 85443566-85446146 |
| chr16: | 85644979-85645923 |
| chr16: | 85997098-85997602 |
| chr16: | 86006071-86007681 |
| chr16: | 86006546-86007681 |
| chr16: | 86008686-86009156 |
| chr16: | 86403110-86404315 |
| chr16: | 86542099-86546007 |
| chr16: | 86627597-86630443 |
| chr16: | 86770061-86771286 |
| chr16: | 86878947-86879862 |
| chr16: | 87196114-87196825 |
| chr16: | 87251199-87252309 |
| chr16: | 88264169-88264744 |
| chr16: | 88463518-88466183 |
| chr16: | 88476191-88477492 |
| chr16: | 88562286-88564545 |
| chr16: | 88567540-88563175 |
| chr16: | 88599814-88601205 |
| chr16: | 88613261-88614471 |
| chr16: | 88613551-88614311 |
| chr16: | 88638823-88642083 |
| chr16: | 88794696-88796386 |
| chr16: | 88807770-88808346 |
| chr16: | 88851126-88852206 |
| chr16: | 88861987-88863458 |
| chr16: | 88862733-88863458 |
| chr16: | 88891388-88892058 |
| chr16: | 88897612-88898492 |
| chr16: | 88995317-88996817 |
| chr16: | 89020703-89022438 |
| chr16: | 89074365-89075795 |
| chr16: | 89095051-89097157 |
| chr16: | 89099871-89101742 |
| chr16: | 89149832-89150793 |
| chr16: | 89258924-89259509 |
| chr16: | 89493837-89496954 |
| chr16: | 89495349-89496954 |
| chr16: | 89637052-89637818 |
| chr16: | 89645878-89647703 |
| chr16: | 89653981-89654611 |
| chr16: | 89685240-89636370 |
| chr16: | 89713920-89714845 |
| chr16: | 89798797-89799582 |
| chr16: | 89811677-89812301 |
| chr16: | 89896098-89898402 |
| chr16: | 89913457-89918982 |
| chr16: | 89918457-89919432 |
| chr16: | 89973743-89975018 |
| chr16: | 90069702-90071025 |
| chr16: | 90074225-90075225 |
| chr17: | 16444-18509 |
| chr17: | 20624-21799 |
| chr17: | 30243-31671 |
| chr17: | 41135-42666 |
| chr17: | 51239-51734 |
| chr17: | 66805-68405 |
| chr17: | 69942-71897 |
| chr17: | 33652-84147 |
| chr17: | 84932-85782 |
| chr17: | 91976-94094 |
| chr17: | 103345-107880 |
| chr17: | 2399509-2402793 |
| chr17: | 2402049-2402793 |
| chr17: | 2626905-2628905 |
| chr17: | 2641645-2645341 |
| chr17: | 2903715-2904905 |
| chr17: | 2934591-2935561 |
| chr17: | 3414758-3416189 |
| chr17: | 3618470-3619523 |
| chr17: | 3771159-3771789 |
| chr17: | 3888087-3888802 |
| chr17: | 3963932-3965472 |
| chr17: | 4042169-4042667 |
| chr17: | 4060495-4061450 |
| chr17: | 4094578-4095947 |
| chr17: | 5143337-5144627 |
| chr17: | 5595739-5597528 |
| chr17: | 5625345-5626215 |
| chr17: | 5653815-5655545 |
| chr17: | 5674832-5677333 |
| chr17: | 5325604-5826114 |
| chr17: | 5972446-5974380 |
| chr17: | 6162621-6163946 |
| chr17: | 6204242-6205289 |
| chr17: | 6536239-6537264 |
| chr17: | 6798619-6799151 |
| chr17: | 7077578-7080343 |
| chr17: | 7141465-7142499 |
| chr17: | 7264766-7265803 |
| chr17: | 7265278-7265803 |
| chr17: | 7337085-7337566 |
| chr17: | 7741356-7742116 |
| chr17: | 8015638-3016322 |
| chr17: | 8649689-8650260 |
| chr17: | 8649709-8650838 |
| chr17: | 8924178-8926876 |
| chr17: | 9179228-9181382 |
| chr17: | 10240763-10241618 |
| chr17: | 11211237-11211996 |
| chr17: | 11306173-11306843 |
| chr17: | 11411561-11412466 |
| chr17: | 13098451-13101604 |
| chr17: | 13213646-13214821 |
| chr17: | 13531491-13532652 |
| chr17: | 13624707-13625431 |
| chr17: | 13699969-13700937 |
| chr17: | 14188236-14191556 |
| chr17: | 14189898-14191556 |
| chr17: | 14190939-14191556 |
| chr17: | 14559948-14561234 |
| chr17: | 14939000-14940910 |
| chr17: | 14939555-14941095 |
| chr17: | 15183484-15135610 |
| chr17: | 31618934-31621095 |
| chr17: | 31755131-31757527 |
| chr17: | 31817852-31818376 |
| chr17: | 32063870-32064648 |
| chr17: | 32177079-32179414 |
| chr17: | 32337918-32339046 |
| chr17: | 32805664-32806482 |
| chr17: | 32872426-32874572 |
| chr17: | 33778004-33780956 |
| chr17: | 35136852-35137762 |
| chr17: | 35236037-35237101 |
| chr17: | 35755840-35758985 |
| chr17: | 39382871-39384158 |
| chr17: | 39729374-39732747 |
| chr17: | 39731090-39731757 |
| chr17: | 39738178-39733928 |
| chr17: | 39738178-39741898 |
| chr17: | 39754010-39755565 |
| chr17: | 40427345-40428983 |
| chr17: | 40489925-40490615 |
| chr17: | 41398187-41401093 |
| chr17: | 41399895-41401223 |
| chr17: | 41437832-41440689 |
| chr17: | 41437967-41439309 |
| chr17: | 41438752-41442275 |
| chr17: | 41517276-41518547 |
| chr17: | 41976744-41977334 |
| chr17: | 42295583-42299041 |
| chr17: | 43001518-43002446 |
| chr17: | 43496222-43497302 |
| chr17: | 45888220-45888871 |
| chr17: | 46400513-46402257 |
| chr17: | 47037073-47037972 |
| chr17: | 47351963-47352880 |
| chr17: | 47826435-47828174 |
| chr17: | 47826990-47827815 |
| chr17: | 47923413-47925248 |
| chr17: | 48047409-48048794 |
| chr17: | 48129766-48131256 |
| chr17: | 49519907-49520374 |
| chr17: | 49624029-49627067 |
| chr17: | 49897968-49899371 |
| chr17: | 52158699-52163066 |
| chr17: | 52160208-52162996 |
| chr17: | 52489330-52494025 |
| chr17: | 53463729-53465323 |
| chr17: | 54599492-54602594 |
| chr17: | 54705087-54706160 |
| chr17: | 54793188-54795857 |
| chr17: | 54794633-54795707 |
| chr17: | 55090033-55092655 |
| chr17: | 55208475-55210333 |
| chr17: | 55208805-55209675 |
| chr17: | 55687846-55689837 |
| chr17: | 55880075-55881881 |
| chr17: | 56168150-56168735 |
| chr17: | 63463807-63464582 |
| chr17: | 64287177-64288769 |
| chr17: | 64457974-64458759 |
| chr17: | 64959657-64961650 |
| chr17: | 64984874-64986080 |
| chr17: | 66677283-66680447 |
| chr17: | 66797847-66799518 |
| chr17: | 67344391-67348779 |
| chr17: | 67882604-67883379 |
| chr17: | 69124883-69125513 |
| chr17: | 70026246-70026940 |
| chr17: | 70537709-70539439 |
| chr17: | 70910723-70911793 |
| chr17: | 70979648-70980596 |
| chr17: | 71640670-71642044 |
| chr17: | 71703448-71704040 |
| chr17: | 71722411-71723030 |
| chr17: | 71735379-71736063 |
| chr17: | 72246407-72249662 |
| chr17: | 72246682-72248137 |
| chr17: | 72334520-72335820 |
| chr17: | 73542643-73543213 |
| chr17: | 73875451-73879169 |
| chr17: | 74235793-74237089 |
| chr17: | 74287402-74289892 |
| chr17: | 74380201-74382231 |
| chr17: | 74564322-74566005 |
| chr17: | 74783536-74788188 |
| chr17: | 74787079-74790112 |
| chr17: | 75248605-75250558 |
| chr17: | 75249953-75250558 |
| chr17: | 75554546-75559026 |
| chr17: | 75557106-75559026 |
| chr17: | 75672894-75673457 |
| chr17: | 75682932-75683619 |
| chr17: | 75755291-75756931 |
| chr17: | 76282527-76282977 |
| chr17: | 76999941-77001350 |
| chr17: | 77061223-77061758 |
| chr17: | 77078099-77080589 |
| chr17: | 77363224-77364344 |
| chr17: | 77491472-77491979 |
| chr17: | 79026584-79028099 |
| chr17: | 79027604-79028099 |
| chr17: | 79051671-79052611 |
| chr17: | 79204200-79204810 |
| chr17: | 79689725-79692733 |
| chr17: | 80107376-80109446 |
| chr17: | 80141576-80142196 |
| chr17: | 80211685-80213199 |
| chr17: | 80316838-80319609 |
| chr17: | 80652042-80653626 |
| chr17: | 80722583-80723358 |
| chr17: | 80759397-80760387 |
| chr17: | 80929394-80930794 |
| chr17: | 80932814-80934859 |
| chr17: | 80941842-80942402 |
| chr17: | 80960564-80962694 |
| chr17: | 80980123-80981288 |
| chr17: | 80989093-80990373 |
| chr17: | 81012463-81013883 |
| chr17: | 81025706-81026991 |
| chr18: | 651992-654019 |
| chr18: | 1199543-1200014 |
| chr18: | 2372269-2377052 |
| chr18: | 3350167-3350947 |
| chr18: | 3560692-3561929 |
| chr18: | 3635526-3637823 |
| chr18: | 3635601-3636398 |
| chr18: | 4506475-4510678 |
| chr18: | 4513869-4515734 |
| chr18: | 4535436-4536716 |
| chr18: | 5209094-5211041 |
| chr18: | 5209822-5210685 |
| chr18: | 5294874-5296604 |
| chr18: | 5324675-5325795 |
| chr18: | 5324675-5326779 |
| chr18: | 6272476-6273541 |
| chr18: | 6871761-6872242 |
| chr18: | 7144225-7146816 |
| chr18: | 7566788-7568888 |
| chr18: | 8704953-8706813 |
| chr18: | 9205217-9206940 |
| chr18: | 9614223-9614938 |
| chr18: | 9815046-9816215 |
| chr18: | 9913879-9914977 |
| chr18: | 10150590-10151050 |
| chr18: | 10754158-10757494 |
| chr18: | 10755754-10757414 |
| chr18: | 11509973-11510808 |
| chr18: | 11509973-11511491 |
| chr18: | 11864390-11866873 |
| chr18: | 12060305-12061210 |
| chr18: | 12060720-12063440 |
| chr18: | 12503702-12504211 |
| chr18: | 12657165-12658579 |
| chr18: | 20715577-20716288 |
| chr18: | 23747858-23751787 |
| chr18: | 23764118-23766428 |
| chr18: | 24408671-24409888 |
| chr18: | 24571642-24572306 |
| chr18: | 25070858-25072162 |
| chr18: | 27489037-27490855 |
| chr18: | 28279458-28233863 |
| chr18: | 28771340-28775873 |
| chr18: | 29003300-29004316 |
| chr18: | 29146941-29148053 |
| chr18: | 29887462-29889647 |
| chr18: | 29888110-29889257 |
| chr18: | 29888294-29889647 |
| chr18: | 29888571-29889242 |
| chr18: | 33160797-33161919 |
| chr18: | 33594884-33596937 |
| chr18: | 34051196-34053643 |
| chr18: | 34509248-34512006 |
| chr18: | 34917949-34918404 |
| chr18: | 35306075-35306647 |
| chr18: | 35550541-35551942 |
| chr18: | 35963779-35967784 |
| chr18: | 36144563-36145299 |
| chr18: | 37566617-37567105 |
| chr18: | 38864861-38868284 |
| chr18: | 39442592-39443633 |
| chr18: | 40054080-40057679 |
| chr18: | 41116889-41118176 |
| chr18: | 42624662-42625988 |
| chr18: | 42625424-42626047 |
| chr18: | 42838115-42838727 |
| chr18: | 43013411-43014202 |
| chr18: | 43913705-43914290 |
| chr18: | 43977083-43978888 |
| chr18: | 44774050-44775630 |
| chr18: | 44925360-44928518 |
| chr18: | 44926638-44929859 |
| chr18: | 45095294-45096085 |
| chr18: | 45736996-45739428 |
| chr18: | 45746668-45748514 |
| chr18: | 46039615-46040594 |
| chr18: | 46396136-46396861 |
| chr18: | 46396216-46397741 |
| chr18: | 47608979-47609448 |
| chr18: | 47634727-47635645 |
| chr18: | 47695039-47698432 |
| chr18: | 47825013-47823810 |
| chr18: | 49196921-49198311 |
| chr18: | 49358151-49359287 |
| chr18: | 49911899-49914726 |
| chr18: | 50462528-50463030 |
| chr18: | 51136445-51137770 |
| chr18: | 51205898-51210735 |
| chr18: | 51754383-51756823 |
| chr18: | 52391201-52394158 |
| chr18: | 53363728-53365782 |
| chr18: | 53757043-53757840 |
| chr18: | 53929188-53931239 |
| chr18: | 53930605-53931239 |
| chr18: | 54812555-54813564 |
| chr18: | 54946737-54948704 |
| chr18: | 56770154-56770840 |
| chr18: | 58492109-58492573 |
| chr18: | 58672229-58672736 |
| chr18: | 59706671-59707551 |
| chr18: | 60189275-60194148 |
| chr18: | 60247700-60249139 |
| chr18: | 61171915-61174140 |
| chr18: | 62035888-62037243 |
| chr18: | 62432666-62433296 |
| chr18: | 63376194-63379614 |
| chr18: | 63635463-63640238 |
| chr18: | 63766925-63769179 |
| chr18: | 63831181-63834769 |
| chr18: | 63907490-63911656 |
| chr18: | 64007150-64008021 |
| chr18: | 64130327-64133306 |
| chr18: | 64219751-64221199 |
| chr18: | 64777244-64779448 |
| chr18: | 65479905-65480433 |
| chr18: | 65947967-65949023 |
| chr18: | 66228676-66229352 |
| chr18: | 66992743-66994075 |
| chr18: | 67149606-67151578 |
| chr18: | 67953694-67955314 |
| chr18: | 68795497-68796408 |
| chr18: | 69905774-69903034 |
| chr18: | 70000615-70002926 |
| chr18: | 70001707-70004730 |
| chr18: | 70364186-70365372 |
| chr18: | 70439208-70439773 |
| chr18: | 70610481-70611522 |
| chr18: | 70610662-70613183 |
| chr18: | 71814375-71818190 |
| chr18: | 71871714-71872784 |
| chr18: | 72076889-72077885 |
| chr18: | 73271681-73272169 |
| chr18: | 73271757-73272689 |
| chr18: | 73563481-73568935 |
| chr18: | 74104897-74106762 |
| chr18: | 74323477-74324150 |
| chr18: | 74845911-74846371 |
| chr18: | 75084719-75085599 |
| chr18: | 75104775-75105355 |
| chr18: | 75267006-75268102 |
| chr18: | 75381675-75383325 |
| chr18: | 75542584-75543375 |
| chr18: | 75681141-75681914 |
| chr18: | 75833100-75834278 |
| chr18: | 76129943-76133283 |
| chr18: | 76169501-76171394 |
| chr18: | 76197122-76198407 |
| chr18: | 76615440-76616287 |
| chr18: | 76637155-76639640 |
| chr18: | 76662561-76666204 |
| chr18: | 76676168-76677567 |
| chr18: | 76739427-76741039 |
| chr18: | 76774314-76777215 |
| chr18: | 76792747-76797707 |
| chr18: | 76904955-76906791 |
| chr18: | 77057741-77059088 |
| chr18: | 77068644-77069790 |
| chr18: | 77087429-77088278 |
| chr18: | 77116597-77119052 |
| chr18: | 77233375-77235335 |
| chr18: | 77264706-77266046 |
| chr18: | 77280490-77232665 |
| chr18: | 77309972-77312107 |
| chr18: | 77311357-77312107 |
| chr18: | 77373439-77380919 |
| chr18: | 77403564-77409149 |
| chr18: | 77451438-77452308 |
| chr18: | 77504834-77507819 |
| chr18: | 77504864-77506474 |
| chr18: | 77524428-77525558 |
| chr18: | 77530788-77531503 |
| chr18: | 77567766-77569681 |
| chr18: | 77622310-77624755 |
| chr18: | 77645839-77647204 |
| chr18: | 77651618-77655877 |
| chr18: | 77679469-77682189 |
| chr18: | 77808225-77809170 |
| chr18: | 77831173-77831853 |
| chr18: | 77833350-77833930 |
| chr18: | 77908809-77910909 |
| chr19: | 1028965-1029535 |
| chr19: | 1033637-1035297 |
| chr19: | 1049440-1050029 |
| chr19: | 1137277-1138293 |
| chr19: | 1148447-1149065 |
| chr19: | 1249083-1251277 |
| chr19: | 1566424-1568402 |
| chr19: | 1627764-1629149 |
| chr19: | 1862345-1865249 |
| chr19: | 1950433-1951640 |
| chr19: | 2014069-2015499 |
| chr19: | 2128443-2129063 |
| chr19: | 2432885-2433730 |
| chr19: | 2692117-2695570 |
| chr19: | 2701479-2702569 |
| chr19: | 2713066-2714451 |
| chr19: | 2772808-2774118 |
| chr19: | 2817651-2818621 |
| chr19: | 2841218-2842149 |
| chr19: | 2909172-2910422 |
| chr19: | 2909624-2910422 |
| chr19: | 2946001-2943250 |
| chr19: | 3172493-3175378 |
| chr19: | 3528562-3530792 |
| chr19: | 3649029-3649899 |
| chr19: | 3937031-3938551 |
| chr19: | 3973264-3974945 |
| chr19: | 4065267-4061669 |
| chr19: | 4246932-4247485 |
| chr19: | 4471340-4472189 |
| chr19: | 4510620-4513210 |
| chr19: | 4512600-4513195 |
| chr19: | 4579235-4582395 |
| chr19: | 4663056-4664031 |
| chr19: | 4692171-4694141 |
| chr19: | 5076094-5076844 |
| chr19: | 5250743-5251257 |
| chr19: | 5510246-5510694 |
| chr19: | 5904363-5904863 |
| chr19: | 6533477-6534628 |
| chr19: | 7251000-7252818 |
| chr19: | 7482902-7484874 |
| chr19: | 7514520-7516227 |
| chr19: | 7515216-7516227 |
| chr19: | 7648267-7652758 |
| chr19: | 7754964-7757524 |
| chr19: | 7934856-7936869 |
| chr19: | 8243075-8244643 |
| chr19: | 8412034-8413600 |
| chr19: | 8454839-8456179 |
| chr19: | 10212175-10214414 |
| chr19: | 11295091-11296445 |
| chr19: | 11319906-11322751 |
| chr19: | 11485189-11486204 |
| chr19: | 12694939-12698475 |
| chr19: | 13698524-13702681 |
| chr19: | 13775994-13776717 |
| chr19: | 14200616-14202374 |
| chr19: | 14226825-14231072 |
| chr19: | 14586911-14587811 |
| chr19: | 14731794-14734049 |
| chr19: | 14732369-14732905 |
| chr19: | 15046340-15048075 |
| chr19: | 15046396-15049599 |
| chr19: | 15046747-15047280 |
| chr19: | 15762030-15763264 |
| chr19: | 15784219-15786242 |
| chr19: | 15787092-15789277 |
| chr19: | 16037674-16040476 |
| chr19: | 16041619-16045679 |
| chr19: | 16103205-16106575 |
| chr19: | 16985026-16986792 |
| chr19: | 17007275-17008550 |
| chr19: | 17141653-17145495 |
| chr19: | 17261340-17261790 |
| chr19: | 17355504-17357436 |
| chr19: | 17377697-17378172 |
| chr19: | 18390503-18393159 |
| chr19: | 18717041-18717785 |
| chr19: | 18746535-18747892 |
| chr19: | 18763872-18765637 |
| chr19: | 19006542-19007176 |
| chr19: | 19174047-19177125 |
| chr19: | 19837791-19839599 |
| chr19: | 27801605-27806428 |
| chr19: | 27961548-27963053 |
| chr19: | 28214998-28219555 |
| chr19: | 28250655-28253690 |
| chr19: | 28333001-28336921 |
| chr19: | 28772638-28776611 |
| chr19: | 29311335-29314355 |
| chr19: | 29336918-29339937 |
| chr19: | 29456670-29458089 |
| chr19: | 29504537-29505212 |
| chr19: | 30002185-30005676 |
| chr19: | 30003872-30006351 |
| chr19: | 30032593-30033668 |
| chr19: | 30056800-30058734 |
| chr19: | 30057935-30058505 |
| chr19: | 30185398-30189322 |
| chr19: | 31286835-31289388 |
| chr19: | 31287507-31289448 |
| chr19: | 31316723-31318665 |
| chr19: | 31783225-31784024 |
| chr19: | 31888599-31892119 |
| chr19: | 31983030-31983515 |
| chr19: | 32189210-32192910 |
| chr19: | 32994238-32994897 |
| chr19: | 33211100-33211863 |
| chr19: | 33236640-33238225 |
| chr19: | 33370544-33371179 |
| chr19: | 33375601-33377056 |
| chr19: | 33684987-33686024 |
| chr19: | 35182513-35183155 |
| chr19: | 35758040-35761056 |
| chr19: | 35758933-35760371 |
| chr19: | 36018030-36019735 |
| chr19: | 37969479-37970064 |
| chr19: | 38341552-38345358 |
| chr19: | 38343838-38345086 |
| chr19: | 38826488-38827974 |
| chr19: | 39138183-39140365 |
| chr19: | 39339686-39341232 |
| chr19: | 39616543-39617282 |
| chr19: | 40385410-40386050 |
| chr19: | 41256551-41257224 |
| chr19: | 41283063-41285263 |
| chr19: | 42400342-42401377 |
| chr19: | 42579999-42582314 |
| chr19: | 42772456-42773408 |
| chr19: | 42786911-42789522 |
| chr19: | 43035285-43037914 |
| chr19: | 44142913-44144340 |
| chr19: | 45110365-45113565 |
| chr19: | 45169959-45170654 |
| chr19: | 45473272-45474019 |
| chr19: | 45681508-45682018 |
| chr19: | 46220423-46221369 |
| chr19: | 46327188-46327739 |
| chr19: | 46498343-46499055 |
| chr19: | 46790766-46792939 |
| chr19: | 47339060-47340263 |
| chr19: | 47732968-47735687 |
| chr19: | 47798306-47803274 |
| chr19: | 48189989-48193207 |
| chr19: | 48413129-48415527 |
| chr19: | 48536767-48537287 |
| chr19: | 48900493-48901627 |
| chr19: | 48945957-48947285 |
| chr19: | 50126926-50127981 |
| chr19: | 50179971-50180451 |
| chr19: | 50269645-50270100 |
| chr19: | 50445800-50447039 |
| chr19: | 50508187-50509252 |
| chr19: | 51077878-51082231 |
| chr19: | 51106142-51109013 |
| chr19: | 51106860-51109663 |
| chr19: | 51314559-51315674 |
| chr19: | 51406607-51408006 |
| chr19: | 51416813-51417948 |
| chr19: | 51459520-51461407 |
| chr19: | 51509454-51511116 |
| chr19: | 51545379-51547329 |
| chr19: | 51891298-51892818 |
| chr19: | 52692555-52696215 |
| chr19: | 52800177-52800862 |
| chr19: | 52862709-52864174 |
| chr19: | 52863563-52864174 |
| chr19: | 52888750-52891129 |
| chr19: | 53316544-53319470 |
| chr19: | 53629595-53632219 |
| chr19: | 53631599-53632219 |
| chr19: | 53734422-53738349 |
| chr19: | 54033402-54034548 |
| chr19: | 54180231-54181558 |
| chr19: | 54345267-54348463 |
| chr19: | 54461539-54466402 |
| chr19: | 54464060-54466372 |
| chr19: | 54555703-54560487 |
| chr19: | 54640883-54641558 |
| chr19: | 54736841-54740017 |
| chr19: | 54736920-54738390 |
| chr19: | 54860861-54861780 |
| chr19: | 55203092-55205437 |
| chr19: | 55344415-55345835 |
| chr19: | 55358473-55361191 |
| chr19: | 55476234-55477885 |
| chr19: | 55544225-55544999 |
| chr19: | 55589613-55590258 |
| chr19: | 55600088-55600754 |
| chr19: | 55607778-55609493 |
| chr19: | 55850619-55851369 |
| chr19: | 56016360-56017716 |
| chr19: | 56223675-56224733 |
| chr19: | 56361020-56363354 |
| chr19: | 56686719-56688658 |
| chr19: | 56745440-56748414 |
| chr19: | 56849972-56851425 |
| chr19: | 56850458-56851297 |
| chr19: | 56866464-56869843 |
| chr19: | 57180874-57181459 |
| chr19: | 57680072-57681227 |
| chr19: | 57683323-57684560 |
| chr19: | 57755520-57756502 |
| chr19: | 57777841-57779512 |
| chr19: | 57835776-57836515 |
| chr19: | 58034665-58035196 |
| chr19: | 59111130-59114781 |
| chr20: | 348844-350842 |
| chr20: | 622529-623151 |
| chr20: | 700504-701194 |
| chr20: | 2198385-2201331 |
| chr20: | 2614141-2616678 |
| chr20: | 2673175-2674407 |
| chr20: | 5789573-5791247 |
| chr20: | 5852022-5852884 |
| chr20: | 6025413-6027507 |
| chr20: | 6969682-6970172 |
| chr20: | 7398742-7403609 |
| chr20: | 7604997-7607560 |
| chr20: | 7699277-7702732 |
| chr20: | 8410097-8411094 |
| chr20: | 9169203-9170173 |
| chr20: | 9320527-9321996 |
| chr20: | 9320842-9321512 |
| chr20: | 9321378-9321894 |
| chr20: | 11647495-11648765 |
| chr20: | 13201379-13202490 |
| chr20: | 14435333-14437206 |
| chr20: | 14477380-14478896 |
| chr20: | 15301756-15303851 |
| chr20: | 15709506-15712363 |
| chr20: | 15711562-15712363 |
| chr20: | 16240175-16240835 |
| chr20: | 16374155-16377056 |
| chr20: | 16375419-16376693 |
| chr20: | 19904678-19905324 |
| chr20: | 20335284-20339330 |
| chr20: | 21026702-21027304 |
| chr20: | 21122268-21123962 |
| chr20: | 22839322-22839797 |
| chr20: | 23593820-23594562 |
| chr20: | 24234682-24236680 |
| chr20: | 24388726-24389645 |
| chr20: | 24411076-24412891 |
| chr20: | 24758946-24759790 |
| chr20: | 26297904-26302831 |
| chr20: | 30478074-30481840 |
| chr20: | 30839397-30843511 |
| chr20: | 31170818-31173401 |
| chr20: | 31525748-31529309 |
| chr20: | 32084331-32085300 |
| chr20: | 33242327-33243723 |
| chr20: | 36791314-36791847 |
| chr20: | 36796996-36800551 |
| chr20: | 37055044-37055849 |
| chr20: | 39882521-39886182 |
| chr20: | 39906563-39907379 |
| chr20: | 40337048-40340280 |
| chr20: | 42107623-42110851 |
| chr20: | 42272167-42273896 |
| chr20: | 42481443-42484024 |
| chr20: | 42893404-42894522 |
| chr20: | 43275129-43278294 |
| chr20: | 43305631-43308774 |
| chr20: | 43307048-43308774 |
| chr20: | 43327321-43329532 |
| chr20: | 44204237-44207302 |
| chr20: | 44792102-44793882 |
| chr20: | 44926136-44928026 |
| chr20: | 50652091-50654356 |
| chr20: | 50759422-50760759 |
| chr20: | 51008382-51009760 |
| chr20: | 51157759-51159154 |
| chr20: | 51219042-51219855 |
| chr20: | 51304662-51305611 |
| chr20: | 52613366-52614476 |
| chr20: | 52774882-52775544 |
| chr20: | 53289505-53294132 |
| chr20: | 53292985-53294741 |
| chr20: | 53603517-53604740 |
| chr20: | 54125396-54127869 |
| chr20: | 54199560-54200691 |
| chr20: | 54237699-54238387 |
| chr20: | 54750841-54754895 |
| chr20: | 54807171-54810710 |
| chr20: | 56100137-56102320 |
| chr20: | 56283924-56285426 |
| chr20: | 57160574-57161614 |
| chr20: | 57465451-57467183 |
| chr20: | 57958947-57960167 |
| chr20: | 58267508-58268067 |
| chr20: | 58744737-58745914 |
| chr20: | 58984052-58985772 |
| chr20: | 59675921-59677994 |
| chr20: | 59776865-59777500 |
| chr20: | 59827152-59828515 |
| chr20: | 59857827-59858527 |
| chr20: | 59925210-59925840 |
| chr20: | 59964903-59965975 |
| chr20: | 60113057-60113652 |
| chr20: | 60185970-60190121 |
| chr20: | 60216706-60217274 |
| chr20: | 60216706-60219476 |
| chr20: | 60298972-60299492 |
| chr20: | 60358509-60359899 |
| chr20: | 60511031-60511689 |
| chr20: | 60545655-60546725 |
| chr20: | 60632668-60634103 |
| chr20: | 60639424-60642008 |
| chr20: | 60718405-60719511 |
| chr20: | 60788047-60788817 |
| chr20: | 61072776-61073858 |
| chr20: | 61191965-61193870 |
| chr20: | 61304168-61305178 |
| chr20: | 61372180-61375000 |
| chr20: | 61597450-61597950 |
| chr20: | 61623905-61625290 |
| chr20: | 61724718-61725597 |
| chr20: | 61765845-61766941 |
| chr21: | 16403191-16404403 |
| chr21: | 16588393-16589894 |
| chr21: | 16588393-16591163 |
| chr21: | 16678132-16678599 |
| chr21: | 17943609-17944569 |
| chr21: | 18612370-18613365 |
| chr21: | 19043939-19047240 |
| chr21: | 19325706-19329045 |
| chr21: | 19326919-19329199 |
| chr21: | 19327429-19328317 |
| chr21: | 19432035-19435744 |
| chr21: | 20393497-20394443 |
| chr21: | 21212481-21213550 |
| chr21: | 21621703-21624006 |
| chr21: | 22000188-22004189 |
| chr21: | 24159173-24160503 |
| chr21: | 24159539-24160163 |
| chr21: | 24373594-24375391 |
| chr21: | 24374796-24375391 |
| chr21: | 24785084-24788082 |
| chr21: | 25333627-25334096 |
| chr21: | 26359909-26364555 |
| chr21: | 26862124-26865407 |
| chr21: | 27175040-27179728 |
| chr21: | 28476934-28477619 |
| chr21: | 29712972-29714163 |
| chr21: | 30987077-30987597 |
| chr21: | 31292430-31293424 |
| chr21: | 32432147-32434379 |
| chr21: | 33556468-33557243 |
| chr21: | 33672755-33673258 |
| chr21: | 34693718-34694194 |
| chr21: | 34693718-34696725 |
| chr21: | 35383916-35387241 |
| chr21: | 35445350-35446724 |
| chr21: | 35530589-35533098 |
| chr21: | 36261096-36262805 |
| chr21: | 36561391-36565832 |
| chr21: | 36601281-36602362 |
| chr21: | 39128851-39129996 |
| chr21: | 39651128-39655123 |
| chr21: | 39973933-39975874 |
| chr21: | 40050052-40051416 |
| chr21: | 40814284-40816976 |
| chr21: | 41344469-41345024 |
| chr21: | 41346366-41347056 |
| chr21: | 41521089-41521810 |
| chr21: | 41554486-41555154 |
| chr21: | 41670393-41674209 |
| chr21: | 42845934-42847731 |
| chr21: | 43037448-43039221 |
| chr21: | 43037459-43040086 |
| chr21: | 43284492-43285567 |
| chr21: | 43350120-43353426 |
| chr21: | 43498241-43498896 |
| chr21: | 43557506-43558111 |
| chr21: | 43933622-43934715 |
| chr21: | 44007534-44010634 |
| chr21: | 44705465-44706185 |
| chr21: | 44718621-44719136 |
| chr21: | 44808566-44809052 |
| chr21: | 44970196-44973492 |
| chr21: | 45001567-45004171 |
| chr21: | 45159263-45160863 |
| chr21: | 45508571-45509106 |
| chr21: | 45618957-45621033 |
| chr21: | 45681654-45683665 |
| chr21: | 45681975-45683050 |
| chr21: | 45719922-45720647 |
| chr21: | 45789163-45739993 |
| chr21: | 45815454-45816905 |
| chr21: | 45828928-45831001 |
| chr21: | 45904010-45905210 |
| chr21: | 45904451-45905356 |
| chr21: | 45942684-45945229 |
| chr21: | 46401513-46402593 |
| chr21: | 46447971-46448726 |
| chr21: | 46654099-46654729 |
| chr21: | 46765045-46766149 |
| chr21: | 46776104-46779708 |
| chr21: | 46973433-46975421 |
| chr21: | 47026008-47027529 |
| chr21: | 47027707-47029860 |
| chr21: | 47052971-47054551 |
| chr21: | 47053051-47056946 |
| chr21: | 47316342-47317187 |
| chr21: | 47318227-47318887 |
| chr21: | 47329554-47330073 |
| chr21: | 47338186-47338851 |
| chr21: | 47343696-47345241 |
| chr21: | 47388056-47390190 |
| chr21: | 47388130-47388749 |
| chr21: | 47425860-47426395 |
| chr21: | 47454139-47455512 |
| chr21: | 47560011-47561065 |
| chr21: | 47589087-47591921 |
| chr21: | 47603360-47604350 |
| chr21: | 47609779-47610834 |
| chr21: | 47657518-47658672 |
| chr21: | 47712420-47714722 |
| chr21: | 47801944-47804584 |
| chr21: | 47823108-47824123 |
| chr22: | 25245604-25246110 |
| chr22: | 25449752-25452479 |
| chr22: | 27704015-27704564 |
| chr22: | 29383848-29386970 |
| chr22: | 29515970-29516620 |
| chr22: | 29633118-29635222 |
| chr22: | 29680263-29681805 |
| chr22: | 30336350-30337198 |
| chr22: | 31420065-31420768 |
| chr22: | 31486508-31487078 |
| chr22: | 32927932-32928517 |
| chr22: | 33662527-33665397 |
| chr22: | 33736047-33737076 |
| chr22: | 33755373-33760446 |
| chr22: | 33780263-33783473 |
| chr22: | 34724321-34725253 |
| chr22: | 34863937-34866241 |
| chr22: | 34900119-34901341 |
| chr22: | 35295028-35299027 |
| chr22: | 35469747-35471058 |
| chr22: | 35645445-35646472 |
| chr22: | 36069890-36071426 |
| chr22: | 36918738-36923490 |
| chr22: | 37143357-37147011 |
| chr22: | 37493688-37494368 |
| chr22: | 37618621-37621144 |
| chr22: | 37983065-37984326 |
| chr22: | 38054434-38055177 |
| chr22: | 39293896-39298665 |
| chr22: | 42716350-42716860 |
| chr22: | 42717400-42720441 |
| chr22: | 42879201-42830111 |
| chr22: | 43431422-43436218 |
| chr22: | 43594738-43595763 |
| chr22: | 43676579-43677609 |
| chr22: | 46491132-46492057 |
| chr22: | 46535015-46537497 |
| chr22: | 46872703-46873298 |
| chr22: | 47027350-47027940 |
| chr22: | 47254779-47256635 |
| chr22: | 47459249-47459949 |
| chr22: | 47606751-47609606 |
| chr22: | 47801028-47801658 |
| chr22: | 47996185-47996815 |
| chr22: | 48128044-48128679 |
| chr22: | 48481462-48483565 |
| chr22: | 48482572-48483330 |
| chr22: | 48594721-48595537 |
| chr22: | 48647627-48649147 |
| chr22: | 48893354-48894129 |
| chr22: | 49014818-49017434 |
| chr22: | 49034097-49034913 |
| chr22: | 49050926-49051616 |
| chr22: | 49062450-49064460 |
| chr22: | 49063500-49064520 |
| chr22: | 49131616-49132301 |
| chr22: | 50329163-50329646 |
| chr22: | 50467091-50467871 |
| chr22: | 50495516-50496091 |
| chr22: | 50674880-50677245 |
| chr22: | 50782140-50784908 |
| chr22: | 50871166-50873706 |
| chr22: | 50976153-50976699 |
| chr22: | 51082134-51082724 |
| chr22: | 51117967-51121392 |
| chr22: | 51123499-51125484 |
| chr22: | 51186721-51187416 |
| chrX: | 2006342-2007441 |
| chrX: | 2292139-2294700 |
| chrX: | 2331747-2336269 |
| chrX: | 2390959-2392269 |
| chrX: | 2419193-2420758 |
| chrX: | 2515135-2516329 |
| chrX: | 2545862-2550743 |
| chrX: | 2565454-2566289 |
| chrX: | 3060615-3061823 |
| chrX: | 3761130-3762262 |
| chrX: | 3799164-3800780 |
| chrX: | 3837863-3839310 |
| chrX: | 4157512-4161894 |
| chrX: | 4157626-4159273 |
| chrX: | 5055404-5057320 |
| chrX: | 5159509-5164153 |
| chrX: | 5362305-5363025 |
| chrX: | 7622203-7623589 |
| chrX: | 8786541-8788892 |
| chrX: | 9432542-9434165 |
| chrX: | 9753741-9754954 |
| chrX: | 9982509-9985831 |
| chrX: | 9982564-9984219 |
| chrX: | 10530516-10531080 |
| chrX: | 14645065-14645876 |
| chrX: | 14797854-14799253 |
| chrX: | 16887834-16889249 |
| chrX: | 17637522-17638135 |
| chrX: | 18277660-18281295 |
| chrX: | 18278683-18281421 |
| chrX: | 19229162-19229770 |
| chrX: | 19374309-19374922 |
| chrX: | 19465688-19469525 |
| chrX: | 20143624-20146498 |
| chrX: | 24042887-24043913 |
| chrX: | 26059771-26063406 |
| chrX: | 26363207-26366294 |
| chrX: | 27714768-27716542 |
| chrX: | 29266817-29268327 |
| chrX: | 32430422-32431602 |
| chrX: | 32644633-32645696 |
| chrX: | 32919548-32920845 |
| chrX: | 32987241-32988323 |
| chrX: | 32987254-32989016 |
| chrX: | 34153555-34156749 |
| chrX: | 34439797-34441842 |
| chrX: | 35441711-35445379 |
| chrX: | 35594372-35595525 |
| chrX: | 35630480-35634416 |
| chrX: | 35714098-35716077 |
| chrX: | 35814752-35817325 |
| chrX: | 37031234-37034388 |
| chrX: | 38386545-38387372 |
| chrX: | 38584381-38587366 |
| chrX: | 39547851-39548742 |
| chrX: | 43944847-43946844 |
| chrX: | 43944847-43947747 |
| chrX: | 46433008-46434874 |
| chrX: | 46771584-46772827 |
| chrX: | 47583975-47584815 |
| chrX: | 55702081-55706752 |
| chrX: | 55702641-55704882 |
| chrX: | 56668707-56672393 |
| chrX: | 63046250-63049783 |
| chrX: | 63179793-63181342 |
| chrX: | 64659173-64662687 |
| chrX: | 67128184-67130234 |
| chrX: | 68723485-68726591 |
| chrX: | 68730873-68731378 |
| chrX: | 69284077-69289063 |
| chrX: | 69731696-69734585 |
| chrX: | 70137905-70139779 |
| chrX: | 70137905-70141317 |
| chrX: | 70375602-70376742 |
| chrX: | 70799156-70800146 |
| chrX: | 78921648-78925951 |
| chrX: | 78921816-78925294 |
| chrX: | 79298842-79303187 |
| chrX: | 80416515-80417443 |
| chrX: | 81173874-81175839 |
| chrX: | 81983127-81987718 |
| chrX: | 84148632-84150257 |
| chrX: | 85674775-85677046 |
| chrX: | 86089624-86090437 |
| chrX: | 88557172-88559847 |
| chrX: | 90226325-90227684 |
| chrX: | 93186836-93188015 |
| chrX: | 93401004-93404141 |
| chrX: | 94591646-94596454 |
| chrX: | 94698170-94701864 |
| chrX: | 95050849-95051472 |
| chrX: | 96606848-96608696 |
| chrX: | 102856941-102858799 |
| chrX: | 104417623-104418075 |
| chrX: | 105807016-105807818 |
| chrX: | 108353196-108356853 |
| chrX: | 109938628-109942201 |
| chrX: | 112149828-112152431 |
| chrX: | 114542555-114544543 |
| chrX: | 114543110-114544104 |
| chrX: | 116469495-116471079 |
| chrX: | 117454203-117454803 |
| chrX: | 120976226-120977717 |
| chrX: | 121946734-121949306 |
| chrX: | 122315627-122316237 |
| chrX: | 122372631-122373391 |
| chrX: | 123595967-123596512 |
| chrX: | 124991870-124994577 |
| chrX: | 126597916-126602440 |
| chrX: | 126598717-126602349 |
| chrX: | 127815565-127816864 |
| chrX: | 128500452-128502522 |
| chrX: | 129243875-129245369 |
| chrX: | 129667629-129668861 |
| chrX: | 129883745-129887979 |
| chrX: | 131005627-131008585 |
| chrX: | 131622967-131623775 |
| chrX: | 131939267-131941552 |
| chrX: | 133394520-133396998 |
| chrX: | 136186515-136189010 |
| chrX: | 142561963-142565186 |
| chrX: | 144422164-144424505 |
| chrX: | 144422234-144423104 |
| chrX: | 145052148-145054975 |
| chrX: | 145892851-145894555 |
| chrX: | 146038405-146041724 |
| chrX: | 146039816-146041436 |
| chrX: | 146040055-146042669 |
| chrX: | 149107022-149109390 |
| chrX: | 149927875-149928845 |
| chrX: | 150268515-150270823 |
| chrX: | 150294440-150295717 |
| chrX: | 150876929-150878609 |
| chrX: | 151083288-151087810 |
| chrX: | 151474441-151476876 |
| chrX: | 154485976-154486960 |
| chrX: | 154555636-154556862 |
| chrX: | 154778514-154782563 |
| chrX: | 154918257-154921714 |
| chrX: | 155089777-155092217 |

TABLE 3
Genomic regions covered by 280 bp oligonucleotide
spacing. Chromosome coordinates correspond
to the NA18507 human genome.

chr1: 8790600-8974770
chr1: 16869520-17090288
chr1: 20309286-20349544
chr1: 21730588-22255436
chr1: 24143221-24341601
chr1: 26761298-27554471
chr1: 31560929-31601751
chr1: 33496651-33537045
chr1: 35796809-35837817
chr1: 38342317-38382559
chr1: 40408910-40819394
chr1: 44549813-46180660
chr1: 50462844-51737264
chr1: 54982569-55023173
chr1: 58493475-58542371
chr1: 77574856-77615487
chr1: 80896457-81584812
chr1: 83663025-83797960
chr1: 87231009-87271289
chr1: 92519303-92560230
chr1: 93724408-93764889
chr1: 96893759-97165761
chr1: 101716151-102273691
chr1: 104119817-104300138
chr1: 109514961-110214902
chr1: 113413360-113462203
chr1: 116378624-117218832
chr1: 151972571-152071117
chr1: 154330611-155142814
chr1: 157023320-157063680
chr1: 160216060-160257271
chr1: 161356677-161397020
chr1: 165625525-165666427
chr1: 172671178-172711825
chr1: 179405008-179446629
chr1: 182890975-182949650
chr1: 191094581-191135870
chr1: 197086207-197127418
chr1: 202861033-202902663
chr1: 204295797-204336523
chr1: 208850547-209428019
chr1: 210420247-210460765
chr1: 212204817-212502971
chr1: 222622463-222713694
chr1: 224076390-224199145
chr1: 226606410-226647196
chr1: 228130876-229727480
chr1: 231233306-231274213
chr1: 235682290-235723344
chr1: 236963118-237003996
chr1: 238084435-238129152
chr1: 241063126-241104735
chr1: 243154810-243283882
chr1: 249200979-249231434
chr2: 10001-194430
chr2: 1763794-1804075
chr2: 4541431-4741730
chr2: 5768759-5809270
chr2: 11471796-12185434
chr2: 18423156-18464745
chr2: 23601547-24574930
chr2: 25915021-25955295
chr2: 27595751-27636560
chr2: 32027593-32068999
chr2: 33841589-33903948
chr2: 36506478-37179063
chr2: 38438748-38479558
chr2: 41363714-41405124
chr2: 42669146-42780319
chr2: 44062185-44835597
chr2: 50085716-50126685
chr2: 54236644-54299000
chr2: 55441955-55483110
chr2: 62027408-62809307
chr2: 63958466-64913416
chr2: 65873840-65915046
chr2: 68332405-68778020
chr2: 69801884-71430789
chr2: 73990988-74555279
chr2: 77881632-78660008
chr2: 85123040-85163995
chr2: 91596826-92287253
chr2: 95326172-96669432
chr2: 97659235-98464915
chr2: 100686338-101228005
chr2: 106843857-107335325
chr2: 117483381-117800878
chr2: 122445946-122486826
chr2: 126549705-127335964
chr2: 128525323-128995977
chr2: 130233855-133139593
chr2: 135703913-135744562
chr2: 138690360-139057285
chr2: 140954885-141001797
chr2: 143827597-143870118
chr2: 152022825-152475694
chr2: 156319411-156360257
chr2: 159689401-160566975
chr2: 162115373-162159276
chr2: 163988691-164029365
chr2: 168381880-168592936
chr2: 171437674-172464464
chr2: 174330365-175605647
chr2: 178063745-178104543
chr2: 181717413-181758350
chr2: 182805824-182846996
chr2: 183916475-183957426
chr2: 188670184-188711508
chr2: 190155866-190197353
chr2: 195032088-195073888
chr2: 197667143-198386305
chr2: 201907721-201949622
chr2: 203884781-204658536
chr2: 207263606-207305140
chr2: 212455769-212496151
chr2: 215691835-215732353
chr2: 217630369-219726940
chr2: 231359848-231400266
chr2: 232404551-232728889
chr2: 235534199-235577595
chr2: 238410055-238452998
chr2: 242685696-243102476
chr3: 1617343-1968162
chr3: 8694913-8755772
chr3: 10022212-10120145
chr3: 11509694-13115407
chr3: 15144793-15437780
chr3: 22403222-22445332
chr3: 23940675-23981067
chr3: 25771131-25811542
chr3: 32011180-32847768
chr3: 35237922-35278813
chr3: 37037981-37078510
chr3: 39356463-40761174
chr3: 41834748-42952228
chr3: 44867563-45221807
chr3: 48077487-48182878
chr3: 50736620-50777661
chr3: 57907597-57948357
chr3: 60655674-60738517
chr3: 63062261-63589687
chr3: 68665507-68706672
chr3: 72958285-73135109
chr3: 75243848-75934807
chr3: 80246223-80287063
chr3: 84940834-84981561
chr3: 87352608-88259138
chr3: 90252916-90293174
chr3: 93593661-93633922
chr3: 95404737-96357714
chr3: 97848170-97946704
chr3: 105772578-105812798
chr3: 110325517-110422005
chr3: 112222416-112264763
chr3: 113549296-113589732
chr3: 116726085-116766674
chr3: 118550486-121233878
chr3: 122362619-122403545
chr3: 123850692-123891475
chr3: 125401476-125670914
chr3: 128463389-128580091
chr3: 129698277-130036885
chr3: 131942325-131983241
chr3: 134050601-134091906
chr3: 136309361-136349700
chr3: 137800973-139234712
chr3: 141169383-141604673
chr3: 143215890-143257451
chr3: 145521937-145562663
chr3: 146974474-147905524
chr3: 149636368-149677784
chr3: 152765081-152806694
chr3: 155685375-155727034
chr3: 156860330-156901463
chr3: 161126824-161167833
chr3: 163398296-163438612
chr3: 166763663-166804624
chr3: 170193156-170234252
chr3: 175415812-175456585
chr3: 178190963-178231549
chr3: 182308405-182349772
chr3: 183585586-183766003
chr3: 184807675-185297180
chr3: 186598137-186859118
chr3: 194546161-197962430
chr4: 10001-487049
chr4: 9324643-9777599
chr4: 13612744-13653905
chr4: 17043530-17934764
chr4: 19795289-19835990
chr4: 22575806-22617061
chr4: 24753221-24794474
chr4: 33837721-33878692
chr4: 34907690-34948364
chr4: 36425831-37843565
chr4: 43391975-43433066
chr4: 53986360-54873661
chr4: 57202480-57597215
chr4: 73653139-73693654
chr4: 74783931-74825753
chr4: 77982717-78930232
chr4: 80488891-81103385
chr4: 83391962-83432921
chr4: 97998755-98967592
chr4: 100891116-100932667
chr4: 103630597-103904991
chr4: 106038422-106427612
chr4: 111300129-111341352
chr4: 113466353-114156493
chr4: 116822427-117241493
chr4: 119319160-120394577
chr4: 128713805-128754889
chr4: 130061511-130102063
chr4: 132574051-132919357
chr4: 135853276-135988208
chr4: 140598693-140639889
chr4: 144250367-144983976
chr4: 151560924-152044514
chr4: 156365126-156407717
chr4: 160840403-160880657
chr4: 165160742-165954138
chr4: 171096835-171137125
chr4: 174320482-174361480
chr4: 185453527-185511760
chr4: 189250538-191026422
chr5: 10001-2167223
chr5: 7270822-7327768
chr5: 13726031-14673599
chr5: 18726424-18766791
chr5: 21264862-22226427
chr5: 23951348-25210158
chr5: 29050889-29467813
chr5: 31802704-31868672
chr5: 34109648-34274237
chr5: 36865258-37507765
chr5: 40780777-40821021
chr5: 42872170-43887470
chr5: 49891744-49957982
chr5: 55220435-55821218
chr5: 59734870-60060971
chr5: 65847877-65888699
chr5: 68831348-70755275
chr5: 72001187-72727180
chr5: 74157251-75558328
chr5: 76821799-77102533
chr5: 78559877-79968419
chr5: 81284637-82293305
chr5: 84275412-84317028
chr5: 85558167-85598985
chr5: 92998203-93039856
chr5: 94141590-94182535
chr5: 96371266-96412210
chr5: 97708529-99753395
chr5: 108904270-108945462
chr5: 110507837-110548770
chr5: 115086506-115574670
chr5: 117094346-117135482
chr5: 118290041-118903878
chr5: 120987178-121028253
chr5: 122716751-122993270
chr5: 133287657-133779187
chr5: 135744768-135786008
chr5: 137789396-138391431
chr5: 141255723-141296361
chr5: 142692147-142733764
chr5: 145088159-146107699
chr5: 149381093-149494793
chr5: 153697393-153737808
chr5: 165435404-165477316
chr5: 167265585-167305979
chr5: 169547608-169589678
chr5: 170773304-170858317
chr5: 172632746-172673757
chr5: 173968810-174369278
chr5: 175439549-177966596
chr5: 179101457-179142126
chr5: 180680472-180904324
chr6: 60001-174870
chr6: 5588676-5630634
chr6: 10098712-10595690
chr6: 16594024-17552226
chr6: 19123834-19634548
chr6: 23982022-25282071
chr6: 26712532-30685395
chr6: 32272974-32314310
chr6: 34564292-35372549
chr6: 37039175-37079973
chr6: 41614760-41655792
chr6: 42833966-43527565
chr6: 50860530-50901275
chr6: 54469513-54510448
chr6: 56716020-58756674
chr6: 63917761-64279577
chr6: 70394777-71176051
chr6: 74207206-74250306
chr6: 76008211-76049364
chr6: 80043296-80084316
chr6: 81063623-81104418
chr6: 84082991-84124090
chr6: 85977849-87800951
chr6: 100478154-100519303
chr6: 107070263-107111189
chr6: 109627541-109669258
chr6: 112657117-112703064
chr6: 114385361-115339376
chr6: 116758412-116800494
chr6: 118300115-118340844
chr6: 121709659-122021607
chr6: 131999932-132052516
chr6: 133965178-134640021
chr6: 151344965-151567576
chr6: 153007254-153047847
chr6: 154565036-154605556
chr6: 159927005-161088762
chr6: 165856715-166770058
chr6: 167781627-168710336
chr7: 2773292-2813773
chr7: 5036885-7061218
chr7: 10485969-11066588
chr7: 15721011-15761511
chr7: 20022289-20063832
chr7: 21266022-21306308
chr7: 22529852-22833644
chr7: 25142591-25183498
chr7: 26941750-27043924
chr7: 30542962-30590017
chr7: 32695400-33011673
chr7: 35029938-36879722
chr7: 39781955-39858212
chr7: 42133046-45882836
chr7: 50891180-51503439
chr7: 53169313-53259342
chr7: 54704174-57958873
chr7: 61054332-66784844
chr7: 72311876-72749446
chr7: 84486118-84634731
chr7: 87130884-87172231
chr7: 88249723-88290046
chr7: 93279072-93319352
chr7: 94304278-94345256
chr7: 97476729-98104062
chr7: 99880192-100867949
chr7: 101955737-102943822
chr7: 106251137-106293291
chr7: 108130637-108171602
chr7: 112595746-113153647
chr7: 120671270-121059660
chr7: 122301346-122341800
chr7: 124920519-124961273
chr7: 128086503-128319061
chr7: 131185016-131460717
chr7: 132833829-132875483
chr7: 136603022-137219082
chr7: 138353841-138394377
chr7: 149011227-149758910
chr7: 151899158-152319699
chr7: 158102620-158135427
chr8: 10001-1541963
chr8: 4908923-4950674
chr8: 9495725-9536049
chr8: 26848016-26891318
chr8: 28137259-30228009
chr8: 32848956-34752759
chr8: 39697174-39738175
chr8: 41349093-43375736
chr8: 47488126-49318327
chr8: 51654214-51695526
chr8: 52710710-52753796
chr8: 54425832-54466711
chr8: 55588530-55629932
chr8: 56993572-58146000
chr8: 59396151-59521801
chr8: 62094909-62984761
chr8: 66269964-66311149
chr8: 68077469-68394565
chr8: 70878973-70920118
chr8: 73939340-75981656
chr8: 79652776-79694815
chr8: 81450985-83224716
chr8: 90235543-90275896
chr8: 93136143-93176788
chr8: 95194125-95622587
chr8: 98616374-98657766
chr8: 101887918-102594869
chr8: 103915679-103957078
chr8: 107581537-107622597
chr8: 115021674-115062490
chr8: 118515403-118556065
chr8: 119753958-119794785
chr8: 125897408-125937777
chr8: 129819287-129859553
chr8: 134400237-134676593
chr8: 137429333-137898647
chr8: 142449097-142490938
chr8: 144776755-144817112
chr8: 146282081-146302361
chr9: 29078-825826
chr9: 4924058-5332824
chr9: 6619111-7618686
chr9: 9070850-9111524
chr9: 14184259-14225133
chr9: 15341470-15382329
chr9: 21146068-21388303
chr9: 27588148-28170472
chr9: 30055316-30852270
chr9: 33377966-33679756
chr9: 78993594-79184829
chr9: 80775052-80815709
chr9: 85025612-85069479
chr9: 86673573-86714704
chr9: 90508425-91046530
chr9: 92954953-93535225
chr9: 94845145-95682336
chr9: 96780418-97270319
chr9: 99874087-99996318
chr9: 102858933-102900057
chr9: 110198467-110240812
chr9: 112767090-112807784
chr9: 114350035-115868680
chr9: 123236976-123277486
chr9: 124261557-124302880
chr9: 125580338-125621237
chr9: 127936912-128379337
chr9: 130998105-131477845
chr9: 135874810-135916660
chr9: 136964079-137004403
chr9: 141045616-141152693
chr10: 60001-1303151
chr10: 3479425-3520998
chr10: 8535835-8577323
chr10: 13079860-13121002
chr10: 14745092-15217462
chr10: 19758194-20057675
chr10: 21382045-21422879
chr10: 23405832-23447365
chr10: 26860959-33367460
chr10: 35787872-39096695
chr10: 56011841-56200170
chr10: 57382581-58218999
chr10: 65642110-65683492
chr10: 69533442-70455040
chr10: 73930028-77114181
chr10: 79474269-79517773
chr10: 85821124-86341174
chr10: 88370560-89423350
chr10: 94415657-95064742
chr10: 97334344-99213610
chr10: 100787351-101893013
chr10: 104312390-104997165
chr10: 107427419-107468059
chr10: 111547449-112717003
chr10: 114288011-114328778
chr10: 116429668-116470546
chr10: 118178730-118219597
chr10: 120611437-120652252
chr10: 122094114-122134808
chr10: 124315929-124951408
chr10: 126536053-126576656
chr10: 127574369-127629646
chr10: 129302740-129343302
chr10: 131884427-131927923
chr10: 134366388-135521019
chr11: 1875765-1916774
chr11: 3253109-6036538
chr11: 7785247-7826119
chr11: 9607634-10552792
chr11: 13611094-13652427
chr11: 14988853-15030924
chr11: 16976097-17270856
chr11: 18263552-18305265
chr11: 20576112-20617005
chr11: 29727369-29768257
chr11: 31281582-31322696
chr11: 35861706-35903248
chr11: 41306004-41346575
chr11: 43524655-43761256
chr11: 45515745-47233324
chr11: 55009895-56145855
chr11: 57323703-57506217
chr11: 58814354-58855288
chr11: 61712013-62903960
chr11: 66335445-66375692
chr11: 67463650-67783577
chr11: 70858724-71635335
chr11: 73271267-74811419
chr11: 76042313-76084192
chr11: 77425368-77600453
chr11: 80253521-80294246
chr11: 86523541-86589062
chr11: 89491310-89902690
chr11: 94626256-94667691
chr11: 100517227-100558850
chr11: 103255575-103301739
chr11: 104923243-104993417
chr11: 108672473-108713225
chr11: 110638444-110680402
chr11: 112117702-112159023
chr11: 114453833-114516484
chr11: 118411365-118452458
chr11: 119454252-119495193
chr11: 123915528-123956122
chr11: 125713209-125733443
chr12: 1846310-3341225
chr12: 6498665-6539303
chr12: 7925616-13195045
chr12: 14353991-14394510
chr12: 17123584-17165320
chr12: 18825764-19630779
chr12: 27396022-27782281
chr12: 29429282-29491718
chr12: 30994800-32121918
chr12: 33587459-34388188
chr12: 37856695-38753700
chr12: 39840413-39881264
chr12: 42600711-44075332
chr12: 45959104-45999405
chr12: 48707461-48748034
chr12: 52086710-55216451
chr12: 56884941-56927044
chr12: 58357127-58398492
chr12: 62253361-62293861
chr12: 63928258-64163520
chr12: 65241788-66452144
chr12: 68926612-69121189
chr12: 70575681-70615931
chr12: 76973954-77074747
chr12: 80402674-80444422
chr12: 83525520-83565990
chr12: 93257462-94055629
chr12: 95840948-95881783
chr12: 98964217-99209552
chr12: 100382762-100589378
chr12: 101798208-101838624
chr12: 104404850-105391956
chr12: 106389713-106432302
chr12: 107645814-107687455
chr12: 112719556-112865595
chr12: 118664010-118704963
chr12: 120789273-121375187
chr12: 122829220-122870368
chr12: 127236920-127277376
chr12: 129852222-129894064
chr12: 131592004-132132569
chr12: 133249449-133270371
chr13: 19020001-20160508
chr13: 21502083-21556515
chr13: 23250002-23290612
chr13: 24496545-25554352
chr13: 27131126-27171969
chr13: 28250146-28810441
chr13: 29861783-29902227
chr13: 31015013-31055712
chr13: 35603920-35666779
chr13: 41308235-42037969
chr13: 45931633-47055444
chr13: 50444946-50768224
chr13: 52461888-53415268
chr13: 54994812-55035573
chr13: 57242547-57768483
chr13: 59083398-59606176
chr13: 60759830-60800097
chr13: 64296428-64432323
chr13: 66341677-66383461
chr13: 67820907-68506250
chr13: 71845283-72270456
chr13: 73257749-73298507
chr13: 75791840-75834832
chr13: 77535198-78258196
chr13: 81259968-82285327
chr13: 90465631-90904095
chr13: 91904044-91944972
chr13: 93305320-94068635
chr13: 96251527-96482049
chr13: 100782636-101212627
chr13: 102895208-102936464
chr13: 105446766-105487670
chr13: 112911651-112990202
chr14: 19036796-21429571
chr14: 23143249-23753111
chr14: 25715393-25756734
chr14: 28198464-28325774
chr14: 31911937-31952842
chr14: 35189453-36862391
chr14: 39557507-39647815
chr14: 47649752-47690470
chr14: 51884378-52625372
chr14: 55451476-56031015
chr14: 58731583-59282095
chr14: 61418098-61459444
chr14: 64014774-64088869
chr14: 65713262-67248475
chr14: 68785488-71082932
chr14: 74450073-74865457
chr14: 77080069-77122246
chr14: 78129482-78170023
chr14: 81268792-81310060
chr14: 84617770-84661099
chr14: 86380539-86421005
chr14: 90812959-90853692
chr14: 92473304-92514721
chr14: 95172510-95287953
chr14: 96669453-96710248
chr14: 100927538-100968555
chr14: 103108455-103861442
chr14: 105282238-107289333
chr15: 32659369-33325081
chr15: 34651150-35715033
chr15: 40166195-42031003
chr15: 43074033-43428977
chr15: 45827959-45869400
chr15: 47404850-48043128
chr15: 49751236-49801017
chr15: 53157825-53251237
chr15: 55453689-55494654
chr15: 57518959-57559438
chr15: 59141755-60717342
chr15: 62522887-62674798
chr15: 64865110-64943826
chr15: 66088113-67313635
chr15: 68526991-68568168
chr15: 71130969-71654331
chr15: 72651450-73213266
chr15: 74337650-79981537
chr15: 89658547-91596892
chr15: 92809054-93276262
chr15: 99804490-100357885
chr15: 102256937-102519489
chr16: 416971-3442423
chr16: 5128325-5362730
chr16: 9230183-9270912
chr16: 11101088-12056855
chr16: 14761396-16509096
chr16: 18006529-18953900
chr16: 21390310-22574821
chr16: 25445209-26064511
chr16: 27124595-27166756
chr16: 28330655-35057751
chr16: 51659661-51701297
chr16: 53376175-53432860
chr16: 55733566-55774469
chr16: 60371491-60411785
chr16: 69372511-70302604
chr16: 72743092-72784033
chr16: 73954474-74447296
chr16: 77906376-77947560
chr16: 81489603-82489368
chr16: 87281789-88194252
chr16: 90136558-90293161
chr17: 117291-2371158
chr17: 3548410-3589080
chr17: 4588103-5063536
chr17: 8443845-8485023
chr17: 11894183-11934799
chr17: 15725691-31270135
chr17: 40884695-41318415
chr17: 42422184-42463250
chr17: 43568345-45639606
chr17: 46748504-46972820
chr17: 48294004-48501715
chr17: 49559766-49600715
chr17: 51163009-52052920
chr17: 56532667-59167786
chr17: 60282779-61569790
chr17: 62834172-62914425
chr17: 73143836-73184302
chr17: 74322125-74362927
chr17: 80609024-80630450
chr18: 10001-125247
chr18: 3386264-3426958
chr18: 4982456-5023393
chr18: 6442077-6483131
chr18: 9658098-9699642
chr18: 11597193-11823805
chr18: 12959889-15401638
chr18: 18678951-20018205
chr18: 21232136-21310254
chr18: 23814242-23855462
chr18: 29971384-30242168
chr18: 36500787-36935754
chr18: 41880930-41921944
chr18: 44522213-44582394
chr18: 47329174-47373385
chr18: 49118270-49159577
chr18: 55485868-56673551
chr18: 57662689-58352887
chr18: 60062942-60103589
chr18: 62256413-62296677
chr18: 68077627-68119086
chr18: 70266275-70307143
chr18: 74526286-74567014
chr18: 76252879-76611924
chr19: 60001-898455
chr19: 2328636-2368850
chr19: 6573383-7075578
chr19: 8818972-9392726
chr19: 11743088-12639474
chr19: 13648956-13689309
chr19: 14932469-15023823
chr19: 17680394-17721031
chr19: 19897290-24330769
chr19: 30372525-30432590
chr19: 34060075-35044663
chr19: 36727158-37814809
chr19: 40133104-40274200
chr19: 41451765-42335438
chr20: 800046-2076643
chr20: 3321241-5582648
chr20: 16188968-16229866
chr20: 17477582-18486382
chr20: 19783991-19825205
chr20: 21126732-22734878
chr20: 23939044-23996755
chr20: 25328625-26278839
chr20: 30554464-30778744
chr20: 32690500-32839286
chr20: 34445840-36179238
chr20: 37585988-37627257
chr20: 40156163-40197206
chr20: 41839415-41880352
chr20: 44249019-44394665
chr20: 45769365-47155351
chr20: 48553421-49768371
chr20: 53671094-53712502
chr20: 55014196-56084286
chr20: 61915592-62946839
chr21: 10865076-11187874
chr21: 14393433-15370222
chr21: 19073347-19113586
chr21: 20210137-20251257
chr21: 29197173-29443591
chr21: 33970240-34014534
chr21: 37368292-37408976
chr21: 40479638-40735277
chr21: 48080444-48111324
chr22: 16050001-25118225
chr22: 26936438-26978194
chr22: 29052424-29110866
chr22: 31968374-32703148
chr22: 36214294-36837789
chr22: 38737297-38786398
chr22: 40482194-42125680
chr22: 43152321-43193667
chr22: 44178404-45692117
chr22: 46942817-46984749
chr22: 49292076-50172759
chr22: 51204731-51242535
chrX: 5661608-5702110
chrX: 9352364-9402723
chrX: 14832191-14873686
chrX: 16196532-16237531
chrX: 24352552-24827311
chrX: 26685422-26726253
chrX: 28656618-28697557
chrX: 30615564-30856393
chrX: 32203986-32245348
chrX: 36372828-36413458
chrX: 39626302-39667771
chrX: 40761563-41555929
chrX: 43246541-43288091
chrX: 44580215-46320320
chrX: 47680150-49476280
chrX: 50628492-50669634
chrX: 52217785-55230468
chrX: 56249270-56290061
chrX: 62387892-62428208
chrX: 64972212-65314875
chrX: 67799979-68415341
chrX: 70896653-74625305
chrX: 76281374-78040188
chrX: 80236390-80279049
chrX: 82982944-83025259
chrX: 83991146-84032852
chrX: 85394917-85436409
chrX: 86938230-87175748
chrX: 88992939-89033274
chrX: 91216800-92563993
chrX: 99390762-100719297
chrX: 106651899-106692851
chrX: 109568889-109821243
chrX: 114581999-116118608
chrX: 118334657-118376210
chrX: 119986660-120137536
chrX: 122628341-123435715
chrX: 125585620-125885925
chrX: 127828902-127869893
chrX: 132876780-132917874
chrX: 134075823-135894109
chrX: 139095404-140693475
chrX: 144238334-144349288
chrX: 147113308-147567450
chrX: 150142235-150212423
chrX: 151627950-154065915
chrX: 155229867-155258041
chrY: 1866748-1907681
chrY: 9653663-10050125
chrY: 13246305-13660991
chrY: 23577104-25184117
chrY: 26295302-28603437
chrY: 58897363-58917605
chr1: 1-847114
chr1: 2522081-2713577
chr1: 12891264-13747803
chr1: 120377389-121162199
chr1: 143424333-149760173
chr1: 206138911-206720864
chr2: 86900414-89385283
chr2: 108343285-114500974
chr4: 3578596-4190530
chr4: 47487410-49591421
chr4: 69102905-70360767
chr5: 16742337-17800241
chr6: 57871736-58567172
chr6: 170060759-171006035
chr7: 35000-160000
chr7: 74072030-76924519
chr7: 141490017-144533146
chr8: 6752221-8096446
chr8: 11811446-12612992
chr8: 86534680-86861303
chr9: 36336401-46748385
chr9: 65494386-70920000
chr10: 18041218-18200091
chr10: 44172511-52383737
chr10: 81272357-81965328
chr11: 40001-207422
chr11: 48997050-51412448
chr13: 114239588-115092802
chr15: 21326212-25244225
chr15: 28000023-31239503
chr15: 82619908-83736106
chr15: 85045806-85789771
chr16: 46385802-46528907
chr17: 34181960-34946274
chr17: 36344876-39280419
chr17: 65821780-66132070
chr17: 77704882-78973933
chr20: 29421942-29652106
chrX: 10000-1781973
chrY: 10000-901974
chrY: 58847818-58908587

TABLE 4
Genomic regions covered by 300 bp oligonucleotide
spacing. Chromosome coordinates correspond
to the NA18507 human genome.

chr1: 4597917-4603368
chr1: 6438194-6445835
chr1: 6787516-6793395
chr1: 8182444-8191375
chr1: 8204084-8211127
chr1: 8673057-8682897
chr1: 8715188-8720239
chr1: 8766443-8774912
chr1: 10369418-10378885
chr1: 11097562-11106051
chr1: 16854043-16863594
chr1: 17175722-17181610
chr1: 24895796-24901144
chr1: 25688689-25695235
chr1: 26459570-26464632
chr1: 30688669-30693813
chr1: 34406948-34412032
chr1: 35102307-35111970
chr1: 39433145-39440637
chr1: 41021207-41028481
chr1: 41991470-41997984
chr1: 46244285-46251346
chr1: 47565590-47574182
chr1: 53580640-53568914
chr1: 55089225-55095974
chr1: 60046731-60052368
chr1: 62113371-62119744
chr1: 69996393-70002209
chr1: 71244106-71249573
chr1: 72755371-72764036
chr1: 77106208-77112305
chr1: 77774744-77781454
chr1: 83807765-83814646
chr1: 90169037-90174165
chr1: 99856525-99865334
chr1: 100194890-100204463
chr1: 104103686-104113237
chr1: 106015813-106023397
chr1: 107095215-107102345
chr1: 107201327-107211256
chr1: 107655304-107663735
chr1: 110252339-110259004
chr1: 111929021-111934897
chr1: 118069772-118075824
chr1: 120104966-120113946
chr1: 120142387-120150241
chr1: 142535521-142542934
chr1: 152185909-152193469
chr1: 159012766-159019831
chr1: 159118351-159123995
chr1: 160953742-160960609
chr1: 174796614-174801850
chr1: 179329828-179335094
chr1: 180750406-180755506
chr1: 182262748-182269906
chr1: 182776637-182781883
chr1: 187399799-187405049
chr1: 187715748-187722278
chr1: 187939362-187947395
chr1: 188325018-188330315
chr1: 189071908-189077598
chr1: 189772130-189777709
chr1: 189777391-189786094
chr1: 190638400-190646807
chr1: 192675218-192683546
chr1: 194936155-194946091
chr1: 195013954-195019705
chr1: 195359744-195365244
chr1: 195526227-195531417
chr1: 198305134-198310536
chr1: 205917508-205922673
chr1: 207741538-207746586
chr1: 210077993-210085962
chr1: 213004863-213013140
chr1: 213314571-213320124
chr1: 218917580-218922769
chr1: 222373943-222380499
chr1: 224199356-224204485
chr1: 225075528-225081091
chr1: 226375850-226384008
chr1: 229812526-229820631
chr1: 232890253-232899241
chr1: 240219317-240225042
chr1: 243120356-243130355
chr1: 249144921-249150547
chr2: 207761-215529
chr2: 1218833-1227243
chr2: 1528334-1535327
chr2: 1533892-1542761
chr2: 6795702-6805395
chr2: 7230036-7235715
chr2: 10150554-10157686
chr2: 10433505-10439183
chr2: 11395030-11400620
chr2: 13087432-13093196
chr2: 13555202-13561869
chr2: 14190096-14199486
chr2: 14704166-14710045
chr2: 17584080-17592046
chr2: 18260251-18267997
chr2: 23153867-23159752
chr2: 29958037-29963288
chr2: 31403351-31411711
chr2: 34303765-34313027
chr2: 36332589-36339555
chr2: 41776008-41781174
chr2: 48851209-48857846
chr2: 49533720-49539282
chr2: 49653686-49662187
chr2: 51772538-51781819
chr2: 53631630-53887764
chr2: 56649400-56655700
chr2: 57842228-57849900
chr2: 59623296-59631007
chr2: 66099629-66105015
chr2: 66978344-66983484
chr2: 67759060-67766216
chr2: 79331533-79339762
chr2: 84100928-84106703
chr2: 92292052-92300176
chr2: 96727246-96733710
chr2: 106347837-106353279
chr2: 123477291-123482469
chr2: 125639339-125645009
chr2: 126378317-126385964
chr2: 126443174-126451968
chr2: 129638490-129646581
chr2: 147941645-147946792
chr2: 150621365-150626697
chr2: 151031148-151038204
chr2: 164438272-164443520
chr2: 165855413-165865029
chr2: 167494647-167501455
chr2: 172832837-172840816
chr2: 177265710-177271922
chr2: 178678786-178684838
chr2: 180067718-180072888
chr2: 180412903-180422173
chr2: 184085446-184090996
chr2: 186741423-186747368
chr2: 187844264-187850418
chr2: 188384126-188389392
chr2: 189267263-189277181
chr2: 189567840-189573327
chr2: 194689555-194697784
chr2: 200178423-200183555
chr2: 205343819-205349083
chr2: 205706215-205711954
chr2: 207860723-207865763
chr2: 208350852-208359404
chr2: 212801927-212809271
chr2: 213183953-213192002
chr2: 226453568-226462957
chr2: 226955605-226962339
chr2: 227165362-227171023
chr2: 227342447-227347452
chr2: 228488428-228494889
chr2: 234492605-234501401
chr2: 234569283-234575116
chr2: 235442849-235450064
chr2: 238554030-238559382
chr2: 241914865-241922196
chr3: 228631-234368
chr3: 528304-534054
chr3: 990695-996780
chr3: 1032760-1039425
chr3: 4192417-4201134
chr3: 4932839-4938174
chr3: 9359852-9365532
chr3: 16580617-16589915
chr3: 18946005-18953294
chr3: 20311112-20317882
chr3: 22279439-22286398
chr3: 22941523-22946888
chr3: 25850020-25855058
chr3: 26432273-28439460
chr3: 28810031-28817818
chr3: 30994742-30999883
chr3: 36711780-36719477
chr3: 37978470-37986876
chr3: 41587741-41595624
chr3: 41779107-41789001
chr3: 43053229-43059805
chr3: 45543287-45552064
chr3: 49721171-49727086
chr3: 49726377-49733587
chr3: 50343466-50349348
chr3: 50945260-50955050
chr3: 58798489-58808349
chr3: 60050980-60057155
chr3: 68631989-68640250
chr3: 68635089-68640250
chr3: 68739499-68747867
chr3: 72779987-72786060
chr3: 74147583-74154361
chr3: 74807887-74812932
chr3: 77337641-77346005
chr3: 85878639-85884972
chr3: 86848288-86855189
chr3: 89670061-89678063
chr3: 97739466-97794710
chr3: 98726549-98736401
chr3: 98943596-98949668
chr3: 99207484-99216623
chr3: 103464803-103470992
chr3: 103593873-103603147
chr3: 104293536-104300500
chr3: 110694073-110699196
chr3: 125672365-125679046
chr3: 125712288-125722078
chr3: 125944521-125952894
chr3: 129076142-129083883
chr3: 131602136-131607141
chr3: 131708261-131713399
chr3: 131989585-131995012
chr3: 133016100-133025982
chr3: 133132004-133137072
chr3: 136020773-136026295
chr3: 146385189-146390341
chr3: 148963614-148969711
chr3: 153465040-153470192
chr3: 160680271-160685991
chr3: 161308602-161816680
chr3: 163658959-183665126
chr3: 164117871-164125977
chr3: 164302588-164311775
chr3: 166086031-166094634
chr3: 169352223-169359647
chr3: 173209418-173214445
chr3: 189092017-189100061
chr3: 189363208-189371038
chr3: 190514429-190521024
chr3: 191064731-191071632
chr3: 191988483-191996689
chr3: 192304880-192312848
chr3: 192373677-192378988
chr3: 192777384-192782563
chr3: 193136250-193142870
chr4: 553618-560788
chr4: 3059756-3069436
chr4: 3468004-3474478
chr4: 5614696-5620033
chr4: 5889661-5895891
chr4: 6895182-6900570
chr4: 7218321-7225836
chr4: 12153942-12162848
chr4: 15760451-15768822
chr4: 19858388-19864369
chr4: 21081080-21086557
chr4: 21137385-21142485
chr4: 21369093-21377090
chr4: 28943964-28953141
chr4: 29034157-29040382
chr4: 32985487-32994374
chr4: 33378223-33386666
chr4: 34683608-34688623
chr4: 35146818-35152167
chr4: 35848130-35856605
chr4: 39116220-39122927
chr4: 42762742-42769519
chr4: 44018298-44027195
chr4: 44323795-44331070
chr4: 45521400-45527595
chr4: 58567133-58573027
chr4: 58811892-58818426
chr4: 60288872-60294403
chr4: 60324904-60330935
chr4: 63669562-63676380
chr4: 70467481-70474320
chr4: 73418782-73426022
chr4: 73552118-73558447
chr4: 75078561-75083647
chr4: 79256788-79263029
chr4: 79421607-79431447
chr4: 90101108-90107506
chr4: 96701137-96708229
chr4: 97319268-97325686
chr4: 97729123-97736626
chr4: 105266318-105273759
chr4: 106577867-106583120
chr4: 106708350-106717147
chr4: 107056530-107063322
chr4: 108152739-103162227
chr4: 110840754-110845757
chr4: 112236730-112242626
chr4: 115175100-115184162
chr4: 115507882-115514003
chr4: 122282239-122290674
chr4: 137359061-137364851
chr4: 138092026-138100754
chr4: 138324141-138333392
chr4: 143616010-143621320
chr4: 143794391-143800941
chr4: 145000210-145009953
chr4: 146918610-146927658
chr4: 154758172-154765406
chr4: 156673835-156678864
chr4: 156957038-156966800
chr4: 156965114-156973966
chr4: 161879032-161884945
chr4: 168109174-168115505
chr4: 172374403-172379633
chr4: 172645715-172651777
chr4: 173425019-173430516
chr4: 173425019-173434070
chr4: 176030572-176039563
chr4: 178052183-178058019
chr4: 178884836-178893123
chr4: 179240667-179246334
chr4: 179286761-179296162
chr4: 179516956-179522448
chr4: 187162949-187168923
chr4: 187844016-187850486
chr5: 2769433-2777414
chr5: 8017606-8024076
chr5: 12810977-12820331
chr5: 13415392-13422923
chr5: 14995242-15000583
chr5: 16650304-16660144
chr5: 19223089-19232232
chr5: 19277926-19287636
chr5: 20419954-20428886
chr5: 20436963-20444354
chr5: 26796702-26801907
chr5: 28216623-28225329
chr5: 28245483-28253082
chr5: 28927599-28932729
chr5: 45004762-45014494
chr5: 46214782-46220269
chr5: 46270448-46276087
chr5: 53053775-53059759
chr5: 59489615-59496332
chr5: 63697966-63703697
chr5: 76317796-76323028
chr5: 83943432-83955098
chr5: 90618564-90623946
chr5: 106248534-106255784
chr5: 108538428-108543579
chr5: 110595369-110600573
chr5: 110905931-110911040
chr5: 111939492-111944907
chr5: 112666841-112672251
chr5: 114255282-114261315
chr5: 114255699-114262577
chr5: 114326170-114334013
chr5: 117141677-117147812
chr5: 117387158-117395999
chr5: 117868237-117878164
chr5: 123134815-123140614
chr5: 126195468-126201782
chr5: 127895818-127904284
chr5: 128627133-128632392
chr5: 134258136-134264715
chr5: 135115252-135120861
chr5: 140096794-140105317
chr5: 140215713-140222054
chr5: 140554460-140559847
chr5: 142016241-142021453
chr5: 147701022-147706063
chr5: 147742531-147749953
chr5: 150203369-150211385
chr5: 155948588-155954267
chr5: 167118388-167126431
chr5: 178258606-178266167
chr6: 601190-607388
chr6: 3253917-3262554
chr6: 3712581-3719680
chr6: 8924868-8932953
chr6: 11477421-11483532
chr6: 11786468-11791797
chr6: 12008060-12013799
chr6: 14582102-14591012
chr6: 18106851-18112902
chr6: 19041269-19049510
chr6: 21825501-21834188
chr6: 23460350-23485391
chr6: 26343849-26351580
chr6: 26677369-26683871
chr6: 30982075-30989830
chr6: 31229143-31235839
chr6: 31270366-31279433
chr6: 31308595-31313661
chr6: 31318916-31325710
chr6: 31336878-31344406
chr6: 31952447-31958916
chr6: 31985141-31991744
chr6: 32468816-32474512
chr6: 32474972-32481643
chr6: 32507644-32513014
chr6: 32540154-32546207
chr6: 32550496-32559809
chr6: 32560862-32570412
chr6: 32587519-32594357
chr6: 32600591-32607907
chr6: 32651571-32657522
chr6: 32664617-32672914
chr6: 32689075-32697955
chr6: 33048360-33054740
chr6: 33289511-33296285
chr6: 33937823-33943036
chr6: 33963108-33969161
chr6: 38495659-38504443
chr6: 40829246-40838674
chr6: 48745206-48753735
chr6: 48840995-48846579
chr6: 48898154-48904303
chr6: 48930877-48938505
chr6: 51040323-51046059
chr6: 51668875-51675620
chr6: 51801347-51808428
chr6: 52797421-52803827
chr6: 53928764-53934834
chr6: 54299305-54305752
chr6: 54848098-54851665
chr6: 55907704-55912800
chr6: 57622861-57631223
chr6: 58618325-58624205
chr6: 58773521-58780132
chr6: 62200459-62206207
chr6: 63219158-63225196
chr6: 67210350-67215491
chr6: 74592011-74601507
chr6: 74708924-74716855
chr6: 76155476-76160886
chr6: 77097591-77103692
chr6: 77439787-77449423
chr6: 79521092-79530501
chr6: 81283740-81293537
chr6: 81507242-81514132
chr6: 94963053-94969110
chr6: 108026135-108035102
chr6: 108029706-108036977
chr6: 121512246-121517842
chr6: 121519481-121526100
chr6: 125588935-125595914
chr6: 131809780-131816116
chr6: 139602605-139607757
chr6: 141774668-141781657
chr6: 142043388-142050538
chr6: 146733815-146739150
chr6: 153924584-153932603
chr6: 165406753-165416511
chr6: 165724709-165731958
chr6: 167613861-167622008
chr6: 167729012-167736167
chr6: 168726791-168733853
chr6: 168778664-168784785
chr7: 54629-60367
chr7: 699727-704942
chr7: 1703807-1710491
chr7: 1855970-1863481
chr7: 3610530-3618955
chr7: 4073333-4082299
chr7: 8230639-8236558
chr7: 12003338-12010123
chr7: 13022272-13028540
chr7: 21763945-21769647
chr7: 26137323-26145721
chr7: 29824540-29831970
chr7: 37931024-37936701
chr7: 39546315-39551777
chr7: 47283653-47290258
chr7: 51591210-51598231
chr7: 51595127-51603727
chr7: 51742923-51751752
chr7: 53322982-53328678
chr7: 54380642-54388276
chr7: 70195944-70203118
chr7: 72270097-72275581
chr7: 81107235-81113960
chr7: 87864416-87869830
chr7: 89520116-89525708
chr7: 91032992-91042557
chr7: 92994313-93000853
chr7: 93326803-93332386
chr7: 96505363-96513731
chr7: 97396010-97402601
chr7: 101001018-101007033
chr7: 110181971-110188439
chr7: 111251272-111257116
chr7: 113652813-113659255
chr7: 115931547-115941121
chr7: 118332903-118391350
chr7: 118590992-118599039
chr7: 122856064-122861804
chr7: 126045909-126051433
chr7: 126271757-126278927
chr7: 126776235-126781390
chr7: 131774499-131780614
chr7: 146133751-148139658
chr7: 146366572-146371757
chr7: 151223180-151231642
chr7: 152574722-152580274
chr7: 154392156-154399657
chr7: 154448379-154455966
chr7: 155135570-155141554
chr7: 155138626-155143663
chr7: 156387081-156394378
chr7: 158496115-158504942
chr7: 159117388-159122697
chr8: 1835429-1840933
chr8: 2077972-2085486
chr8: 2129537-2134915
chr8: 2253695-2263666
chr8: 2316905-2323505
chr8: 3994899-4004472
chr8: 4953041-4962888
chr8: 8583122-8589371
chr8: 13599265-13609200
chr8: 14262187-14267720
chr8: 14501601-14506603
chr8: 14507036-14515214
chr8: 16201251-16207498
chr8: 17309962-17315589
chr8: 21329823-21337031
chr8: 23202200-23207249
chr8: 24145153-24151166
chr8: 25410668-25416618
chr8: 35195413-35201496
chr8: 36273816-36279095
chr8: 37390142-37395444
chr8: 40182784-40189967
chr8: 47062983-47068987
chr8: 47372645-47377775
chr8: 51031112-51038335
chr8: 54510310-54518345
chr8: 61218121-61227873
chr8: 63034999-63040375
chr8: 63215345-63225085
chr8: 64059438-64065076
chr8: 70858351-70864644
chr8: 78831591-78840271
chr8: 79180575-79187753
chr8: 80315841-80321840
chr8: 85260988-85269165
chr8: 87188259-87195171
chr8: 88522810-88529042
chr8: 94072142-94077206
chr8: 94237473-94243038
chr8: 96049101-96054739
chr8: 99017130-99025235
chr8: 102619280-102626751
chr8: 104906431-104912684
chr8: 109103048-109111256
chr8: 111494281-111500232
chr8: 114040471-114046513
chr8: 115633844-115643484
chr8: 120019379-120027798
chr8: 120153083-120161219
chr8: 125101681-125110686
chr8: 129756802-129766376
chr8: 130435196-130442658
chr8: 135059619-135068046
chr8: 135474056-135479979
chr8: 136621505-136627582
chr8: 145078505-145087086
chr8: 145686297-145692418
chr9: 11210749-11216089
chr9: 15815327-15822312
chr9: 17401160-17406972
chr9: 22486721-22496557
chr9: 22496557-22502830
chr9: 23910743-23916185
chr9: 24555934-24561311
chr9: 28840417-28847122
chr9: 29092325-29098033
chr9: 32628220-32636697
chr9: 71738126-71743370
chr9: 75804224-75810379
chr9: 76018466-76027596
chr9: 78004335-78011956
chr9: 79770402-79777393
chr9: 82028174-82033402
chr9: 99694600-99704486
chr9: 104714737-104724427
chr9: 106780537-106787406
chr9: 107361609-107369324
chr9: 112949066-112956816
chr9: 113024518-113029917
chr9: 117216616-117223475
chr9: 125313731-125319766
chr9: 128971072-128978470
chr9: 134260564-134265801
chr9: 135622520-135627582
chr9: 136128331-136133613
chr9: 136408800-136418510
chr9: 140735489-140740872
chr9: 140735695-140744147
chr9: 140974766-140982248
chr10: 6654432-6664201
chr10: 7627561-7633510
chr10: 11104432-11110983
chr10: 12528602-12533743
chr10: 12542527-12551342
chr10: 16284959-16291617
chr10: 17094457-17101612
chr10: 19177834-19186767
chr10: 20850573-20857449
chr10: 22609123-22617566
chr10: 23699287-23708458
chr10: 53203716-53212090
chr10: 54558282-54566467
chr10: 54783079-54788916
chr10: 54929338-54938105
chr10: 55318849-55328766
chr10: 64425664-64430909
chr10: 67306949-67315330
chr10: 67330860-67337297
chr10: 70612585-70622298
chr10: 78255613-78261009
chr10: 78457472-78464478
chr10: 80825979-80831421
chr10: 83883604-83889070
chr10: 84712311-84717625
chr10: 85547844-85552887
chr10: 87800959-87808419
chr10: 87952333-87959792
chr10: 90794988-90803055
chr10: 100334287-100339880
chr10: 102354737-102362123
chr10: 107057057-107062403
chr10: 110302904-110312792
chr10: 115207583-115216931
chr10: 126068891-126074429
chr10: 126188375-126196450
chr11: 397562-403191
chr11: 1263589-1272764
chr11: 3237378-3244077
chr11: 6114243-6122520
chr11: 9503476-9508855
chr11: 13105229-13114133
chr11: 13309693-13314781
chr11: 20844562-20849955
chr11: 22115220-22122929
chr11: 24443020-24452316
chr11: 24658410-24864318
chr11: 29007150-29012726
chr11: 33048624-33055726
chr11: 35536628-35541880
chr11: 36647350-36654814
chr11: 38457245-38463922
chr11: 42967960-42976150
chr11: 54694268-54702353
chr11: 54883977-54891951
chr11: 57755622-57761480
chr11: 57760689-57767758
chr11: 57851535-57856666
chr11: 61025460-61034934
chr11: 65266587-65273530
chr11: 65573764-65580076
chr11: 65933942-65939538
chr11: 66907429-66917095
chr11: 70074807-70083680
chr11: 76142373-76148527
chr11: 79390434-79395844
chr11: 81473113-81478397
chr11: 81856416-81864120
chr11: 83128488-83135939
chr11: 83532806-83538775
chr11: 93683253-93688561
chr11: 93695267-93702056
chr11: 96821926-96829326
chr11: 101888827-101894040
chr11: 104267656-104273258
chr11: 105293505-105298919
chr11: 112163598-112168627
chr11: 119950867-119956926
chr11: 120659070-120665253
chr11: 124070724-124077178
chr11: 125075432-125085341
chr11: 126961750-126966930
chr11: 131924234-131930336
chr11: 134032897-134037937
chr11: 134601923-134607643
chr11: 134742499-134748742
chr11: 134794930-134800366
chr12: 147109-155538
chr12: 377587-384807
chr12: 866732-874998
chr12: 6242393-6248034
chr12: 6255426-6260562
chr12: 15568294-15573592
chr12: 18385343-18393744
chr12: 22418956-22424245
chr12: 26108846-26114910
chr12: 29558508-29566219
chr12: 30014747-30022341
chr12: 30237233-30243568
chr12: 30290207-30296633
chr12: 30330755-30338534
chr12: 30395367-30404636
chr12: 33299311-33307468
chr12: 39477962-39483489
chr12: 44586517-44593277
chr12: 45903142-45909996
chr12: 57344349-57352033
chr12: 57374348-57379880
chr12: 64399479-64405306
chr12: 67295958-67301314
chr12: 67728435-67733753
chr12: 70872154-70878189
chr12: 72758024-72765117
chr12: 80153258-80162897
chr12: 80157028-80162112
chr12: 80894838-80902628
chr12: 82215508-82225307
chr12: 86426094-86433836
chr12: 86695700-86703035
chr12: 90890467-90899162
chr12: 98953705-98959633
chr12: 99793970-99802788
chr12: 103839074-103846962
chr12: 104279566-104287336
chr12: 108219833-108225072
chr12: 111137848-111146971
chr12: 113360314-113367311
chr12: 114632099-114637993
chr12: 127487761-127495171
chr12: 128338874-128343999
chr12: 131236017-131245086
chr13: 21893096-21899439
chr13: 23631624-23637308
chr13: 27303352-27308782
chr13: 32532622-32539038
chr13: 34135739-34144838
chr13: 35493907-35503836
chr13: 36631328-36640715
chr13: 43599429-43608401
chr13: 49438693-49447756
chr13: 51069346-51075130
chr13: 54234773-54240264
chr13: 54981601-54988098
chr13: 55652880-55659328
chr13: 56020039-56025268
chr13: 56159866-56166458
chr13: 58203393-58209019
chr13: 64935358-64943378
chr13: 70401982-70407255
chr13: 74954890-74962908
chr13: 80454929-80460045
chr13: 80681026-80686542
chr13: 83165945-83172152
chr13: 85275068-85282928
chr13: 85874740-85882354
chr13: 87410775-87418497
chr13: 87831334-87837066
chr13: 92386548-92392301
chr13: 92577389-92586920
chr13: 96919835-96926010
chr13: 103985308-103995158
chr13: 110433668-110440305
chr13: 114029077-114035564
chr13: 114029397-114037918
chr14: 22026513-22035999
chr14: 22050007-22058826
chr14: 23093541-23099284
chr14: 24476336-24484388
chr14: 28667417-28673167
chr14: 35114637-35122518
chr14: 38358854-38365766
chr14: 40609844-40617718
chr14: 42987466-42992914
chr14: 44714462-44721006
chr14: 45329279-45335096
chr14: 48303856-48309594
chr14: 54023454-54028723
chr14: 56454088-56460759
chr14: 61148678-61155015
chr14: 65014784-65020415
chr14: 65432340-65438052
chr14: 73998780-74004922
chr14: 74240149-74245681
chr14: 74977323-74983530
chr14: 79159520-79165627
chr14: 80108280-80114993
chr14: 80691137-80697781
chr14: 84434221-84440941
chr14: 85297015-85302251
chr14: 96527316-96535690
chr14: 99572867-99580532
chr14: 101771398-101778064
chr14: 102783858-102790554
chr15: 25472308-25477545
chr15: 26847642-26856072
chr15: 27403199-27409736
chr15: 27919080-27927994
chr15: 32509148-32517332
chr15: 39364074-39373276
chr15: 42403702-42409107
chr15: 42989012-42995070
chr15: 45154177-45159258
chr15: 46163170-46172592
chr15: 48260708-48266508
chr15: 52265256-52273371
chr15: 54490191-54496351
chr15: 54960675-54966858
chr15: 55129551-55137977
chr15: 56705729-56714821
chr15: 56789892-56799449
chr15: 65642378-65648928
chr15: 71708011-71714661
chr15: 85884428-85893236
chr15: 86340554-86349995
chr15: 94886423-94892329
chr15: 97261396-97269079
chr15: 97808048-97815360
chr15: 98798299-98803712
chr15: 100416735-100421995
chr16: 60072-69544
chr16: 222083-227514
chr16: 3492826-3498744
chr16: 4822530-4831608
chr16: 4898228-4905232
chr16: 5572058-5580264
chr16: 14499743-14505676
chr16: 16725497-16731694
chr16: 19001374-19009025
chr16: 19349712-19355571
chr16: 19406426-19413044
chr16: 20541999-20550237
chr16: 23933905-23942232
chr16: 24536735-24546255
chr16: 24540910-24546370
chr16: 26822964-26829675
chr16: 35245740-35251886
chr16: 47872598-47878432
chr16: 48073692-48084956
chr16: 58945790-58953073
chr16: 59549624-59556208
chr16: 62544371-62550663
chr16: 63216594-63223270
chr16: 68787573-68793936
chr16: 70650573-70660117
chr16: 76661549-76671182
chr16: 77749355-77754764
chr16: 78525165-78530718
chr16: 78972969-78978373
chr16: 80903661-80913633
chr16: 80976802-80983432
chr16: 81246128-81252840
chr16: 85437601-85446396
chr16: 88310313-88316278
chr16: 89066030-89075935
chr17: 16444-21699
chr17: 66139-71897
chr17: 3257497-3266821
chr17: 6871584-6877107
chr17: 10761664-10766716
chr17: 10890347-10895682
chr17: 11028232-11036510
chr17: 12380995-12387689
chr17: 15590147-15595367
chr17: 39384376-39390821
chr17: 39532301-39539624
chr17: 41436594-41442401
chr17: 41861227-41869555
chr17: 53545954-53553610
chr17: 54790683-54795690
chr17: 56207576-56213435
chr17: 61952233-61961185
chr17: 61992965-61999918
chr17: 65438392-65443542
chr17: 70789985-70795175
chr17: 70815169-70821126
chr17: 72499405-72508207
chr17: 75265634-75272576
chr17: 77049053-77056338
chr17: 77486001-77491672
chr17: 79456533-79462306
chr18: 4330259-4335790
chr18: 5927649-5934123
chr18: 5955923-5962557
chr18: 7108710-7113772
chr18: 15405391-15410866
chr18: 25974343-25981420
chr18: 30495710-30503537
chr18: 32759746-32767794
chr18: 34253176-34259839
chr18: 38259897-38266706
chr18: 41976689-41982037
chr18: 46997742-47005316
chr18: 61812963-61822771
chr18: 63200808-63207255
chr18: 63723667-63732675
chr18: 64959015-64967478
chr18: 66202370-66209628
chr18: 67208394-67217471
chr18: 70721315-70726820
chr18: 76793267-76799991
chr18: 77111264-77118922
chr19: 1465058-1470715
chr19: 2085035-2090634
chr19: 2952464-2961355
chr19: 3475233-3480366
chr19: 7754964-7763062
chr19: 9863461-9871586
chr19: 14695453-14704392
chr19: 16037624-16044884
chr19: 17443249-17450279
chr19: 19832965-19839599
chr19: 24459113-24464126
chr19: 27731835-27741199
chr19: 28360883-28367128
chr19: 28405267-28411010
chr19: 35140398-35148102
chr19: 35661185-35666238
chr19: 42547869-42553974
chr19: 43627757-43637403
chr19: 44913296-44920831
chr19: 44957502-44964253
chr19: 45588911-45595377
chr19: 45734893-45740384
chr19: 46622580-46628261
chr19: 46789635-46795752
chr19: 46957030-46963124
chr19: 48407011-48413051
chr19: 50554817-50561656
chr19: 50634803-50640327
chr19: 51106142-51111826
chr19: 53316544-53322605
chr19: 54419684-54429205
chr19: 54739497-54747869
chr19: 55282343-55287902
chr19: 55334160-55340144
chr19: 56713379-56722952
chr19: 56745506-56750701
chr19: 57202898-57211930
chr19: 58538016-58543046
chr19: 59050134-59055169
chr19: 59106282-59114781
chr20: 9317654-9324532
chr20: 11980855-11989333
chr20: 14272053-14278825
chr20: 15310943-15320723
chr20: 23168907-23176009
chr20: 36791314-36799811
chr20: 38641910-38647024
chr20: 39529999-39536976
chr20: 52285691-52292074
chr20: 52331839-52337078
chr20: 52474800-52484346
chr20: 58670192-58675763
chr20: 59474758-59483045
chr20: 60517815-60524291
chr20: 62949257-62958441
chr21: 14343414-14351683
chr21: 20836862-20844283
chr21: 22005639-22010665
chr21: 25258240-25263281
chr21: 25539237-25546833
chr21: 28195250-28201822
chr21: 28227783-28234286
chr21: 31639126-31644252
chr21: 38151275-38157727
chr21: 40981640-40987483
chr21: 41203032-41208686
chr21: 44699610-44706185
chr21: 45232877-45241349
chr21: 45254689-45264263
chr21: 45326239-45334057
chr22: 25956792-25965135
chr22: 29783258-29791250
chr22: 36943282-36949682
chr22: 37744420-37750160
chr22: 38956160-38964907
chr22: 39044664-39050505
chr22: 42523627-42531095
chr22: 50781725-50787037
chr22: 51117967-51125408
chrX: 3983026-3990952
chrX: 6340765-6348422
chrX: 8171057-8179543
chrX: 17273972-17280645
chrX: 17787639-17793339
chrX: 22036030-22041139
chrX: 26516250-26522559
chrX: 30301130-30309650
chrX: 37337724-37343977
chrX: 39964121-39969286
chrX: 43573124-43579834
chrX: 44299823-44305769
chrX: 62578258-62587129
chrX: 63871455-63881383
chrX: 66107886-66113066
chrX: 67123385-67130234
chrX: 79211451-79218632
chrX: 88455722-88463632
chrX: 90969623-90979064
chrX: 92796310-92801483
chrX: 101055334-101060870
chrX: 105523370-105531311
chrX: 108012036-108017924
chrX: 119057290-119065913
chrX: 137242328-137248001
chrX: 141315417-141320722
chrX: 143628694-143637970
chrX: 145891204-145897502
chrX: 146179898-146185881
chrX: 146359714-148369327
chrX: 146843715-146849068
chrX: 149082100-149087710
chrX: 150707124-150713261
chrX: 150722277-150731784
chrX: 151080438-151090155
chrX: 154790170-154797579
chrX: 155179516-155185223

TABLE 5
Genomic regions covered by 500 bp oligonucleotide
spacing. Chromosome coordinates correspond
to the NA18507 human genome.

chr1: 1567881-1683706
chr1: 4393668-4404190
chr1: 6476628-6521516
chr1: 8216828-8240818
chr1: 8671618-8685734
chr1: 14311609-14371586
chr1: 16346942-16390883
chr1: 16833086-16343681
chr1: 17175722-17195780
chr1: 17175722-17206132
chr1: 17175722-17281753
chr1: 17206162-17220630
chr1: 17229613-17280888
chr1: 17229920-17259337
chr1: 19599632-19614527
chr1: 22293885-22342339
chr1: 22320722-22340752
chr1: 25585225-25665195
chr1: 25610133-25646986
chr1: 25688611-25751772
chr1: 41346422-41380988
chr1: 44095567-44107276
chr1: 62437799-62452376
chr1: 64839620-64852107
chr1: 72786282-72811969
chr1: 85974018-86005661
chr1: 87580408-87614258
chr1: 88879477-88906799
chr1: 89798969-89812208
chr1: 95134798-95156000
chr1: 106104918-106122404
chr1: 106164403-106214528
chr1: 106307571-106317929
chr1: 108311070-108325460
chr1: 108760471-109097308
chr1: 108778622-108853925
chr1: 108914669-109000909
chr1: 110215012-110244931
chr1: 110216166-110259108
chr1: 111378562-111389136
chr1: 112692629-112704793
chr1: 112693290-113248263
chr1: 113246318-113350775
chr1: 113862952-114901117
chr1: 114919330-115547919
chr1: 115563302-116102691
chr1: 121350196-121485194
chr1: 121351637-121435549
chr1: 121406885-121426119
chr1: 121473109-121485194
chr1: 152185869-152196724
chr1: 152274215-152287539
chr1: 152555610-152590091
chr1: 152648867-152660425
chr1: 152760089-152771114
chr1: 153069195-153087963
chr1: 153671644-153695309
chr1: 155180489-155221243
chr1: 155227059-155262674
chr1: 158159222-158214160
chr1: 161409063-161442720
chr1: 161411989-161544650
chr1: 161479945-161646758
chr1: 166180432-166196987
chr1: 169059628-169621268
chr1: 169226853-169242132
chr1: 170625064-170641390
chr1: 171016086-171726961
chr1: 171128845-171414611
chr1: 171556291-171726822
chr1: 176590614-176613264
chr1: 178449098-178481285
chr1: 181147037-181213705
chr1: 188984277-189038124
chr1: 189705238-189780469
chr1: 191826563-191868462
chr1: 196710318-196825282
chr1: 196737356-196749660
chr1: 196821982-196880841
chr1: 196880054-196919270
chr1: 202337476-202401225
chr1: 202414924-202530388
chr1: 205894531-205922673
chr1: 207896481-207754886
chr1: 213131581-213156027
chr1: 221743956-221754422
chr1: 223626904-223642146
chr1: 224204343-224241314
chr1: 229817084-229829777
chr1: 230502053-230523061
chr1: 231431283-231444667
chr1: 234911945-234958266
chr1: 242954573-242984842
chr1: 243120334-243152955
chr1: 245057981-245196900
chr1: 245636794-245647974
chr1: 243604260-248635197
chr1: 248609916-248625792
chr1: 248729106-248743545
chr1: 248738015-248795518
chr1: 248865885-248876072
chr2: 1090699-1111412
chr2: 1141529-1154686
chr2: 1525712-1542066
chr2: 1730446-1749394
chr2: 4204794-4223770
chr2: 6298327-6308710
chr2: 13499731-13567350
chr2: 13579581-13598202
chr2: 24600228-24611405
chr2: 30615374-30627773
chr2: 33983236-33994164
chr2: 34695430-34736585
chr2: 35580770-35616409
chr2: 35976255-35997482
chr2: 36101229-36113833
chr2: 36121925-36134023
chr2: 37958078-38005884
chr2: 38871950-38882521
chr2: 38955877-38972830
chr2: 41238373-41250918
chr2: 43523379-43534540
chr2: 46967820-46978960
chr2: 47429316-47472670
chr2: 52749417-52785316
chr2: 53671457-53687640
chr2: 55323841-55336774
chr2: 55910848-55938746
chr2: 56720459-56748522
chr2: 60771820-60785782
chr2: 66185933-66197069
chr2: 73270284-73280427
chr2: 74579111-74608543
chr2: 75777740-75813043
chr2: 83235809-83864013
chr2: 98856422-98886491
chr2: 104393067-104406991
chr2: 106508989-106546685
chr2: 115390792-115402680
chr2: 133280684-133308806
chr2: 146882598-146877112
chr2: 155818973-155830150
chr2: 165828132-165857436
chr2: 167447880-167462030
chr2: 169135368-169146055
chr2: 178837784-178847855
chr2: 179518009-179528400
chr2: 180060110-180083212
chr2: 180066730-180081432
chr2: 185099581-185139440
chr2: 185246565-185264597
chr2: 189567840-189580339
chr2: 203295334-203312346
chr2: 205898770-206127984
chr2: 206272092-206525287
chr2: 206283097-206671319
chr2: 209233686-209247332
chr2: 227908538-227922128
chr2: 228241147-228258447
chr2: 230340156-230358409
chr2: 234648159-234659534
chr3: 313663-326550
chr3: 611991-663369
chr3: 3956893-4004684
chr3: 6219115-6240583
chr3: 13744272-13758036
chr3: 16579903-16657470
chr3: 16635713-16652881
chr3: 19535653-20638501
chr3: 22078044-22092344
chr3: 26425785-26439404
chr3: 27068441-27083380
chr3: 27637157-27655063
chr3: 28187000-28197678
chr3: 46794424-46851520
chr3: 49720751-49735851
chr3: 51344873-51415825
chr3: 53027067-53039086
chr3: 56607723-56621531
chr3: 60832980-60843228
chr3: 62664337-62695407
chr3: 65188921-65214735
chr3: 75985380-75998678
chr3: 78460435-78476012
chr3: 89394021-89418291
chr3: 114657752-114668329
chr3: 116098946-116109977
chr3: 128379304-128404004
chr3: 128404063-128414199
chr3: 131595148-131607666
chr3: 144281519-144292534
chr3: 145626322-145662143
chr3: 146071361-146085768
chr3: 151511489-151551000
chr3: 155479247-155512774
chr3: 159794041-159814966
chr3: 162129709-162143255
chr3: 162512139-162525707
chr3: 162512139-162626613
chr3: 164168079-164188485
chr3: 165040990-165083355
chr3: 165266793-165296640
chr3: 165427158-165469281
chr3: 167139852-167154907
chr3: 174847782-174858661
chr3: 175887928-175913730
chr3: 175894038-175945952
chr3: 178550206-178568168
chr3: 184766850-184786757
chr3: 186386577-186424582
chr3: 186407577-186420150
chr3: 186906378-186968680
chr3: 189364906-189391843
chr3: 192875347-192885399
chr3: 194384096-194400303
chr4: 7214065-7225591
chr4: 8622050-8638959
chr4: 10099819-10234566
chr4: 10136109-10153485
chr4: 10174154-10234566
chr4: 10192625-10205135
chr4: 10211321-10234566
chr4: 10391951-10403481
chr4: 12371636-12385731
chr4: 21448552-21467379
chr4: 23591417-23605498
chr4: 34780061-34828809
chr4: 52880191-52683414
chr4: 58050201-58100690
chr4: 58722784-58738685
chr4: 59977391-59988256
chr4: 64134293-64154884
chr4: 64694135-64714151
chr4: 66394427-66430783
chr4: 66588799-66628622
chr4: 68788730-69016101
chr4: 71946100-71956214
chr4: 73424118-73449218
chr4: 76948569-76977780
chr4: 90580433-90592288
chr4: 91885542-91899572
chr4: 92946774-92966992
chr4: 104200223-104248841
chr4: 104560866-104574617
chr4: 108063452-108076040
chr4: 112297331-112309505
chr4: 116166955-116178282
chr4: 129588899-129598971
chr4: 130126384-130137965
chr4: 133787552-133810493
chr4: 135818244-135837994
chr4: 137914173-137953216
chr4: 138179982-138191850
chr4: 145934041-145955418
chr4: 152762084-152778976
chr4: 152778545-152790136
chr4: 156947790-156966136
chr4: 156956902-156973966
chr4: 157406057-157421983
chr4: 161047495-161072143
chr4: 164804987-164820063
chr4: 167875942-167892836
chr4: 168024645-168080542
chr4: 168476112-168490969
chr4: 168808583-168843407
chr4: 168808583-168992985
chr4: 178375249-178386550
chr4: 178467260-178488396
chr4: 178594075-178610710
chr4: 184355090-184389693
chr4: 185158014-185169181
chr4: 187093611-187111440
chr4: 187880472-187894796
chr4: 188653718-188683531
chr5: 3809831-4002533
chr5: 7176867-7200482
chr5: 8013215-8024076
chr5: 8702908-8748681
chr5: 12675510-12759105
chr5: 19385379-19396257
chr5: 23884245-23905186
chr5: 29621789-29637423
chr5: 34074329-34084702
chr5: 45949801-45971776
chr5: 46166065-46185948
chr5: 46271528-46405359
chr5: 49405763-49442098
chr5: 49405763-49485157
chr5: 49405763-49562327
chr5: 57323526-57333742
chr5: 61501951-61555756
chr5: 67417197-67430908
chr5: 73979961-73993853
chr5: 84420918-84451702
chr5: 100551009-100649385
chr5: 101093096-101108930
chr5: 101166750-101207276
chr5: 104217558-104255023
chr5: 104432909-104503663
chr5: 108537620-108555452
chr5: 109459520-109475317
chr5: 111915807-111944317
chr5: 112369563-112410775
chr5: 116682395-116693314
chr5: 120298249-120416752
chr5: 131315694-131439226
chr5: 132576593-132679043
chr5: 138710207-138727419
chr5: 140222149-140238885
chr5: 140868116-140909299
chr5: 147320290-147337996
chr5: 148820125-148975254
chr5: 149021197-149100635
chr5: 150058714-150079999
chr5: 150203369-150223430
chr5: 150941705-150955343
chr5: 155476656-155495022
chr5: 155576941-155591365
chr5: 165838926-165849965
chr5: 180176013-180195763
chr5: 180374610-180432609
chr6: 238018-248744
chr6: 255650-384973
chr6: 256416-296014
chr6: 372472-384523
chr6: 18721397-18732996
chr6: 23711548-23746486
chr6: 25506093-25519529
chr6: 31219861-31235969
chr6: 31276526-31289437
chr6: 31285501-31319932
chr6: 31285646-31301620
chr6: 31285646-31351653
chr6: 31302807-31319195
chr6: 31336878-31352378
chr6: 31354329-31453049
chr6: 31948122-32014041
chr6: 31958876-31985522
chr6: 31991589-32016526
chr6: 32411907-32779836
chr6: 32412272-32429847
chr6: 32432637-32455578
chr6: 32443285-32455578
chr6: 32443285-32662922
chr6: 32458282-32494610
chr6: 32489779-32512224
chr6: 32491718-32505192
chr6: 32505330-32594357
chr6: 32507644-32522716
chr6: 32522111-32551944
chr6: 32537784-32549005
chr6: 32550505-32594392
chr6: 32581704-32594392
chr6: 32591684-32610890
chr6: 32592915-32604184
chr6: 32602686-32635674
chr6: 32603512-32664534
chr6: 32603750-32722627
chr6: 32604006-32779836
chr6: 32627032-32664199
chr6: 32657577-32675010
chr6: 32672987-32698070
chr6: 32674346-32779836
chr6: 32685892-32722627
chr6: 33023069-33039991
chr6: 33025058-33054420
chr6: 35754516-35766780
chr6: 38391599-38404785
chr6: 40196301-40210354
chr6: 46795547-46831976
chr6: 47622270-47652871
chr6: 48349931-48365346
chr6: 49281885-49295108
chr6: 52220162-52231392
chr6: 55826123-55846698
chr6: 57255468-57266677
chr6: 57618703-57631956
chr6: 57695737-57707602
chr6: 58714399-58773619
chr6: 61880260-61969732
chr6: 61880260-62128238
chr6: 62178788-62919415
chr6: 62178821-62288093
chr6: 63141271-63155169
chr6: 65843155-65878849
chr6: 67008761-67048866
chr6: 68238248-68248625
chr6: 69230104-69242009
chr6: 70188989-70277993
chr6: 72863860-72874518
chr6: 74564769-74614549
chr6: 74610209-74646506
chr6: 77016724-77029130
chr6: 77439258-77460349
chr6: 77865181-77876113
chr6: 78879462-78899584
chr6: 78967468-79036288
chr6: 80461435-80476237
chr6: 81040322-81055387
chr6: 103736692-103764186
chr6: 104905477-104943368
chr6: 115703974-115718364
chr6: 116181321-116218030
chr6: 117509122-117565572
chr6: 119251007-119272087
chr6: 120628982-120642503
chr6: 122676203-122698042
chr6: 124432576-124470985
chr6: 131397221-131418195
chr6: 132106620-132118530
chr6: 147324012-147334530
chr6: 161261102-161279717
chr6: 162496365-162506976
chr6: 165220423-165238765
chr6: 168794864-168812027
chr7: 68972-81961
chr7: 3723272-3737147
chr7: 7326731-7426329
chr7: 8826507-8866206
chr7: 13104708-13130563
chr7: 19613957-19628138
chr7: 24300778-24312539
chr7: 29671940-29767315
chr7: 29671957-29687687
chr7: 34895856-34914728
chr7: 38285259-38311187
chr7: 38387663-38397946
chr7: 38387926-38407761
chr7: 40051874-40063660
chr7: 48581423-48592171
chr7: 52730103-52745307
chr7: 53455198-53466514
chr7: 67428639-67440330
chr7: 76769667-76786762
chr7: 76778716-76789098
chr7: 78803960-78854070
chr7: 79471617-79482876
chr7: 85489362-85518712
chr7: 88522102-88730866
chr7: 88917192-89250651
chr7: 89161984-89371045
chr7: 89519486-89531461
chr7: 89812643-90193186
chr7: 97329371-97352156
chr7: 99564133-99625411
chr7: 101000829-101012960
chr7: 104465454-104476590
chr7: 109430510-109453848
chr7: 109441297-109453923
chr7: 110818063-110883472
chr7: 110820423-110835780
chr7: 111022673-111100065
chr7: 111048229-111083413
chr7: 113565832-113601235
chr7: 118132835-118163268
chr7: 119374573-119386624
chr7: 126514673-126533162
chr7: 126531997-126552956
chr7: 128414489-128593205
chr7: 129379923-129445465
chr7: 129984276-130028371
chr7: 133785001-133798015
chr7: 134262755-134276288
chr7: 134268699-134306007
chr7: 140193474-140203499
chr7: 147271448-147285044
chr7: 149862533-149882792
chr7: 150249312-150312843
chr7: 150292485-150307048
chr7: 153478745-153498963
chr7: 154392156-154404572
chr7: 156298579-156313899
chr7: 159117412-159128541
chr8: 1574852-1590095
chr8: 2253695-2270696
chr8: 2319093-2329687
chr8: 5150393-5161603
chr8: 5595389-5605737
chr3: 5750384-5765400
chr8: 8334431-8348655
chr8: 9642195-9712807
chr8: 11236398-11247076
chr8: 13614187-13651528
chr8: 15401845-15413138
chr8: 16262054-16274657
chr8: 16788560-16822237
chr8: 16798468-16812441
chr8: 22999124-23022454
chr8: 24972402-24990893
chr8: 25577777-25593905
chr8: 32680057-32691191
chr8: 35666429-35680427
chr8: 37404863-37477281
chr8: 38577156-38587880
chr8: 39231662-39387419
chr8: 39232353-39242893
chr8: 43415270-43820710
chr8: 43819904-43838857
chr8: 46838946-46857964
chr8: 46954213-46992148
chr8: 46992500-47190161
chr8: 47285021-47360478
chr8: 50330931-50348468
chr8: 51182892-51195695
chr8: 52023759-52033873
chr8: 53961879-53992755
chr8: 54510310-54529246
chr8: 58357122-58368516
chr8: 60996528-61007764
chr8: 65244457-65256991
chr8: 70619615-70633412
chr8: 77781097-77817086
chr8: 96206151-96243934
chr8: 99169690-99199971
chr8: 100213556-100228531
chr8: 104182119-104245529
chr8: 105015114-105025274
chr8: 107535827-107547049
chr8: 108577087-108602455
chr8: 111891496-111908496
chr8: 113078409-113089318
chr8: 115151653-115174839
chr8: 122318815-122339523
chr8: 122400708-122430090
chr8: 135474653-135501685
chr8: 139053657-139063772
chr8: 140759282-140770035
chr8: 144700491-144714743
chr9: 10485-25682
chr9: 5385326-5403173
chr9: 11630285-11682559
chr9: 11645505-11720969
chr9: 17581146-17634284
chr9: 23362830-23377665
chr9: 24502114-24519104
chr9: 28740061-28767224
chr9: 33792711-33804828
chr9: 72034725-72049025
chr9: 81437947-81471210
chr9: 84527569-84566607
chr9: 88113071-88129254
chr9: 91522086-91625517
chr9: 91963403-92343382
chr9: 92678855-92698160
chr9: 105968615-105989355
chr9: 110584585-110604045
chr9: 117082836-117098233
chr9: 118562519-118574831
chr9: 122291445-122304641
chr9: 123849613-123860183
chr9: 124874636-124901377
chr9: 130622568-130646494
chr9: 135944496-135957645
chr9: 138147265-138310299
chr9: 139663637-139674967
chr9: 141019512-141033726
chr10: 2064190-2092498
chr10: 2688857-3134214
chr10: 7094209-7139426
chr10: 13701384-13712432
chr10: 18378907-18402738
chr10: 18747626-18761494
chr10: 18841137-18862749
chr10: 18852387-18862559
chr10: 21857163-21867961
chr10: 26345494-26361303
chr10: 42354975-42396556
chr10: 42532988-42546570
chr10: 56446076-56469571
chr10: 56469623-56503557
chr10: 56950069-56996305
chr10: 57219473-57291499
chr10: 58513061-58526913
chr10: 59520466-59566694
chr10: 59840220-59892584
chr10: 60854127-60908437
chr10: 71280786-71291054
chr10: 85221103-85250546
chr10: 92708763-92723506
chr10: 93305214-93361590
chr10: 95594800-95609228
chr10: 96856006-96996043
chr10: 96864858-96874954
chr10: 96942869-97074756
chr10: 100334287-100344560
chr10: 100686440-100697698
chr10: 100688852-100703742
chr10: 102191937-102211601
chr10: 107615437-107685915
chr10: 115540274-115550561
chr10: 122769751-122787516
chr10: 123433437-123458753
chr10: 129488612-129553456
chr10: 129503068-129520740
chr10: 131562778-131576387
chr10: 133175550-133191834
chr10: 133183673-133194737
chr10: 133643781-133666751
chr11: 363975-403432
chr11: 2062876-2088085
chr11: 6076999-6117545
chr11: 6104402-6121639
chr11: 18138662-18200717
chr11: 18165268-18194893
chr11: 18925223-18999313
chr11: 18941820-18965820
chr11: 21188356-21215920
chr11: 25123939-25295835
chr11: 25606635-25628337
chr11: 25702274-25720912
chr11: 36730247-36788022
chr11: 37283631-37303212
chr11: 39741445-39788053
chr11: 51567562-51594097
chr11: 51581147-51594097
chr11: 54956929-54973121
chr11: 58614671-58633269
chr11: 60964605-61021031
chr11: 64001265-64018114
chr11: 70830303-70849553
chr11: 79972706-79983283
chr11: 81500492-81521688
chr11: 88620667-88664736
chr11: 99490974-99512639
chr11: 103369583-103394212
chr11: 104757749-104781659
chr11: 107231516-107243470
chr11: 107238737-107249703
chr11: 107653021-107672290
chr11: 108256476-108267672
chr11: 124109057-124122118
chr11: 126235211-126250907
chr11: 127193924-127203985
chr11: 134575178-134607523
chr11: 134795622-134806663
chr12: 147109-190102
chr12: 169632-186546
chr12: 172767-188610
chr12: 5769633-5788275
chr12: 14634100-14647438
chr12: 20892854-21029179
chr12: 22571513-22584682
chr12: 25455511-25469350
chr12: 30496609-30508640
chr12: 33380281-33395477
chr12: 34484300-34509480
chr12: 34697979-34713311
chr12: 34699518-34856651
chr12: 34748481-34760877
chr12: 39458222-39478357
chr12: 40301693-40315894
chr12: 40867874-40884927
chr12: 40873057-40885157
chr12: 41494646-41517821
chr12: 49676002-49690399
chr12: 57331182-57379580
chr12: 57331527-57352203
chr12: 59925488-59945796
chr12: 59935811-59946596
chr12: 62199825-62210977
chr12: 106162101-106182216
chr12: 111226874-111238171
chr12: 122634975-122649886
chr12: 123178106-123210671
chr12: 126991364-127004802
chr12: 126997819-127013399
chr12: 132811317-132821692
chr12: 133494730-133533926
chr12: 133637506-133734930
chr12: 133717202-133779425
chr13: 20460189-20471255
chr13: 23400518-23420631
chr13: 27038326-27052101
chr13: 38072041-38085553
chr13: 55622263-55635283
chr13: 57803501-57824387
chr13: 57865893-57892460
chr13: 58589166-58599767
chr13: 61068566-61088065
chr13: 63599578-63652860
chr13: 64224656-64237607
chr13: 66746811-66759444
chr13: 69244710-69268730
chr13: 70735733-70747927
chr13: 70735733-70775484
chr13: 75363251-75391485
chr13: 76107773-76119573
chr13: 84160831-84184110
chr13: 103683881-103701612
chr13: 108540707-108561345
chr13: 113310352-113326595
chr14: 22907992-22918086
chr14: 24914740-24951857
chr14: 24932115-24947813
chr14: 27280768-27329091
chr14: 40354008-40373562
chr14: 41472033-41483728
chr14: 41515318-41526061
chr14: 41608601-41669570
chr14: 41814193-41859645
chr14: 45821203-46020734
chr14: 47707591-47717843
chr14: 49893688-50942527
chr14: 58133563-58148413
chr14: 58242535-58259794
chr14: 73994388-74051967
chr14: 74001783-74015709
chr14: 74019536-74041528
chr14: 74033959-74050302
chr14: 80900600-80939903
chr14: 82309889-82321693
chr14: 99034754-99057332
chr14: 101761120-101790010
chr14: 102689563-102715292
chr14: 102702936-102715292
chr15: 25294529-25334107
chr15: 25420149-25430709
chr15: 25422499-25496629
chr15: 31908441-32019557
chr15: 32444256-32509148
chr15: 43852144-43988641
chr15: 45131798-45145856
chr15: 45154177-45175380
chr15: 45224488-45235491
chr15: 46102589-46118104
chr15: 47361529-47396555
chr15: 51072875-51086217
chr15: 51705468-51735025
chr15: 56851808-56868691
chr15: 61686975-61698541
chr15: 63379905-63545462
chr15: 64997675-65007724
chr15: 69503597-69522651
chr15: 72353301-72395288
chr15: 82098396-82113891
chr15: 87671272-87689759
chr15: 89456419-89544859
chr15: 92588543-92606763
chr15: 94225455-94236334
chr15: 97808395-97832871
chr16: 69379-84503
chr16: 3875339-4165746
chr16: 3875754-3930051
chr16: 3875754-4320693
chr16: 3946756-4017307
chr16: 4166962-4302948
chr16: 4390814-4526245
chr18: 4826249-4905232
chr16: 6763953-6784276
chr16: 8596634-8607181
chr16: 8926797-8939700
chr16: 8939807-8988283
chr16: 12674207-12693522
chr16: 12674207-12734557
chr16: 12691586-12706911
chr16: 19945543-19966502
chr16: 20501101-20545196
chr16: 27336350-27351077
chr16: 35243037-35257000
chr16: 35245230-35285750
chr16: 50463383-50499675
chr16: 54408013-54419626
chr16: 55795744-55823668
chr16: 55842125-55865379
chr16: 70847248-71202529
chr16: 72088500-72112297
chr16: 78371619-78384952
chr16: 78527053-78540007
chr16: 79026434-79039202
chr16: 80997245-81028812
chr16: 84651924-84670939
chr16: 86109427-86119539
chr17: 41-21799
chr17: 6101903-6126856
chr17: 9254201-9271032
chr17: 11249707-11259789
chr17: 15632283-15677380
chr17: 15676903-15688164
chr17: 33705204-33716191
chr17: 39382773-39396083
chr17: 39383451-39407073
chr17: 39421637-39432205
chr17: 39506945-39525624
chr17: 54160262-54172717
chr17: 62915636-62926025
chr17: 63927678-63989878
chr17: 65388196-65401678
chr17: 74702781-74716763
chr17: 75216084-75236311
chr17: 79455958-79468592
chr17: 81012068-81060000
chr18: 5930491-5962158
chr18: 5955923-5970711
chr18: 10134666-10145472
chr18: 20054187-20067385
chr18: 20239565-20302756
chr18: 29370464-29391297
chr18: 44222960-44263857
chr18: 61382356-61945364
chr18: 62181737-62193457
chr18: 65296099-65317333
chr18: 74996366-75010936
chr19: 4885263-4900658
chr19: 4997958-5015266
chr19: 8337134-8366327
chr19: 10489139-10505017
chr19: 15778056-15837007
chr19: 24551540-24631644
chr19: 24603100-24631644
chr19: 29309220-29325557
chr19: 33469567-33523332
chr19: 35849254-35865601
chr19: 39811570-39886212
chr19: 40362630-40411824
chr19: 40386050-40401043
chr19: 41337301-41393256
chr19: 43308866-43547145
chr19: 43465605-43491399
chr19: 43505920-43636036
chr19: 43547802-43800679
chr19: 43616520-43648439
chr19: 43659296-43701055
chr19: 43699805-43766599
chr19: 44892380-44913200
chr19: 44928244-44938721
chr19: 48406290-48463376
chr19: 48416454-48429144
chr19: 48432209-48460874
chr19: 48450363-48462360
chr19: 49518848-49559939
chr19: 50592944-50643496
chr19: 51254643-51267041
chr19: 51263359-51275079
chr19: 52131804-52150223
chr19: 53322989-53361358
chr19: 53517167-53529678
chr19: 53517167-53554243
chr19: 53537411-53553720
chr19: 54356155-54369469
chr19: 54578162-54590294
chr19: 54723919-54749032
chr19: 54724539-54743633
chr19: 54732882-54743633
chr19: 54800397-54811362
chr19: 55239710-55301831
chr19: 55245819-55257585
chr19: 55245819-55379001
chr19: 55301626-55334160
chr19: 55329252-55377048
chr19: 55354282-55379205
chr19: 56267485-56286338
chr19: 57988560-58003720
chr20: 12627634-12841804
chr20: 13233949-13336340
chr20: 13279946-13336560
chr20: 13283535-13447810
chr20: 14422471-14439945
chr20: 14771533-14939565
chr20: 16566460-16586787
chr20: 26298168-26319513
chr20: 29804062-29833443
chr20: 29814555-29832573
chr20: 51157759-51168996
chr20: 52464720-52486135
chr20: 52647106-52658080
chr20: 53426647-53443224
chr20: 54281438-54291996
chr20: 59113602-59124879
chr20: 59396497-59409726
chr20: 59567854-59590016
chr21: 10697941-10774701
chr21: 10766657-10861866
chr21: 14338455-14368624
chr21: 21610135-21620816
chr21: 21800414-21845326
chr21: 23303924-23318945
chr21: 23654928-23665979
chr21: 24175831-24211114
chr21: 24423325-24435206
chr21: 25446467-25467682
chr21: 37922877-37938746
chr21: 40918970-40965975
chr21: 44822523-44838545
chr21: 47293464-47303825
chr22: 25619034-25928990
chr22: 29783647-29794087
chr22: 30280400-30298271
chr22: 31461316-31474644
chr22: 35457951-35469677
chr22: 39355807-39392500
chr22: 39426703-39443920
chr22: 42517258-42541967
chr22: 42520935-42550520
chr22: 42527065-42541189
chr22: 42879261-42954881
chr22: 42897738-43005214
chr22: 42962616-42977833
chr22: 51067664-51078724
chrX: 2365874-2399200
chrX: 3734030-3856690
chrX: 3734575-3761060
chrX: 3739947-3799384
chrX: 3800585-3855985
chrX: 3839019-3857055
chrX: 8454001-8518025
chrX: 26810657-26821041
chrX: 27265919-27500092
chrX: 30806559-30325841
chrX: 34042986-34072685
chrX: 39949255-39969615
chrX: 43572184-43585716
chrX: 56655690-56672223
chrX: 56794101-56807918
chrX: 57600617-57618003
chrX: 57746379-57756613
chrX: 58505727-58526280
chrX: 58507310-58581854
chrX: 58558297-58581854
chrX: 61682187-61726512
chrX: 61682187-61769705
chrX: 61682187-61918208
chrX: 62346911-62385163
chrX: 63722947-63735218
chrX: 66103939-66138092
chrX: 78914283-78924581
chrX: 79160790-79180498
chrX: 80214189-80232301
chrX: 80748634-80780706
chrX: 81395939-81415502
chrX: 101055334-101067767
chrX: 103210756-103225013
chrX: 103258654-103305529
chrX: 119034815-119058340
chrX: 121304874-121316011
chrX: 125101270-125168874
chrX: 125177002-125226847
chrX: 125233398-125290643
chrX: 130230896-130274637
chrX: 140775845-140789431
chrX: 143160940-143295598
chrX: 148643925-148654642
chrX: 148877193-149031805
chrX: 148878343-148902155
chrX: 154299852-154445592
chrX: 154782659-154797504
chrX: 154790028-154803714
chrX: 154929773-154963593

### Example 3

### Pilot Study

The microarray chips of Example 2 were used to detect genomic regions that have undergone complex structural rearrangements predisposing subjects to Developmental Neurocognitive Disorders (DND) and Complex Autoimmune Disorders.

Four families afflicted with Autism Spectrum Disorder (ASD), two families afflicted with psoriasis (Ps) and one family afflicted with Ankylosing Spondylitis (AS) were studied.

Genomic DNA samples were obtained from the subjects. The samples were first cleaned using QIAamp DNA Micro kit (Qiagen Cat#56304, lot#433156339). Each sample was eluted in a final volume of 95 μl in the Buffer AE provided with the kit. Then each sample was submitted to Nanodrop absorbance measurements for quantitation and quality analysis. A 2% agarose 48 wells EGeI® (E-gel, Invitrogen#G800802) was done to control the quality of gDNA.

According to NanoDrop results, 1.5 μg of each sample (in duplicate) were prepared and also 1.5 μg of control associated to each sample (including duplicate). The sample labeling was done by adding Random Primer to the samples before denaturation and fragmentation in a thermal cycler (AB Applied Biosystems #GeneAmp PCR system 9700) at 95° C. for 10 minutes, 4° C. for 5 minutes then move on ice for 5 minutes incubation. The Labeling Master mix was added to each tube (Cy3 for sample and Cy5 for control). Samples were transferred to a thermal cycler for 2 hours at 37° C., 10 minutes at 65° C. and 4° C. holding. The samples were then moved to ice and cleaned using Amicon 30 kD filter unit.

The cleaning was done with 1×TE buffer from Promega (TE Buffer, 1×, Molecular Grade (pH 8.0), a buffer composed of 10 mM Tris-HCl containing 1 mM EDTA Na2, pH at 25° C. The final volumes obtained were around 21 μl. Each duplicate (sample and control) were combined and the volume adjusted to 161 μl. 1.5 μl of each sample (combined) were used to determine yield and specific activity using Nanodrop spectrophotometer with the function MicroArray Measurement for DNA-50.

After yield determination, each sample was mixed with its corresponding control for a total volume of (319 μl). The total mixture was split into 2 tubes for hybridization. The hybridization master mix was added to each tube. Sample tubes were transferred into incubator with 1.5 ml tube heat block (SciGene #1057-30-0, SciGene#1057-34-0) set at 95° C. for exactly 3 minutes and immediately transferred into a second block heater set at 37° C. for 30 minutes.

Removing sample from 37° C. two by two (duplicate), the duplicates were mixed and loaded on the corresponding array then placed into hybridization oven for the week-end (86 hours).

After hybridization, the arrays were removed from the oven 8 by 8 and washed with wash buffer 1, wash buffer 2, acetonitrile and stabilization & drying solution. Arrays were installed into slide holder and cover with ozone barrier. Immediately after, the arrays were scanned with Agilent Sure Scan C scanner with a resolution of 3 μm.

### Detection of Previously Identified Genomic Aberrations

The custom microarray was able to detect previously reported pathogenic aberrations associated with Autism Spectrum Disorder (ASD).

A 700 kb deletion known to be associated with ASD was detected on chromosome 16p11,2. The detected aberration was de novo as it was only detected in the affected family member and not in unaffected parents or siblings.

### Novel ASD Loci

DNA from 17 subjects representing four families was analyzed. Seven subjects with ASD and 10 controls were analyzed.

A. Complex Aberration with PGAP1 Gene

Both deletion and duplication events were detected within this region in three families. In each family, the complex aberration was detected in affected family members but not in unaffected family members.

The PGAP-1 gene aberration is located on chromosome 2 between nucleotides 197707345-197776074 (NA18507 human genome).

Without being bound by theory, PGAP1 (post-GPI attachment to proteins 1) catalyzes glycosylphosphatidylinositol (GPI) biosynthesis and PGAP1 may function as a novel component of the Wnt pathway during forebrain development [Zoltwicz et al, 2009]. In addition, knockout of PGAP1 in mice results in complete loss of GPI synthesis and disrupts neurodevelopment [UEDA et al. 2007]. This is the first report linking aberrations within PGAP1 with ASD.

B. Complex Aberration within C7orf58

Multiple deletions were detected within the C7orf58 gene in three families. In each family, the aberration was detected in affected family members but not in unaffected family members.

Without being bound by theory, while the specific function of c7orf58 is unknown, disruption of the c7orf58 gene has been previously reported in a single patient with mental retardation, anxiety disorder and ASD (Dauwerse et al., 2009).

C. Complex Aberration within LNX1 Gene

An aberration was observed within the LNX1 gene. A complex aberration pattern was observed involving two deletions within the same gene within multiple patients with autism spectrum disorders.

The LNX1 gene aberration is located on chromosome 4 between nucleotides 54436284-5433277 (NA18507 human genome).

### Novel Ankylosing Spondylitis Loci

DNA from 10 members of a single family were analysed. The family pedigree included 5 members affected with ankylosing spondylitis (AS), 2 with systemic lupus and 1 with psoriasis.

A complex aberration (multiple duplications within and adjacent to UGT2B17 and UGT2B25 genes) were detected in all family members affected with AS but was not detected in unaffected family members. This aberration was also detected in one family member affected with systemic lupus.

The UGT2B17 gene aberration is located on chromosome 4 between nucleotides 69399539-69430016 (NA18507 human genome) and the UGT2B15 gene aberration is located on chromosome 4 between nucleotides 69518934-69530196 (NA18507 human genome).

The UGT2B17 gene encodes a key enzyme responsible for glucuronidation of androgens and their metabolites in humans. Without being bound by theory, changes in copy number within the UGT2B17 gene have been previously reported to be involved in bone formation, a characteristic of AS (Yang et al, Giroux et al).

### Example 4

### Atypical Micro-Duplications Located at 2q21.1-21.2 Co-Segregate with Tourette Syndrome in a Three Generation Family

### Introduction

Tourette syndrome (TS) is a developmental neuropsychiatric disorder characterized by the presence of motor (simple and/or complex) and verbal tics with duration longer than one year [Pauls et al. 1991; Price et al. 1985; State 2011]. TS often manifests with features associated with obsessive compulsive disorder (OCD); attention deficit hyperactivity disorder (ADHD), poor impulse control and other behavioural abnormalities, the pathophysiology of which remain to be elucidated [Robertson 2012]. The prevalence of TS is between 0.3-1% in any given population [Centers for Disease Control and Prevention 2009; Robertson 2008; Robertson et al. 2009], and consistently affects males more than females [Robertson 2012]. Twin studies consistently show higher concordance rates in monozygotic compared with dizygotic twins [Pauls et al. 1991; Price et al. 1985; Walkup et al. 1988] suggestive of a strong genetic component underpinning disease pathogenesis. Although early segregation analyses suggested an autosomal dominant inheritance pattern [Eapen et al. 1993], recent evidence suggests a heterogeneous complex genetic architecture underpins the pathogenesis of TS [Eapen et al. 1993; Pauls and Leckman 1986; State 2010; State 2011].

Structural variations are a risk factor for neuropsychiatric diseases. Recent analysis of copy number variants (CNV) in TS have demonstrated an association with genes previously implicated in autism spectrum disorders (ASD) and other neuropsychiatric disorders [Fernandez et al. 2012; Lawson-Yuen et al. 2008]. A rare deletion of exons located 5′ in the neurexin 1 (NRXN1) gene was identified in two unrelated TS patients [Sundaram et al. 2010]. A second deletion in the α-T catenin (CTNNA3) gene was identified in two independent TS studies [Fernandez et al. 2012; Sundaram et al. 2010]. Interestingly, deletions encompassing both the NRXN1 and CTNNA3 genes have been reported in ASD and schizophrenia [Fernandez et al. 2012; Sundaram et al. 2010]. Another rare deletion comprising the NLGN4 gene (Le. exons 4, 5 and 6) has been previously reported in TS and ASD [Lawson-Yuen et al. 2008]. An insertion/translocation between chromosomes 2 and 7 was reported to disrupt the CNTNAP2 gene in a two generation pedigree with a father and two offspring affected with TS [Verkerk et al. 2003]. The identification of CNVs implicated in neuropsychiatric disorders complicates genotype-phenotype analysis. That no single CNV has been reported to segregate uniquely with TS in affected families provides a great opportunity to detect novel CNVs specific to TS through the study of multiplex families.

### Clinical Reports

Family A.

The proband (FIG. 6; ID3000) presented at 9 years to a developmental pediatrician following a referral from a school counsellor and was diagnosed with TS. His three siblings were subsequently diagnosed at 6, 8 and 9 years respectively (FIG. 6; ID3001, ID3002, ID3003). The mother of these boys had anxiety, obsessive traits and vocal and motor tics since childhood, however was only diagnosed as having TS at 44 years. (Table 6). The father of the four affected boys has mild anxiety but no tics. The maternal grandfather has no diagnosis, but has manifested both verbal and motor tics through his lifespan according to family information, although these were not obvious during a recent psychiatric assessment. The maternal grandmother was diagnosed with bipolar disorder in her 70s and is treated effectively. She has no tics currently, nor any history of tics.

Proband 10003.

The proband presented at 12 years to a pediatric psychiatrist and was diagnosed with TS (Table 6). Extended family history is limited due to adoption.

Families B and C.

These families have no known extended history of TS or other co-morbidities.

### Methods

TS Population.

Probands and families were ascertained through a prospective study of TS in Newfoundland and Labrador (NL), from the Department of Child and Adolescent Psychiatry and the Child Development Clinic in the Janeway Child Health Centre, the Provincial Children's Hospital. Extended family histories and in-depth clinical information were obtained. The study was approved by the Human Research Ethics Board (#07-71). To date, 28 probands have been recruited and eight multi-generational family histories completed. DNA samples were collected from all affected subjects, their parents and extended family members (in multipex pedigrees) following consent and completion of multiple rating scales. The primary focus of this study was a single multiplex pedigree (FIG. 6, Family A).

Control Population.

To assess the population frequency of the CNV detected using the custom aCGH microarray, 590 control samples were used from the NL population with no clinical report of TS and performed real time quantitative fluorescence polymerase chain reactions (QF-PCR). Custom Microarray. To assess the presence of CNVs on a genome-wide scale, a custom genome-wide microarray was designed based on breakpoints in regions that are susceptible to genomic rearrangements previously identified [Uddin et al. 2011]. The microarray comprised 2×1 million probes covering the genome with a mean spacing of 280 bp. DNA from the TS multiplex family was applied to the custom aCGH microarray which was performed at Genome Quebec (GQ) using an Agilent platform. Prior to CNV analysis, QC measures were applied and the derivative of the log ratio spread (DLRS) <0.25 was considered the threshold and CNVs were detected using the built-in Aberration Detection Method-2 (ADM-2) algorithm DNA Analytics v.4.0.85 (Agilent Technologies) using the following criteria: 1) at least five (5) probes for a CNV call on GC-corrected intensity; 2) nested filter was set to 2; and 3) log intensity >0.24 for duplications and <−0.24 for deletions. A custom script was applied to detect gene-enriched CNVs (i.e., overlaps or consists of a gene) that segregated (at least three cases) with affected status in the family.

QF-PCR.

To confirm the duplication detected using the custom 2M aCGH microarray, a Taqman copy number assay (Hs03417816; Life Technologies) was performed using the manufacturer's recommended protocol. The assay was performed in quadruplicate on 10 ng of genomic DNA for each sample in a 96-well plate. The 10 μl reaction mix consisted of 2 μl 2× Taqman Genotyping Master Mix (Life Technologies), 0.5 μl of 20× copy number assay (described above), 0.5 μl of TaqMan RNAse P Copy Number Reference Assay (Life Technologies, part 4403326), 2 μl of water and 2 μl of 5 ng/μl genomic DNA. Cycling conditions for the reaction were 95° C. for 10 min, followed by 40 cycles of 95° C. for 15 sec and 60° C. for 1 min. Samples were analyzed using the ViiA™ 7 Real-Time PCR System (Life Technologies) and analyzed using CopyCaller Software (Life Technologies, PN 4412907). Three reference (calibrator) DNA HapMap samples (NA10851, NA15510 and NA07048; Coriell Institute) plus one non-template control were included with the test samples.

### Results

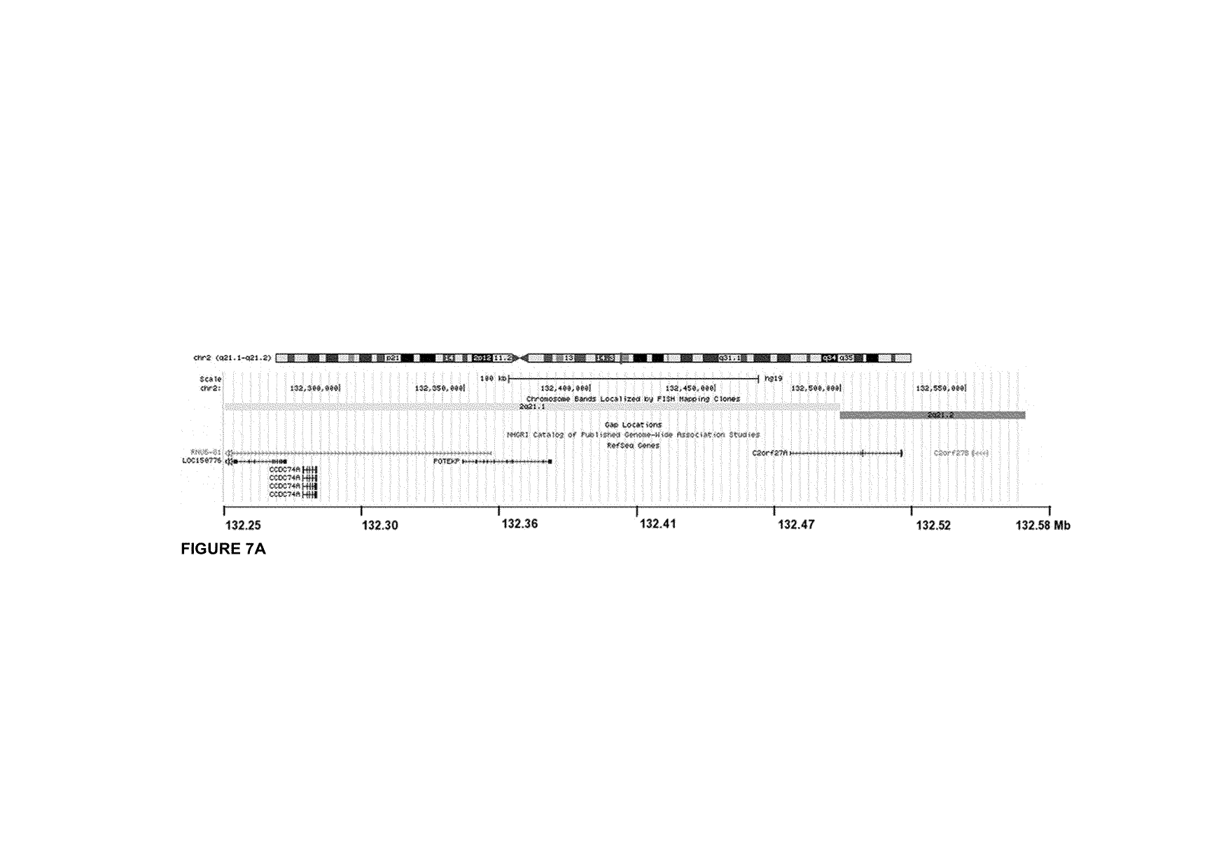

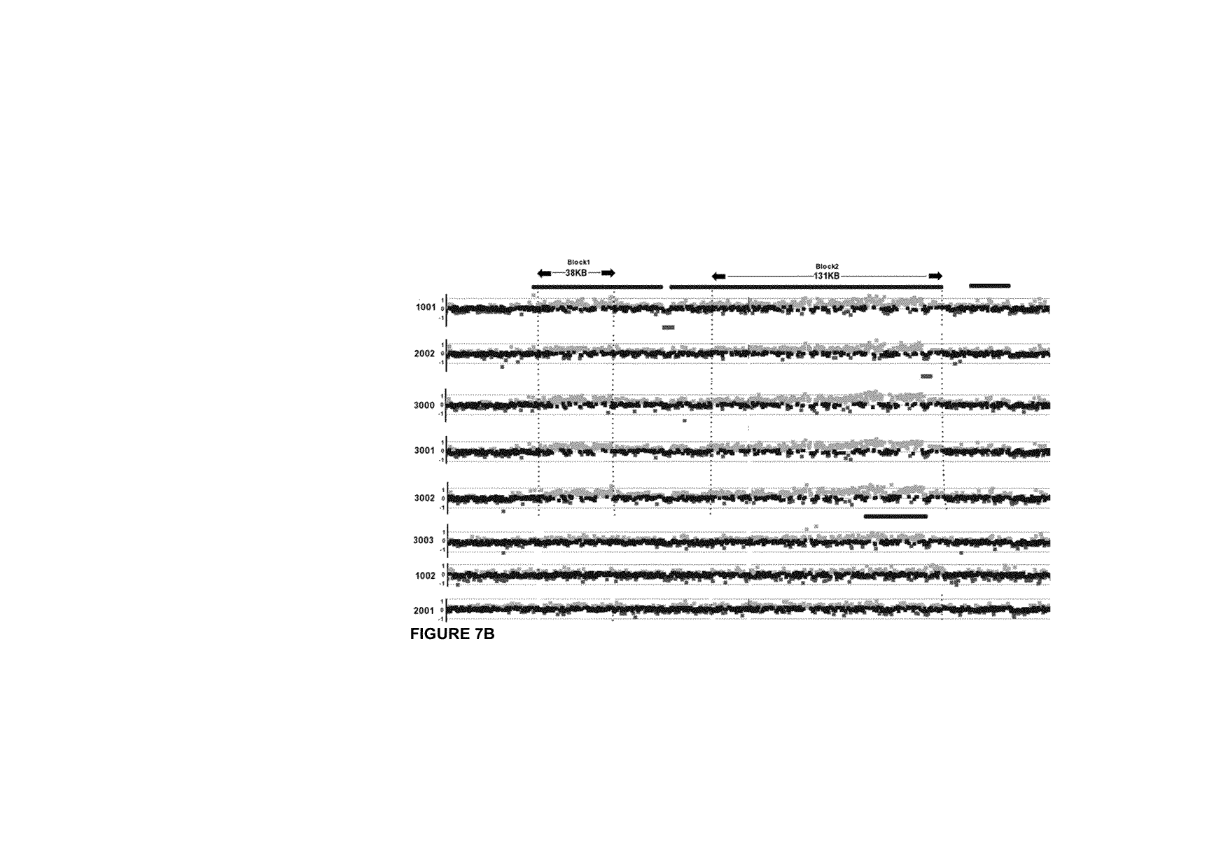

The custom high-density aCGH microarray yielded approximately 2000 genomic aberrations. Comprehensive data analysis revealed atypical, rare micro-duplications located at chromosome 2q21.1 which segregated with affected members in family A. Large de novo variants were not detected in affected family members (data not shown). Within a 221 kb (chr2:132305299-132526804) region, two common blocks of micro-duplications were identified that segregated together in five of the six affected individuals (FIG. 2), with a smaller region of overlap of block2 in the remaining affected individual (FIG. 1, ID3003). These duplication blocks are 38 kb (block1-chr2:132305299-132343808) and 131 kb (block2-chr2:132395155-132526804) in length, respectively (FIG. 7), separated by 51 kb. All affected family members had nearly identical breakpoints except the fourth affected sibling (ID3003) who had a partial 30 kb duplication within block2 (chr2:132480185-132510827), All affected family members carried a micro-duplication which encompassed the C2orf27A gene. Complex rearrangements were also detected in block2 for individuals ID1001 and ID2002 comprising small micro-deletions of 4.1 kb and 2.4 kb, respectively.

The presence of block2 among five of the six affected family members was validated using a QF-PCR assay which demonstrated a relative copy number of four within the affected siblings and mother whereas unaffected members had a copy number of two or three (data not shown). QF-PCR analysis was performed on two additional families and 10 unrelated individuals with TS. The block2 micro-duplication with a copy number of 4 was detected in one additional affected individual (ID10003), but absent in all other unrelated affected or unaffected samples tested. Of the 590 control individuals analyzed using QF-PCR, only CNV predictions calls on 443 samples had a 95% confidence interval. The frequency of a copy number of four was observed in 4/443 (0.009) individuals.

### Discussion

The salient characteristics of TS segregate in subjects with the micro-duplications including multiple motor and vocal tics, and common co-morbidities including ADHD, OCD, major depression, anxiety, behavioural problems, and learning disability [Termine et al, 2006]. Migraine and sleep difficulties which have been reported in association with TS are also present in several affected family members [Abelson et al. 2005; Freeman et al. 2000; Kwak et al. 2003; Lespérance et al, 2004; Singer 2005]. Although TS segregates with the micro-duplications described, the morbidity of disease is variable. The proband (FIG. 6; ID3001) and his three siblings exhibit various features of TS, with the three oldest siblings manifesting the greatest phenotypic morbidity. The mother (ID2002) was diagnosed with TS at 44 y.o. Her past medical and school history however suggests that the TS diagnosis would have been made in childhood were she to present today as a child. However both the mother (ID2002) and the father (ID2001) function at a high level socially and occupationally. The maternal grandparents (ID1001 & ID1002) function well and have done so throughout life, with the maternal grandfather (ID1001) manifesting tics throughout his adult life. The youngest sibling (ID3003) who carries a partial 30 kb micro-duplication within block2 presents with a more benign phenotype compared with his older siblings. However, an unrelated TS proband (ID10003) also carries the same partial micro-duplication and has a phenotypic presentation similar to the older three siblings in family A, and the two common micro-duplication blocks are also present in the mother (ID2002) and maternal grandfather (ID1001), neither of whom present with the same burden of disease. Although TS has been considered a single clinical diagnosis, it is usually considered a ‘spectrum’, and evidence from factor analysis suggests that the disorder can be separated into symptom groupings with potentially different etiologies [Cavanna et al. 2011] further complicating genotype-phenotype correlation as illustrated by Family A.

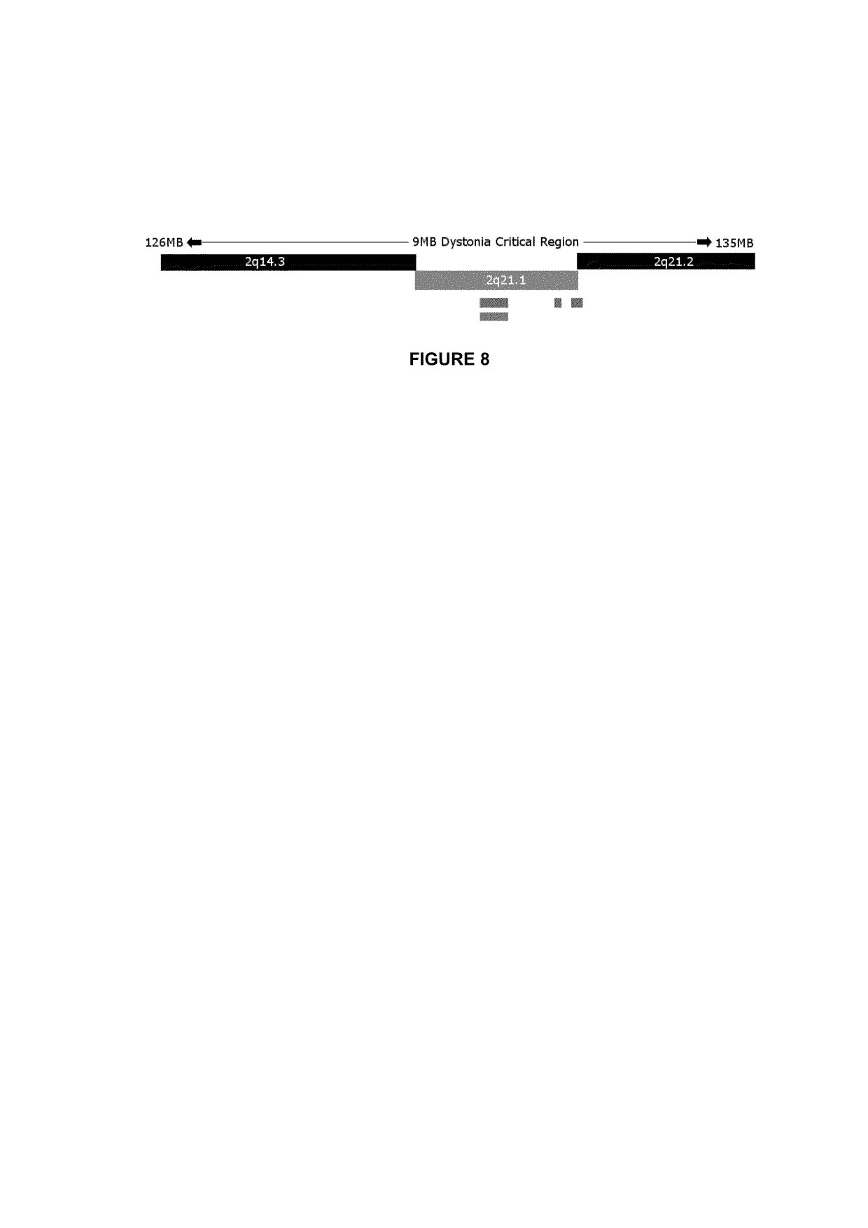

A genomic region containing two micro-duplication blocks, the larger of which (131 kb) segregates with TS status in a three generation family has been identified. This micro-duplication encompasses the C2orf27A gene, which belongs to the C2orf27 gene family, and encodes an uncharacterized protein. Although the function of this gene is unknown, it was derived from a guanine nucleotide exchange factor protein [Toll-Riera et al. 2011]. Guanine nucleotide exchange factors are expressed in the basal ganglia [Kawasaki at al. 1998] which is associated with a variety of functions, including voluntary motor control, procedural learning relating to routine behaviors or “habits” such as eye movements, and cognitive functions [Albin at al. 1995]. Unlike previous reports of CNV associations with TS [Fernandez et al. 2012; Sundaram et al. 2010], these micro-duplications have not been reported with any other neuropsychiatric disorder and thus the candidate region is specific to TS. Interestingly, a larger region which encompasses the CNVs here detected in this study was previously identified as a locus through linkage analysis for dystonia in a four generation family [Norgren at al. 2011]. In that study, a critical 8.9 MB region correlated with the highest LOD score (FIG. 8). This critical region represents a hotspot for genomic aberrations correlating with multiple neuropsychiatric conditions.

Given that the micro-duplications identified in this study are atypical CNVs, the population frequency was determined. From the Newfoundland population, it was observed that the larger micro-duplication represents an atypical, rare genomic aberration. Previously published high-density (42 million probes) genomic tiling microarray data (Conrad et al., 2010) have revealed the presence of common micro-deletions interspersed within the micro-duplicated regions identified in this study. Very low frequency (0.01-0.07) typical duplications have been reported in the Database of Genomic Variants (DGV) within this region. The DGV demonstrated typical CNV gains with low frequencies and of the reported studies, no single individual carries two duplication blocks within this region. However, the breakpoints reported in the DGV have not been validated. These breakpoints are also absent within the large study that investigated 15,767 children with various types of intellectual disability [Cooper et al. 2011]. Thus, this unusual segregation of the two micro-duplication blocks within the TS family is a rare event and is highly correlated with TS pathogenesis.

These findings underline the impact of CNVs with respect to human health and genomic susceptibility to TS. The larger micro-duplication that segregates with the affected individuals of Family A encompasses the C2orf27A gene. The rare frequency of this micro-duplication within the control population shows a link between the 2q21.1-21.2 locus and TS pathogenesis.

TABLE 6
Clinical Features of Family A and Proband B.

| | | | Presentation | | | | | |
|---|---|---|---|---|---|---|---|---|
| | | Age | and | | | | Co- | Other |
| | Birth | TS | Developmental | IQ/Cognitive: | | | Morbiality: | Medical |
| | Hx. | dx. | History | Profile | Tics | Treatment^{β} | age of dx. | Problems |
| 3000 | C | 9 y | Normal early | WISC (8 y): | Vocal and | Melatonin | ADHD | Allergic to |
| | section | | milestones. | FSIQ 73%, VIQ | motor tics | Risperidone | combined | dust mites 9 y. |
| | Breech | | Referred to | 94%, PIQ 32%. | since 6 y. | Ritalin, | type 8 y | Mild asthma |
| | Breast | | Developmental | Overall high- | Increased tics | Concerta | OCD 9 y | 12 y. |
| | fed 10 | | Paediatrician | average IQ. | at 12 y. Worst | Strattera | Anxiety | Gynecomastia |
| | months | | (8 y) by | Large spilt | tics reduced | Prozac | 9 y school | 13 y. Chronic |
| | | | School | between verbal | by 14 y. | Adderall | refusal | headache 13 y. |
| | | | Guidance | and performance | | Accutane | 14 y. | Sleep disorder |
| | | | Counsellor | IQ. | | | Addiction | 14 y (difficulty |
| | | | for possible | Psychoeducational | | | to gaming | falling asleep, |
| | | | ADHD | assessment | | | 19 y | waking early, |
| | | | | (15 y): written | | | | snoring: sleep |
| | | | | output learning | | | | study |
| | | | | disability. | | | | negative). |
| | | | | WAIS IV (18 y): | | | | Acne 16 y |
| | | | | FSIQ 61%; | | | | Possible mood |
| | | | | VCI 70%; PRI | | | | disorder, |
| | | | | 86%; WMI 55%; | | | | (very |
| | | | | PS; 6% | | | | apathetic) |
| | | | | WIATII (18 y): | | | | ongoing |
| | | | | spelling superior, | | | | assessment |
| | | | | listening | | | | with |
| | | | | comprehension | | | | Psychiatrist |
| | | | | high average, | | | | 19 y. |
| | | | | other areas | | | | |
| | | | | average. | | | | |
| 3001 | C | 6 y | Normal early | WISC (8 y) | Vocal and | Melatonin | ADHD 7 y | Nocturnal |
| | section | | milestones. | FSIQ: 32%, VIQ | motor tics | Risperidone | Dyslexia | enuresis 6 y |
| | Placental | | Referred to | 37%, PIQ: 77%, | since 6 y | Ritalin | (severe) | Sleep disorder |
| | failure | | Developmental | PS: 21%, WMI | | Concerta | 9 y | (? sleep |
| | Breast | | Paediatrician | 18%. Overall | | Strattera | Sensory | apnea) 8 y |
| | fed 1 | | at 4 yrs for | average | | Clonidine | integration | Acne 13 y |
| | year | | assessment of | WIATII (8 y) | | Accutane | disorder | |
| | | | speech | composite score: | | | 9 y | |
| | | | problems | extremely low | | | OCD 9 y | |
| | | | aggression | for reading and | | | Auditory | |
| | | | frustration | writing, low | | | processing | |
| | | | and fine | average for oral | | | disorder | |
| | | | motor skill | language and | | | 14 y | |
| | | | delay. | borderline for | | | | |
| | | | Possible | math. | | | | |
| | | | ADHD/TS | | | | | |
| 3002 | C | 8 y | Normal early | WISC (10 y): | Vocal and | Melatonin | ADHD 7 y | Trace |
| | section | | milestones. | Overall high- | motor tics | Concerta | OCD 9 y | tricuspid and |
| | at 37 | | Referred to | average IQ. | since 6 y. | Strattera | Anxiety | pulmonic |
| | weeks | | developmental | FSIQ 87%; VCI | complex tics | Prozac | 9 y | regurgitation |
| | Breast | | paediatrician | 91%; PRI 91%; | at 8 y | Adderall | | 9 y |
| | fed 8 | | at 5 y for | WMI 42%; PS | | Paxil | | Sleep |
| | months | | concerns | 73% | | | | disturbance 5 y |
| | | | about fine | WIATII (10 y) | | | | |
| | | | motor skills, | Spelling skills | | | | |
| | | | speech | borderline, | | | | |
| | | | development, | listening | | | | |
| | | | hyperactivity | comprehension | | | | |
| | | | poor impulse | average | | | | |
| | | | control and | | | | | |
| | | | aggression. | | | | | |
| 3003 | C | 9 y | Normal early | WISC (8 y): | Vocal and | Melatonin | OCD 6 y | Sleep |
| | section | | milestones. | FSIQ 34%; VCI | motor tics | | Dyslexia | disturbance 7 y |
| | at 37 | | Referred to | 88% PRI 61%; | since 6 y | | 9 y | Sensory issues |
| | weeks | | developmental | WMI 9%; PS 9% | | | Anxiety | 5 y |
| | Breast | | paediatrician | WIATII (8 y): | | | Fearful of | |
| | fed 8 | | at 5 y for | borderline word | | | school | |
| | months | | aggressive | reading, | | | 10 y | |
| | | | behaviour | numerical | | | | |
| | | | and | operations | | | | |
| | | | obsessions. | spelling and oral | | | | |
| | | | Easily | expression | | | | |
| | | | frustrated | | | | | |
| 2002 | NVD | 44 y | Normal early | Not done | Vocal and | Concerta | ADHD | Cervical |
| | | | milestones | | motor tics | Strattera | 38 y | dysplasia 22 y |
| | | | | | since | | Self | Renal colic |
| | | | | | childhood | | manages | 25 y |
| | | | | | | | anxiety | Sensory issues |
| | | | | | | | and OCD. | and |
| | | | | | | | | Migraine |
| | | | | | | | | headaches 31 y |
| 1001 | N/A | N/A | N/A | Not done | Vocal and | | Has OCD | Atrial |
| | | | | | motor tics* | | traits* | Fibrillation |
| | | | | | Easily | | | 69 y |
| | | | | | frustrated* | | | Basal cell |
| | | | | | | | | carcimoma |
| | | | | | | | | 78 y |
| | | | | | | | | Colon Cancer |
| | | | | | | | | 79 y |
| | | | | | | | | Abdominal |
| | | | | | | | | aortic |
| | | | | | | | | aneurysm 80 y |
| 10003 | NVD | 12 y | Normal | Not done | Motor tics | Risperidonc | ADHD | tympanostomy |
| | | | early | | 5 y | | 12 y | tubes 6 y |
| | | | milestones | | Vocal | Strattera | OCD | Systolic |
| | | | Referred to | | tics 10 y | Clonidine | 13 y | heart |
| | | | pediatrician | | OCD | Seroquel | Anxiety | murmur 11 y |
| | | | 12 y. History | | worsening 15 | Prozac | NOS 17 y | Sleep |
| | | | of | | y, resolving | Celexa | | problems |
| | | | hyperactivity, | | 19 y | Haldol | | (difficulty |
| | | | ADHD, | | | Clonazepam | | falling |
| | | | short | | | Luvox | | asleep) 13 y |
| | | | attention | | | | | Equinus |
| | | | span, anger | | | | | deformity of |
| | | | issues, sleep | | | | | foot 14 y |
| | | | problems | | | | | |

TS: Tourette Syndrome,
ADHD: Attention Deficit and Hyperactivity Disorder,
FSIQ: Full Scale Intelligent Quotient,
VIQ: Verbal Intelligent Quotient,
PIQ: Performance Intelligent Quotient,
VCI: Verbal Comprehension Index,
PRI: Perceptual Reasoning Index,
WNI: Working Memory Index,
PS: Processing Speed,
y: years,
NVD: normal vaginal delivery,
N/A: Not available
*Family information only
^{β}Includes all treatments from diagnosis to present day: not all currently prescibed.

### REFERENCES

Bailey J A, Eichler E E (2006) Primate segmental duplications: crucibles of evolution, diversity and disease. Nat Rev Genet 7: 552-564.
Redon R, Ishikawa S, Fitch K R, Feuk L, Perry G H, el al. (2006) Global variation in copy number in the human genome. Nature 444: 444-454.
Bailey J A, Cu Z, Clark R A, Reinert K, Samonte R V, et al. (2002) Recent segmental duplications in the human genome. Science 297: 1003-1007.
Alkan C, Coe B P, Eichler E E (2011) Genome structural variation discovery and genotyping. Nat Rev Genet 12: 363-376.
Sharp A J, Locke D P, McGrath S D, Cheng Z, Baily J A, et al. (2005) Segmental duplications and copy-number variation in the human genome. Am J Hum Genet 7: 78-88.
Gu W, Zhang F, Lupsky J R (2008) Mechanisms for human genomic rearrangements. PathoGenetics 1:4.
Lieber M R, Ma Y, Pannicke U, Schwarz K (2003) Mechanism and regulation of human non-homologous DNA end-joining. Nat Rev Mol Cell Biol 4: 712-720.
Zhang F, Khajavi M, Connolly A M, Towne C F, Batish S D, et al. (2009) The DNA replication FoSTeS/MMBIR mechanism can generate genomic, genic and exonic complex rearrangements in humans. Nat Genet 41: 849-853.
Conrad F D, Bird C, Blackburn B, Lindsay S, Mamanova L, et al. (2010) Mutation spectrum revealed by breakpoint sequencing of human germline CNVs. Nat Genet 42: 385-391.
Mills R E, Walter K, Stewart C, Handsaker R E, Chen K, et al. (2011) Mapping copy number variation by population-scale genome sequencing. Nature 470: 59-65.
Shaikh H T, O'Connor J R, Pierpont E M, McGrath J, Hacker M A, et al. (2007) Low copy repeats mediate distal chromosome 22q11.2 deletions: Sequence analysis predicts breakpoint mechanisms. Genome Res 17: 482-491.
DECIPHER Genomic Aberration Database: http://decipher.sanger.ac.uk/
Turner D J, Miretti M. Rajan D, Fiedler H, Carter N P, et al. (2007) Germline rates of de novo meiotic deletions and duplications causing several genomic disorders. Nat Genet 40: 90-95.
Ballif B C, Theisen A, Coppinger J, Gowans G C, Hersh J H, et al. (2008) Expanding the clinical phenotype of the 3q29 microdeletion syndrome and characterization of the reciprocal microduplication. Mol Cytogenet 1: 1-7.
Koscinski I, Ellnati E, Fossard C, Rodin C, Muller J, et al. (2011) DPY19L2 deletion as a major cause of Globozoospermia. Am J Hum Genet 88: 344-350.
Bayes M, Magano L F, Rivera N, Flores R. Jurado L A P (2003) Mutational mechanism of Williams-Beuren Syndrome deletions. Am J Hum Genet 73: 131-151.
Conrad, D. F. Pinto, D., Radon, R., Feuk, L., Gokcumen, at al. (2010) Origin and functional impact of copy number variation in the human genome. Nature 464: 704-712.
Perry H G, Ben-Dor A, Tsalenko A, Sarnpas N, Rodriguez-Revenga L, at al. (2008) The fine-scale and complex architecture of human copy-number variation. Am J Hum Genet 82: 685-695.
Sudmant P H, Kitzman J O, Antonacci F, Alkan C, Malig M, et al. (2010) Diversity of Human Copy Number Variation and Multicopy Genes. Science 330: 641-645,
Alkan C, Kidd J M, Marques-Bonet T, Antonacci F, Hormozdiari F, et al, (2009) Personalized copy number and segmental duplication maps using next-generation sequencing. Nat Genet 41: 1061-1067.
Ho M R, Tsai K W, Chen C, and Lin W (2010) dbDNV: a resource of duplicated gene nucleotide variants in human genome. Nucleic Acids Res 39: D920-D925.
Mefford H, Trask B J (2002) The complex structure and dynamic evolution of human subtelomeres. Nat Rev Genet 3: 91-102.
She X, Horvath J E, Jiang Z, Liu G, Furey T S, et al. (2004) The structure and evolution of centromeric transition regions within the human genome. Nature 430: 857-864.
Skaletsky H, Kuroda-Kawaguchi T, Minx P J, Cordurn H S, Hillier L, et al. (2003) The male-specific region of the human Y chromosome is a mosaic of discrete sequence classes. Nature 423: 825-837.
Park H, Kim J I, Ju Y S, Gokcumen O, Mills R E et al. (2010) Discovery of common Asian copy number variants using integrated high-resolution array CGH and massively parallel DNA sequencing. Nat Genet 42: 400-405.
Antonacci F, Kidd J M, Marques-Bonet T, Teague B, Ventura M, at al. (2010) A large and complex structural polymorphism at 16p12.1 underlies microdeletion disease risk. Nat Genet 42: 745-750.
Nagamani S C, Erez A, Bader P, Lalani S R, Scott D A, et al. (2011) Phenotypic manifestations of copy number variation in chromosome 16p13.11. Eur J Hum Genet 19: 280-286.
Heinzen E L, Radtke R A, Urban T J, Cavalleri G L, Depondt C, et al. (2010) Rare deletions at 16p13.11 predispose to a diverse spectrum of sporadic epilepsy syndromes. Am J Hum Genet 86: 707-718.
Weiss L A, Shen Korn J M, Arking D E, Miller D T, et al. (2008) Association between microdeletion and microduplication at 16p11.2 and autism. N Engl J Med 358: 667-675.
Walters R G, Jacquemont S, Valsesia A, Smith A J-de, Martinet D, et al. (2010) A new highly penetrant form of obesity due to deletions on chromosome 16p11.2. Nature 463: 671-675.
Ballif B C, Hornor S A, Jenkins E, Madan-khetarpal S, Surti U, et al. (2007) Discovery of a previously unrecognized microdeletion syndrome of 16p11.2-p12.2, Nat Genet 39: 1071-1073.
Tokutomi T, Wada T, Nakagawa E, Saitoh S, Sasaki M (2009) A de novo direct duplication of 16p22.1-q23.1 in a boy with midface hypoplasia and mental retardation. Am J Med Genet 149A: 2560-2563.
Ensenauer R E, Adeyinka A, Flynn H C, Michels W, Lindor N M, et al. (2003) Microduplication 22q11.2, an emerging syndrome: clinical, cytogenetic, and molecular analysis of thirteen patients. Am J Hum Genet 73: 1027-1040.
Huang X S, Xiao L, Li X, Xie Y, Jiang H O, et al. (2010) Two neighboring microdeletions of 5q13.2 in a child with oculo-auriculo-vertebral spectrum. Eur J Med Genet 53: 153-158.
Diskin S J, Hou C. Glessner J T, Attiyeh E F, Laudenslager M, et al. (2009) Copy number variation at 1q21.1 associated with neuroblastoma. Nature 459: 987-992.
Brunetti-Pierri N, Berg J S, Scaglia F, Belmont J, Bacino C A, et al. (2008) Recurrent reciprocal 1q21.1 deletions and duplications associated with microcephaly or macrocephaly and developmental and behavioural abnormalities, Nat Genet 40: 1466-1471.
Hach F, Hormozdiari F, Alkan C, Hormozdiari F, Bird I et al. (2010) mrsFAST: a cache-oblivious algorithm for short-read mapping. Nat Methods 7: 576-577.
Li Heng, Ruan J, and Durbin R. (2008) Mapping short DNA sequencing reads and calling variants using mapping quality scores. Genome Res 18: 1851-1858.
Alkan C, Sajjadian S, and Eichler EE (2011) Limitations of next-generation genome sequence assembly. Nat Methods 8: 61-65.
Tucker T, Montpetit A, Chai D, Chan S, Chenier S, et al. (2011) Comparison of genome-wide array genomic hybridization platforms for the detection of copy number variants in idiopathic mental retardation. BMC Med Genomics 4: 25-35.
Bently D R, Balasubramanian, Swerdlow H P, Smith G P, Milton J, et al. (2008) Accurate whole genome sequencing using reversible terminator chemistry. Nature 456: 53-59.
Smith T F, Waterman M S (1981) Identification of common molecular subsequences. J Mol Bid 147: 195-197.
Altschul S F, Gish W, Miller W, Myers E W, Lipman D J (1990) Basic local alignment search tool. Journal of Molecular Biology 215: 403-441.
Kent W J (2002) BLAT—the BLAST-like alignment tool. Genome Res 12: 656-664.
Yanovsky V, Rumble S R, Brudno M (2008) Read Mapping Algorithms for Single Molecule Sequencing Data. in Workshop on Algorithms in Bioinformatics (WABI), Springer-Verlag, Karlsruhe, Germany.
Mi H, Dong Q, Muruganujan A, Gaudet P, Lewis S, et al. (2010) PANTHER version 7: improved phylogenetic trees, orthologs and collaboration with the gene ontology consortium. Nucleic Acids Res 38: D204-D210.
Abelson J, Kwan K, O'Roak B, Back D, Stillman A, Morgan T, Mathews C, Pauls D, Rasin M. Gunel M, Davis N. Ercan-Sencicek A, Guez D, Spertus J, Leckrnan J, Dure Lt, Kurlan R, Singer H, Gilbert D, Farhi A, Louvi A, Lifton R, Sestan N, State M. 2005. Sequence variants in SLITRK1 are associated with Tourette's syndrome. Science 310:317-320.
Albin R, Young A, Penney J. 1995. The functional anatomy of disorders of the basal ganglia. Trends Neurosci 18:63-64.
Cavanna A, Critchley H, Orth M, Stern J, Young M, Robertson M. 2011. Dissecting the Giles de la Tourette spectrum: a factor analystic study on 639 patients J Neurol Neurosurg Psychiatry 82:1320-1323.
Centers for Disease Control and Prevention. 2009. Prevalence of diagnosed Tourette syndrome in persons aged 6-17 years-United States, 2007. MMWR Morb Mortal Wkly Rep. p 581-585.
Cooper G, Coe B, Girirajan S, Rosenfeld J, Vu T, Baker C, Williams C, Stalker H, Hamid R, Hannig V. Abdel-Hamid H, Bader P, McCracken E, Niyazov D, Leppig K, Thiese H, Hummel M, Alexander N. Gorski J, Kussmann J, Shashi V, Johnson K, Rehder C, Ballif B, Shaffer L, Eichler E. 2011 A copy number variation morbidity map of developmental delay. Nat Gen 43:838-846.
Dharmadhikari A, Kang S, Szafranski P, Person R, Sampath S, Prakash S, Bader P, Phillips Jr, Hannig V, Williams M, Vinson S, Wilfong A, Reimschisel T, Craigen W, Patel A, Bi W, Lupski J, Belmont J. Cheung S, Stankiewicz P. 2012 Small rare recurrent deletions and reciprocal duplications in 2q21.1, including brain-specific ARHGEF4 and GPR148. Hum Mol Genet 21:3345-3355.
Eapen V, Pauls D, Robertson M. 1993 Evidence for autosomal dominant transmission in Tourette's syndrome. United Kingdom cohort study. Br J Psychiatry 162:593-596.
Ercan-Sencicek A, Stillman A, Ghosh A, Bilguvar K, O'Roak B, Mason C, Abbott T, Gupta A, King R, Pauls D, Tischfield J, Heiman G, Singer H, Gilbert D, Hoekstra P, Morgan T, Loring E, Yasuno K, Fernandez T, Sanders S, Louvi A, Cho J, Mane S, Colangelo C, Biederer T, Lifton R, Gunel M. State M. 2010 L-histidine decarboxylase and Tourette's syndrome. N Engl J Med 362:1901-1908.
Fernandez T, Sanders S, Yurkiewicz I, Ercan-Sencicek A, Kim Y, Fishman D, Raubeson M, Song Y, Yasuno K, Ho W, Bilguvar K, Glessner J, Chu S, Leckman J, King R, Gilbert D, Heiman G, Tischfield J, Hoekstra P, Devlin B, Hakonarson H, Mane S, Günel M, State M. 2012 Rare Copy Number Variants in Tourette Syndrome Disrupt Genes in Histaminergic Pathways and Overlap with Autism. Biol Psychiatry 71:392-402.
Freeman R D, Fast D K, Burd L, Kerbeshian J, Robertson M M, Sandor P. 2000. An international perspective on Tourette syndrome: selected findings from 3500 individuals in 22 countries. Developmental Medicine & Child Neurology 42(7):436-447.
Kawasaki H, Springett G, Toki S, Canales J, Harlan P, Blumenstiel J. Chen E, Bany I, Mochizuki N, Ashbacher A, Matsuda M, Housman D, Graybiel A. 1998. A Rap guanine nucleotide exchange factor enriched highly in the basal ganglia. Proc Natl Acad Sci 95:13278-13283.
Kwak C, Vuong K, Jankovic J. 2003. Migraine headache in patients with tourette syndrome. Archives of Neurology 60(11):1595-1598.
Lawson-Yuen A, Saldivar J, Sommer S, Picker J. 2008. Familial deletion within NLGN4 associated with autism and Tourette syndrome. European Journal of Human Genetics 16:614-618.
Lei J. Deng X, Zhang J, Su L, Xu H, Liang H, Huang X, Song Z, Deng H. 2012. Mutation Screening of the HDC Gene in Chinese Han Patients With Tourette Syndrome. Am J Med Genet B Neuropsychiatr Genet 159B:72-76.
Lespérance P, Djerroud N, Diaz Anzaldua A, Rouleau G A, Chouinard S, Richer F. 2004. Restless legs in Tourette syndrome. Movement Disorders 19(9):1084-1087.
Norgren N. Mattson E, Forsgren L, Holmberg M. 2011. A high-penetrance form of late-onset torsion dystonia maps to a novel locus (DYT21) on chromosome 2q14.3-q21.3. Neurogenetics 12:137-143.
O'Roak B, Morgan T, Fishman D, Saus E, Alonso P, Gratacòs M, Estivill X, Teltsh O, Kohn Y, Kidd K, Cho J, Lifton R, State M. 2010. Additional support for the association of SLITRK1 var321 and Tourette syndrome. Molecular Psychiatry 15:447-450.
Pauls D, Leckman J. 1986. The inheritance of Gilles de la Tourette's syndrome and associated behaviors. Evidence for autosomal dominant transmission. N Engl J Med 315:993-997.
Pauls D, Raymond C, Stevenson J, Leckman J. 1991. A family study of Gilles de la Tourette syndrome. Am J Hum Genet 48:154-163.
Price R, Kidd K, Cohen D, Pauls D, Leckman J. 1985. A twin study of Tourette syndrome. Arch Gen Psychiatry 42:815-820.
Pringsheim T, Freeman R, Lang A. 2007 Tourette syndrome and dystonia. J Neurol Neurosurg Psychiatry 78:544.
Robertson M. 2008. The prevalence and epidemiology of Gilles de la Tourette syndrome. Part 1: The epidemiological and prevalence studies. J Psychosom Res 65(461-472).
Robertson M. 2012 The Giles de la Tourette Syndrome: the current status. Arch Dis Child Educ Pract Ed doi 10, Epub ahead of print:1136.
Robertson M, Eapen V, Cavanna A. 2009. The international prevalence, epidemiology, and clinical phenomenology of Tourette syndrome: A cross-cultural perspective. J Psychosom Res 67:475-483.
Rommelse N, Arias-Vasquez A, Altink M, Buschgens C, Fliers E, Asherson P. Faraone S, Buitelaar J, Sergeant J, Oosterlaan J, Franke B. 2008. Neuropsychological endophenotype approach to genome-wide linkage analysis identifies susceptibility loci for ADHD on 2q21.1 and 13q12.11. Am J Hum Genet 83:99-105.
Sato D, Lionel A, Leblond C, Prasad A, Pinto D, Walker S. O'Connor I, Russell C, Drmic I, Hamdan F, Michaud J, Endris V, Roeth R, Delorme R, Huguet G, Leboyer M. Rastam M, Gillberg C, Lathrop M, Stavropoulos D, Anagnostou E, Weksberg R, Fombonne E, Zwaigenbaum L, Fernandez B, Roberts W, Rappold G, Marshall C, Bourgeron T, Szatmari P, Scherer S. 2012, SHANK1 deletions in males with autism spectrum disorder Am J Hum Genet 90:879-887.
Scharf J, Moorjani P, Fagerness J, Platko J, Illmann C, Galloway B, Jenike E, Stewart S, Pauls D, Tourette Syndrome International Consortium for Genetics. 2008. Lack of association between SLITRK1var321 and Tourette syndrome in a large family-based sample. Neurology 70:1495-1496.
Singer H S. 2005. Tourette's syndrome: from behaviour to biology. The Lancet Neurology 4(4149-159.
State M. 2010. The genetics of child psychiatric disorders: Focus on autism and Tourette syndrome. Neuron 68:254-269.
State M. 2011 The genetics of Tourette disorder. Curr Opin Genet Dev 21:302-309.
Sundaram S, Hug A, Wilson B, Chugani H. 2010. Tourette syndrome is associated with recurrent exonic copy number variants. Neurology 74:1583-90.
Termine C, Balottin U, Rossi G, Maisano F, Salini S, Di Nardo R, Lanzi G. 2006. Psychopathology in children and adolescents with Tourette's syndrome: A controlled study. Brain and Development 28(469-75.
Toll-Riera M. Laurie S, Radò-Trilla N, Alba M. 2011. Partial Gene Duplication and the Formation of Novel Genes. In: Friedberg F, editor. Gene Duplication: Intech.
Uddin M, Sturge M, Peddle L, O'Rielly D, Rahman P. 2011. Genome-wide signatures of ‘rearrangement hotspots’ within segmental duplications in humans. PLoS One 6:e28853.
Verkerk A, Mathews C, Joosse M, Eussen B, Heutink P. Oostra B, Tourette Syndrome Association International Consortium for Genetics. 2003. CNTNAP2 is disrupted in a family with Gilles de la Tourette syndrome and obsessive compulsive disorder Genomics 82:1-9.
Walkup J, Leckman J, Price R, Hardin M, Ort S, Cohen D. 1988. The relationship between obsessive-compulsive disorder and Tourette's syndrome: A twin study Psychopharmacol Bull 24:375-379.

We claim:

1. A microarray chip system comprising at least: 500, 750, 1000 or 1500 distinct oligonucleotide probes bound to a solid support, wherein each oligonucleotide probe comprises a nucleotide sequence complementary to a rearrangement indicator sequence region of a human genome, the rearrangement indicator sequence regions comprising a rearrangement indicator set that is indicative of risk, or occurrence, of genomic rearrangements.

2. The system of claim 1, wherein each oligonucleotide probe hybridizes under medium or high stringency conditions to the corresponding complementary rearrangement indicator sequence region.

3. The system of claim 1, wherein the oligonucleotide probes are complementary to genomic sequences not more than 100, 150, 280, 300 or 500 base pairs apart within a single rearrangement indicator sequence region.

4. The system of claim 1, wherein the oligonucleotides probes are 30 to 100 base pairs in length, optionally 45 to 65 base pairs in length.

5. The system of claim 1, wherein the oligonucleotides are complementary to at least 5, 10, 15 or 30 contiguous nucleotides of the rearrangement indicator sequence regions.

6. The system of claim 1, wherein at least 5, 10 or 15 oligonucleotide probes comprise a nucleotide sequence complementary to a single rearrangement indicator sequence region.

7. The system of claim 1, wherein the rearrangement indicator sequence regions comprise at least one rearrangement hotspot and optionally, the rearrangement hotspots are within segmental duplications.

8. The system of claim 7, wherein the rearrangement hotspots are selected from the genomic regions listed in Table 1.

9. The system of claim 1, wherein the rearrangement indicator sequence regions are selected from the genomic regions listed in Tables 2-5.

10. The system of claim 1 wherein the solid support comprises at least one or two microarray chips and the oligonucleotide probes are arrayed on the at least one or two microarray chips.

11. The use of the microarray system of claim 1 for detecting a genomic rearrangement or the risk of a genomic rearrangement, wherein the genomic rearrangement is optionally a copy number variant (CNV).

12. The use of claim 11, wherein the genomic rearrangement indicates a genetic disease in a subject or the risk of a genetic disease in a subject.

13. The use of claim 12, wherein the genetic disease is Autism Spectrum Disorder, Psoriasis or Ankylosing Spondylitis.

14. The use of claim 12, wherein the genetic disease is Autism Spectrum Disorder and the genomic rearrangement is detected in the genes PGAP 1 or LNX1.

15. The use of claim 12, wherein the genetic disease is Ankylosing Spondylitis and the genomic rearrangement is detected in the genes UGT2B17 or UGT2B15.

16. A method of detecting genomic rearrangements in a subject comprising:
a. labeling a DNA test sample from a subject with a first fluorophore;
b. labeling a DNA reference sample with a second fluorophore;
c. contacting the labeled samples with the microarray system of claim 1 and hybridizing the labeled samples to the oligonucleotide probes of claim 1; and
d. identifying a genomic rearrangement, wherein a non-equal signal ratio between first fluorophore and the second fluorophore identifies a putative genomic rearrangement.

17. The method of claim 16, wherein the labeled samples are hybridized to the oligonucleotide probes of claim 1 under medium or high stringency hybridization conditions.

18. The method of claim 16, wherein a log 2 ratio of >0.25 or <−0.25 identifies a putative genomic rearrangement.

19. The method of claim 16, wherein the genomic rearrangement indicates a genetic disease or the risk of a genetic disease.

20. A method of constructing a microarray chip for detecting copy number variations comprising:
a. identifying at least 500, 1000, 1500 rearrangement indicator sequence regions;
b. designing oligonucleotide probes complementary to the rearrangement indicator sequence regions; and
c. arraying the oligonucleotide probes on at least one microarray chip.

21. A method of screening for, diagnosing and/or detecting an increased risk of developing Tourette Syndrome in a human subject comprising:
a) obtaining a sample from the subject;
b) assaying the sample for the presence of and detecting a Tourette Syndrome copy number variant in a Tourette Syndrome critical region thereby identifying the subject as having Tourette Syndrome or an increased risk of developing Tourette Syndrome, the assaying comprising hybridizing a probe and/or primer to the Tourette Syndrome copy number variant.

22. The method of claim 21, wherein the Tourette Syndrome critical region is on chromosome 2, optionally located at 2q21.1-21.2.

23. The method of claim 21, wherein the Tourette Syndrome copy number variant is a duplication or a deletion.

24. The method of claim 23, wherein the duplication is a duplication of genomic sequence corresponding to: chr2:132305299-132343808, chr2:132395155-132526804 or chr2:132305299-132343808 human genome assembly 19 or a portion thereof.

25. The method of claim 21, wherein detecting a Tourette Syndrome copy number variant with an increased copy number compared to a reference sequence identifies the subject as having Tourette Syndrome or an increased risk of developing Tourette Syndrome.

26. The method of claim 21, wherein the subject is presymptomatic, has one or more clinical symptoms or clinical features associated with Tourette Syndrome, has been diagnosed with Tourette syndrome and/or has at least one blood relation with Tourette Syndrome.

27. An isolated nucleic acid, wherein the nucleic acid hybridizes to:
a. a Tourette Syndrome copy number variant or a portion thereof;
b. a nucleic acid sequence complementary to a); and/or
c. a nucleic acid sequence corresponding to a).

28. The isolated nucleic acid of claim 27, wherein the Tourette Syndrome copy number variant comprises genomic sequence corresponding to chr2:132395155-132526804, chr2:132305299-132343808 or chr2:132480185-132510827 of human genome assembly 19 or a portion thereof.

29. The isolated nucleic acid of claim 27, wherein the isolated nucleic acid is a primer.

30. A kit for screening for, diagnosing or detecting an increased risk of developing Tourette Syndrome comprising:
a. a Tourette Syndrome copy number variant detection agent comprising an isolated nucleic acid of claim 27; and
b. instructions for use or a container for holding the detection agent of (a).
